(19) United States
(12) Patent Application Publication
(10) Pub. No.: US 2024/0310723 A1
(43) Pub. Date: Sep. 19, 2024

(54) ONIUM SALT, RESIST COMPOSITION AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 18/436,721

(22) Filed: Feb. 8, 2024

(30) Foreign Application Priority Data
Feb. 21, 2023 (JP) 2023-024819

(51) Int. Cl. G03F 7/004; G03F 7/038; G03F 7/039

(52) U.S. Cl. CPC G03F 7/0045; G03F 7/0048; G03F 7/0382; G03F 7/0388; G03F 7/0397

(57) ABSTRACT

An onium salt is provided. The chemically amplified resist composition can be processed by DUV or EUV lithography to form a resist pattern with improved resolution, reduced LWR, and collapse resistance. A resist composition comprising an onium salt having a nitrogen-containing aliphatic heterocycle and an aromatic carboxylic acid structure as a quencher is provided. When processed by deep-UV or EUV lithography, the resist composition exhibits a high resolution and reduced LWR and prevents the resist pattern from collapsing.

### CROSS-REFERENCE TO RELATED APPLICATIONS

This non-provisional application claims priority under 35 U.S.C. § 119(a) on Patent Application No. 2023-024819 filed in Japan on Feb. 21, 2023, the entire contents of which are hereby incorporated by reference.

### TECHNICAL FIELD

This invention relates to an onium salt, a resist composition comprising the same, and a patterning process using the composition.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using EUV of wavelength 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

A triangular tradeoff relationship among sensitivity, resolution, and edge roughness (LER, LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

Studies have also been made on quenchers or acid diffusion inhibitors. Amines are typically used as the quencher. Many problems associated with line width roughness (LWR) as an index of pattern roughness and pattern profile are left unsolved. Also the use of weak acid onium salts as the quencher is under study. For example, Patent Document 1 describes that patterns with minimal roughness can be formed using a compound capable of generating a carboxylic acid having a boiling point of at least 150° C. Patent Document 2 reports improvements in sensitivity, resolution and exposure margin by the addition of ammonium salts of sulfonic acids or carboxylic acids. Also, Patent Document 3 describes that a resist composition for KrF or EB lithography comprising a PAG capable of generating a fluorinated carboxylic acid is improved in resolution and process latitude such as exposure margin and depth of focus. Further, Patent Document 4 describes a positive photosensitive composition for ArF excimer laser lithography comprising a carboxylic acid onium salt.

Patent Document 5 describes an onium salt of fluoroalkanesulfonamide as the weak acid onium salt. When this onium salt is applied to the upcoming generation of ultrafine processing using ArF lithography or ArF immersion lithography, the LWR as an index of pattern roughness and resolution are yet insufficient. There is still a need for a weak acid onium salt having better quencher function. Also Patent Document 6 describes an onium salt of α,α-difluorocarboxylic acid as the carboxylic acid onium salt. On use of this onium salt, it can also act as an acid generator in some cases because the carboxylic acid resulting from proton exchange with strong acid has an acidity which is not fully low. Such low quencher function leads to unsatisfactory LWR and resolution. Also reported is an attempt to use an onium salt of aromatic carboxylic acid, which has not been positively applied in the ArF lithography, in the EUV lithography on which development efforts are recently concentrated.

Also, carboxylic acid onium salts containing a nitrogen-containing structure in the molecule are known. Patent Documents 7 to 9 describe carboxylic acid onium salts having an indole, indoline or piperidine carboxylic acid structure which is a nitrogen-containing heterocyclic compound. Patent Document 10 describes a carboxylic acid onium salt having an aminobenzoic acid structure. Patent Document 11 describes a carboxylic acid onium salt having an amide bond. Although these compounds act as the quencher, some problems remain. Since the aromatic amine and amide bond are not highly basic, the acid diffusion controlling ability is not sufficient. Since the piperidine carboxylic acid is highly water soluble, its industrial manufacture is difficult.

These series of weak acid onium salts are based on the mechanism that a salt exchange occurs between a weak acid onium salt and a strong acid (sulfonic acid) which is generated by another PAG upon exposure, to form a weak acid and a strong acid onium salt. That is, the strong acid (α,α-difluorosulfonic acid) having high acidity is replaced by a weak acid (alkanesulfonic acid or carboxylic acid), thereby suppressing acid-aided elimination reaction of acid labile group and reducing or controlling the distance of acid diffusion. The onium salt apparently functions as a quencher. However, as the microfabrication technology is currently further advanced, the resist compositions using such weak acid onium salts become unsatisfactory with respect to resolution, roughness, depth of focus (DOF) and the like, particularly when processed by the EUV lithography. The alkanesulfonic acid salts have a low quencher capability because their acidity is not fully low. The carboxylic acid salts are not only insufficient in the above-referred properties, but also suffer from a swell problem because they are highly hydrophilic and thus have a high affinity to alkaline developer so that the developer is sucked in the exposed area. Particularly in forming small-size line patterns, the swell causes the resist pattern to collapse down. To comply with the requirement for further miniaturization, it is desired to have a quencher which has a high sensitivity and excellent acid diffusion controlling ability, and prevents the resist patterns from collapsing as a result of swelling in alkaline developer.

### CITATION LIST

Patent Document 1: JP-A H11-125907
Patent Document 2: JP-A H11-327143
Patent Document 3: JP-A 2001-281849
Patent Document 4: JP 4226803
Patent Document 5: JP-A 2012-108447
Patent Document 6: JP-A 2015-054833 (U.S. Pat. No. 9,221,742)
Patent Document 7: JP 6217561
Patent Document 8: JP 6874738
Patent Document 9: JP 6512049
Patent Document 10: JP 6323302
Patent Document 11: WO 2019/087626

### SUMMARY OF THE INVENTION

An object of the invention is to provide a chemically amplified resist composition which is processed by DUV or EUV lithography to form a resist pattern with improved resolution, reduced LWR, and collapse resistance, an onium salt for use therein, and a pattern forming process using the resist composition.

The inventors have found that a resist composition comprising as the quencher an onium salt having a nitrogen-containing aliphatic heterocycle and an aromatic carboxylic acid structure can be processed by lithography to form a resist pattern with improved resolution and reduced LWR. Since the swell of the resist film during development is suppressed, which leads to collapse resistance, the resist composition is quite useful in high accuracy micropatterning.

In one aspect, the invention provides an onium salt having the formula (1).

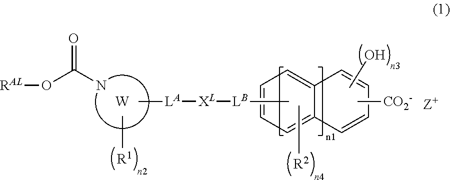

Herein n1 is 0 or 1, n2 is an integer of 0 to 6, n3 is an integer of 0 to 3, n4 is an integer of 0 to 4,
W is a C₂-C₂₀ nitrogen-containing aliphatic heterocycle which may contain a heteroatom,
L^{A} and L^{B} are each independently a single bond, ether bond, ester bond, amide bond, sulfonic ester bond, carbonate bond or carbamate bond,
X^{L} is a single bond or a C₁-C₄₀ hydrocarbylene group which may contain a heteroatom,
R¹ is a C₁-C₂₀ hydrocarbyl group which may contain a heteroatom, and when n2 is 2 or more, a plurality of R¹ may bond together to form a ring with the carbon atoms on W to which they are attached,
R² is halogen or a C₁-C₂₀ hydrocarbyl group which may contain a heteroatom, and when n4 is 2 or more, a plurality of R² may bond together to form a ring with the carbon atoms on the aromatic ring to which they are attached,
R^{AL} forms an acid labile group with the adjacent —O—, and
Z⁺ is an onium cation.

Preferably, R^{AL} is a group having the formula (AL-1) or (AL-2).

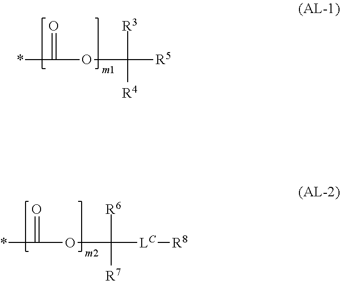

Herein L^{C} is —O— or —S—,
R³, R⁴ and R⁵ are each independently a C₁-C₁₀ hydrocarbyl group, any two of R³, R⁴ and R⁵ may bond together to form a ring,
R⁶ and R⁷ are each independently hydrogen or a C₁-C₁₀ hydrocarbyl group, R⁸ is a C₁-C₂₀ hydrocarbyl group in which —CH₂— may be replaced by —O— or —S—, R⁷ and R⁸ may bond together to form a C₃-C₂₀ heterocyclic group with the carbon atom and L^{C} to which they are attached, —CH₂— in the heterocyclic group may be replaced by —O— or —S—,
m1 and m2 are each independently 0 or 1, and
* designates a point of attachment to the adjacent —O—.

Also preferably, Z⁺ is an onium cation having any one of the formulae (cation-1) to (cation-3).

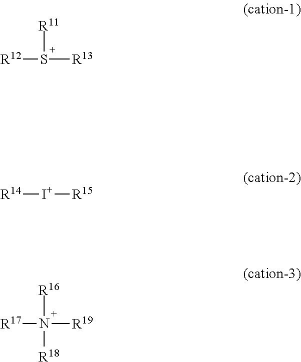

Herein R¹¹ to R¹⁹ are each independently a C₁-C₃₀ hydrocarbyl group which may contain a heteroatom, R¹¹ and R¹² may bond together to form a ring with the sulfur atom to which they are attached.

In a preferred embodiment, the onium salt has the formula (1A):

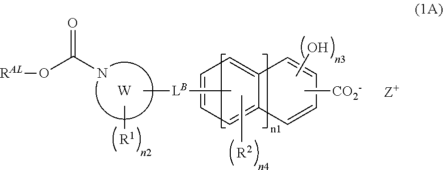

wherein n1 to n4, W, L^{B}, R¹, R², R^{AL} and Z⁺ are as defined above.

In a more preferred embodiment, the onium salt has the formula (1B):

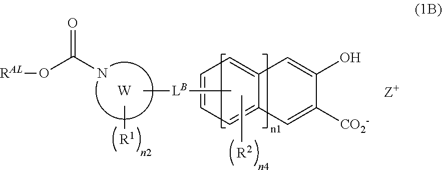

wherein n1, n2, n4, W, L^{B}, R¹, R², R^{AL} an Z⁺ are as defined above.

In another aspect, the invention provides a quencher comprising the onium salt defined herein.

In a further aspect, the invention provides a resist composition comprising the quencher defined herein.

The resist composition may further comprise an organic solvent.

The resist composition may further comprise a base polymer. The base polymer comprises repeat units having the formula (a1).

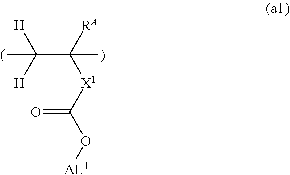

Herein R^{A} is hydrogen, fluorine, methyl or trifluoromethyl,

X¹ is a single bond, phenylene group, naphthylene group or *—C(═O)—O—X¹¹—, the phenylene group and naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, a phenylene group or naphthylene group, * designates a point of attachment to the carbon atom in the backbone, and
AL¹ is an acid labile group.

In a preferred embodiment, the base polymer further comprises repeat units having the formula (a2).

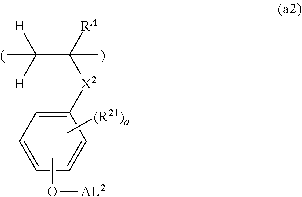

Herein R^{A} is hydrogen, fluorine, methyl or trifluoromethyl,
X² is a single bond or *—C(═O)—O—, wherein * designates a point of attachment to the carbon atom in the backbone,
R²¹ is halogen, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
AL² is an acid labile group, and
a is an integer of 0 to 4.

In a preferred embodiment, the base polymer further comprises repeat units having the formula (b1) or (b2).

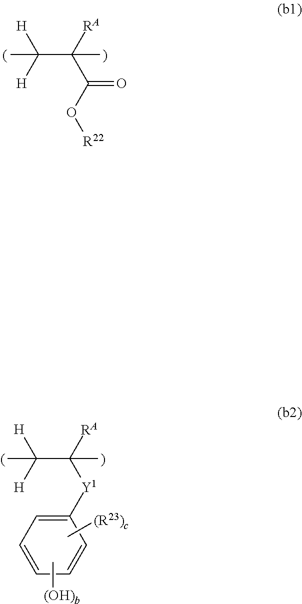

Herein R^{A} is each independently hydrogen, fluorine, methyl or trifluoromethyl,
Y¹ is a single bond or *—C(═O)—O— wherein * designates a point of attachment to the carbon atom in the backbone,
R²² is hydrogen, or a C₁-C₂₀ group containing at least one moiety selected from hydroxy moiety other than phenolic hydroxy, cyano moiety, carbonyl moiety, carboxy moiety, ether bond, ester bond, sulfonic ester bond, carbonate bond, lactone ring, sultone ring, and carboxylic anhydride (—C(═O)—O—C(═O)—),
R²³ is halogen, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
b is an integer of 1 to 4, c is an integer of 0 to 4, and b+c is from 1 to 5.

In a preferred embodiment, the base polymer further comprises repeat units of at least one type selected from repeat units having the formulae (c1) to (c4).

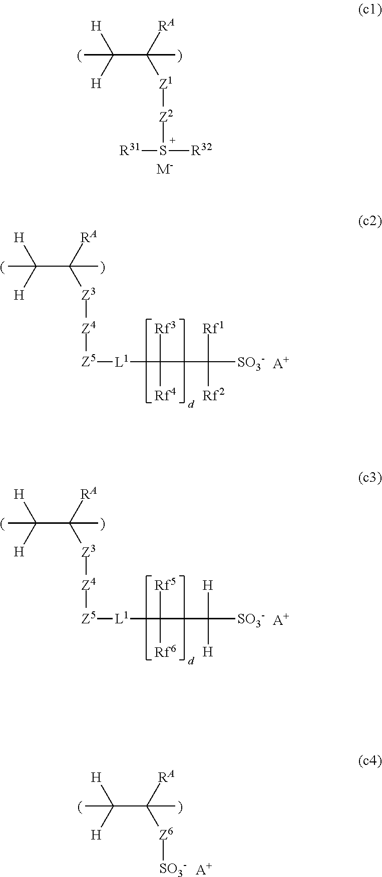

Herein R^{A} is each independently hydrogen, fluorine, methyl or trifluoromethyl,
Z¹ is a single bond or phenylene group,
Z² is *—C(═O)—O—Z²¹—, *—C(═O)—NH—Z²¹—, or *—O—Z²¹—, wherein Z²¹ is a C₁-C₆ aliphatic hydrocarbylene group, phenylene, or divalent group obtained by combining the foregoing, which may contain a carbonyl moiety, ester bond, ether bond or hydroxy moiety,
Z³ is each independently a single bond, phenylene group, naphthylene group or *—C(═O)—O—Z³¹—, wherein Z³¹ is a C₁-C₁₀ aliphatic hydrocarbylene group which may contain a hydroxy moiety, ether bond, ester bond or lactone ring, or a phenylene group or naphthylene group,
Z⁴ is each independently a single bond, *—Z⁴¹—C(═O)—O—, *—C(═O)—NH—Z⁴¹— or *—O—Z⁴¹—, wherein Z⁴¹ is a C₁-C₂₀ hydrocarbylene group which may contain a heteroatom,
Z⁵ is each independently a single bond, *—Z⁵¹—C(═O)—O—, *—C(═O)—NH—Z⁵¹— or *—O—Z⁵¹—, wherein Z⁵¹ is a C₁-C₂₀ hydrocarbylene group which may contain a heteroatom,
Z⁶ is a single bond, methylene, ethylene, phenylene, fluorinated phenylene, trifluoromethyl-substituted phenylene, *—C(═O)—O—Z⁶¹—, *—C(═O)—N(H)—Z⁶¹— or *—Z⁶¹—, wherein Z⁶¹ is a C₁-C₆ aliphatic hydrocarbylene group, phenylene, fluorinated phenylene or trifluoromethyl-substituted phenylene group, which may contain a carbonyl moiety, ester bond, ether bond or hydroxy moiety,
* designates a point of attachment to the carbon atom in the backbone,
R³¹ and R³² are each independently a C₁-C₂₀ hydrocarbyl group which may contain a heteroatom, R³¹ and R³² may bond together to form a ring with the sulfur atom to which they are attached,

L¹ is a single bond, ether bond, ester bond, carbonyl group, sulfonic ester bond, carbonate bond or carbamate bond,

Rf¹ and Rf² are each independently fluorine or a C₁-C₆ fluorinated saturated hydrocarbyl group,

Rf³ and Rf⁴ are each independently hydrogen, fluorine or a C₁-C₆ fluorinated saturated hydrocarbyl group,

Rf⁵ and Rf⁶ are each independently hydrogen, fluorine or a C₁-C₆ fluorinated saturated hydrocarbyl group, excluding that all Rf⁵ and Rf⁶ are hydrogen at the same time,
M⁻ is a non-nucleophilic counter ion,
A⁺ is an onium cation, and
d is an integer of 0 to 3.

The resist composition may further comprise a photoacid generator, a quencher other than the quencher defined herein, and/or a surfactant.

In a still further aspect, the invention provides a process for forming a pattern comprising the steps of applying the resist composition defined herein to a substrate to form a resist film thereon, exposing the resist film to high-energy radiation, and developing the exposed resist film in a developer.

In a preferred embodiment, the high-energy radiation is KrF excimer laser, ArF excimer laser, EB, or EUV of wavelength 3 to 15 nm.

### Advantageous Effects of Invention

The onium salt exerts a satisfactory quencher function in a resist composition. A resist film formed from the resist composition comprising the onium salt has a high sensitivity and improved dissolution contrast. As a result, the CDU of hole patterns and the LWR of line patterns are improved. A resist pattern of good profile with a high resolution and good rectangularity can be formed from the resist composition.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, Me stands for methyl, Ac for acetyl. Both the broken line (---) and the asterisk (*) designate a point of attachment or valence bond. As used herein, the term “fluorinated” refers to a fluorine-substituted or fluorine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
EL: exposure latitude
DOF: depth of focus
CDU: critical dimension uniformity

### Onium Salt

One embodiment of the invention is an onium salt having the formula (1).

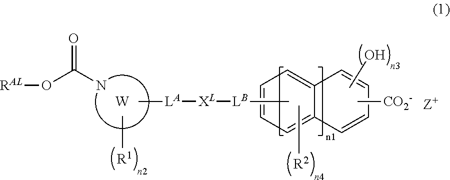

In formula (1), n1 is an integer of 0 or 1. The relevant structure represents a benzene ring in case of n1=0, and a naphthalene ring in case of n1=1. From the aspect of solvent solubility, a benzene ring corresponding to n1=0 is preferred. The subscript n2 is an integer of 0 to 6. The subscript n3 is an integer of 0 to 3, preferably 0 to 2, more preferably 0 or 1. When n3 is 1, 2 or 3, the aromatic hydroxy group(s) on the aromatic ring functions as an acid diffusion controlling group and when it is adjacent the carboxylate group on the aromatic ring, solvent solubility is improved by the resultant hydrogen bond. The subscript n4 is an integer of 0 to 4.

In formula (1), W is a C₂-C₂₀ nitrogen-containing aliphatic heterocycle which may contain a heteroatom. Exemplary structures of W are shown below, but not limited thereto. It is noted that in each structure, * designates a point of attachment to LA, and ** designates a point of attachment to R^{AL}—O—C(O)—.

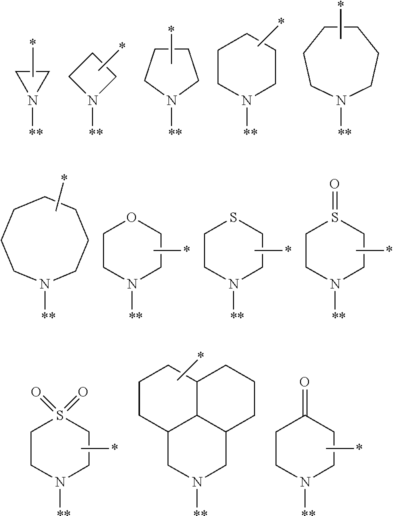

In formula (1), L^{A} and L^{B} are each independently a single bond, ether bond, ester bond, amide bond, sulfonic ester bond, carbonate bond or carbamate bond. Of these, a single bond, ether bond, ester bond and amide bond are preferred, with a single bond, ester bond and amide bond being more preferred.

In formula (1), X^{L} is a single bond or a C₁-C₄₀ hydrocarbylene group which may contain a heteroatom. The hydrocarbylene group may be straight, branched or cyclic and examples thereof include alkanediyl groups and cyclic saturated hydrocarbylene groups. Suitable heteroatoms include oxygen, nitrogen, and sulfur.

Examples of the optionally heteroatom-containing C₁-C₄₀ hydrocarbylene group X^{L} are shown below, but not limited thereto. Herein * each designates a point of attachment to L^{A} or L^{B}.

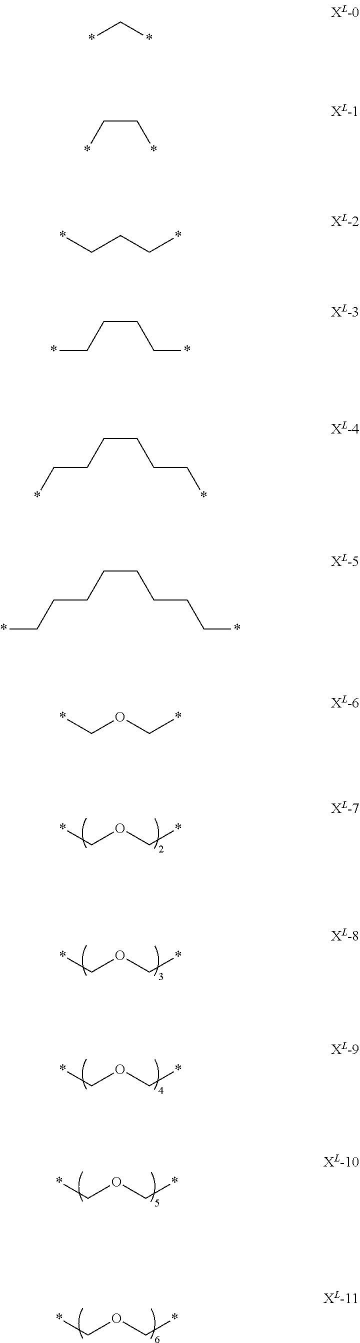

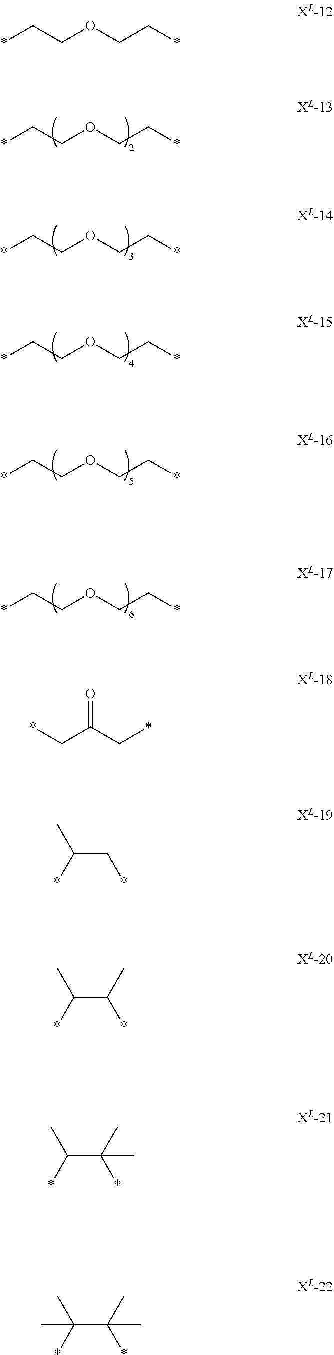

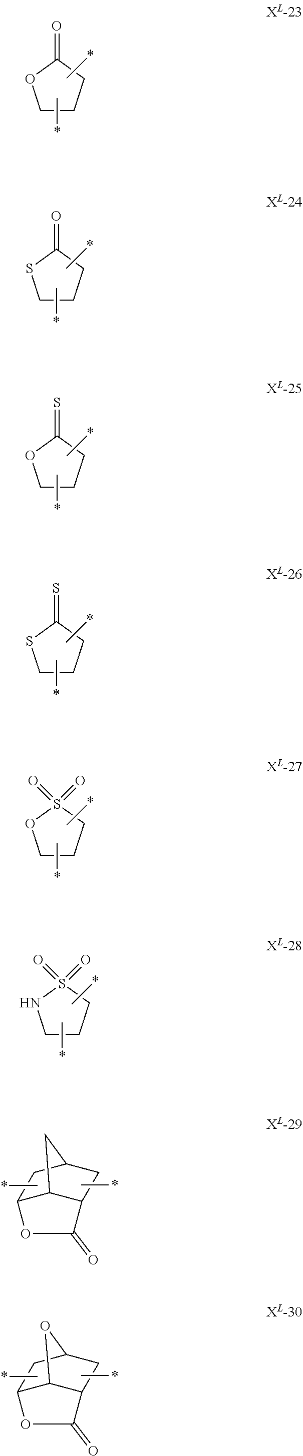

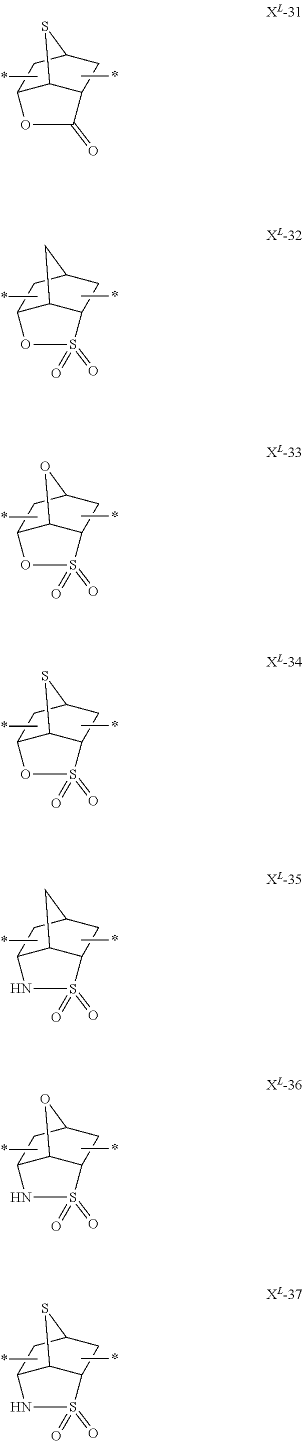

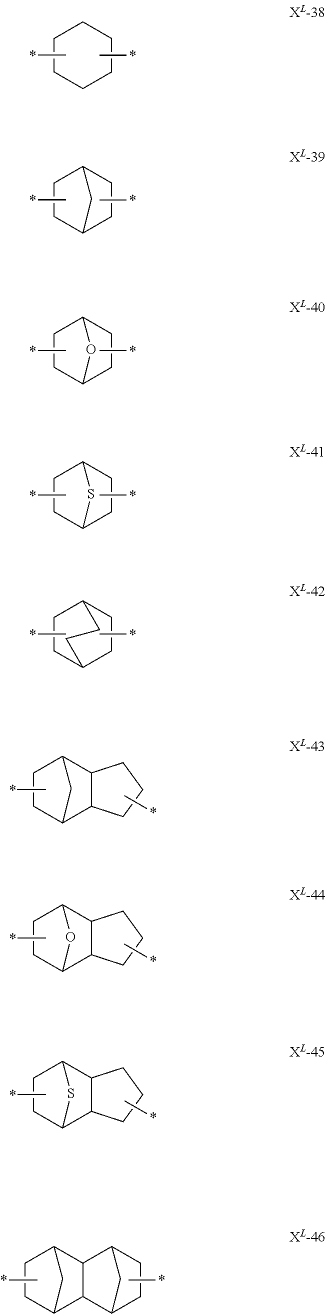

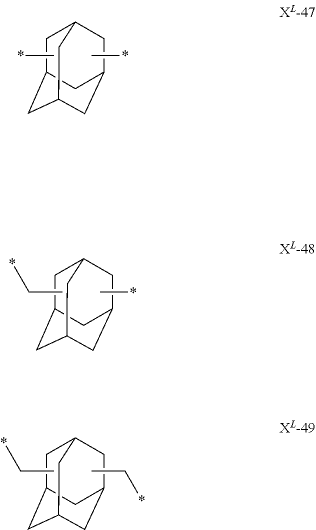

Of these, X^{L}-0 to X^{L}-22 and X^{L}-47 to X^{L}-49 are preferred, with X^{L}-0 to X^{L} 17 being more preferred.

In formula (1), R¹ is a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₂₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, adamantyl, and adamantylmethyl; C₆-C₂₀ aryl groups such as phenyl, naphthyl and anthryl, and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonic ester bond, carbonate bond, carbamate bond, amide bond, imide bond, lactone ring, sultone ring, thiolactone ring, lactam ring, sultam ring, carboxylic anhydride (—C(═O)—O—C(═O)—) or haloalkyl moiety.

When n2 is 2 or more (i.e., n2≥2), a plurality of R¹ may bond together to form a ring with the carbon atoms on W to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety. Also, two groups R¹ may bond with a common atom on W and bond together to form a ring, i.e., spiro ring.

In formula (1), R² is halogen or a C₁-C₂₀ hydrocarbyl group which may contain a heteroatom. Suitable halogen atoms include fluorine, chlorine, bromine, and iodine. The hydrocarbyl group may be saturated or unsaturated and straight, branched or cyclic. Examples thereof are as exemplified above for the hydrocarbyl group R¹. When n4 is 2 or more (i.e., n4≥2), a plurality of R² may bond together to form a ring with the carbon atoms on the aromatic ring to which they are attached. Examples of the ring are as exemplified above for the ring that a plurality of R¹ bond together and form with carbon atoms on W.

In formula (1), R^{AL} forms an acid labile group with the adjacent —O—. Preferably, R^{AL} is a group having the formula (AL-1) or (AL-2).

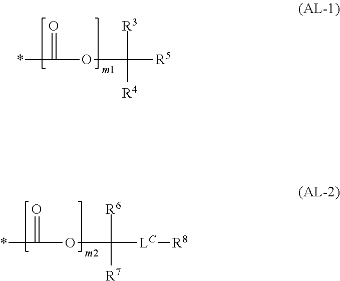

Herein * designates a point of attachment to the adjacent oxygen atom.

In formula (AL-1), R³, R⁴ and R⁵ are each independently a C₁-C₁₀ hydrocarbyl group. Any two of R³, R⁴ and R⁵ may bond together to form a ring. The subscript m1 is 0 or 1.

In formula (AL-2), L^{C} is —O— or —S—. R⁶ and R⁷ are each independently hydrogen or a C₁-C₁₀ hydrocarbyl group. R⁸ is a C₁-C₂₀ hydrocarbyl group in which —CH₂— may be replaced by —O— or —S—. R⁷ and R⁸ may bond together to form a C₃-C₂₀ heterocyclic group with the carbon atom and L^{C} to which they are attached. Some constituent —CH₂— in the heterocyclic group may be replaced by —O— or —S—. The subscript m2 is 0 or 1.

Examples of the acid labile group having formula (AL-1) are shown below, but not limited thereto. Herein * designates a point of attachment to the adjacent —O—.

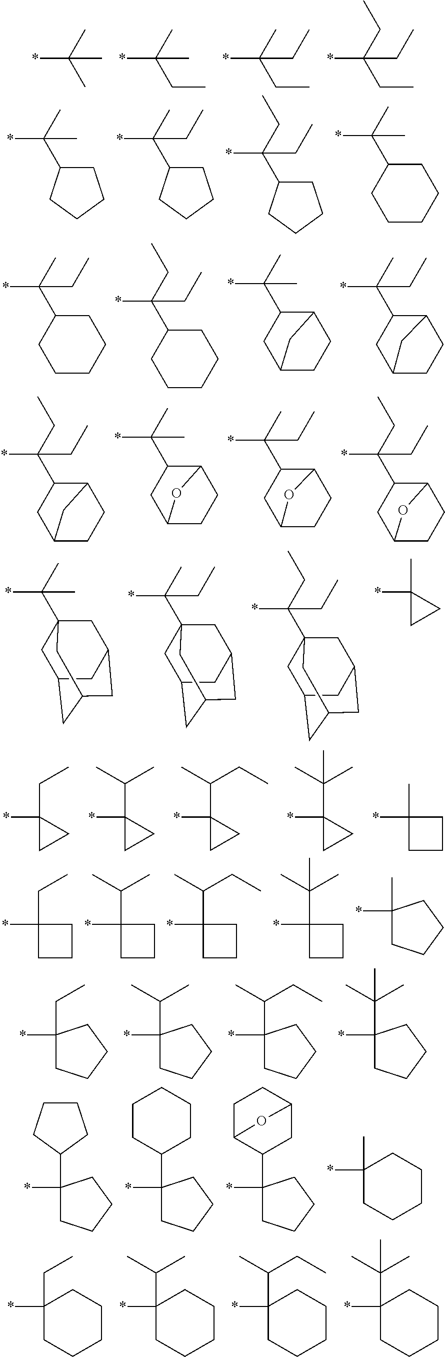

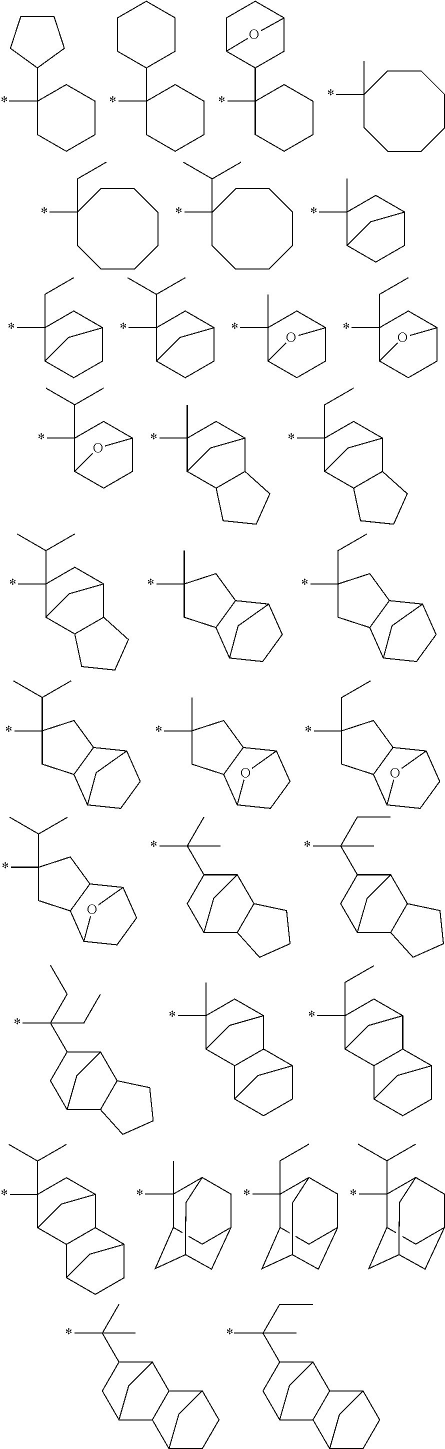

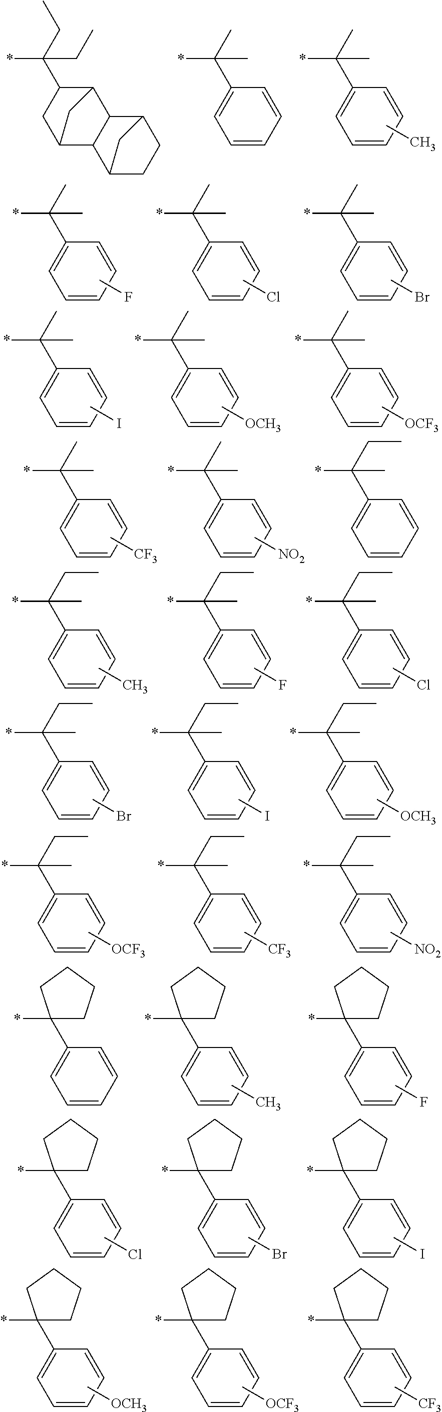

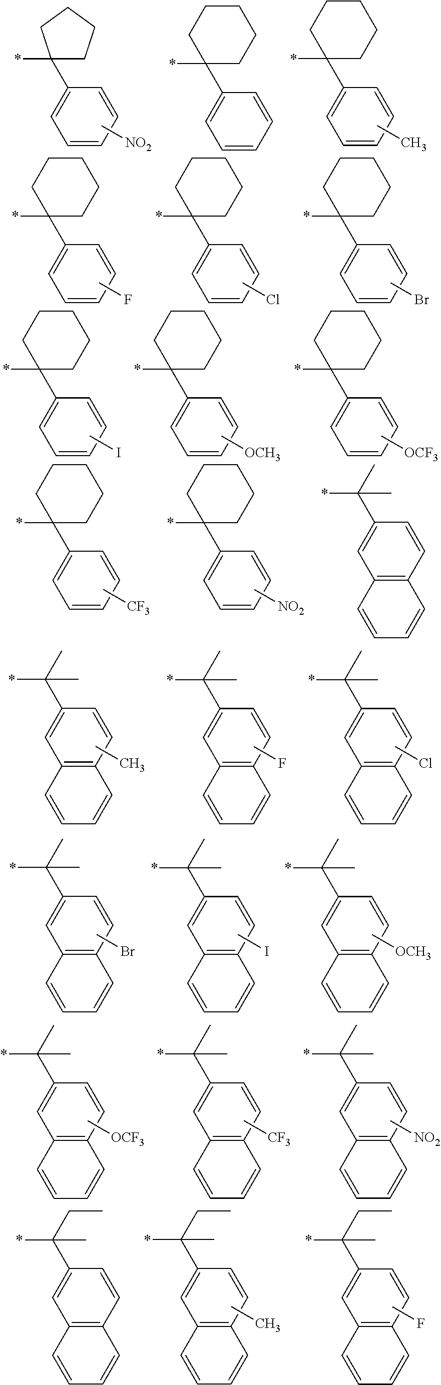

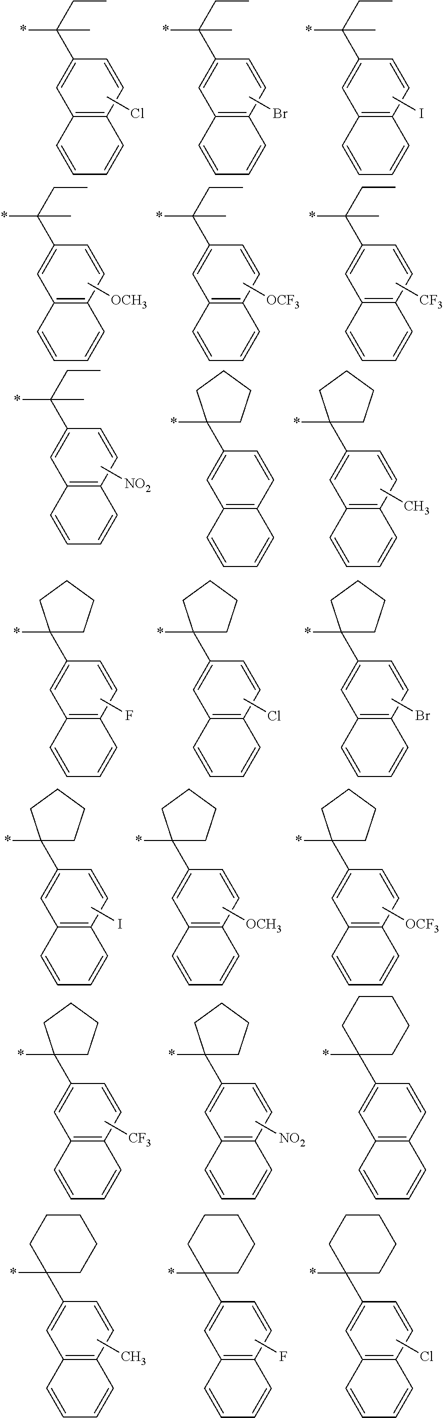

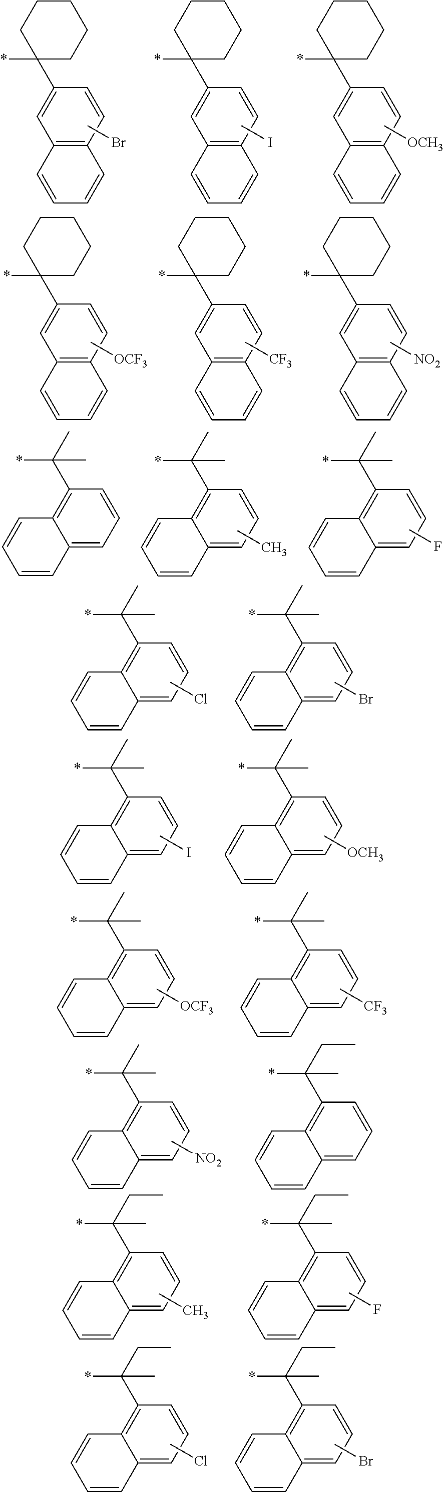

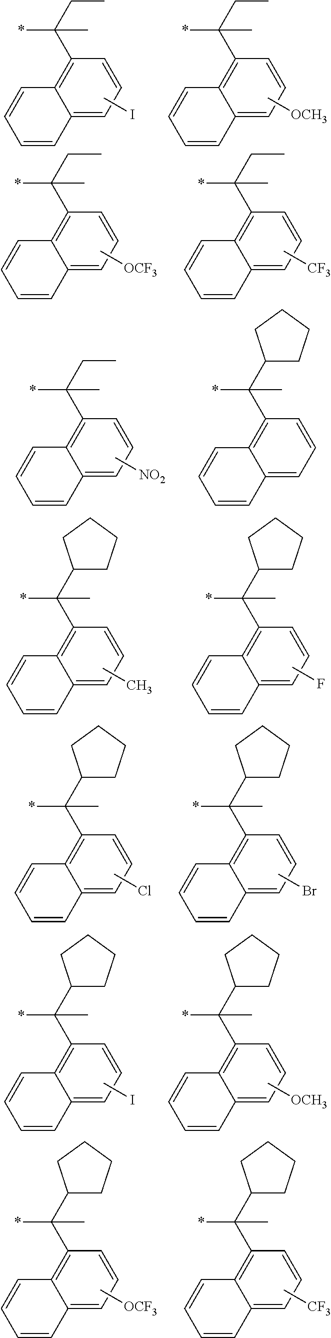

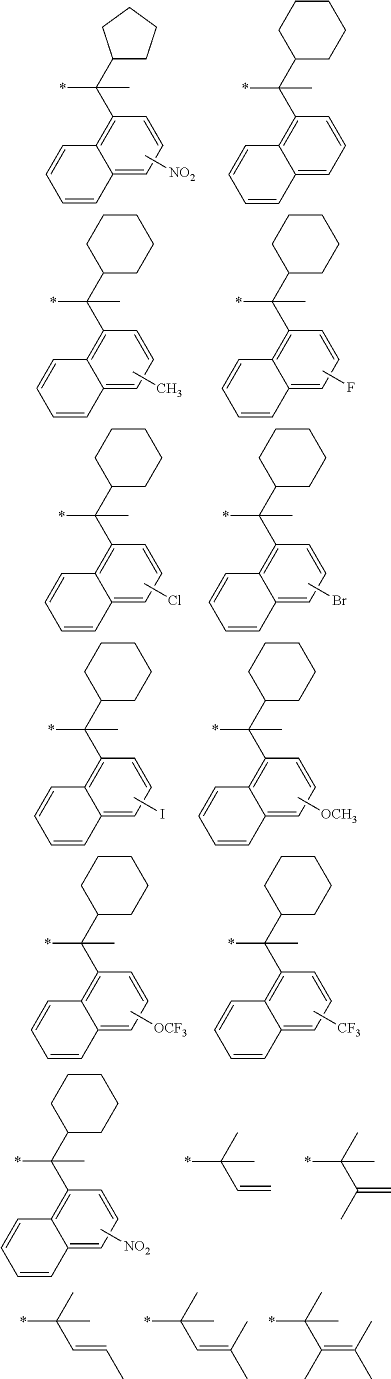

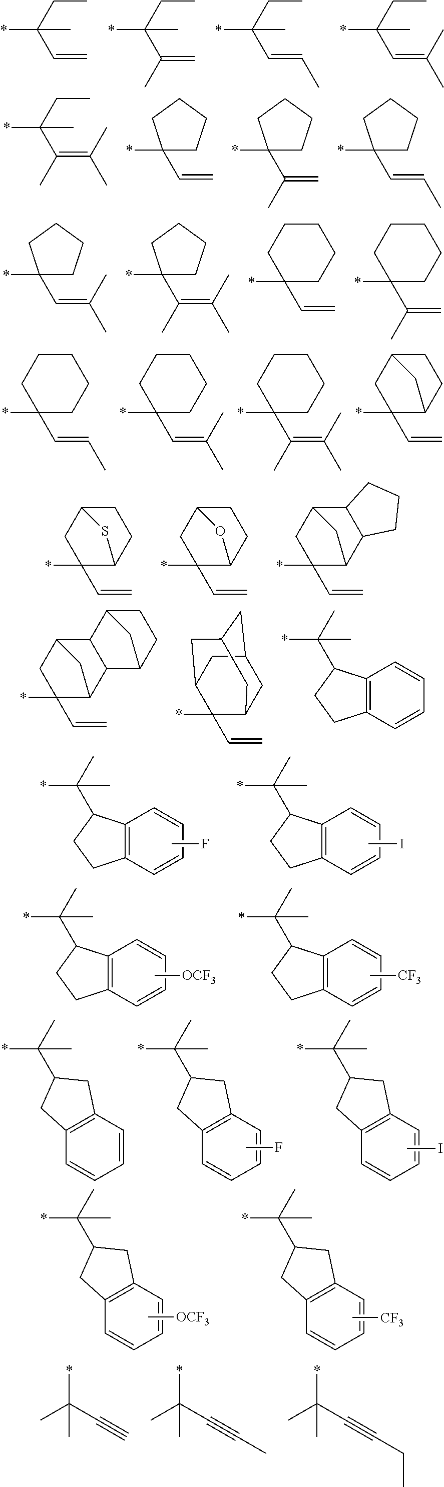

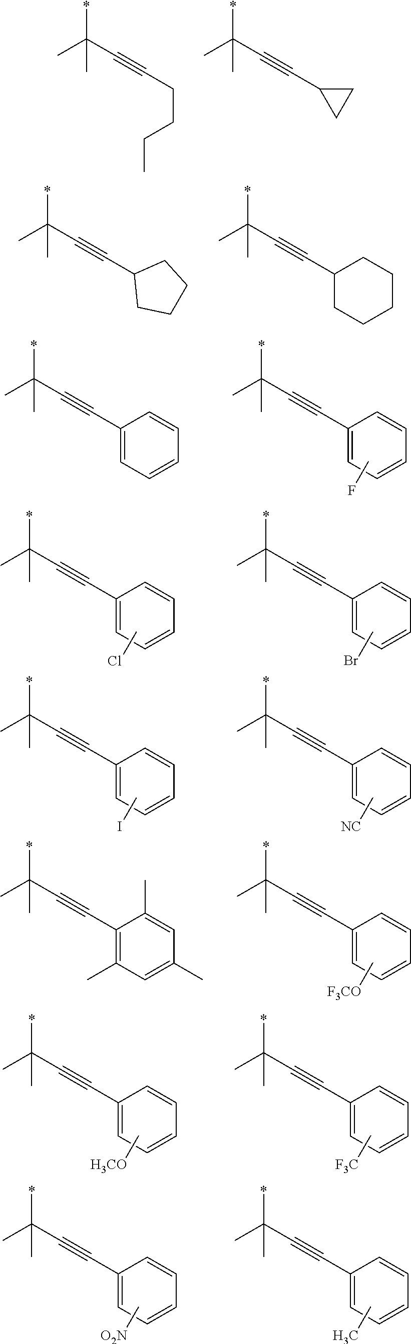

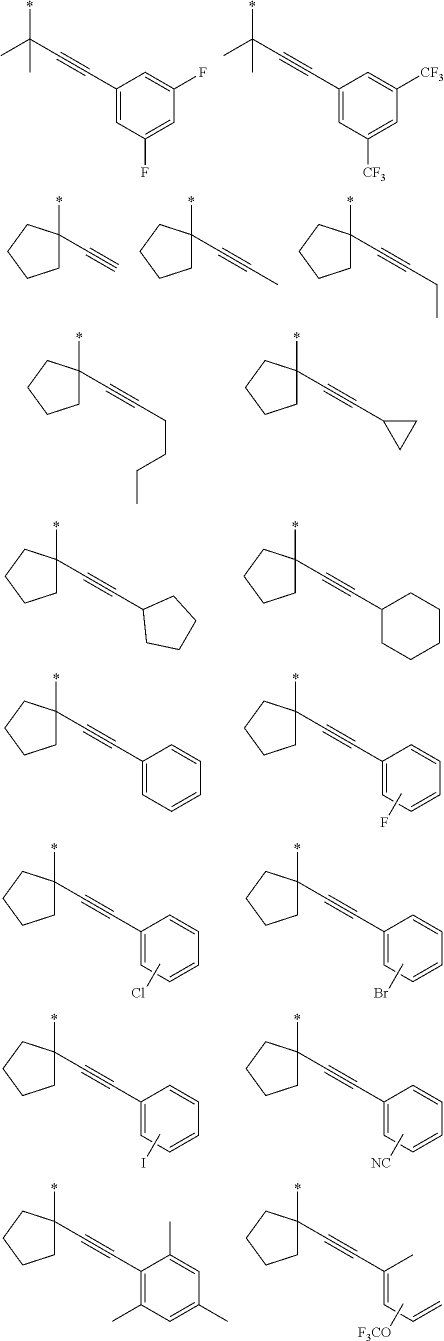

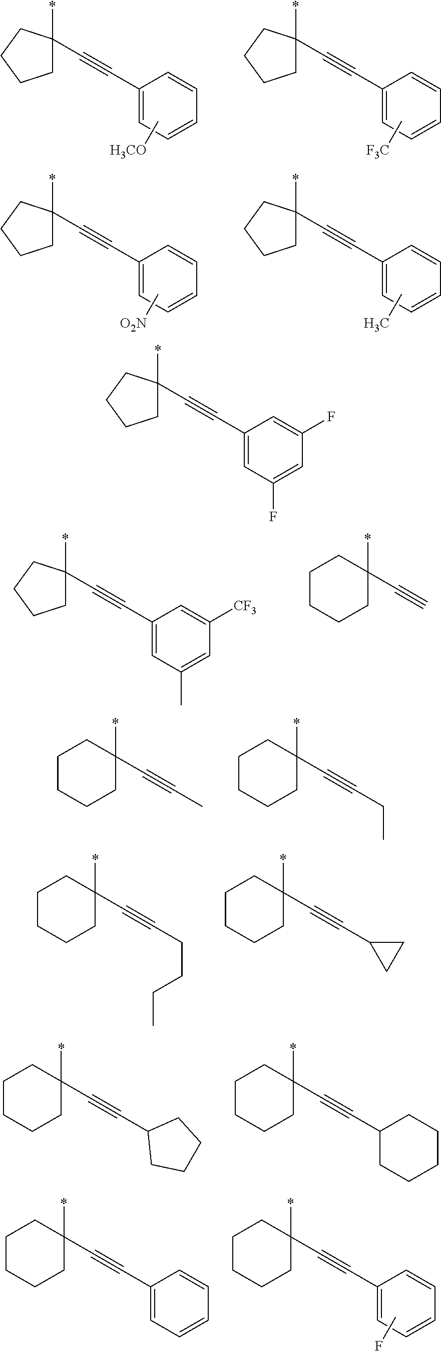

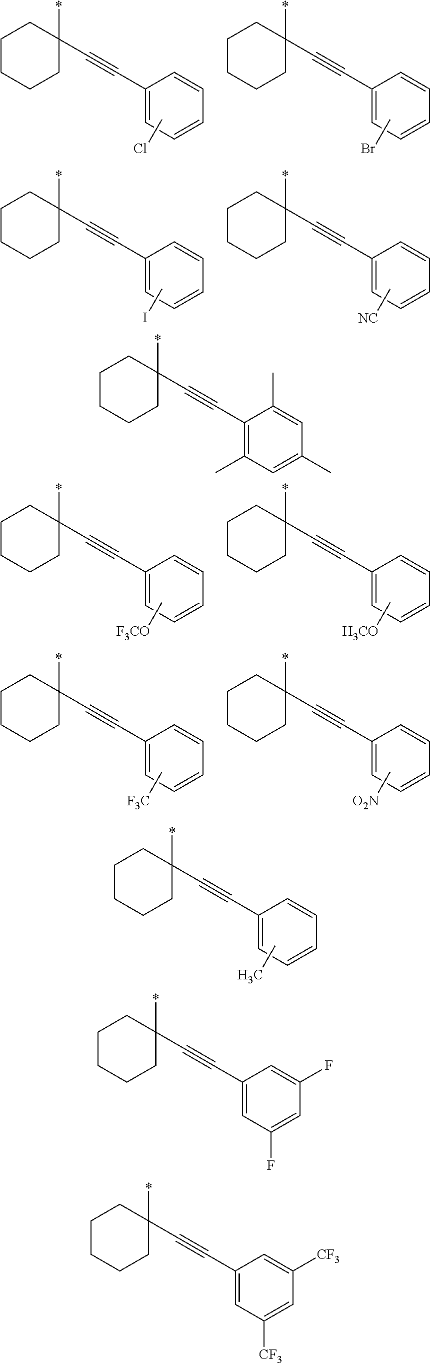

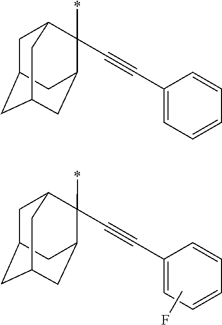

Examples of the acid labile group having formula (AL-2) are shown below, but not limited thereto. Herein * designates a point of attachment to the adjacent —O—.

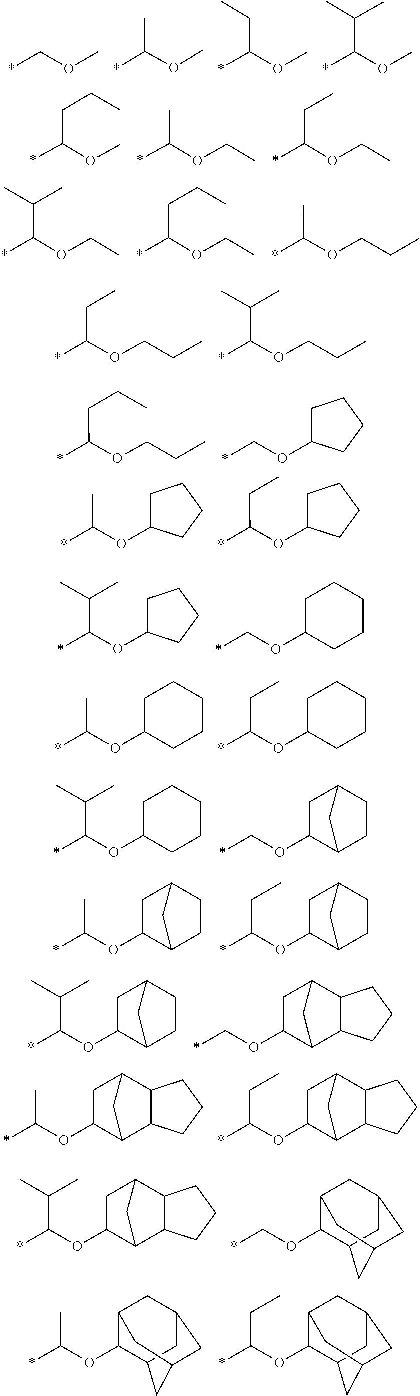

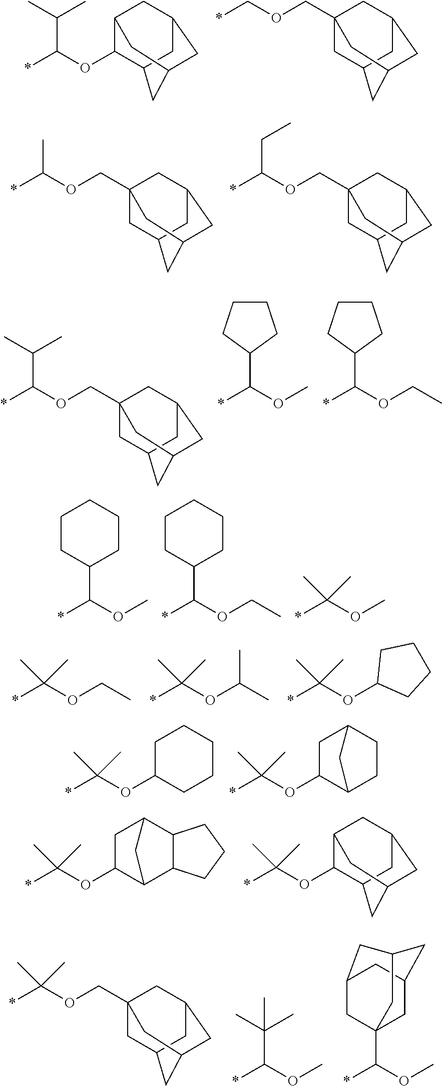

Of the oniumn salts having formula (1), those having the formula (1A) are preferred.

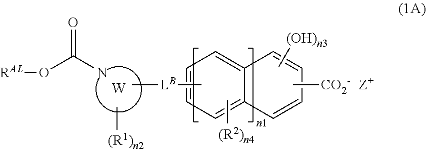

Herein n1 to n4, W, L^{B}, R¹, R², R^{AL} and Z⁺ are as defined above.

Of the onium salts having formula (1A), those having the formula (1B) are more preferred.

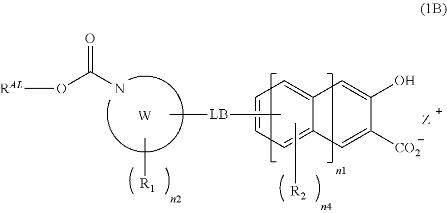

Herein n1, n2, n4, W, L^{B}, R¹, R², R^{AL} and Z⁺ are as defined above.

Examples of the anion in the onium salt having formula (1) are shown below, but not limited thereto.

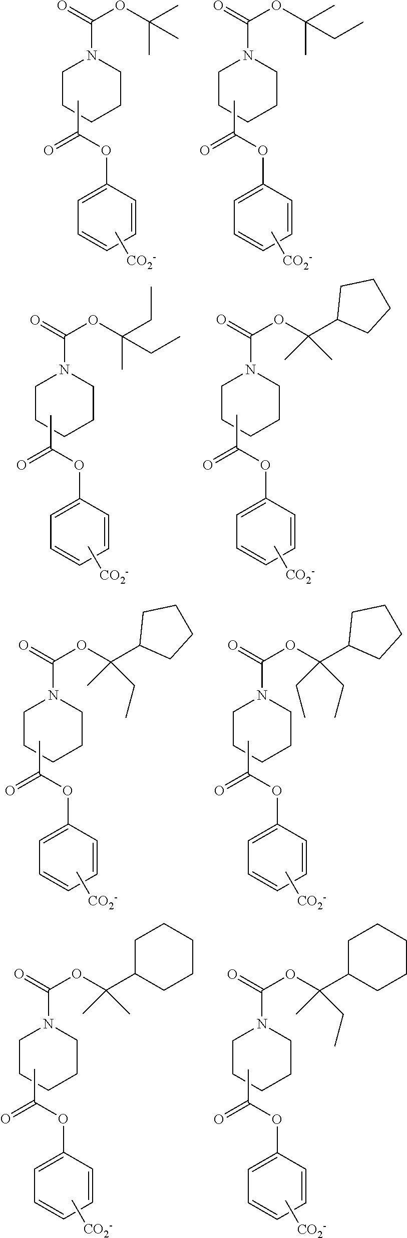

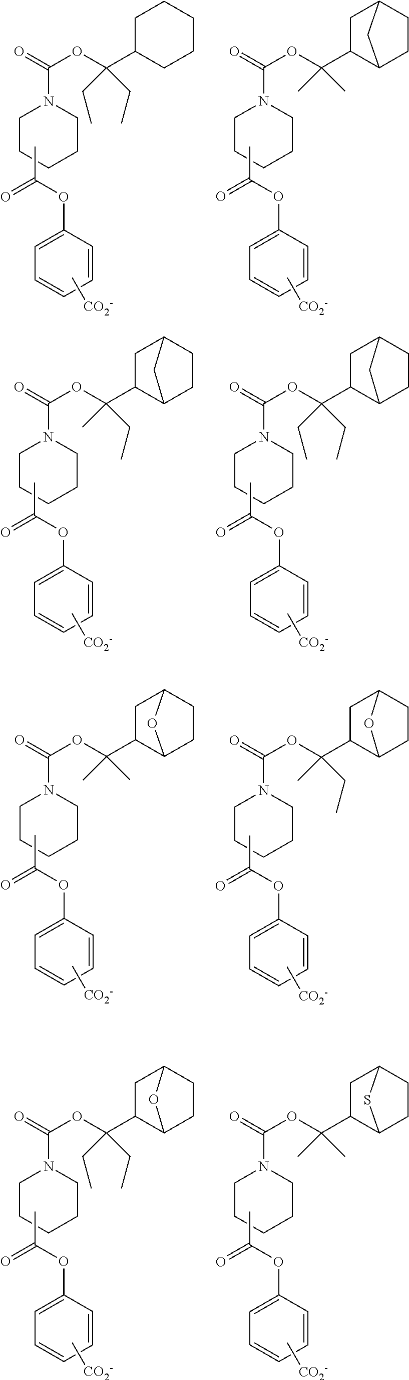

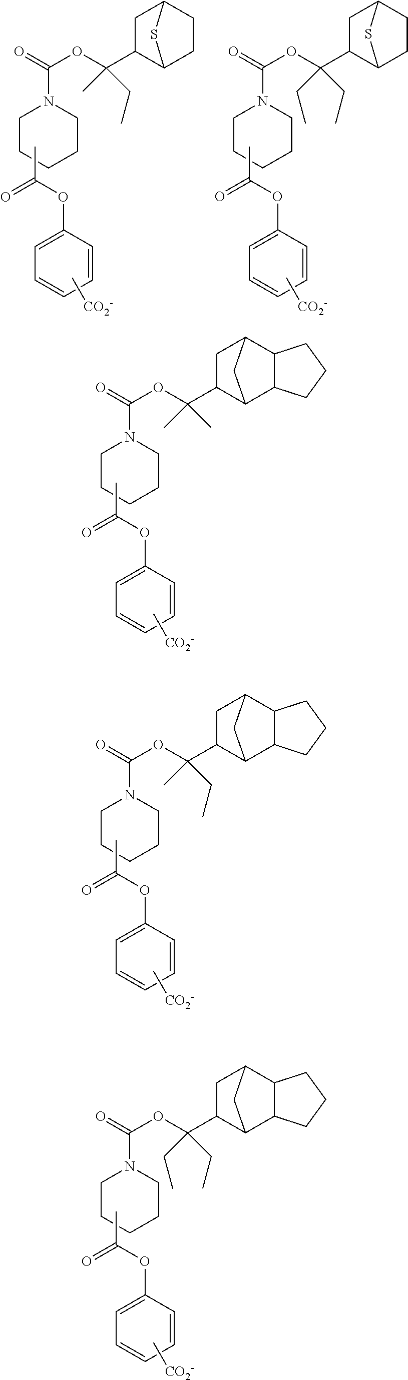

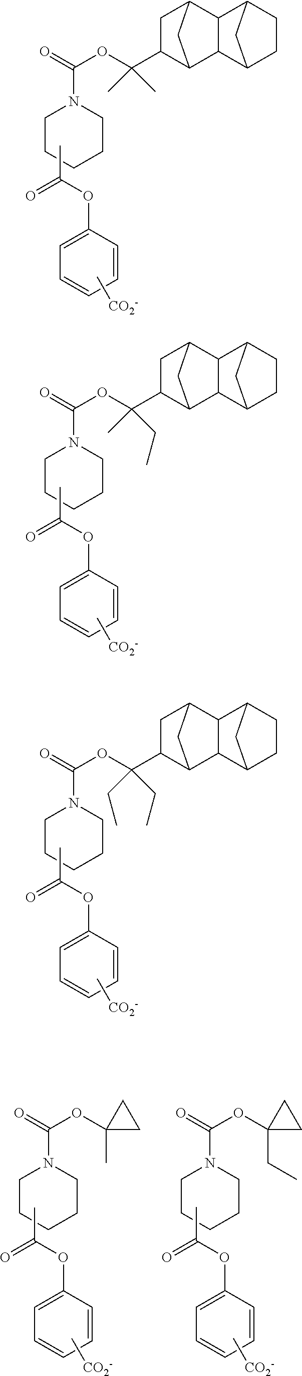

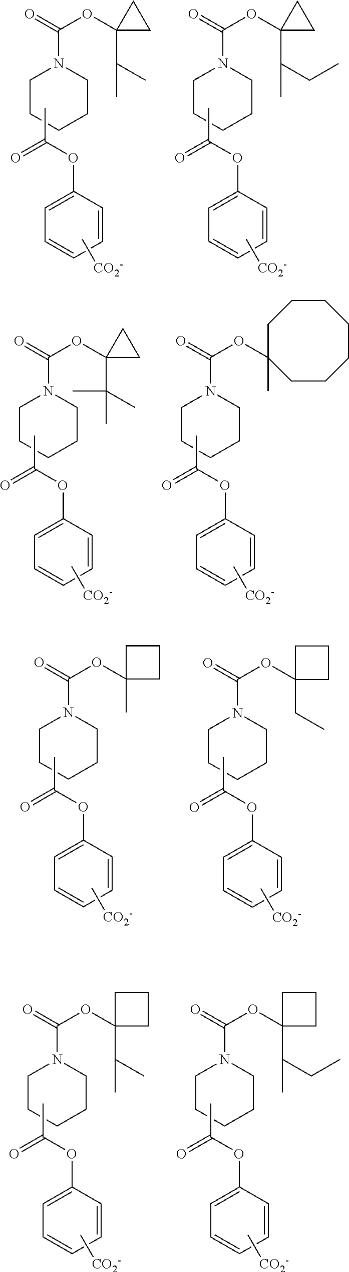

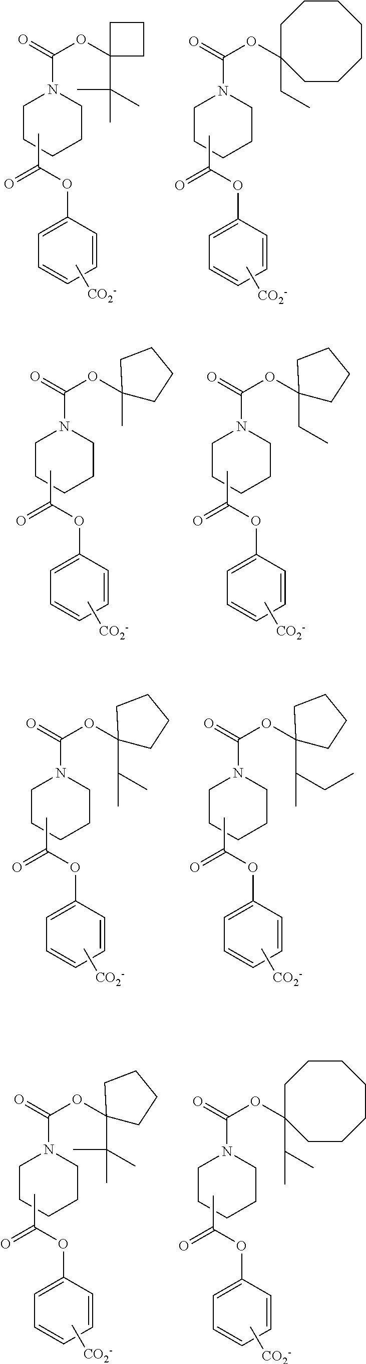

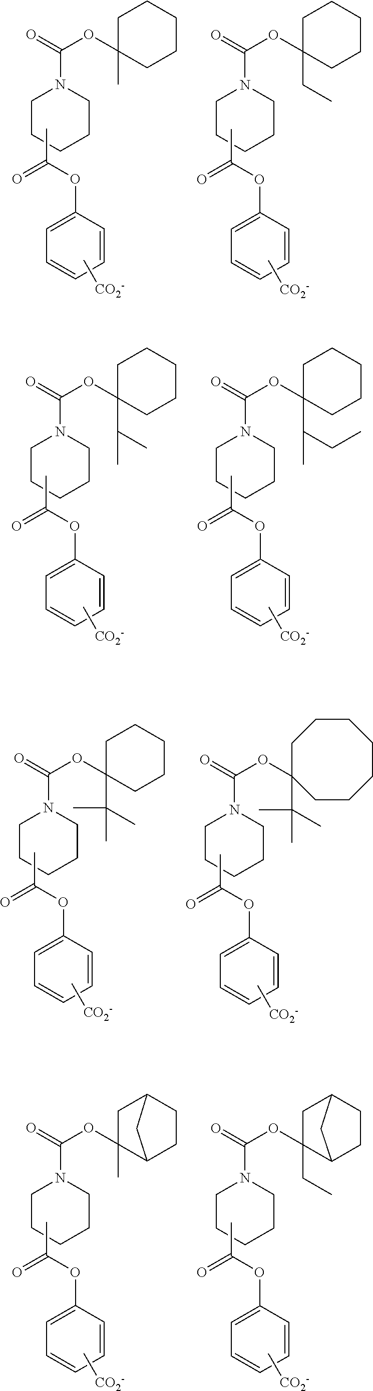

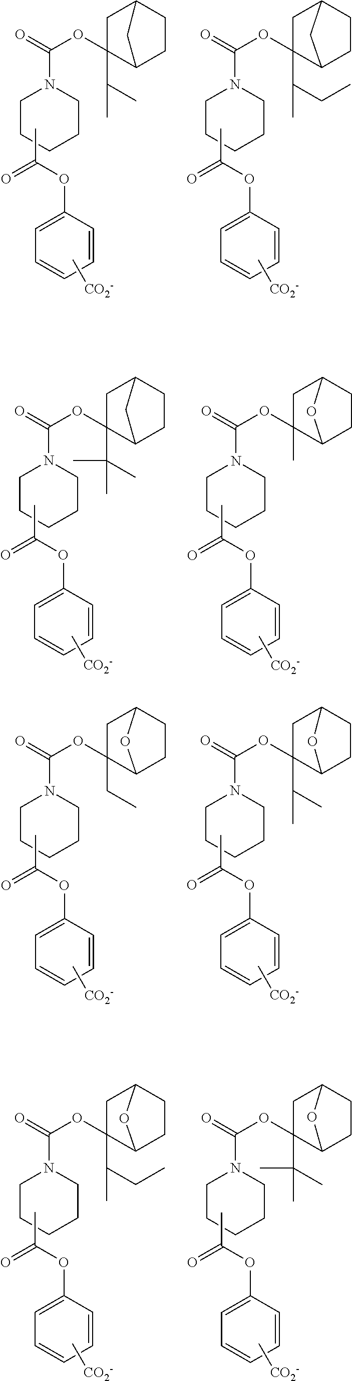

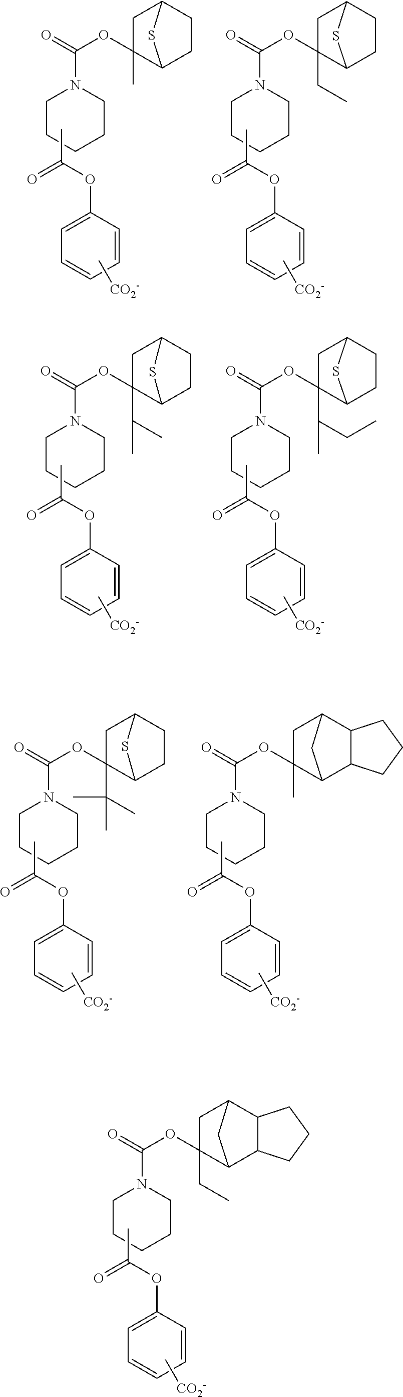

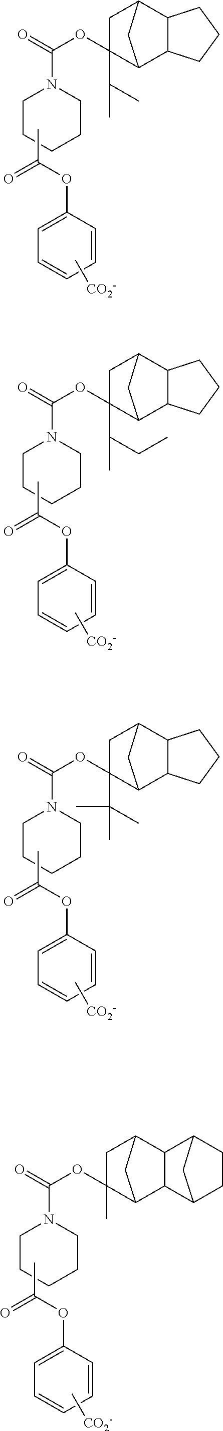

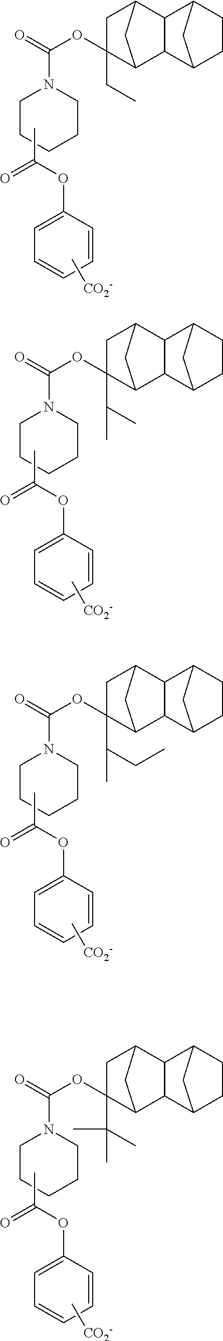

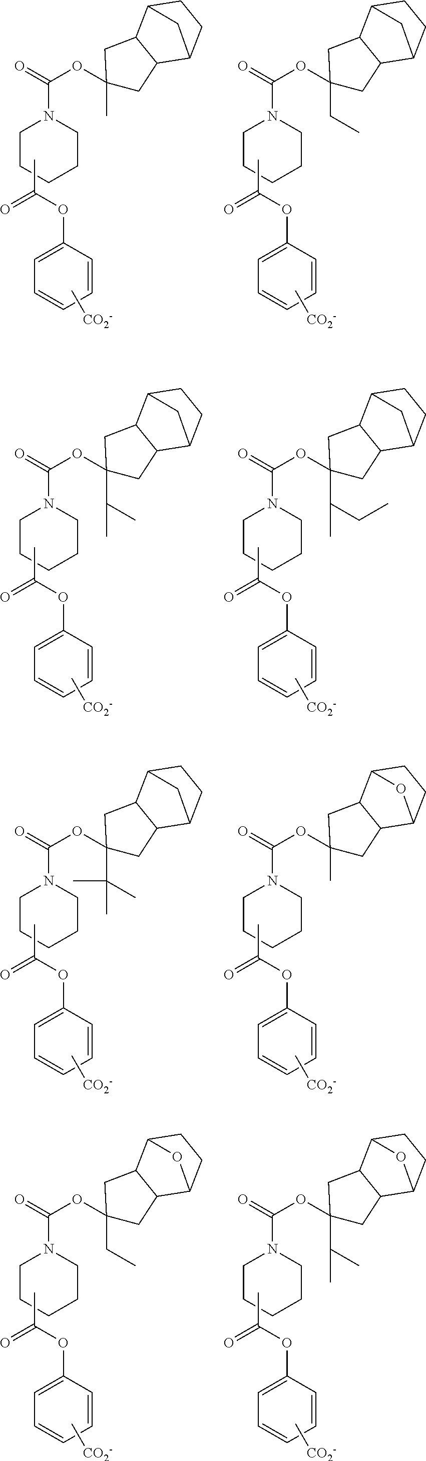

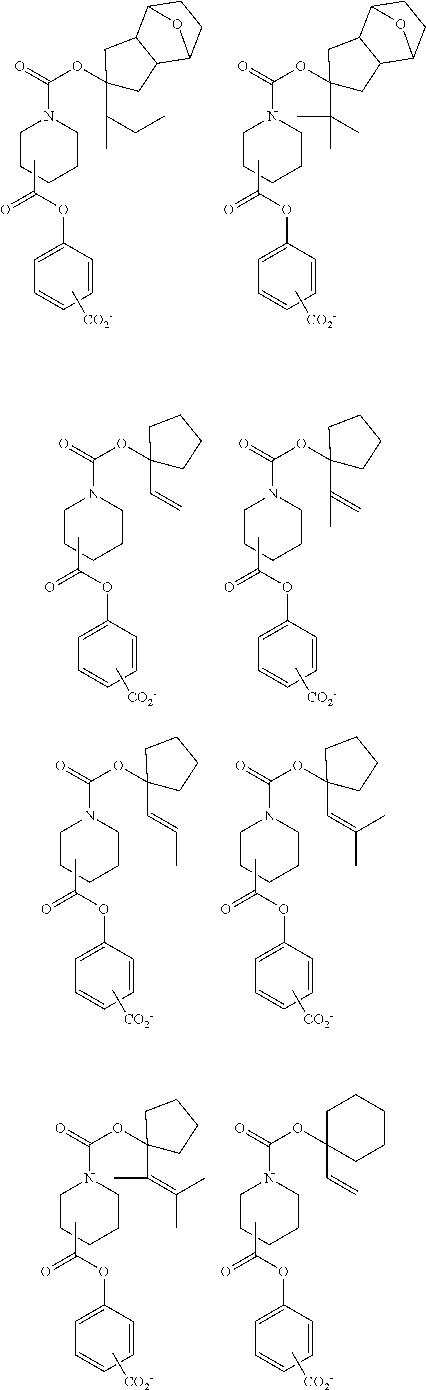

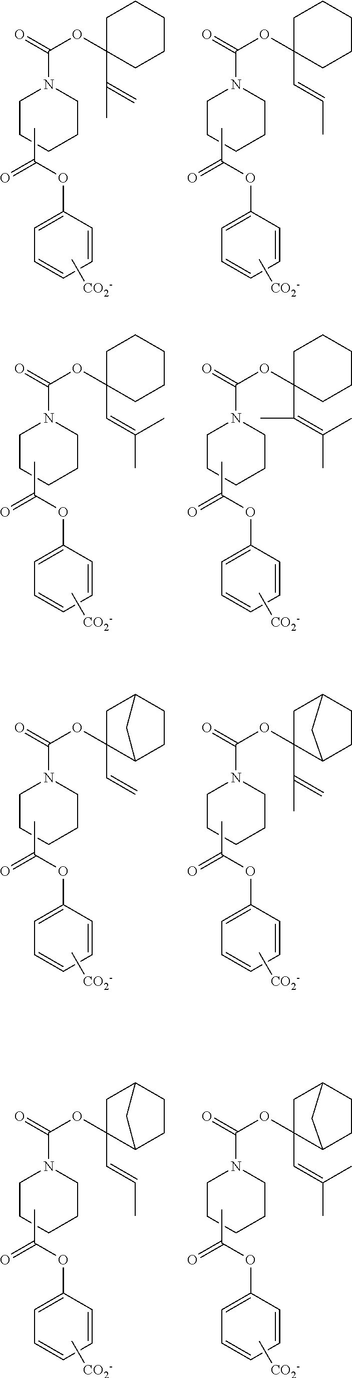

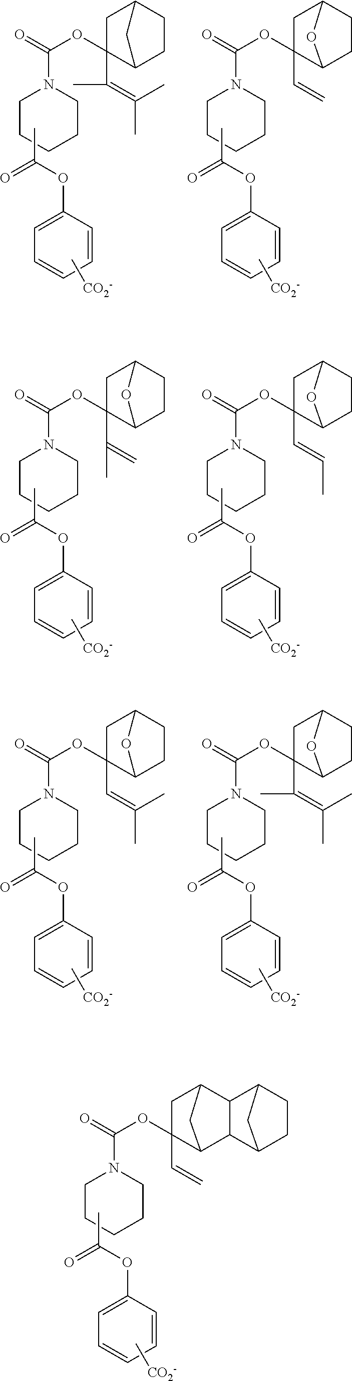

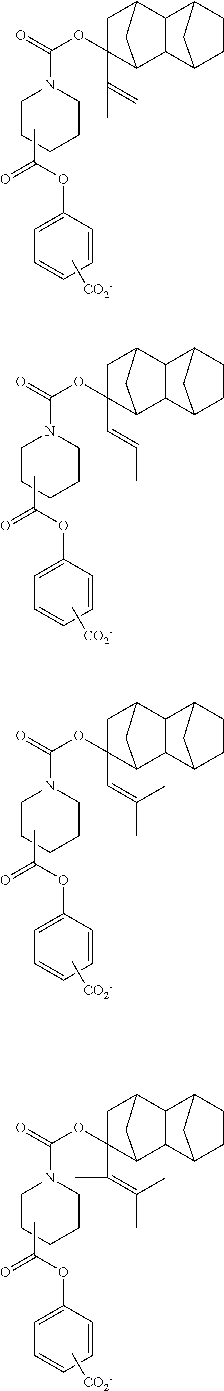

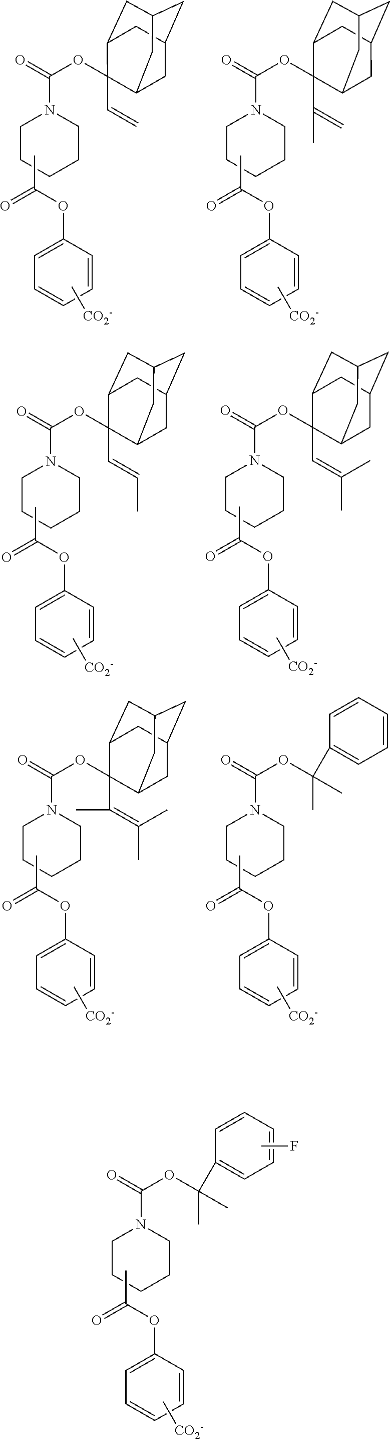

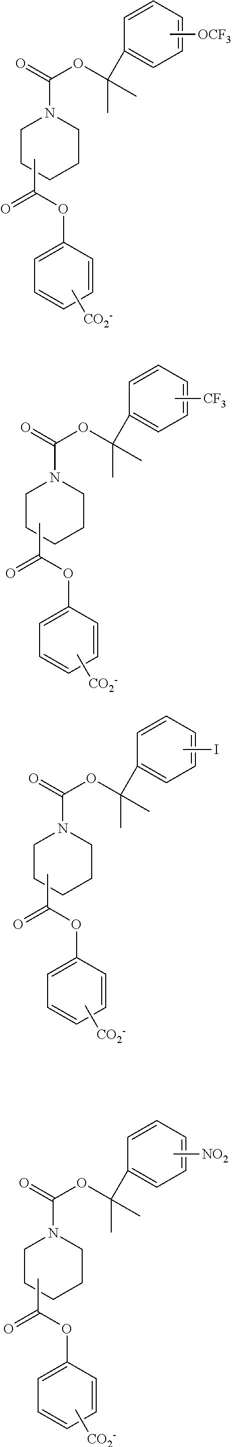

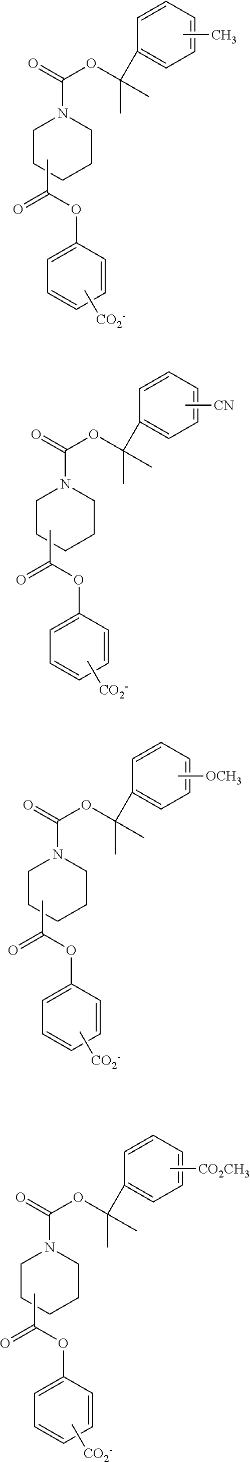

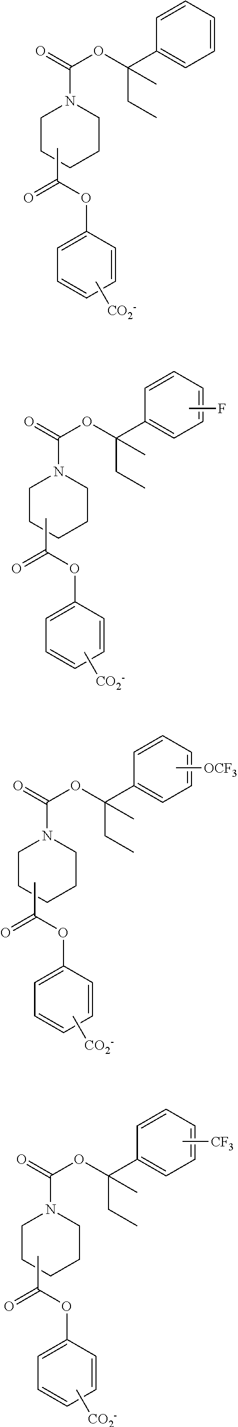

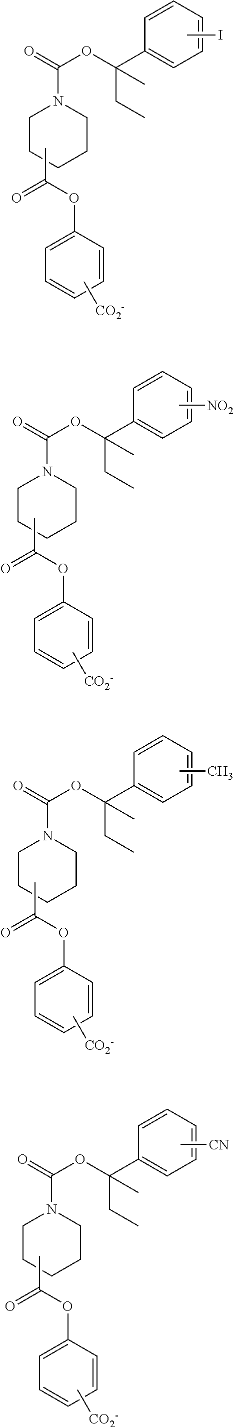

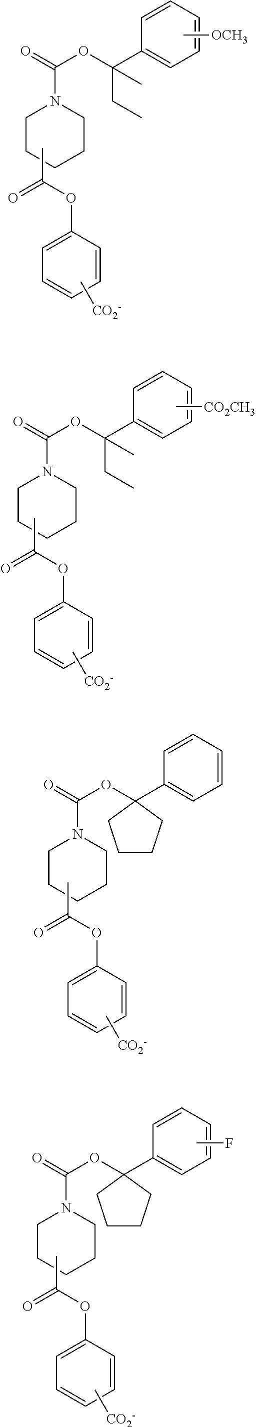

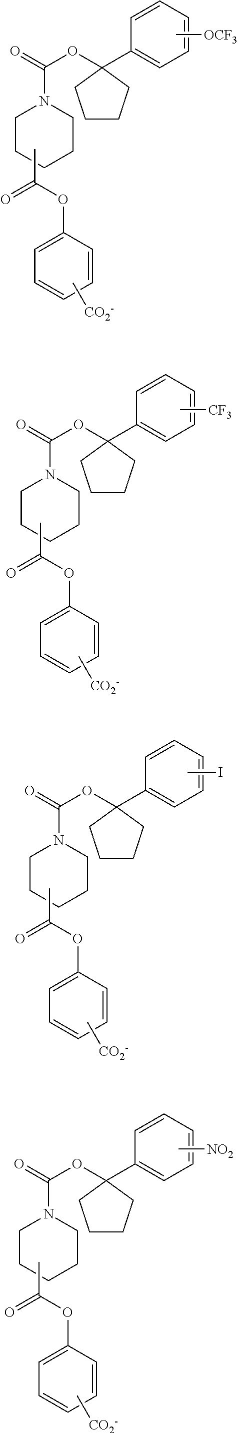

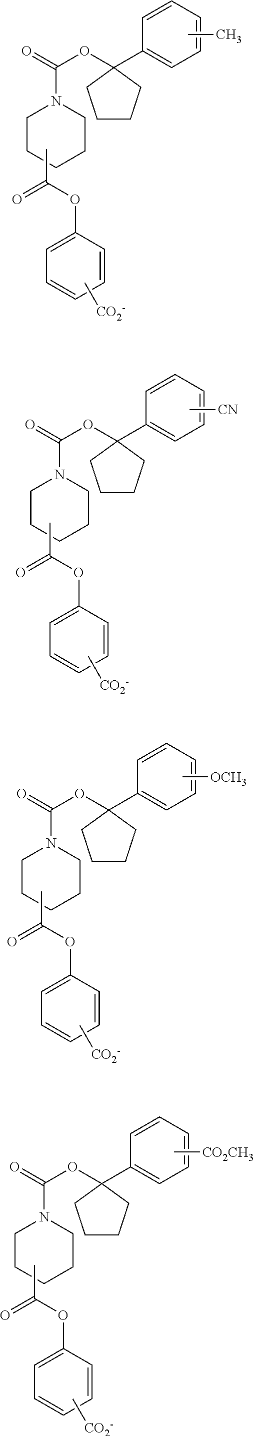

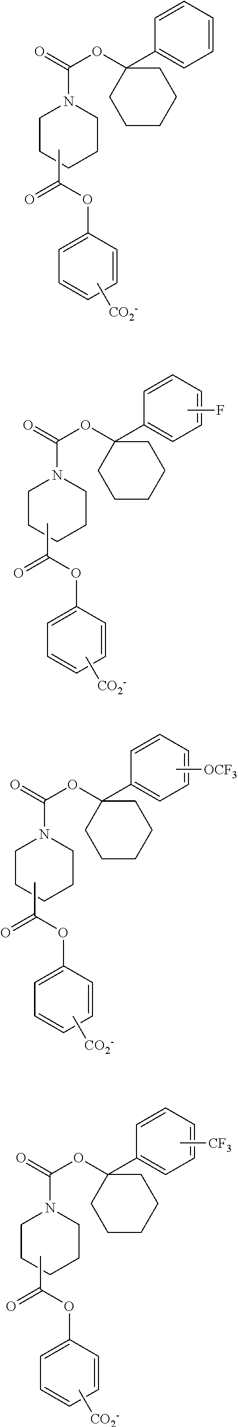

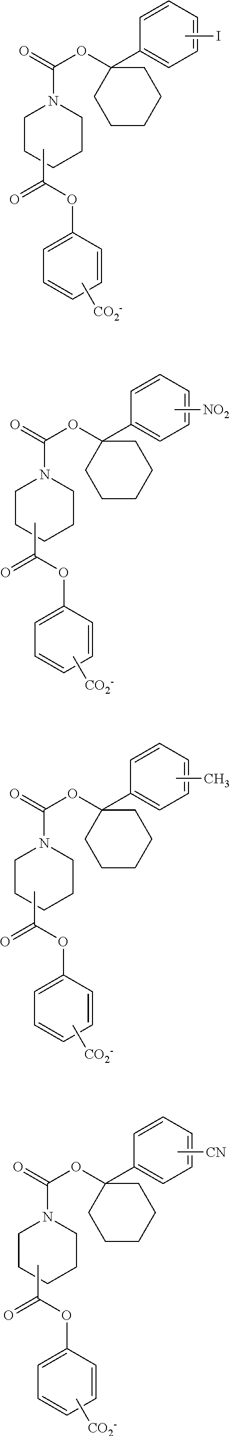

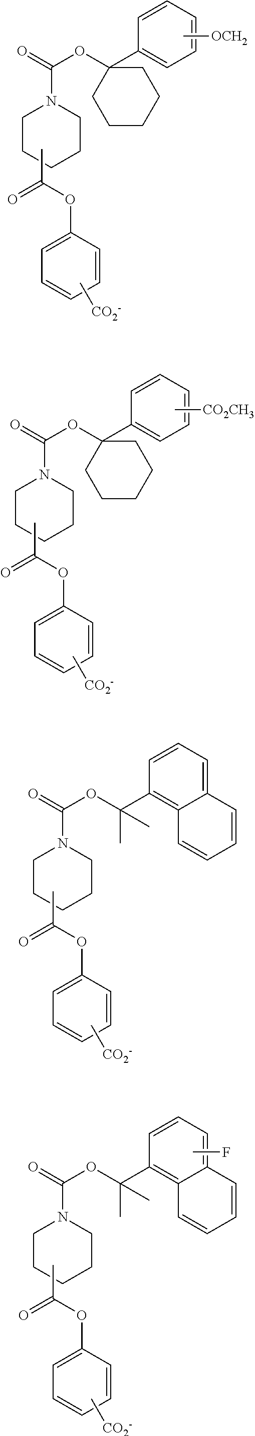

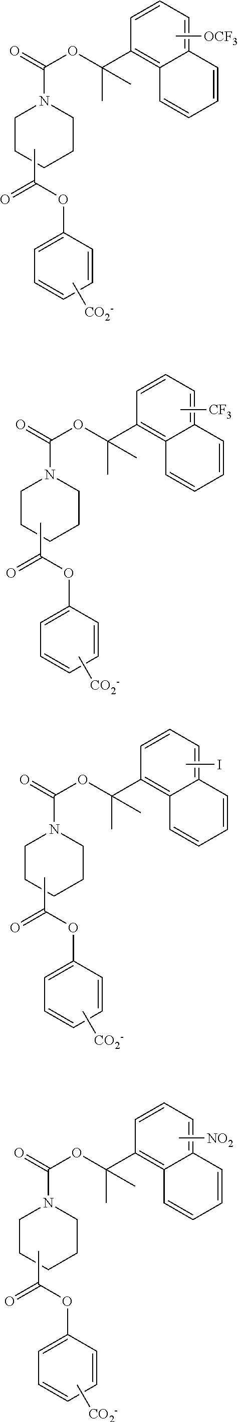

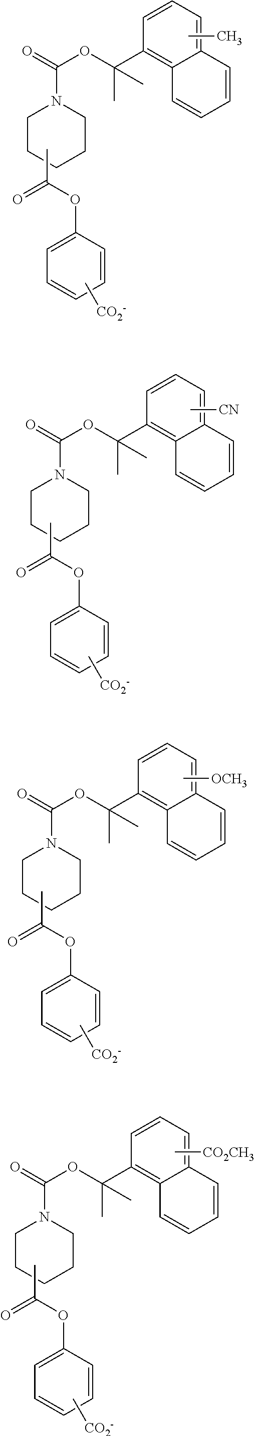

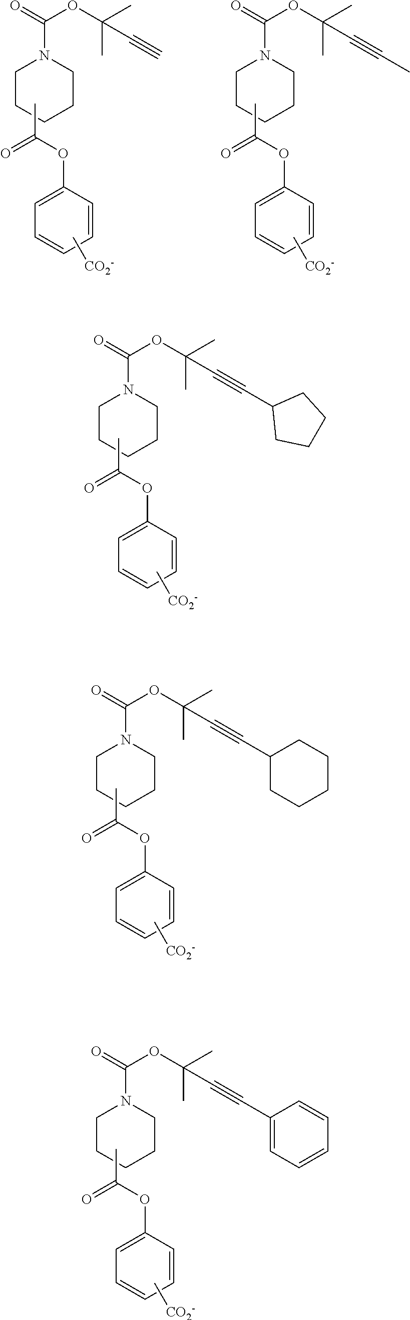

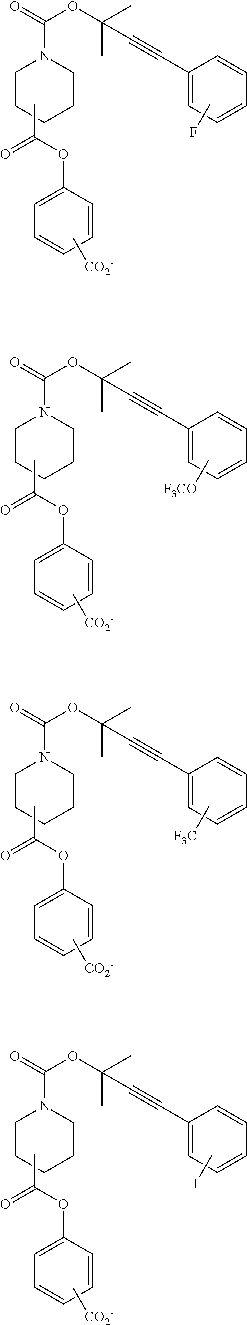

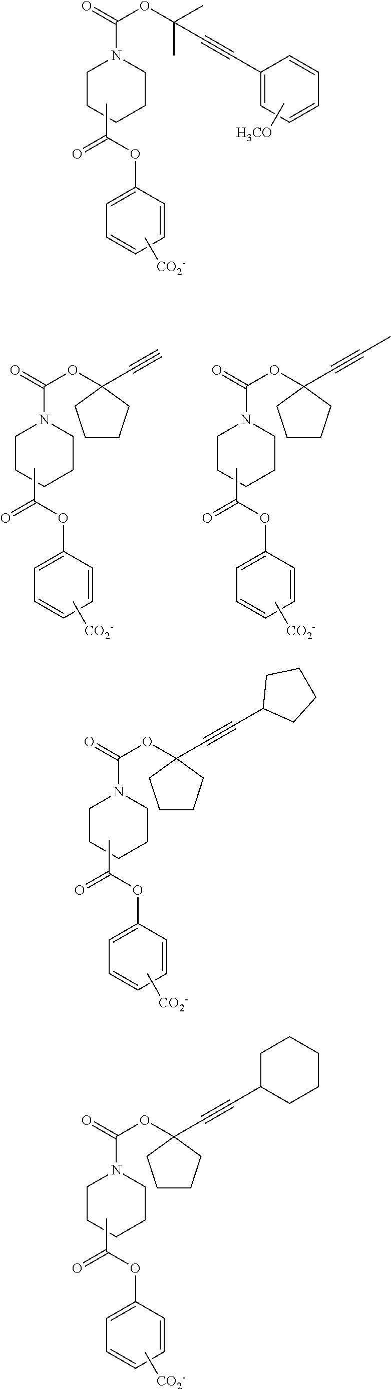

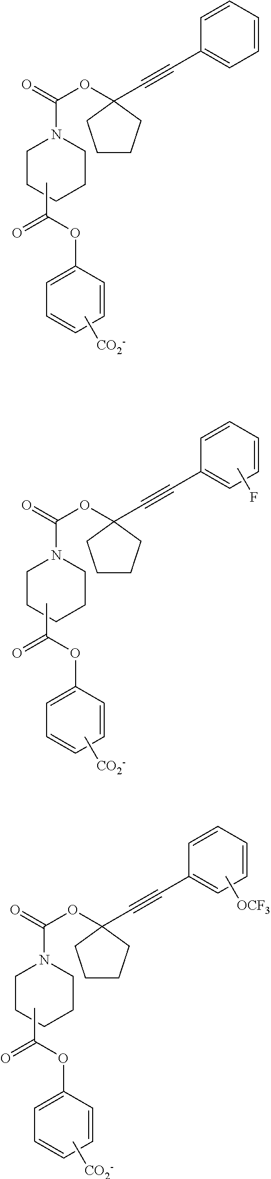

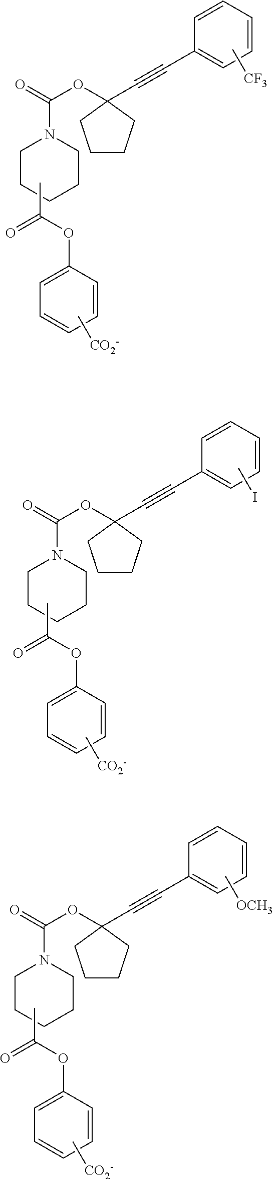

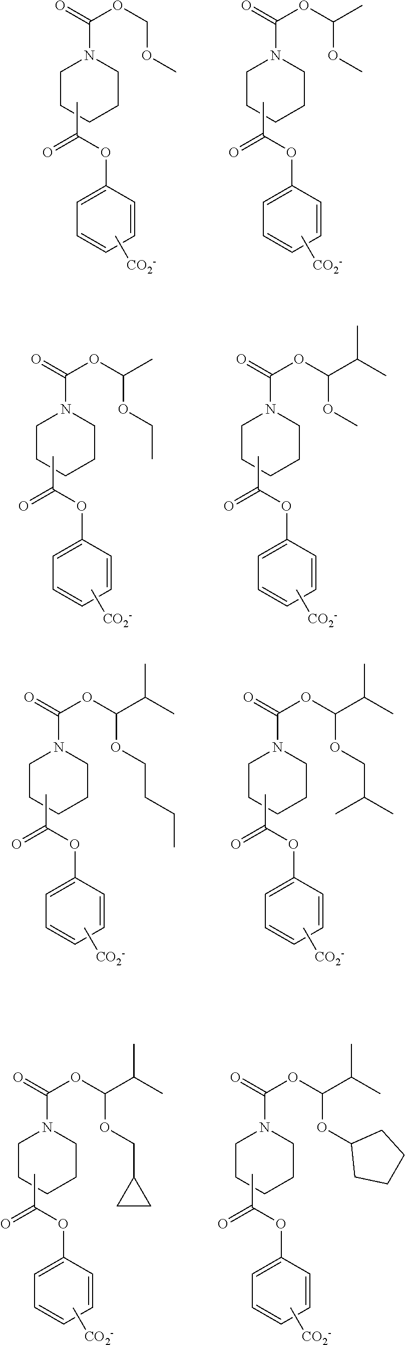

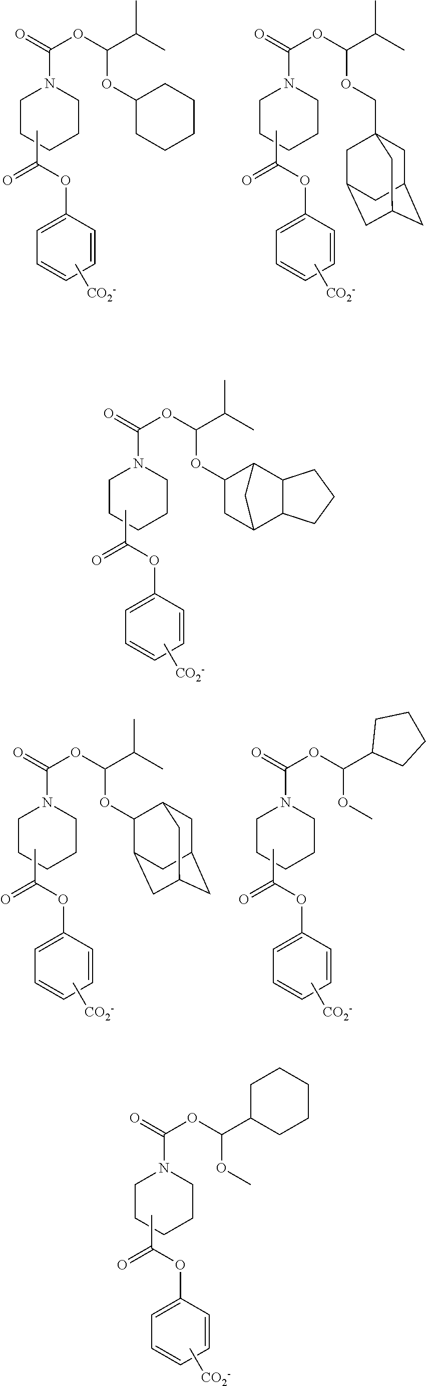

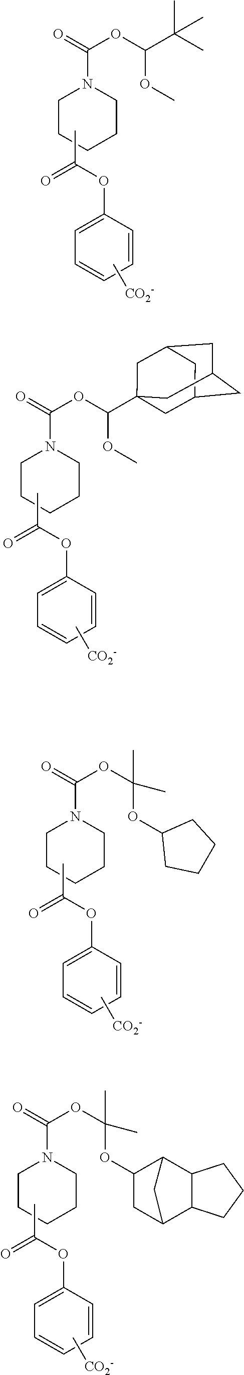

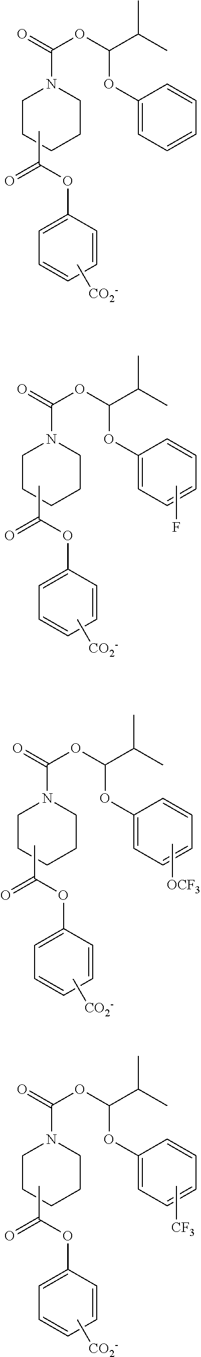

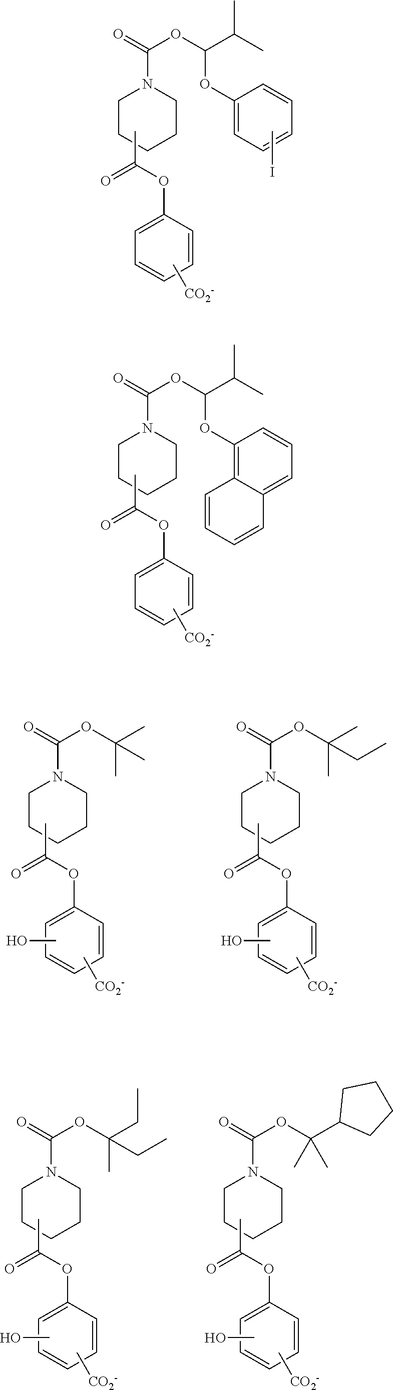

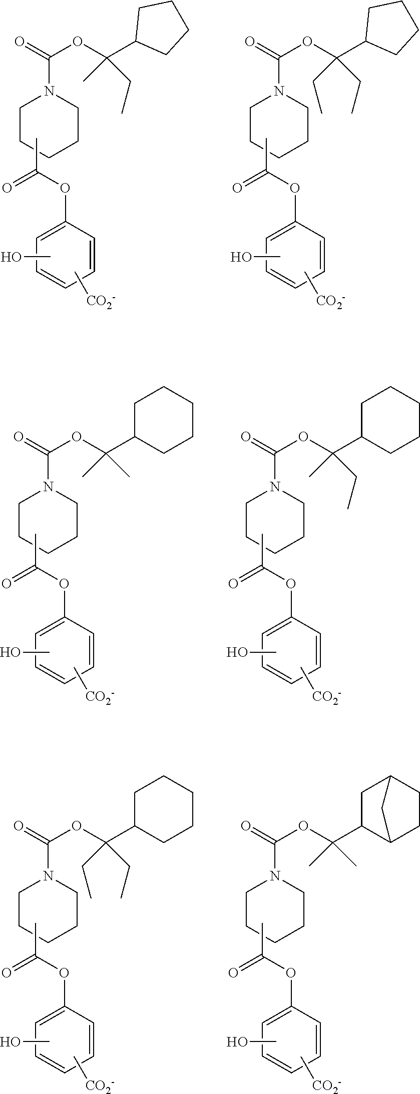

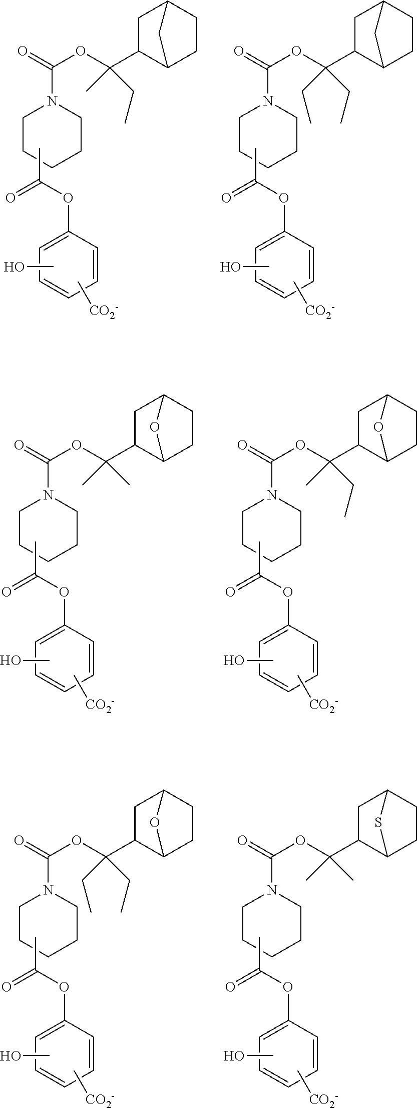

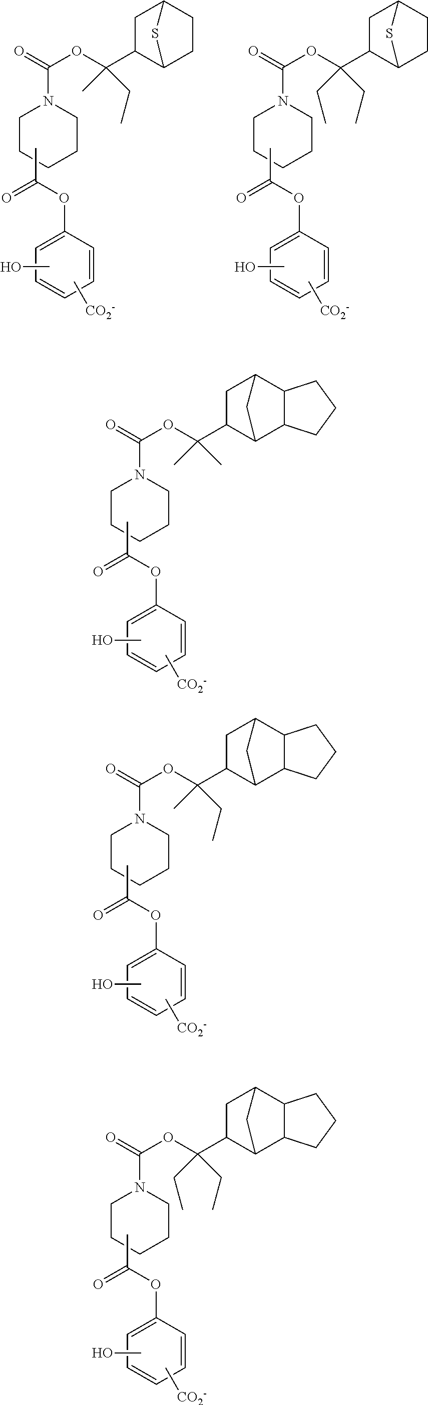

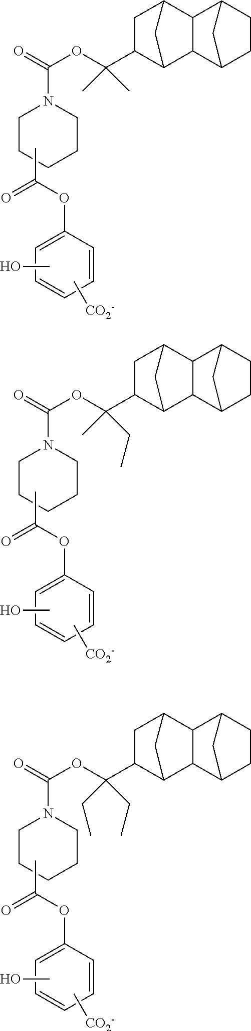

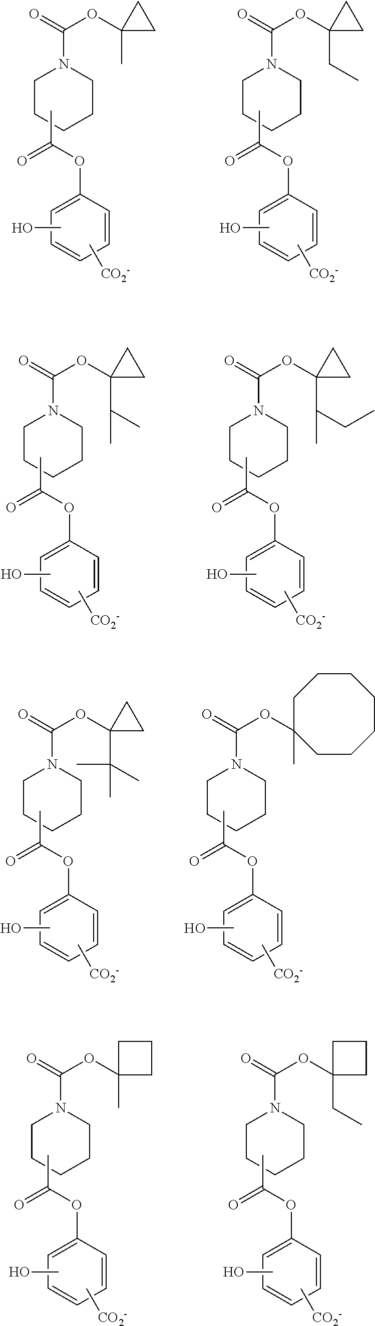

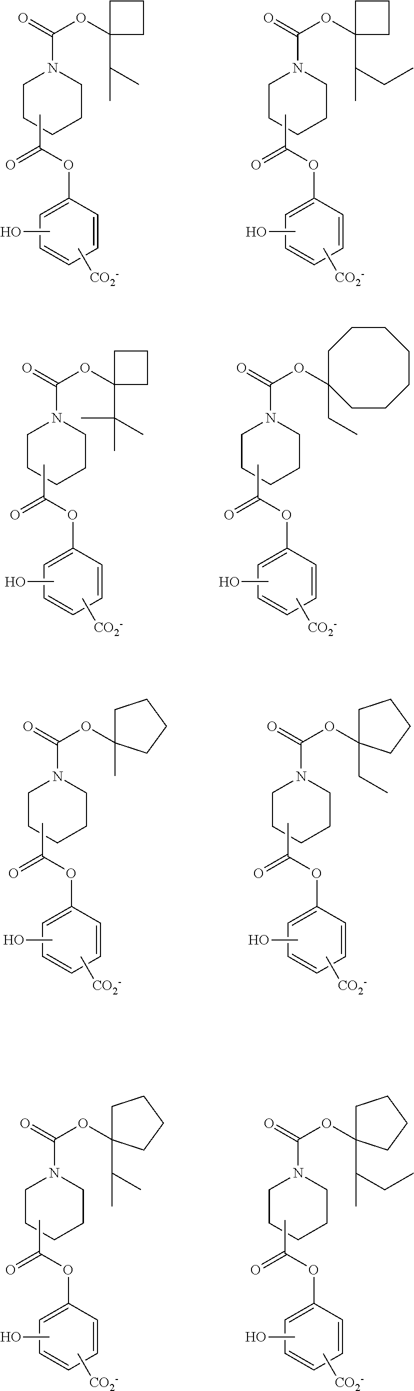

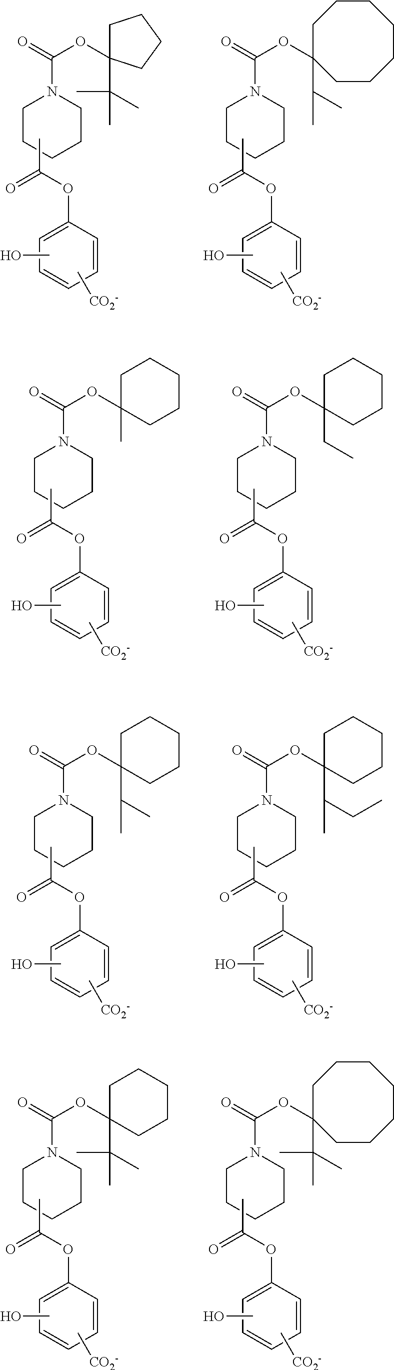

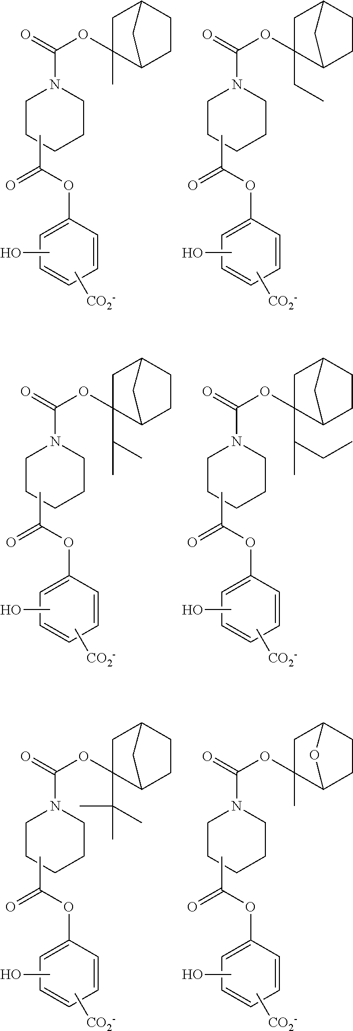

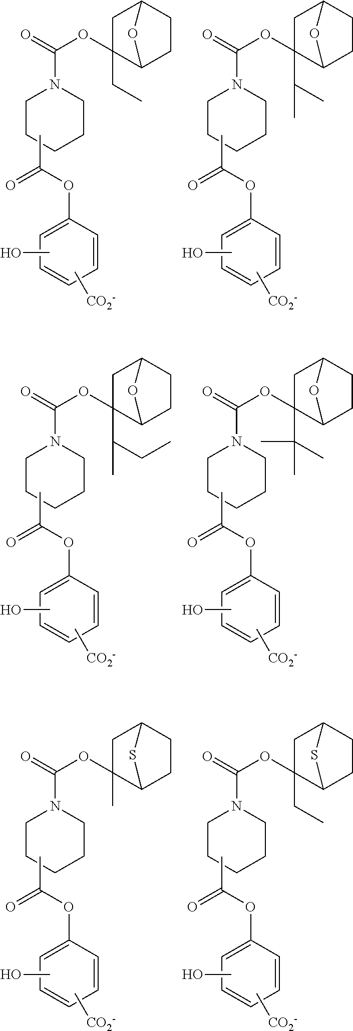

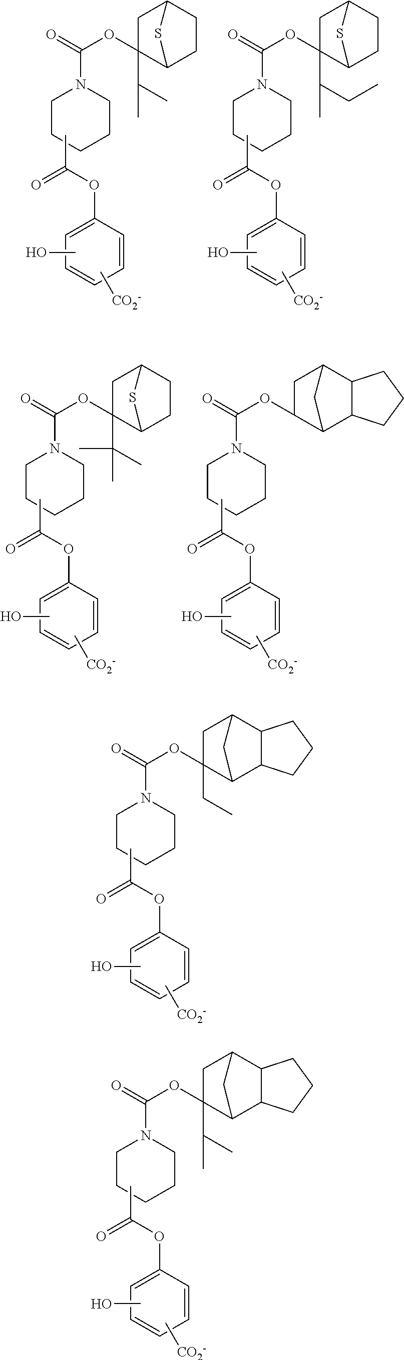

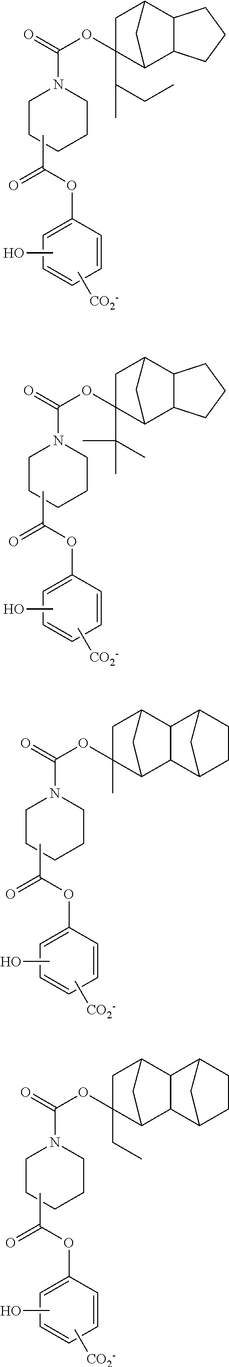

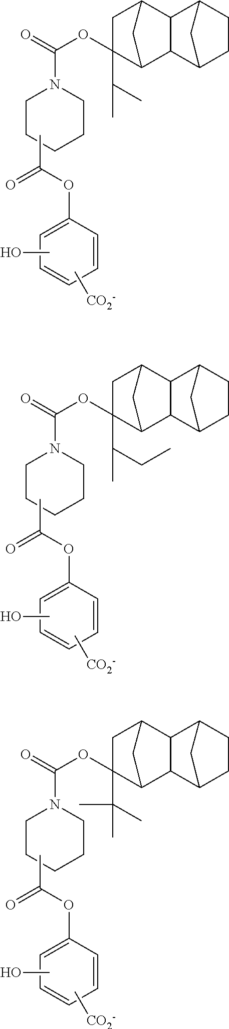

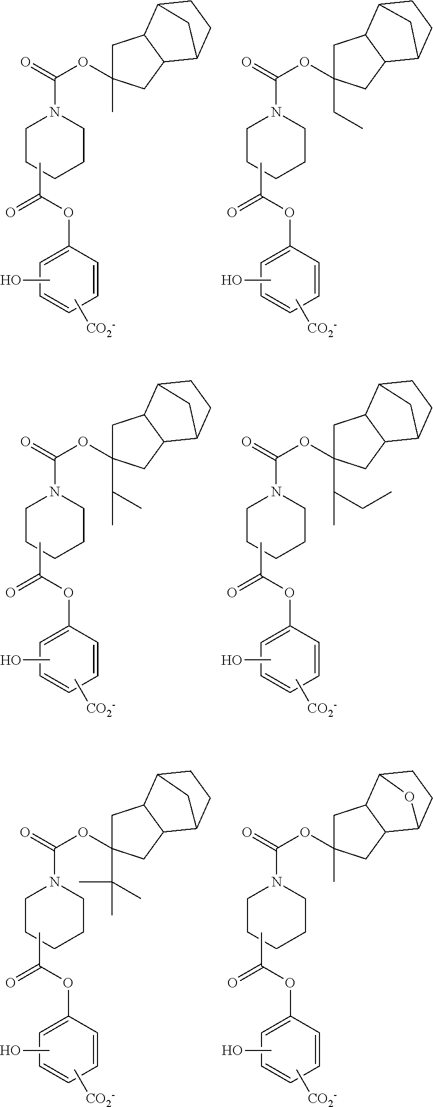

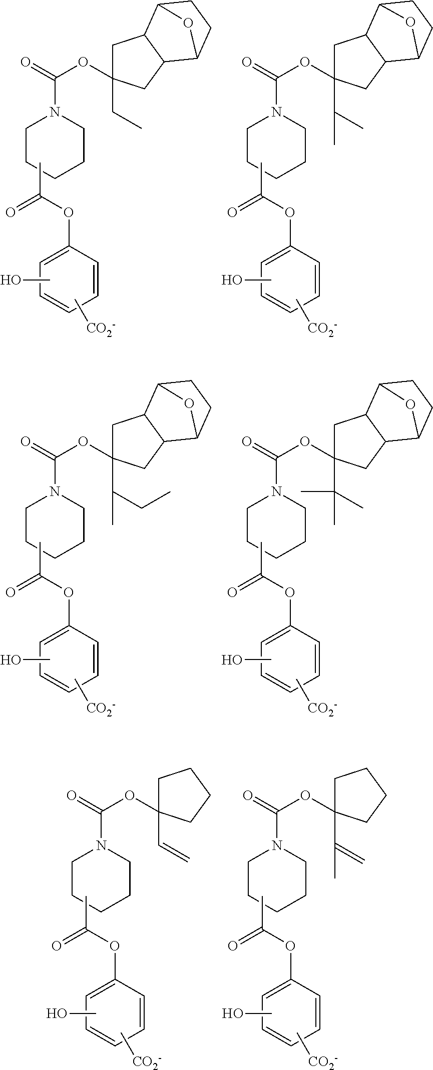

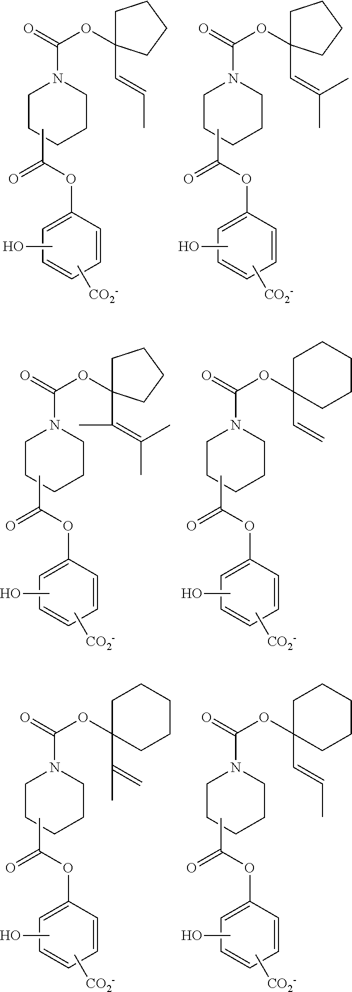

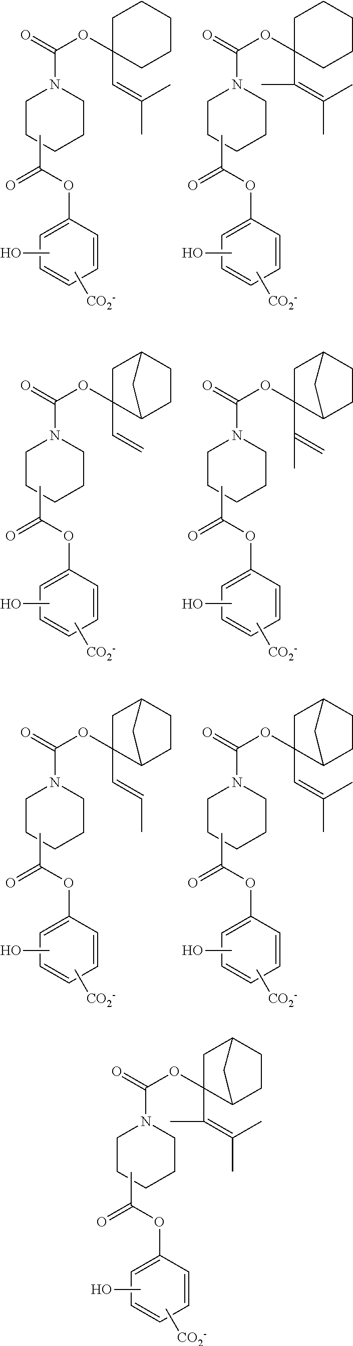

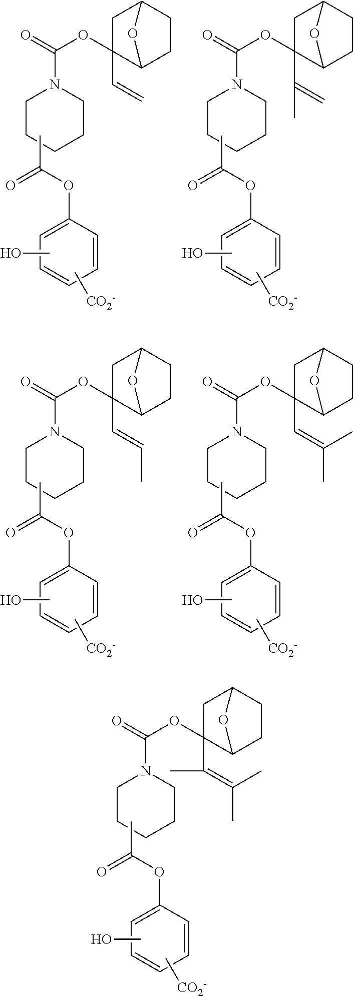

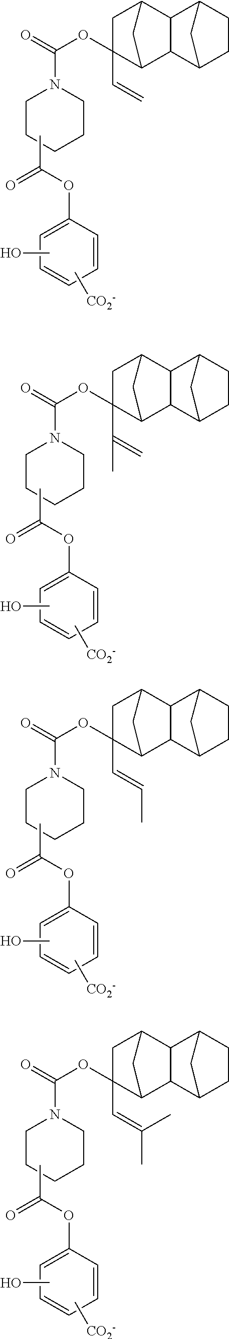

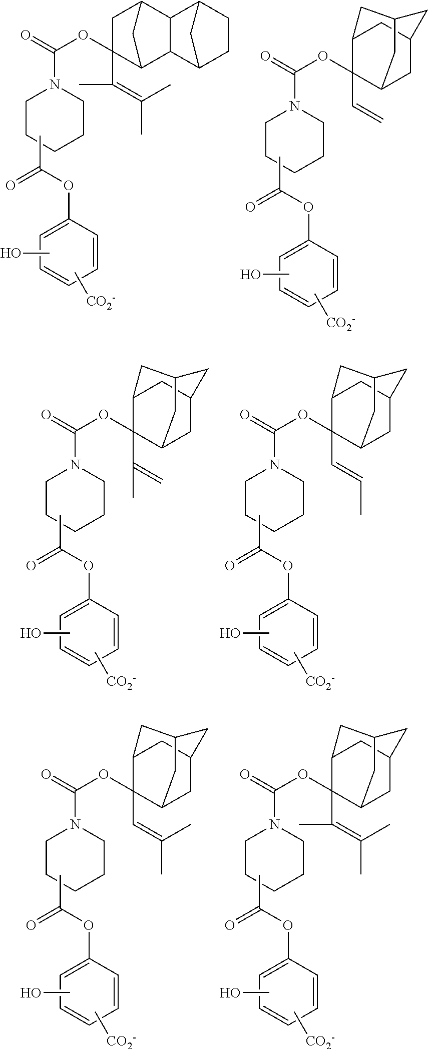

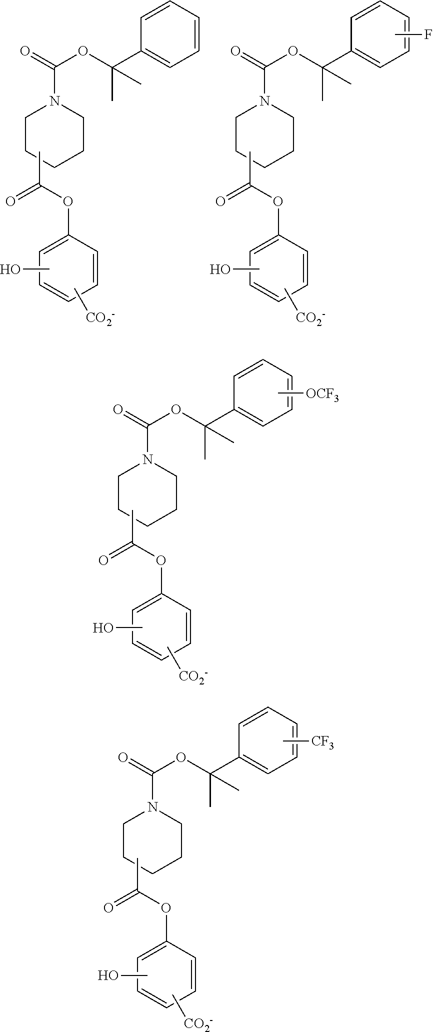

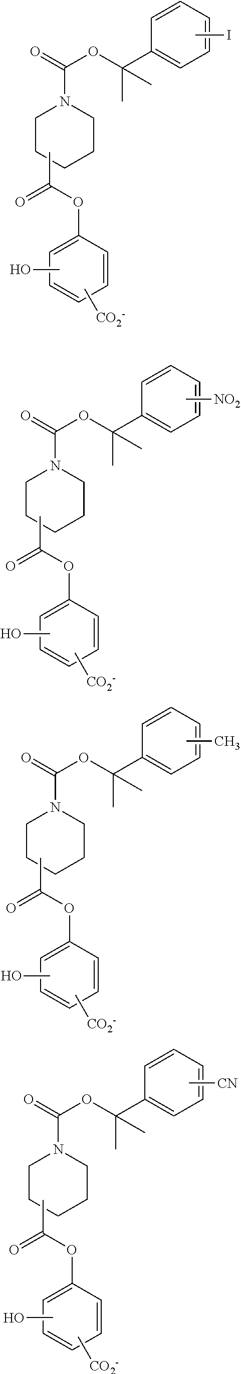

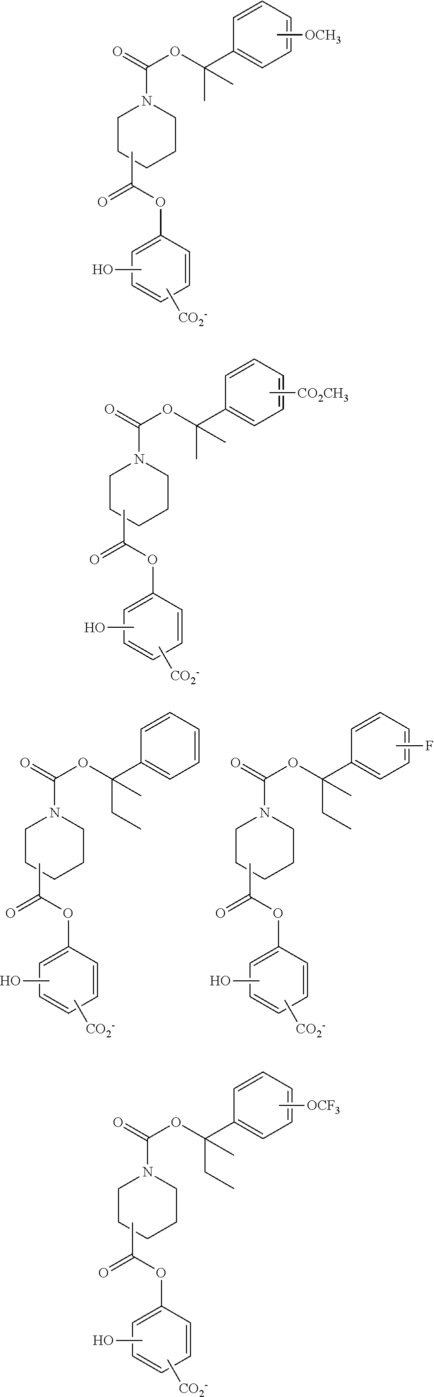

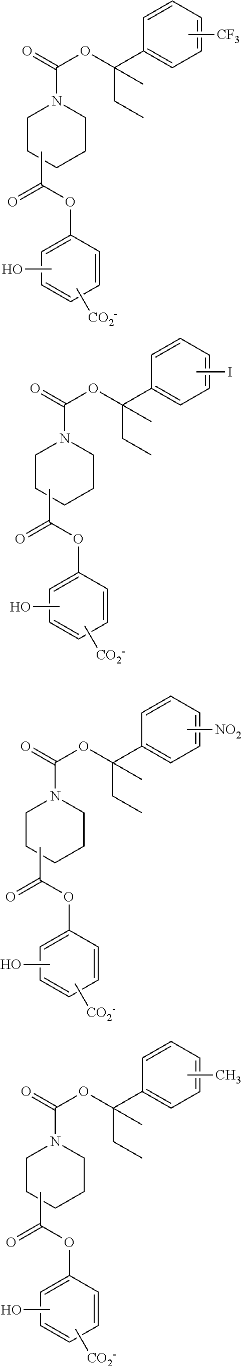

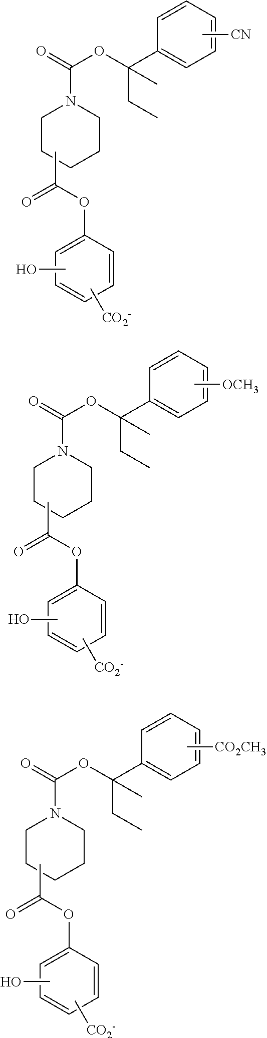

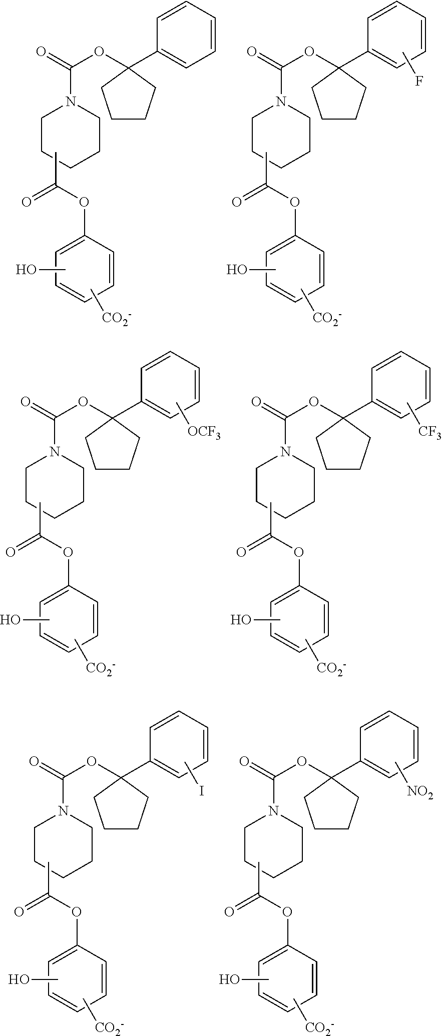

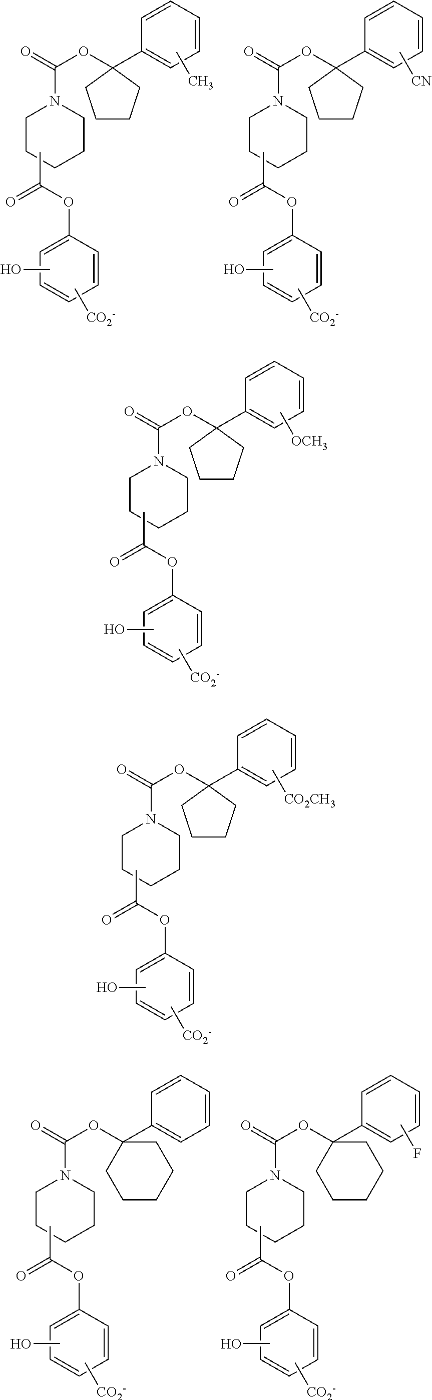

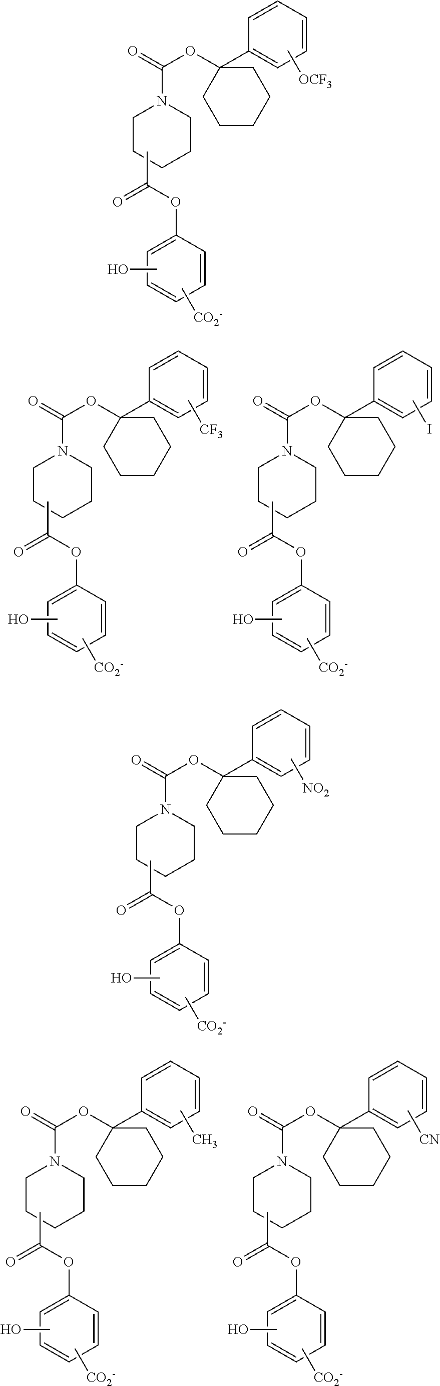

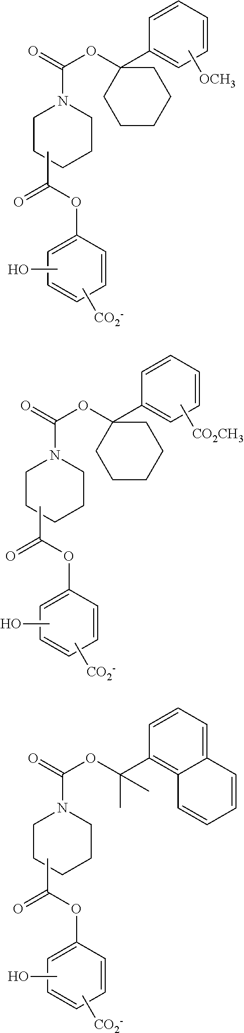

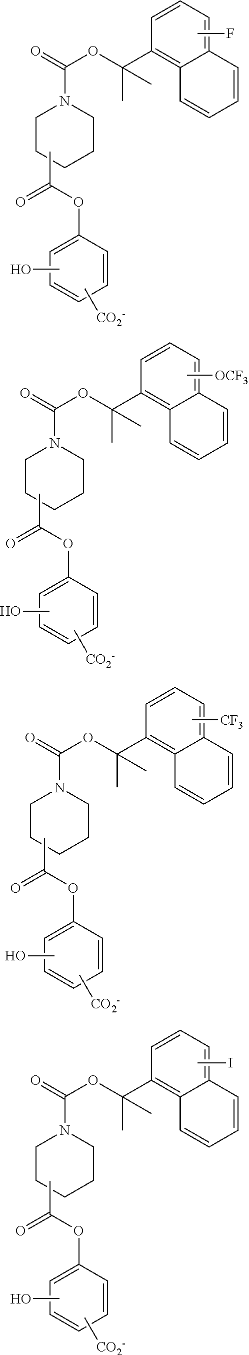

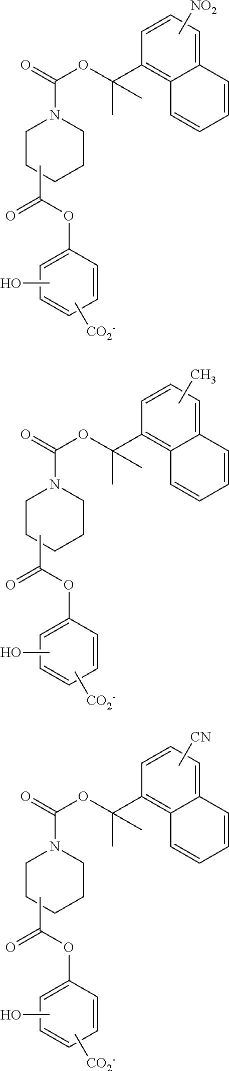

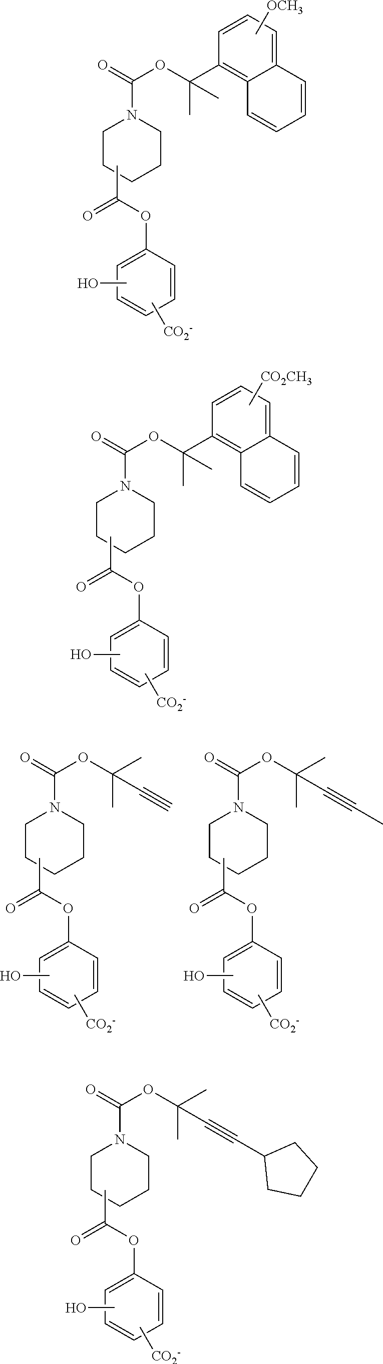

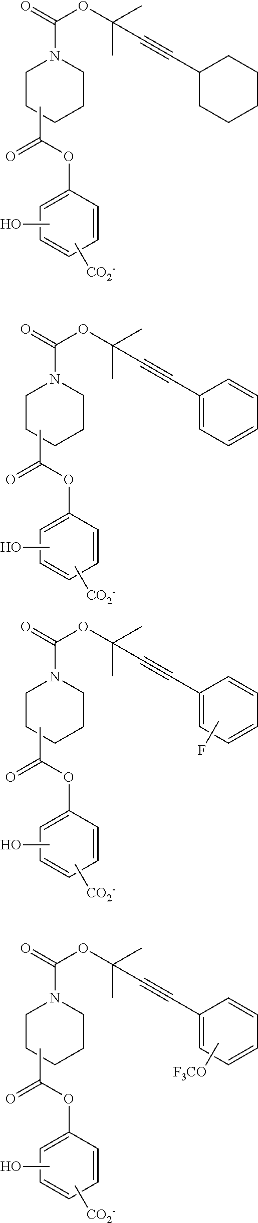

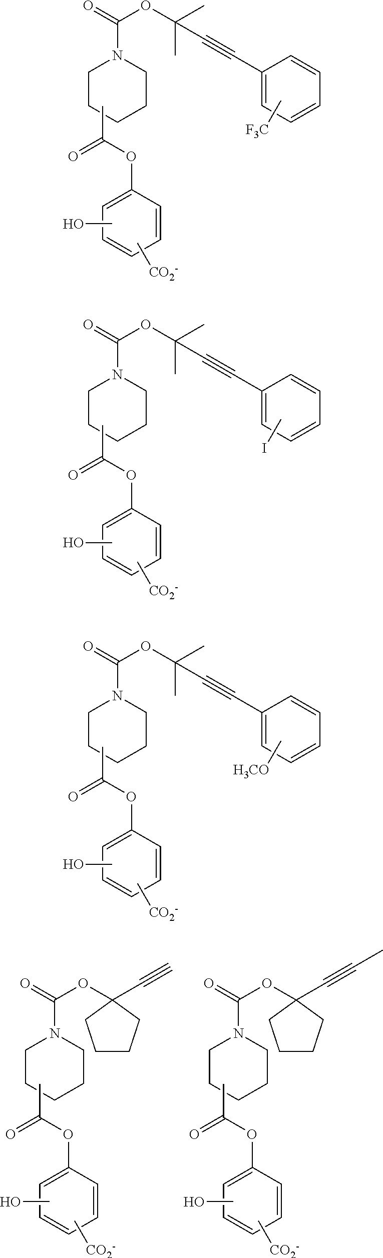

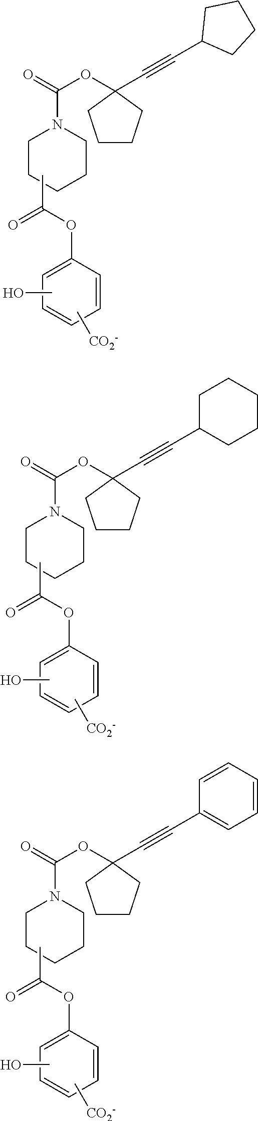

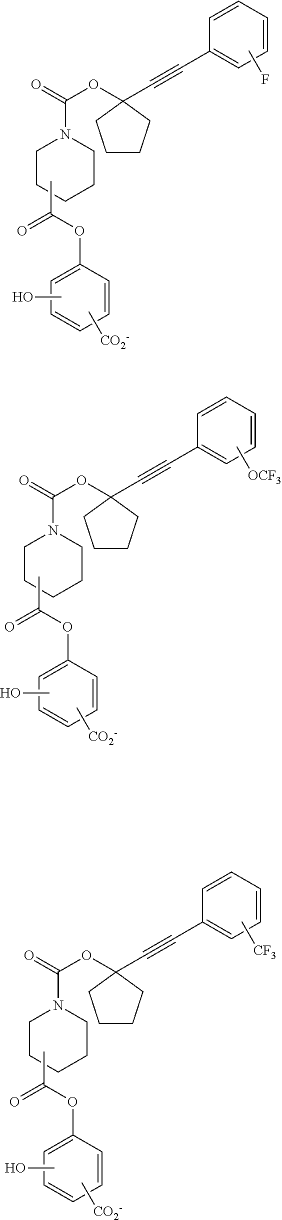

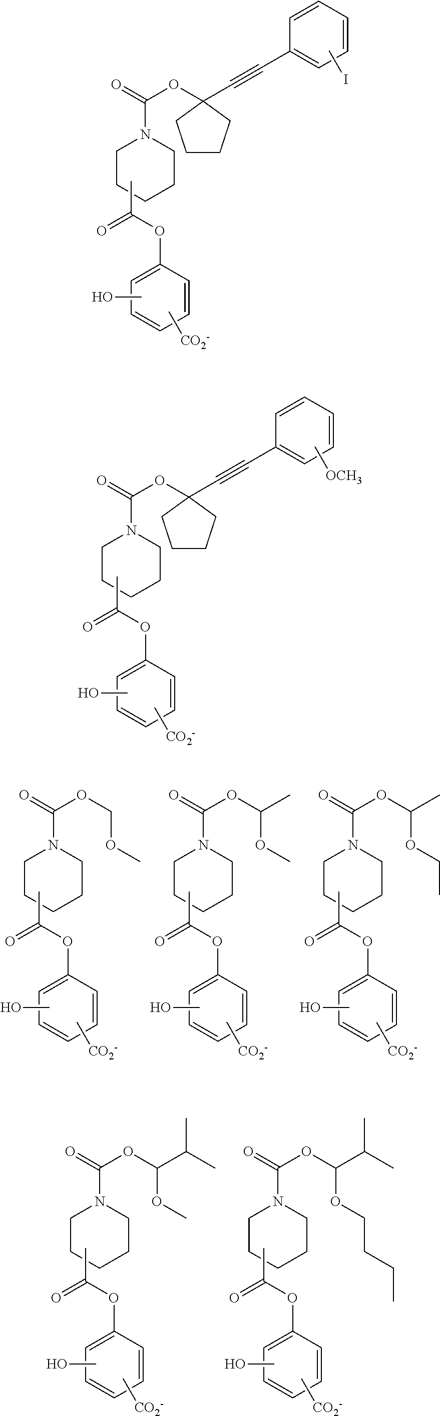

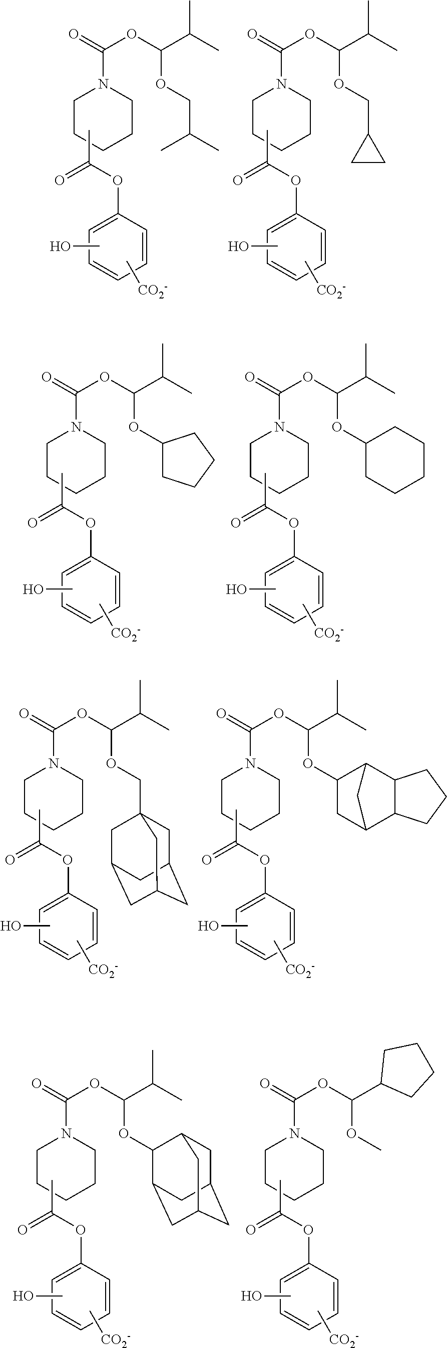

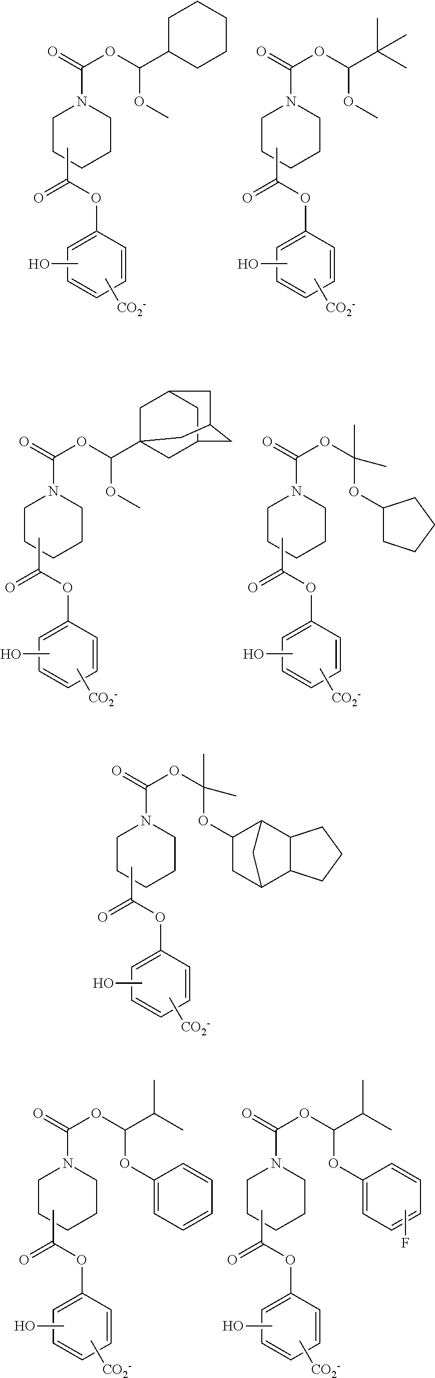

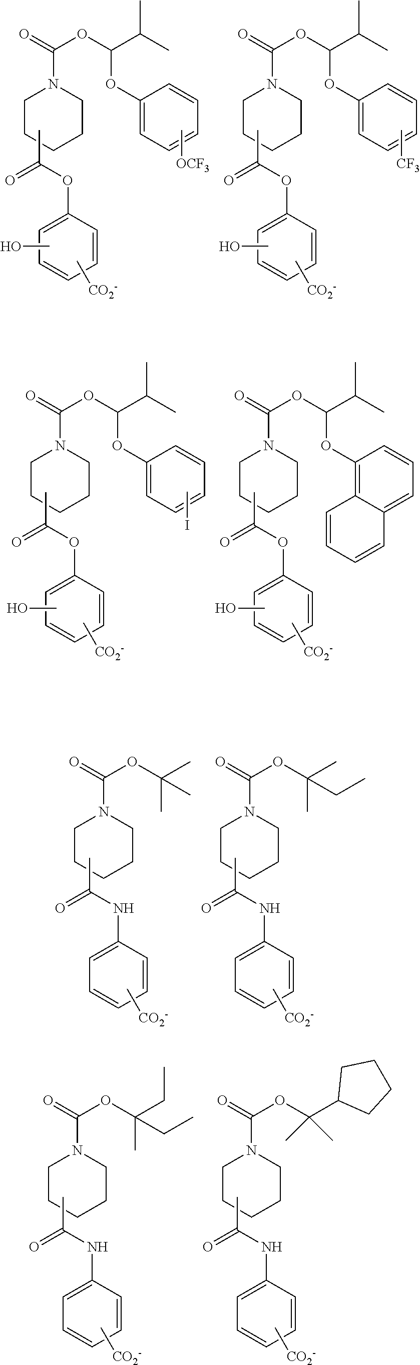

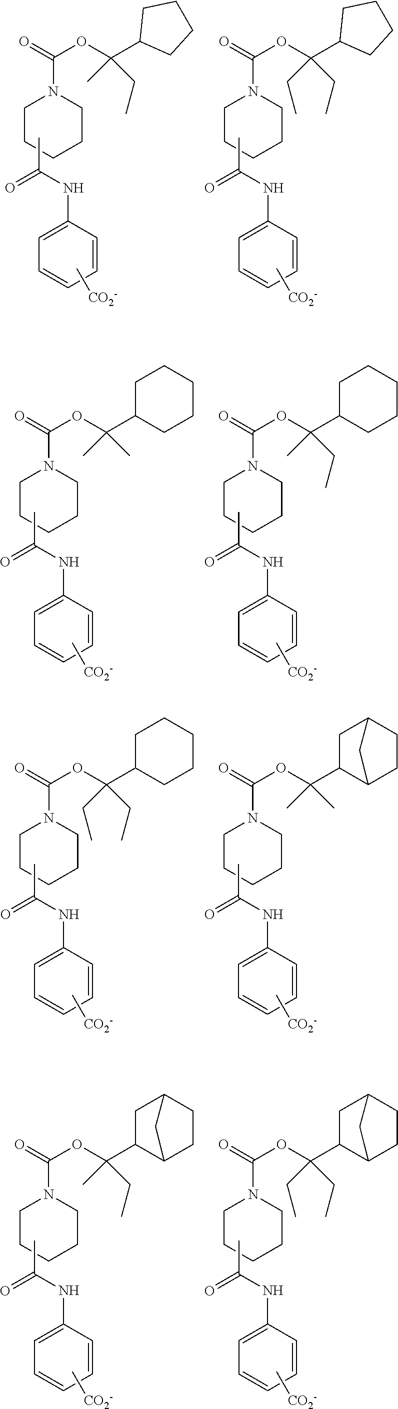

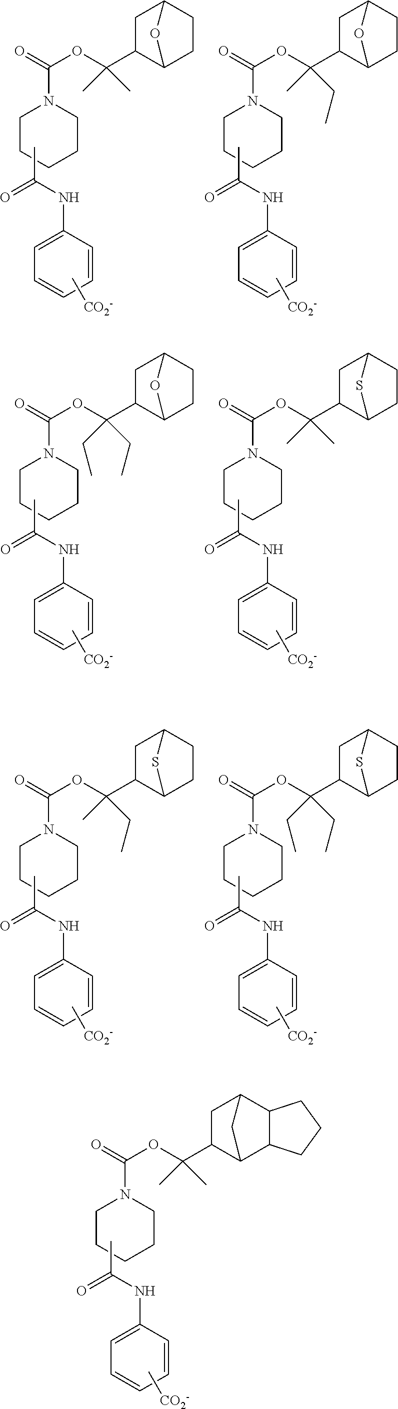

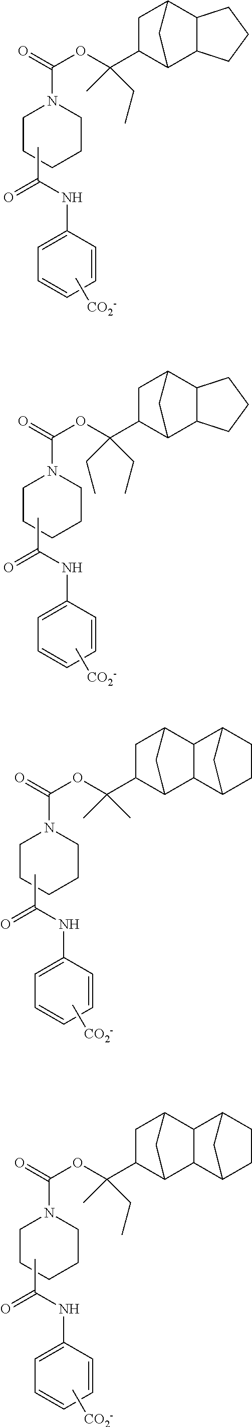

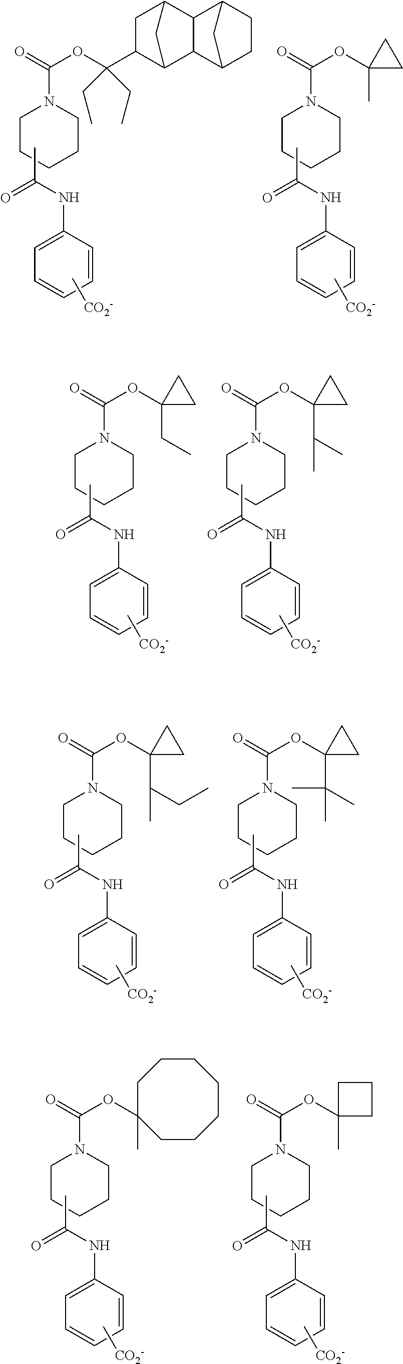

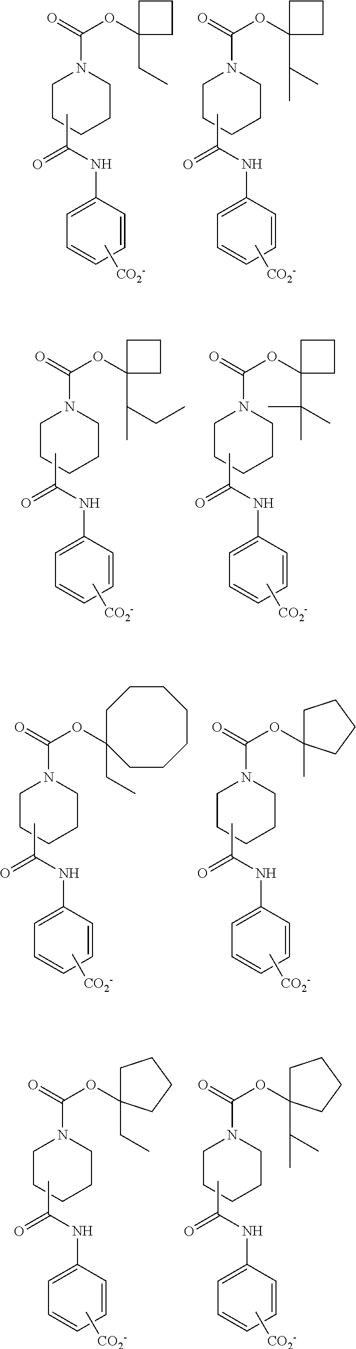

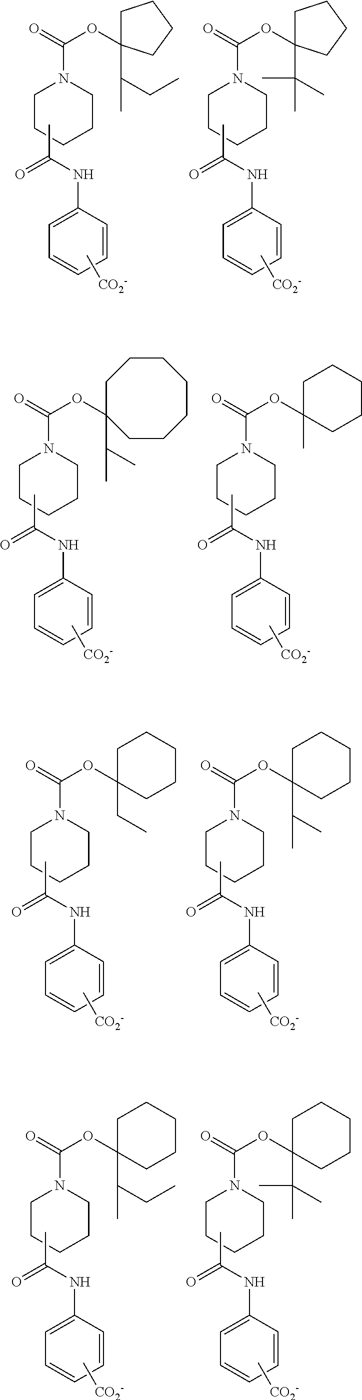

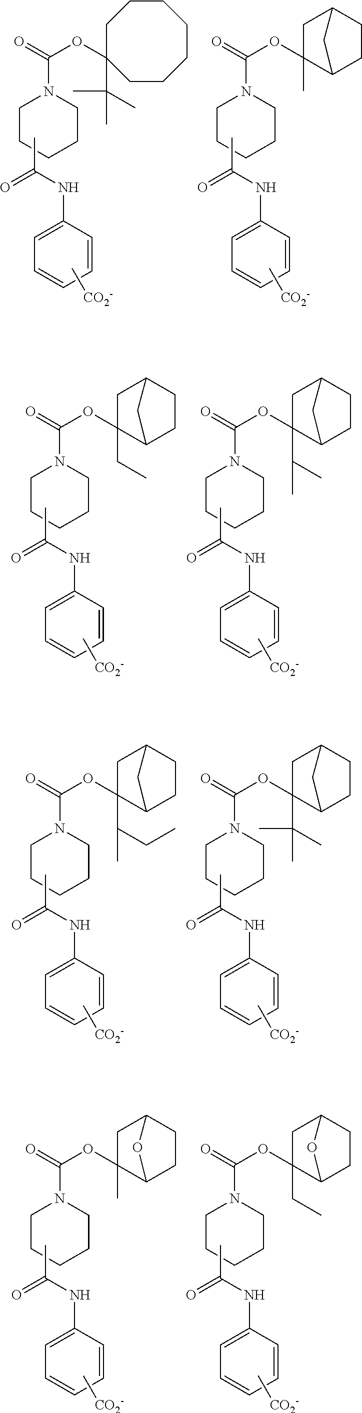

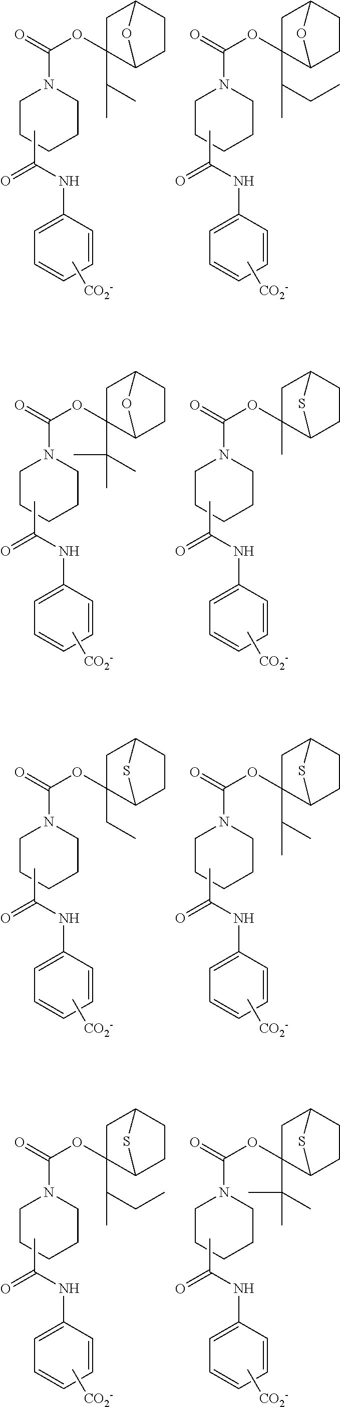

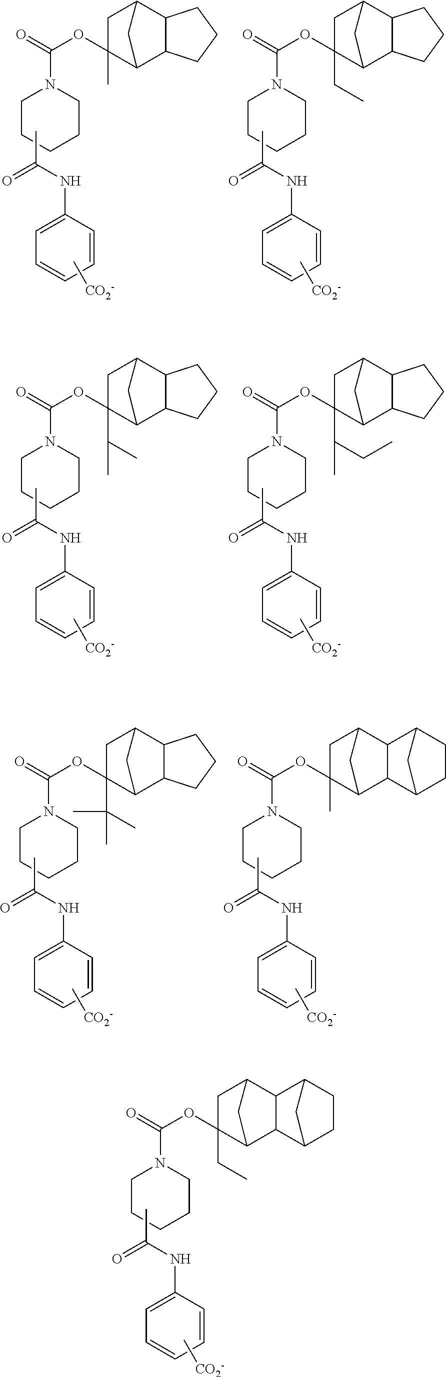

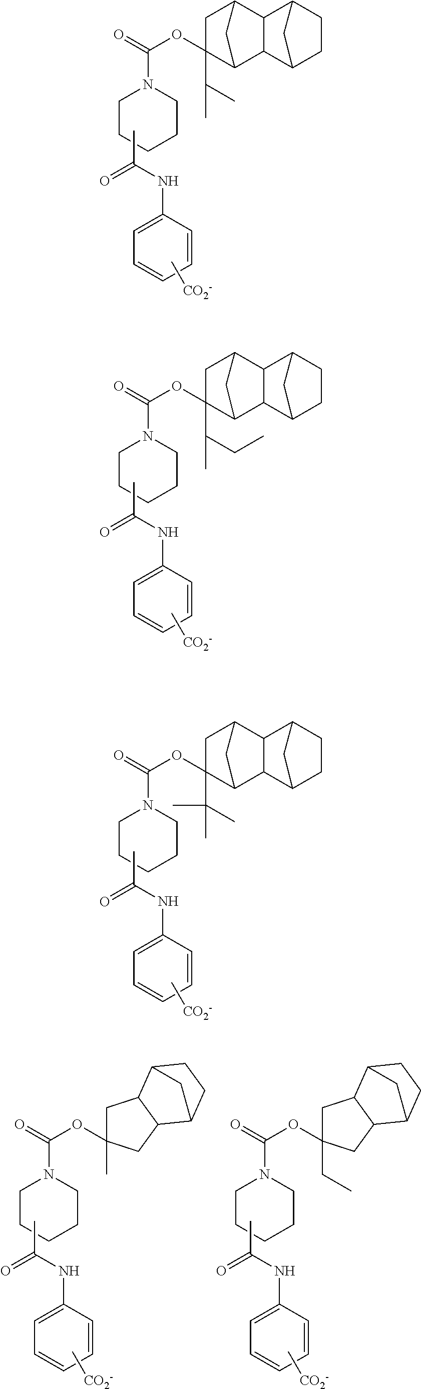

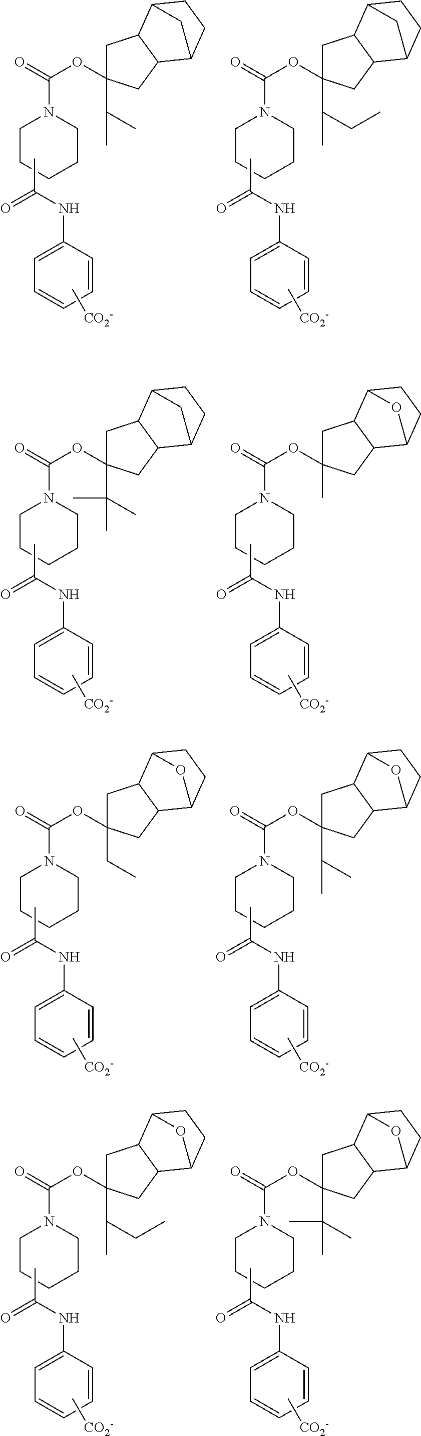

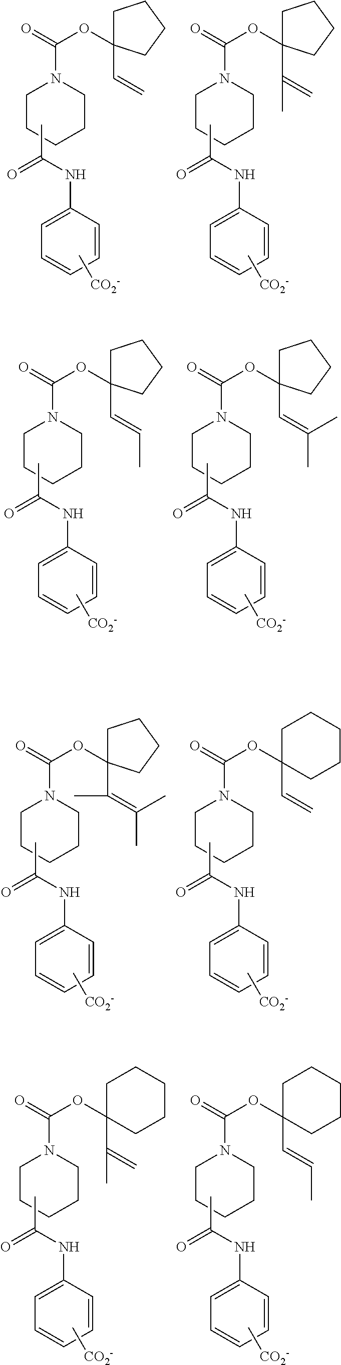

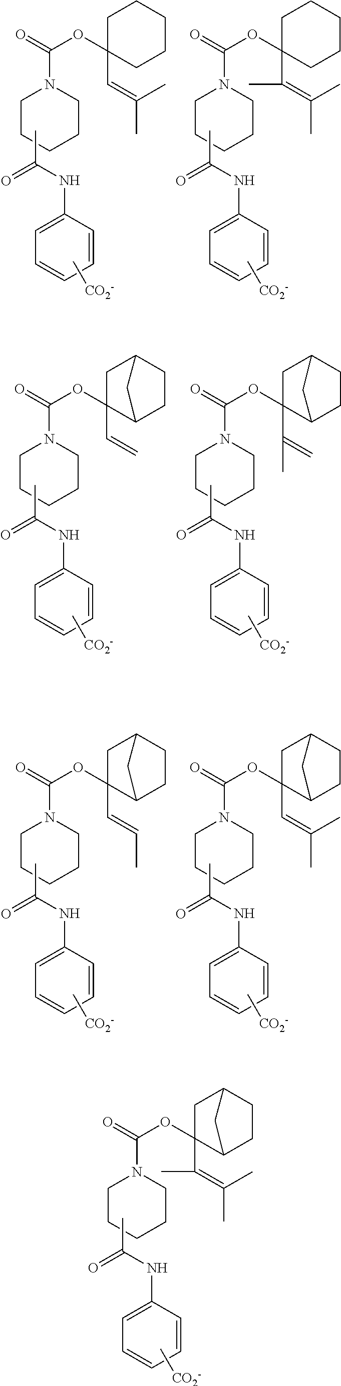

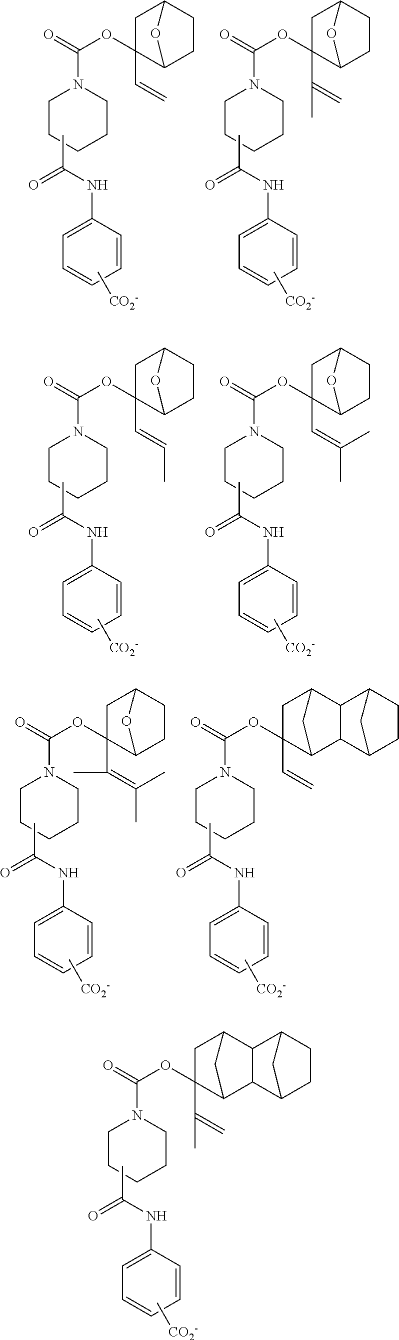

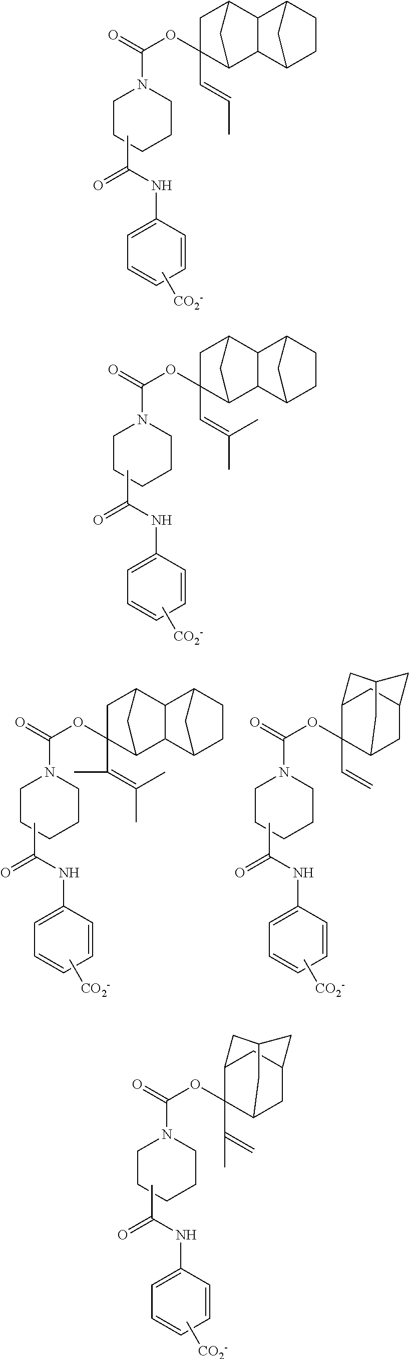

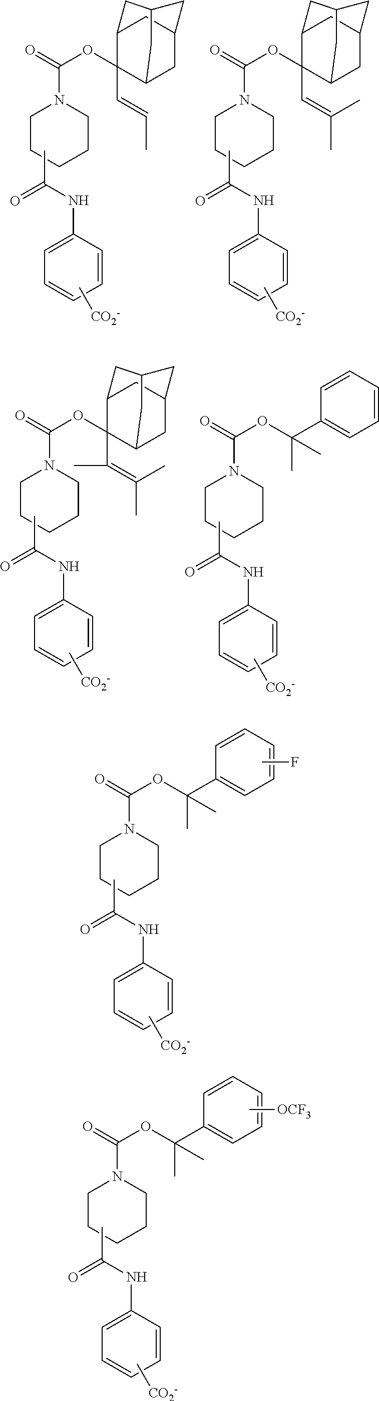

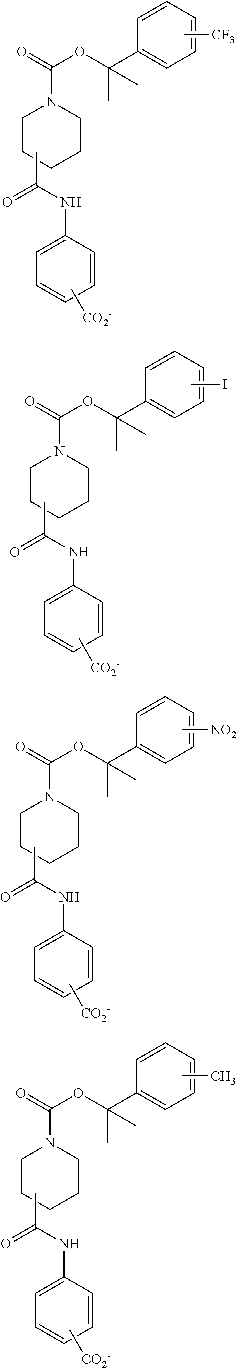

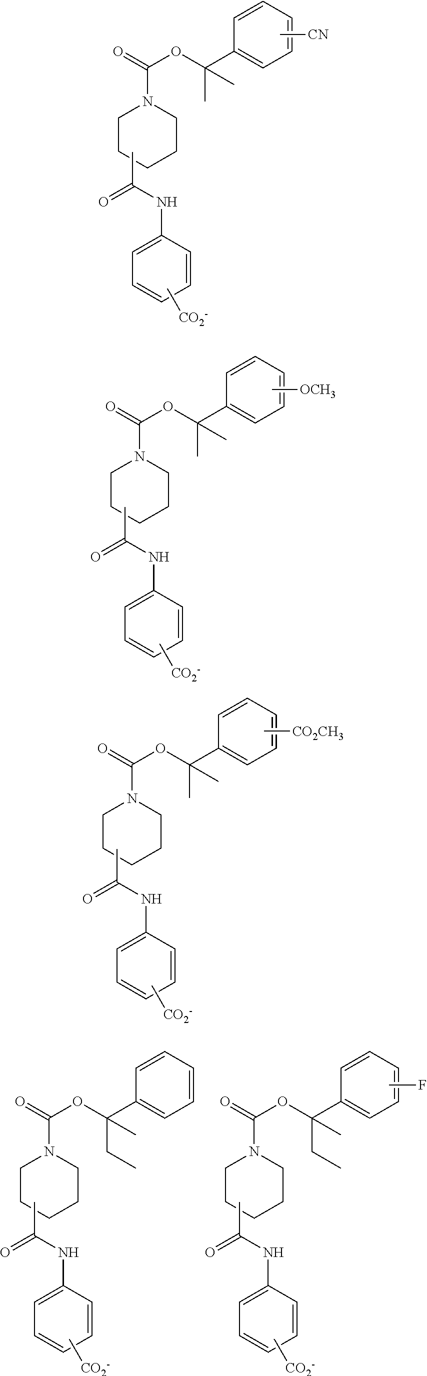

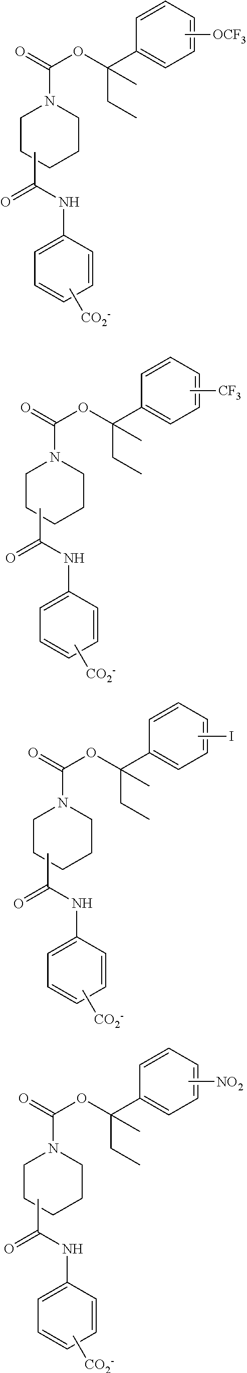

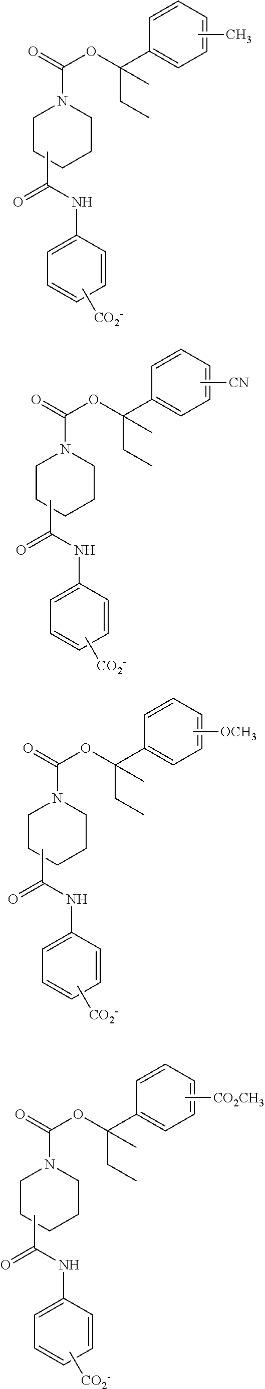

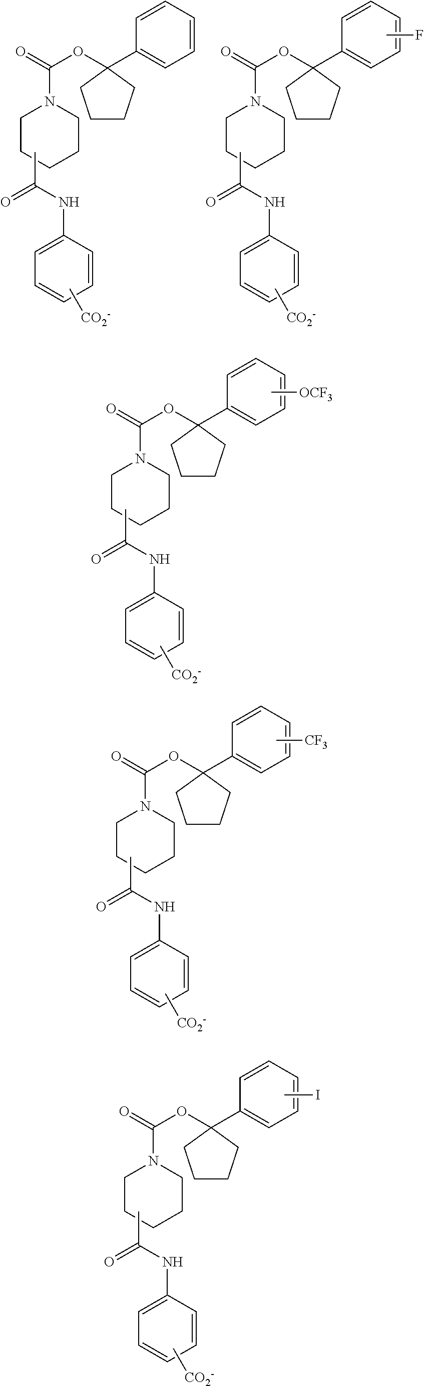

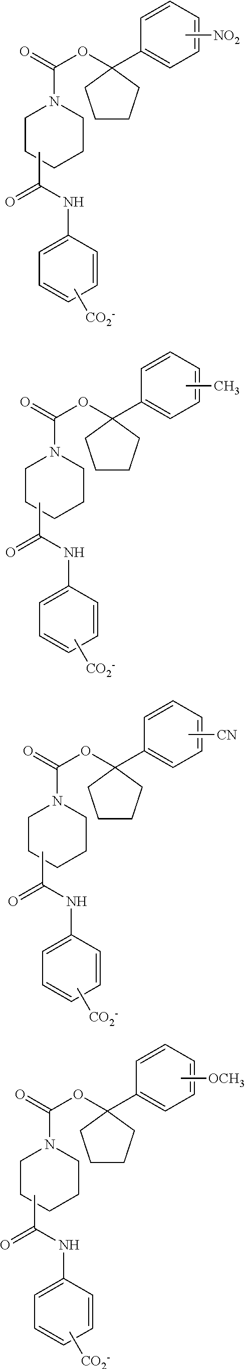

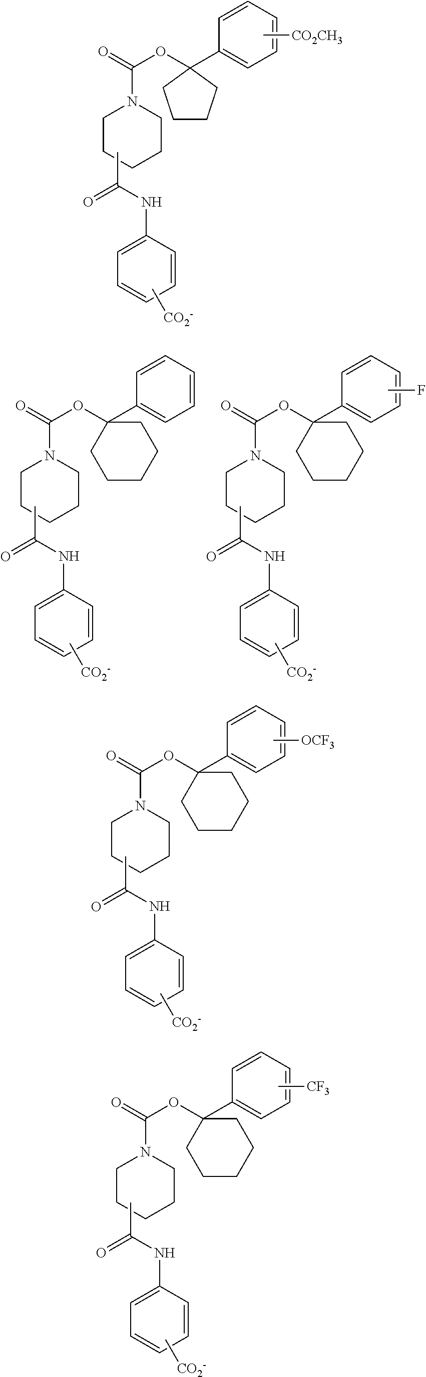

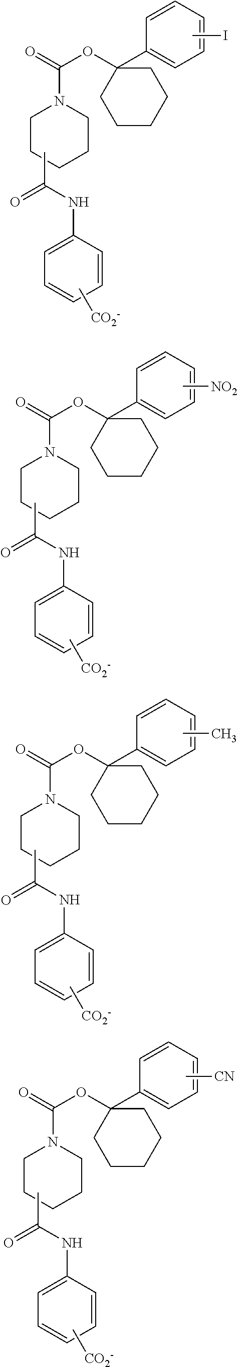

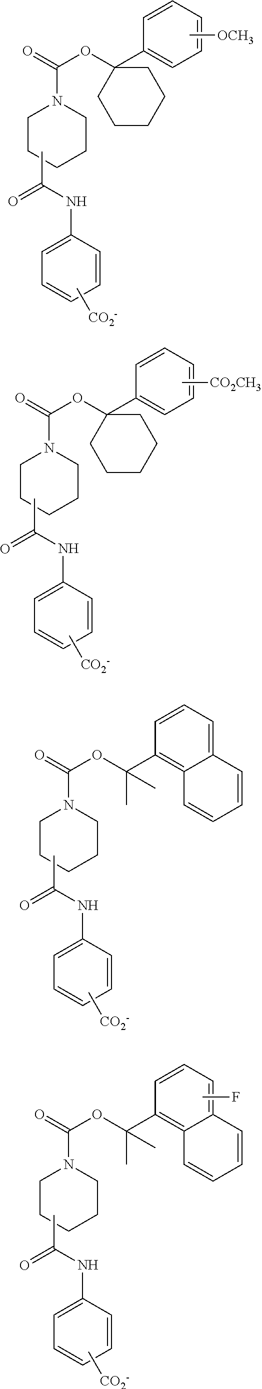

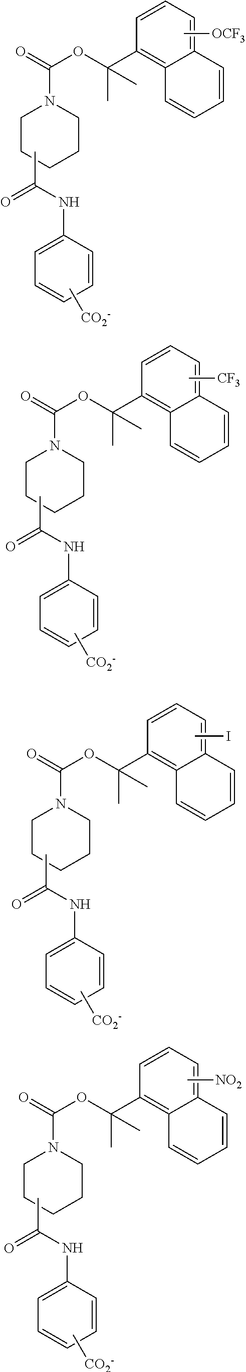

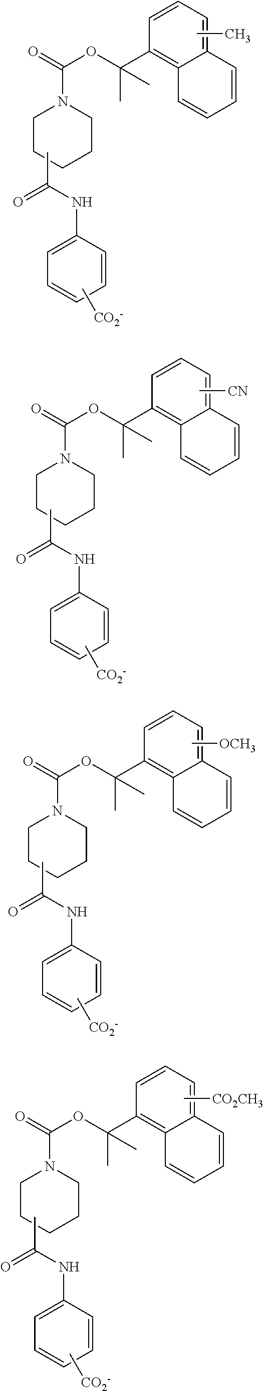

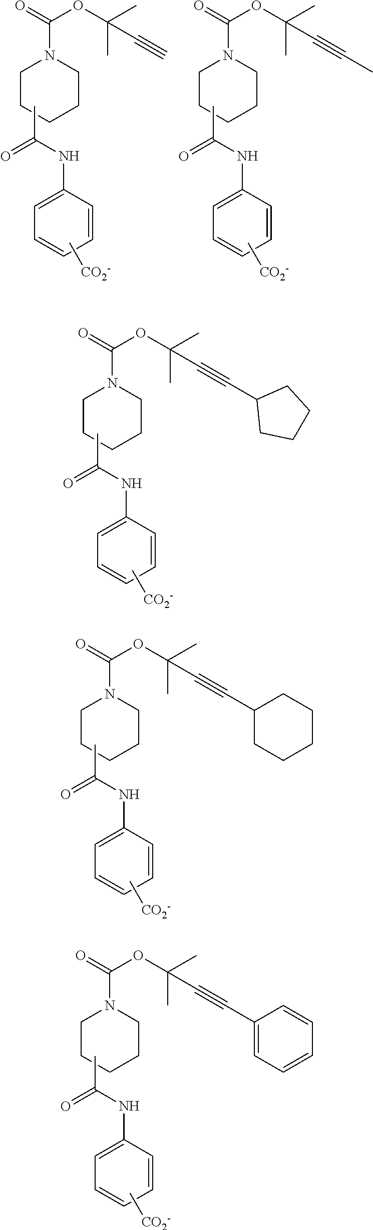

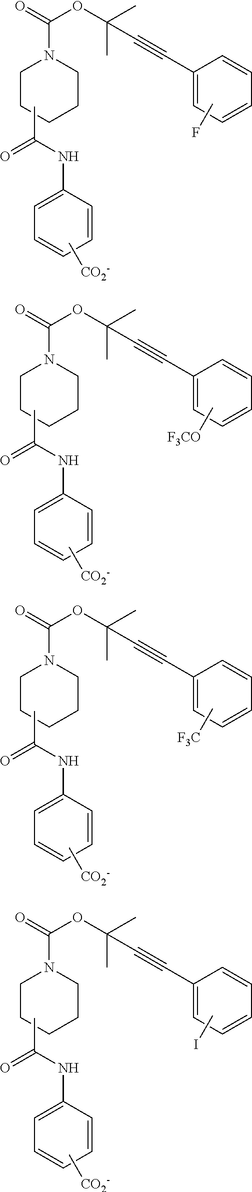

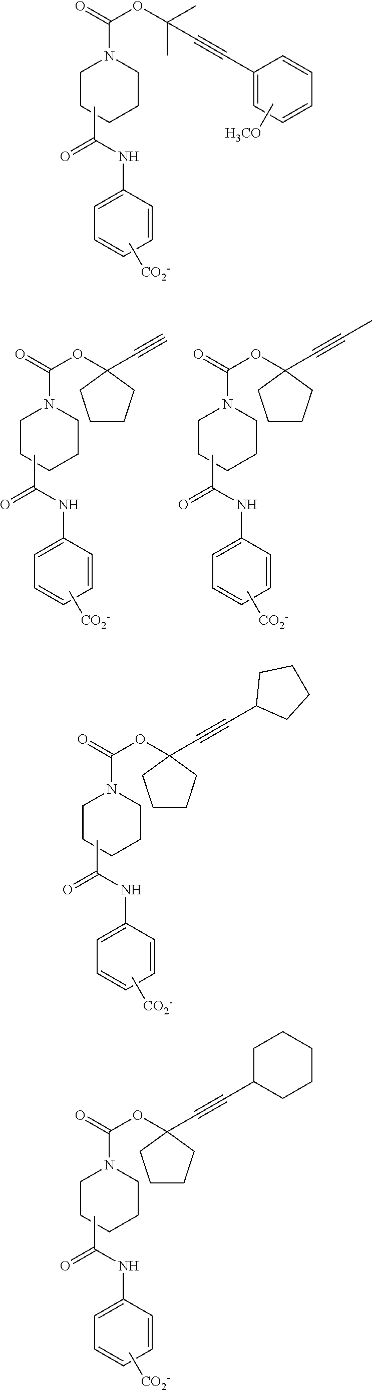

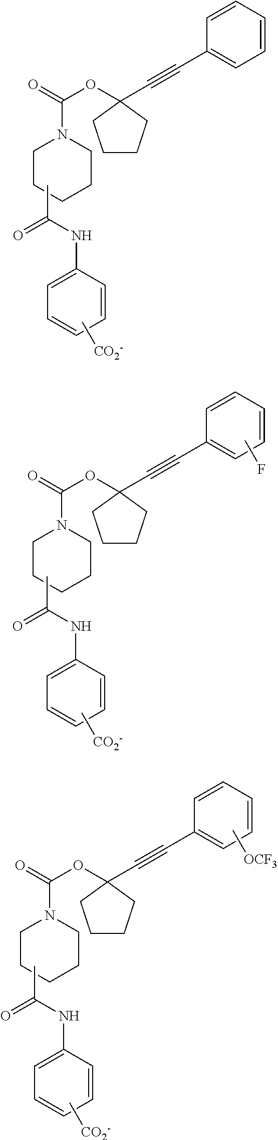

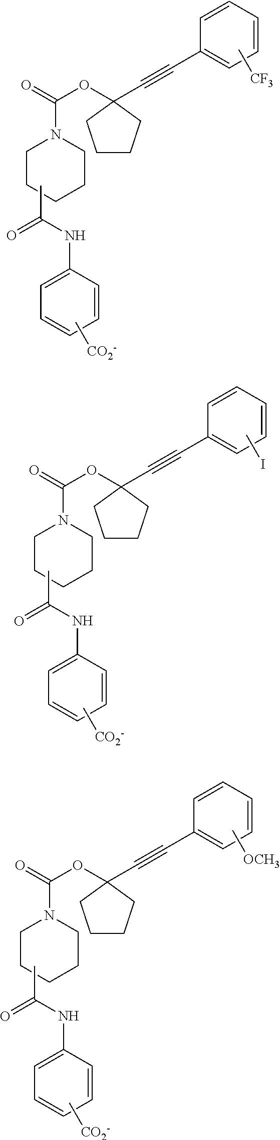

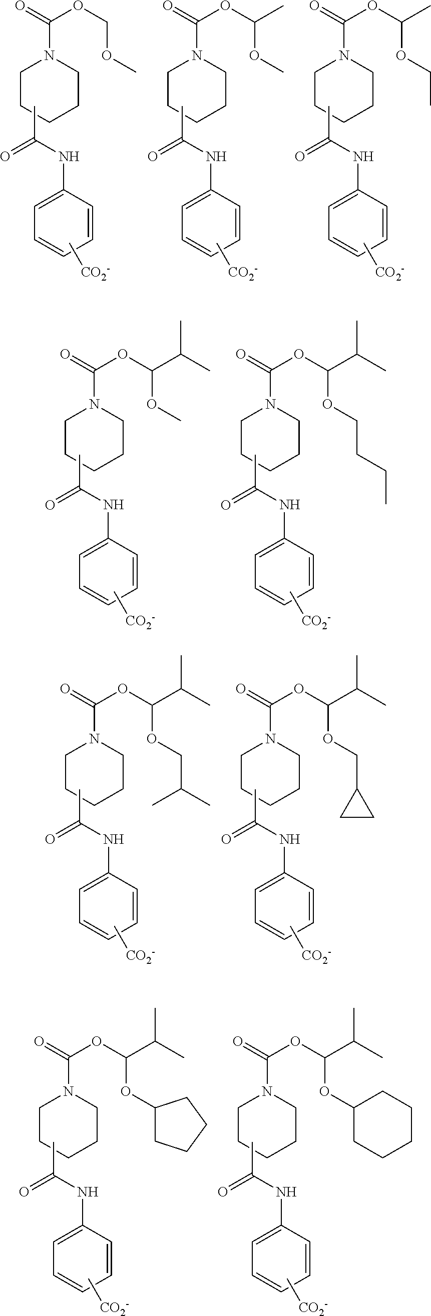

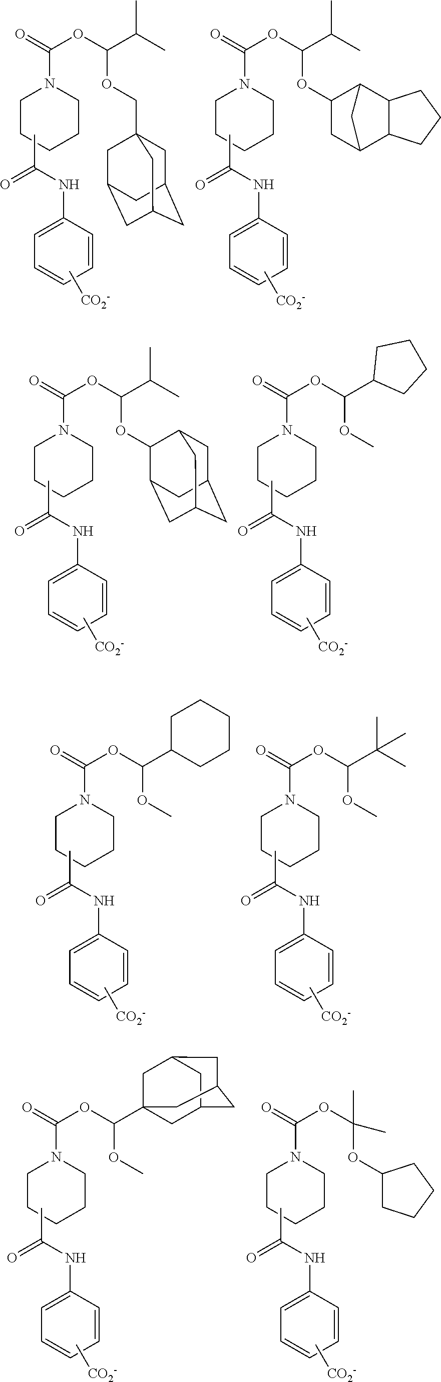

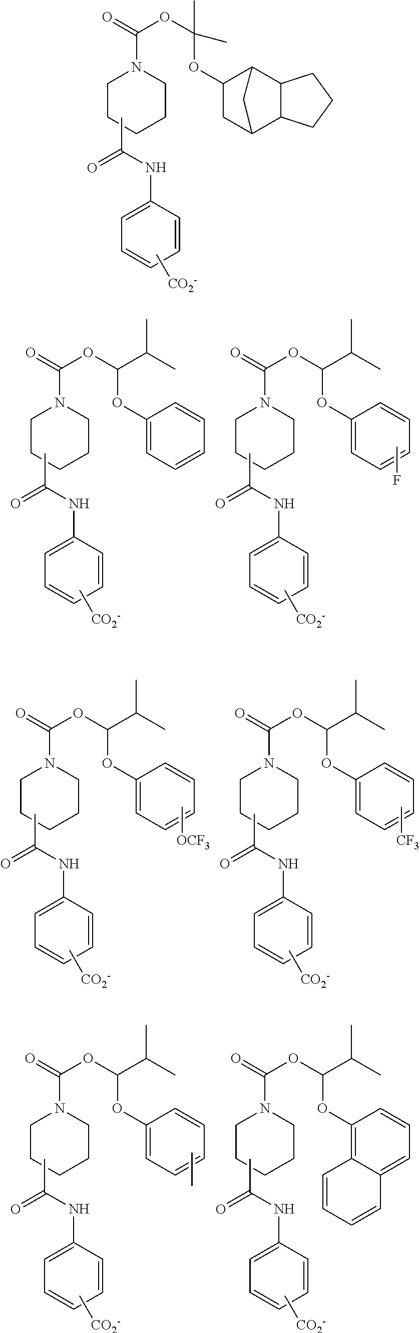

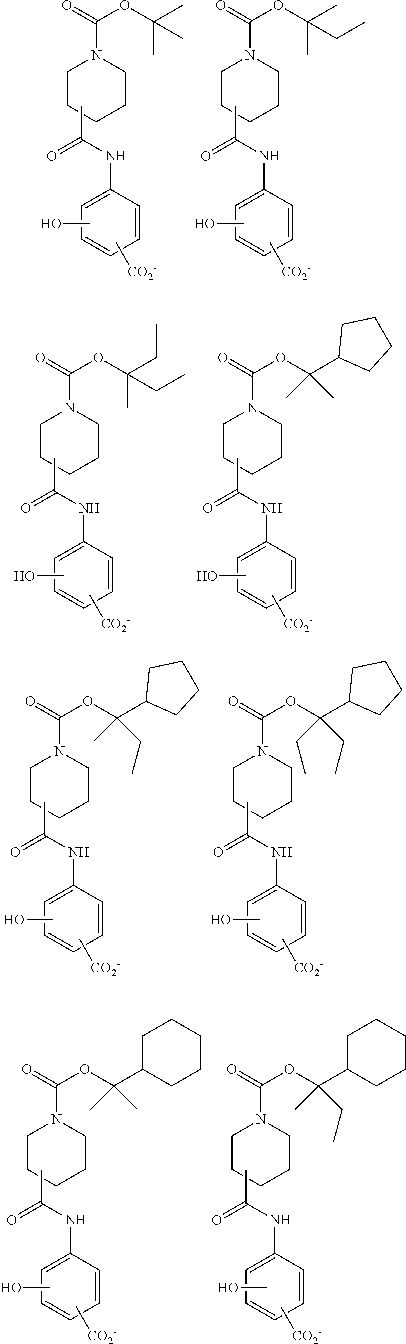

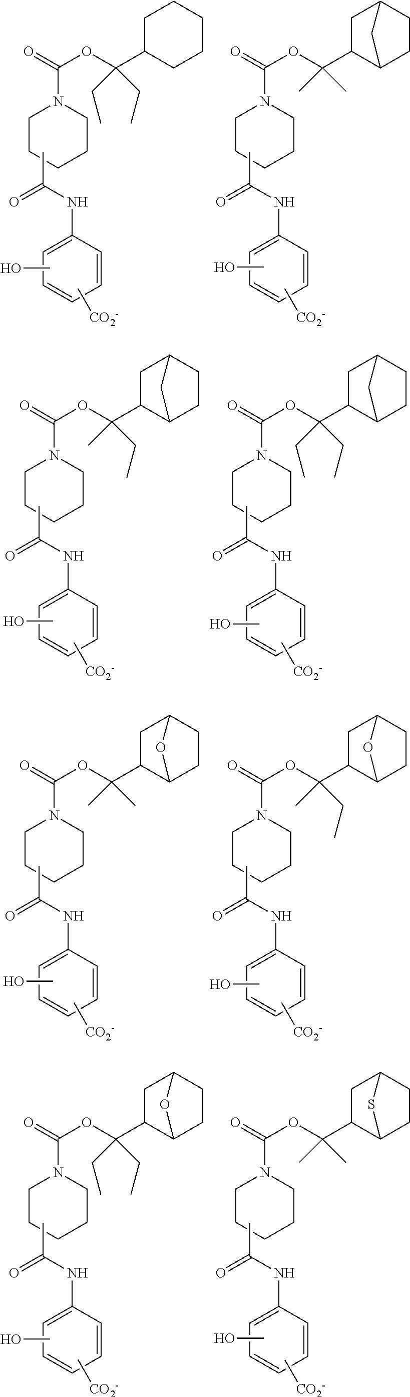

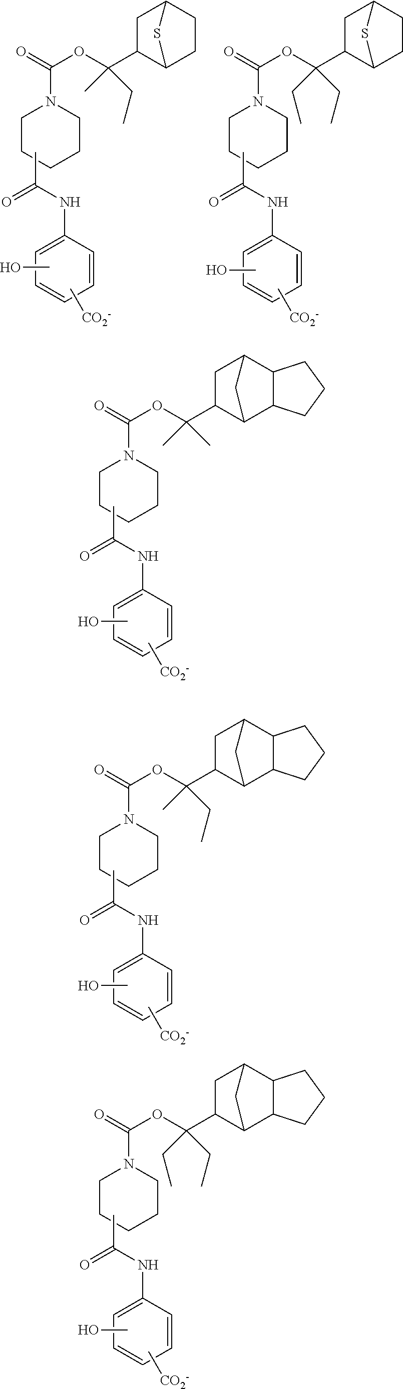

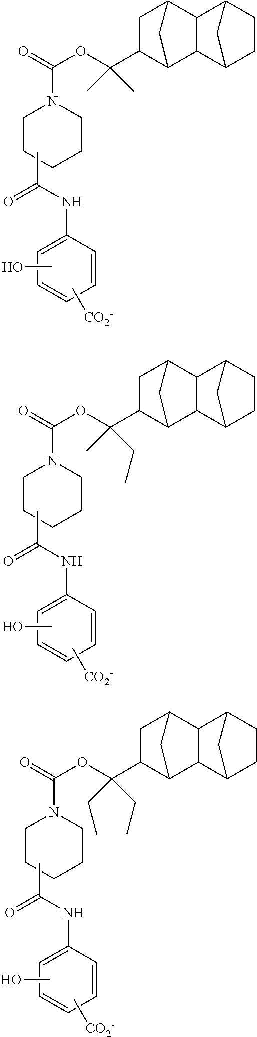

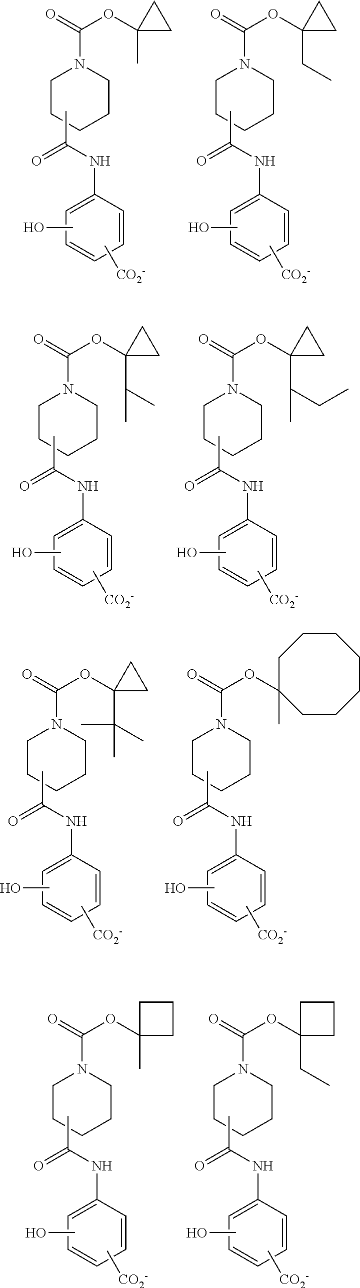

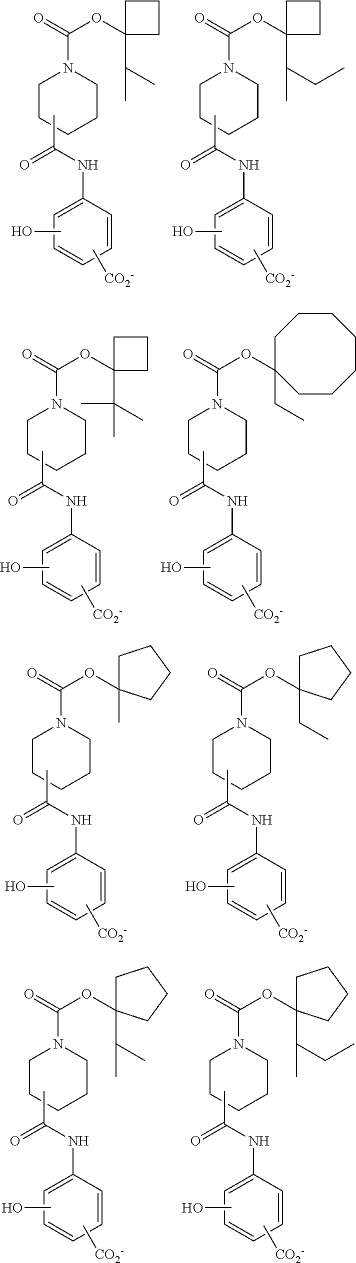

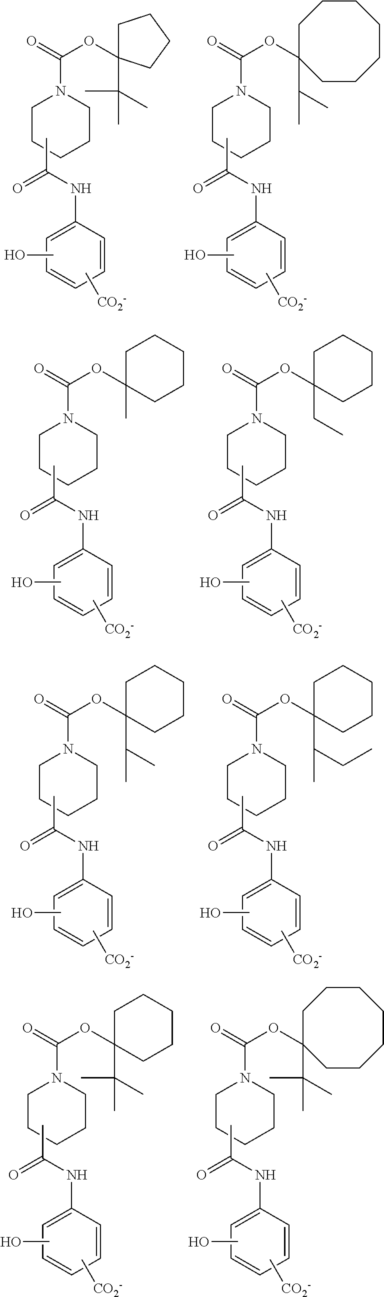

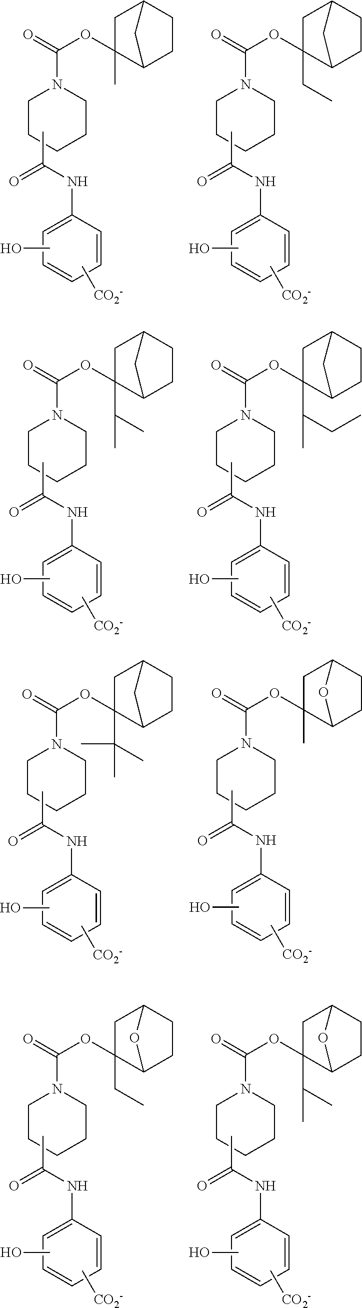

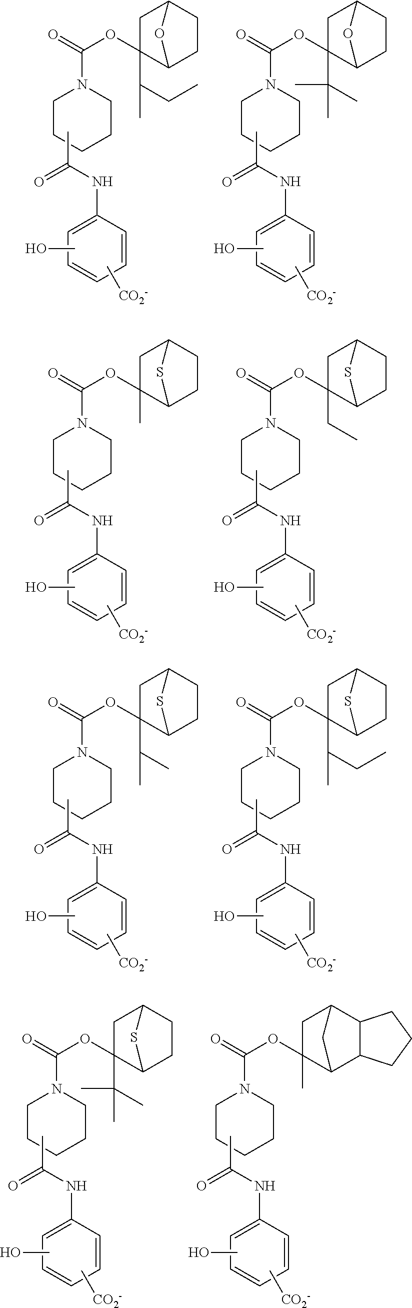

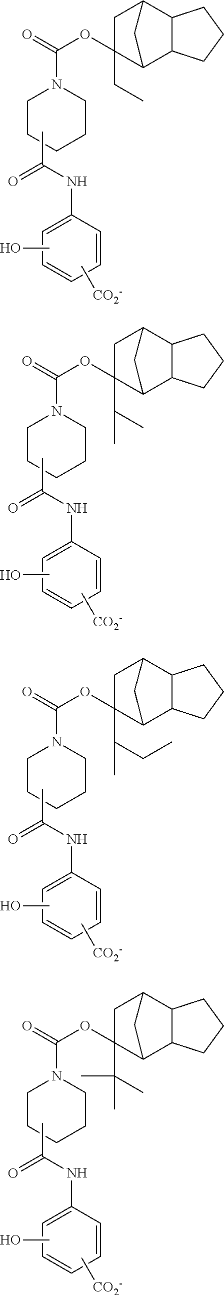

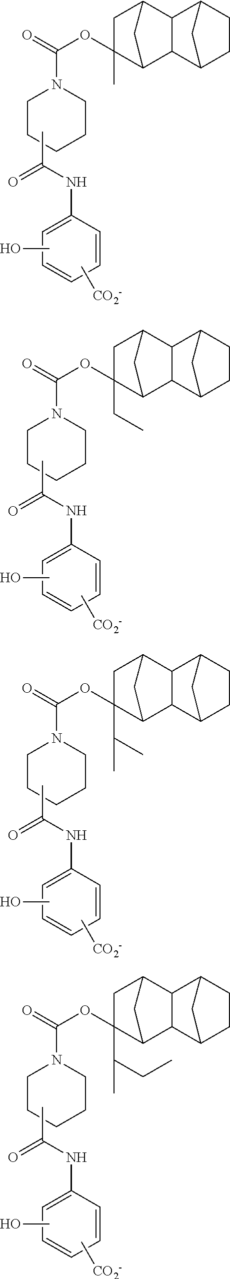

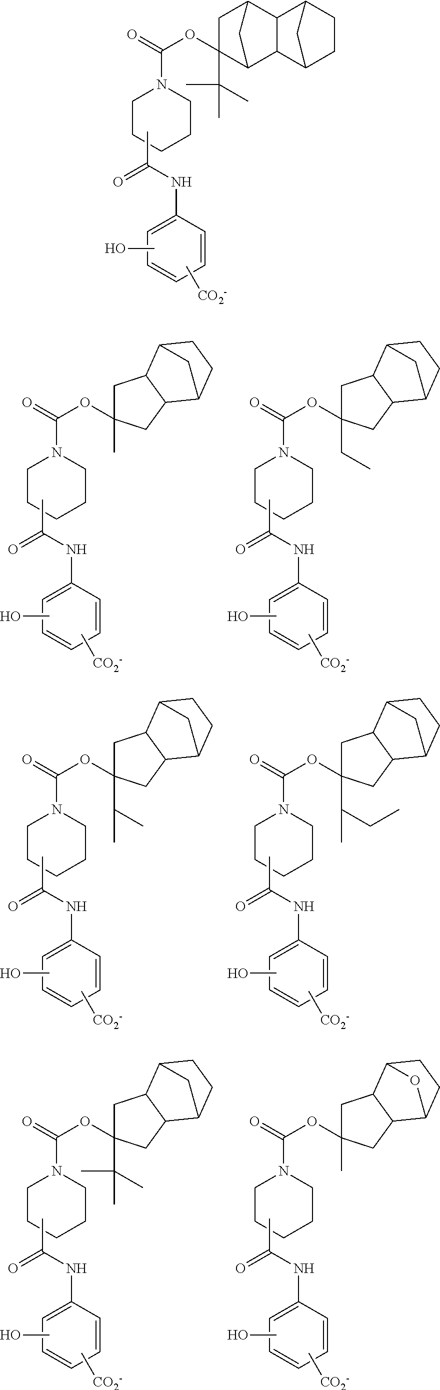

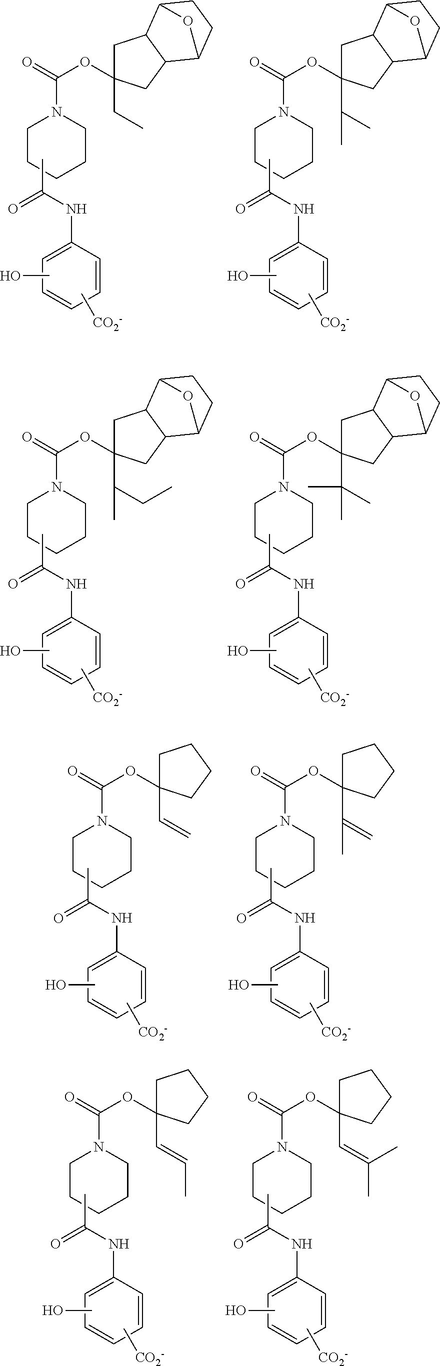

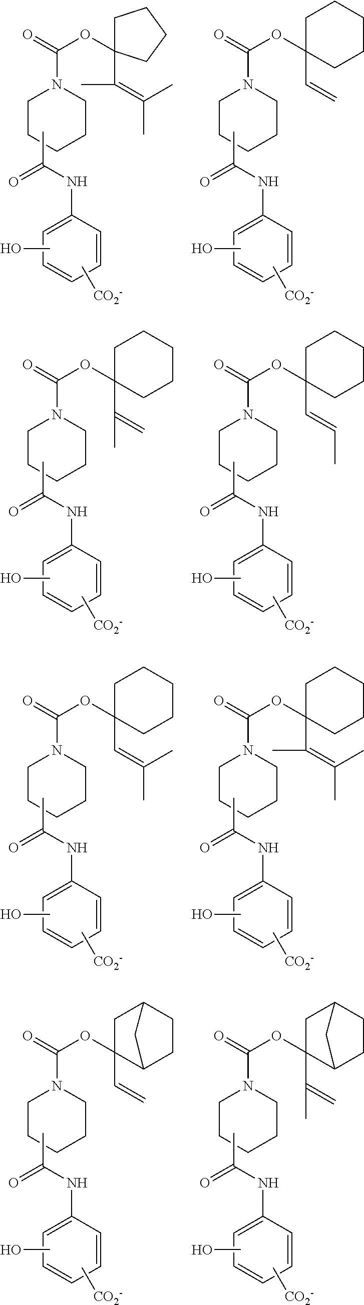

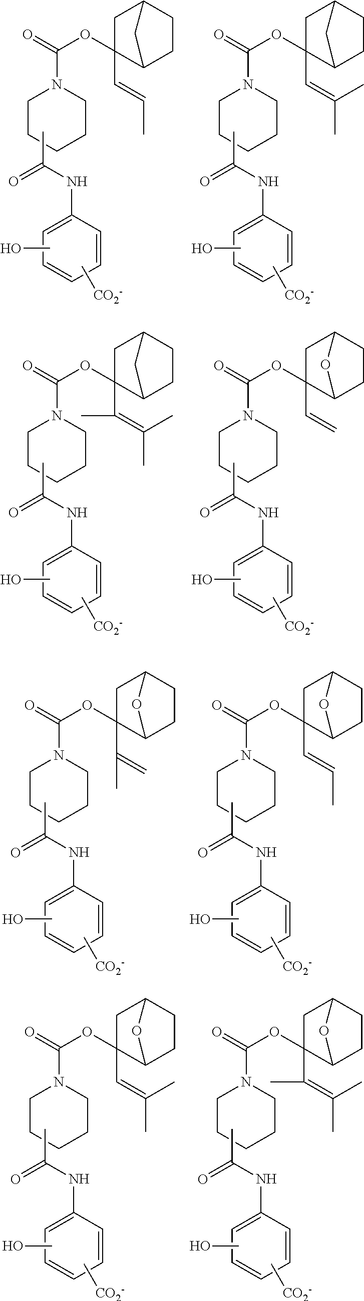

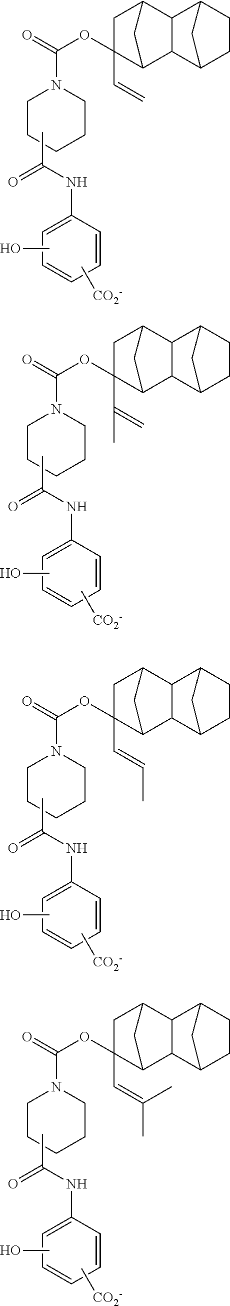

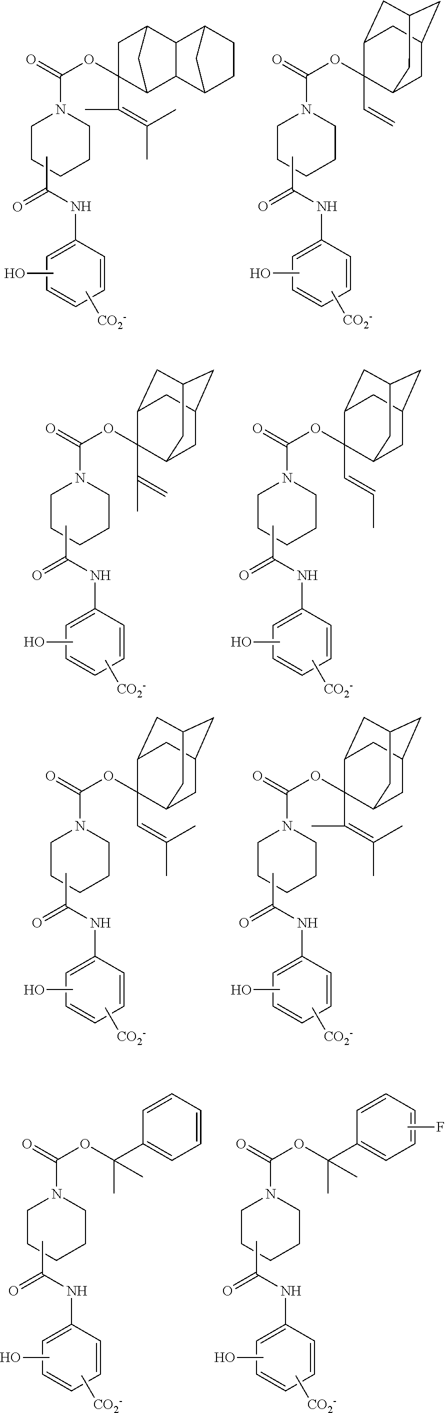

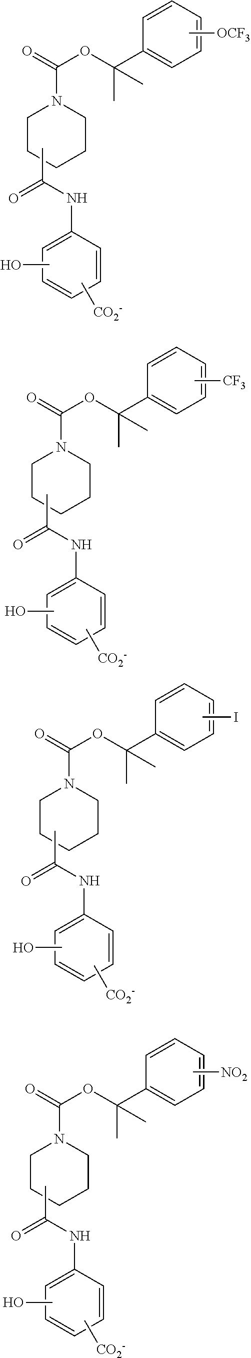

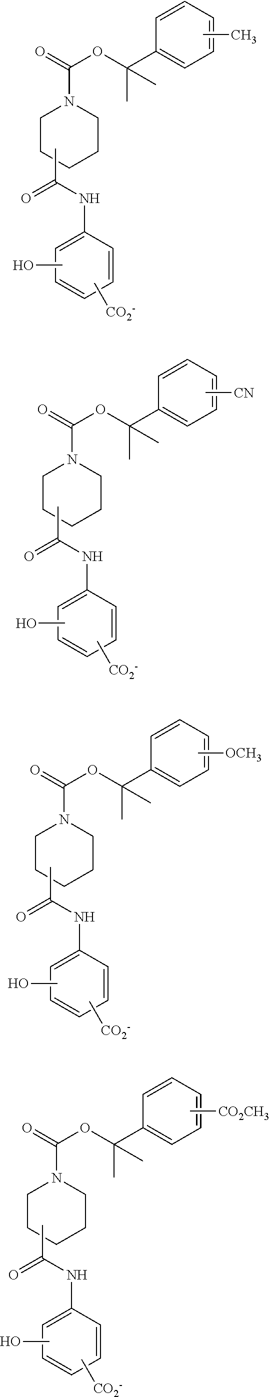

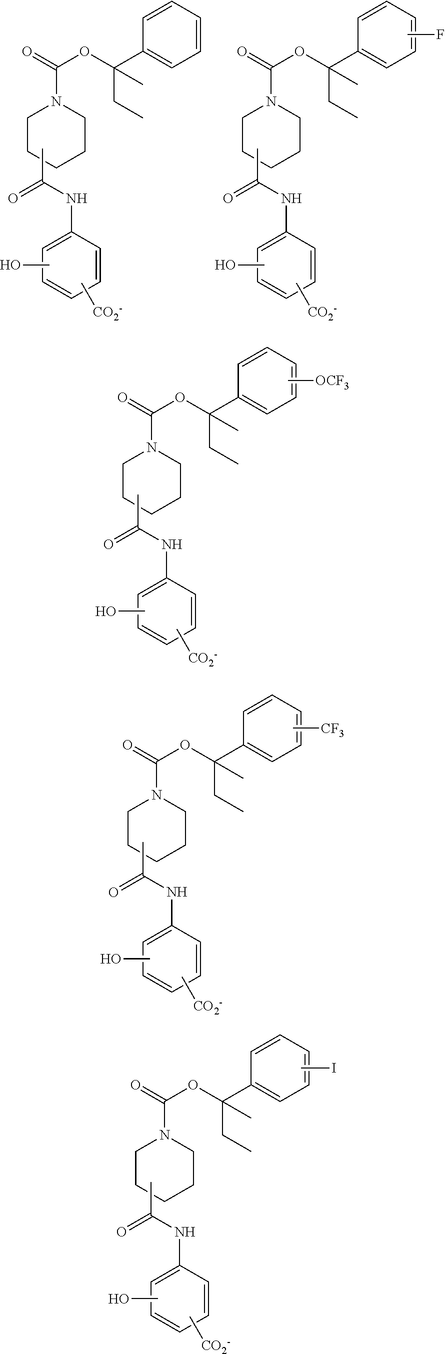

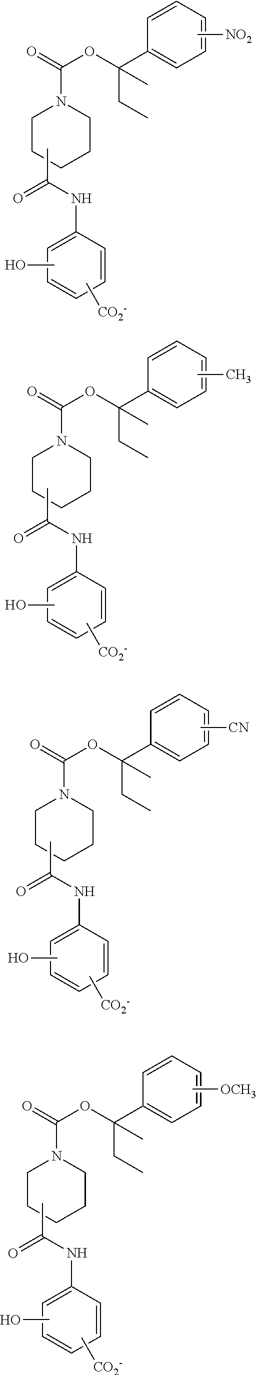

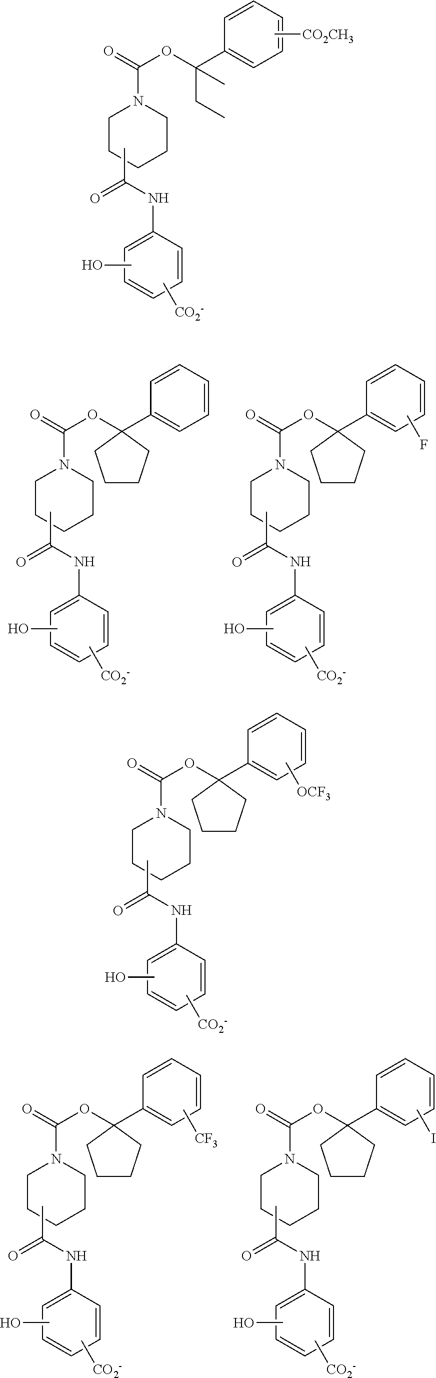

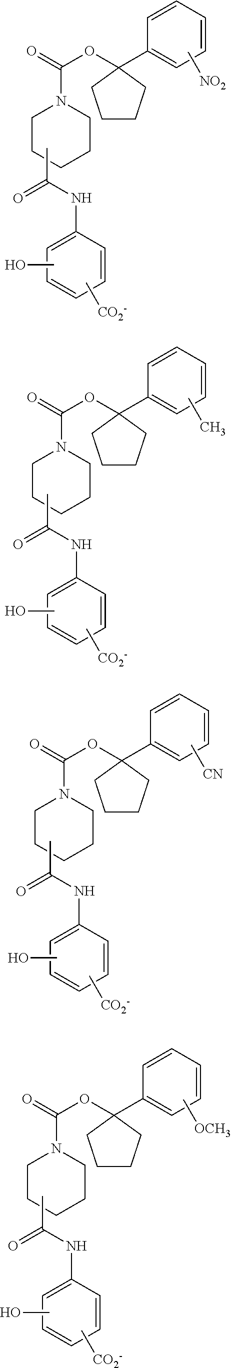

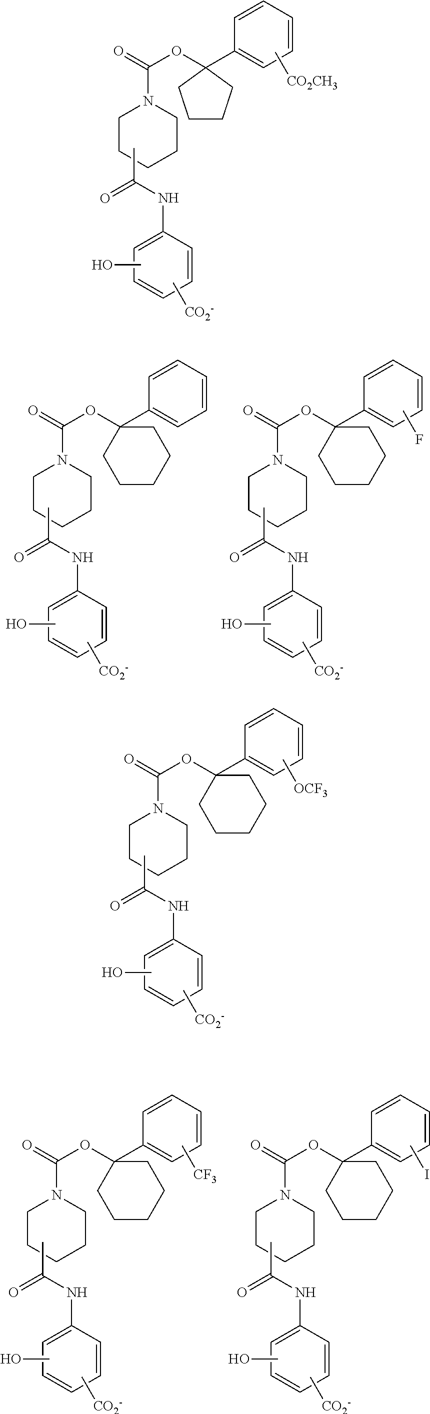

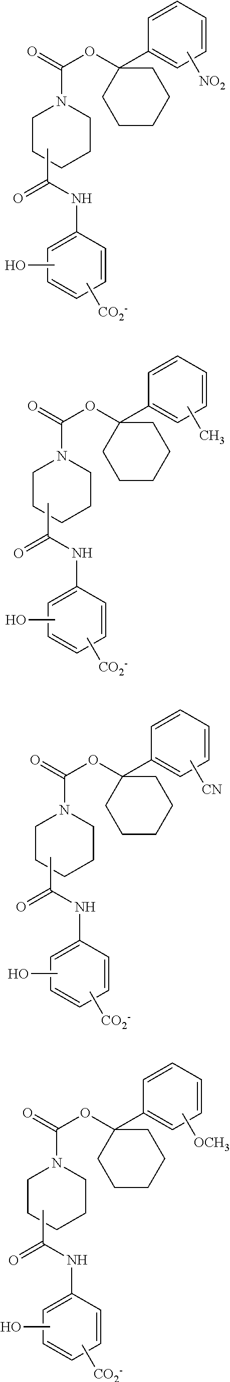

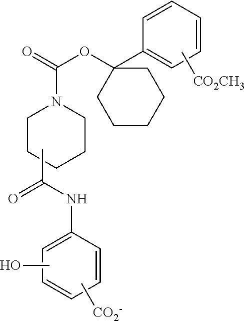

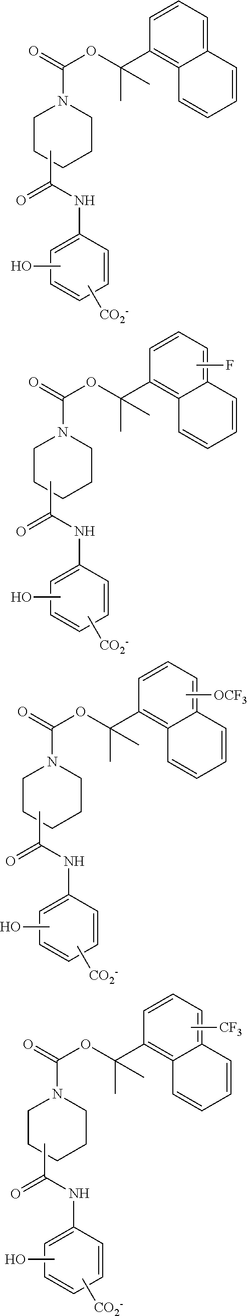

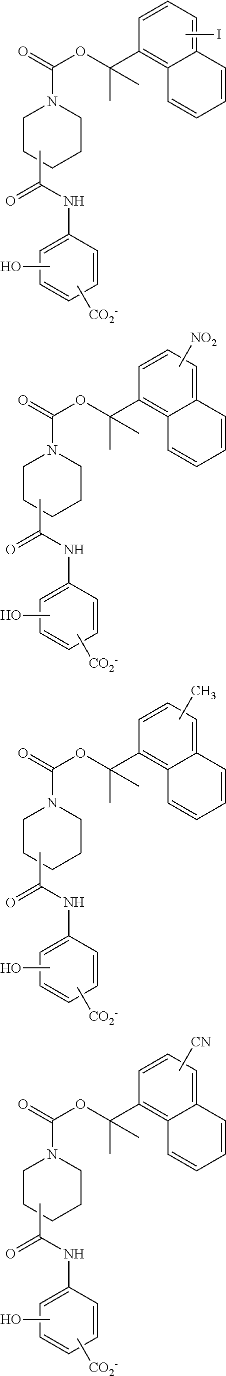

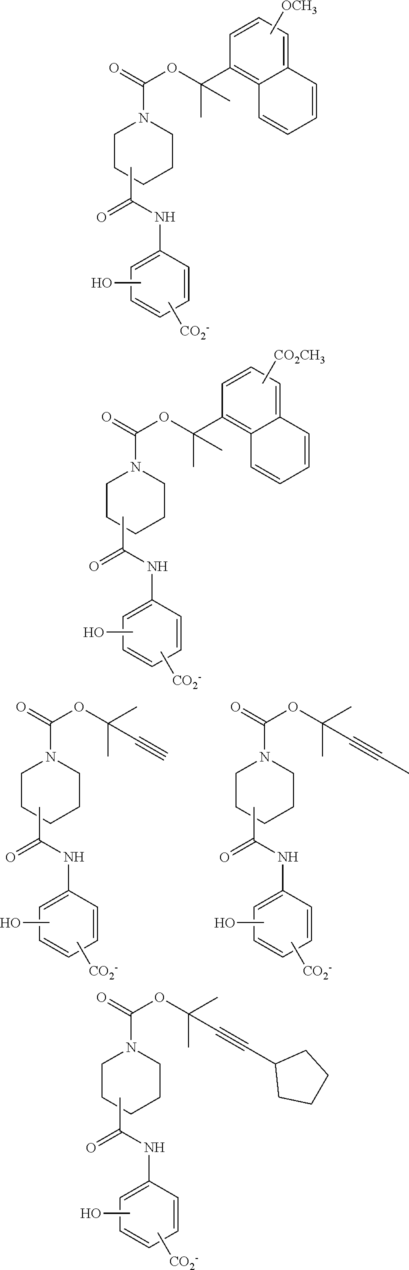

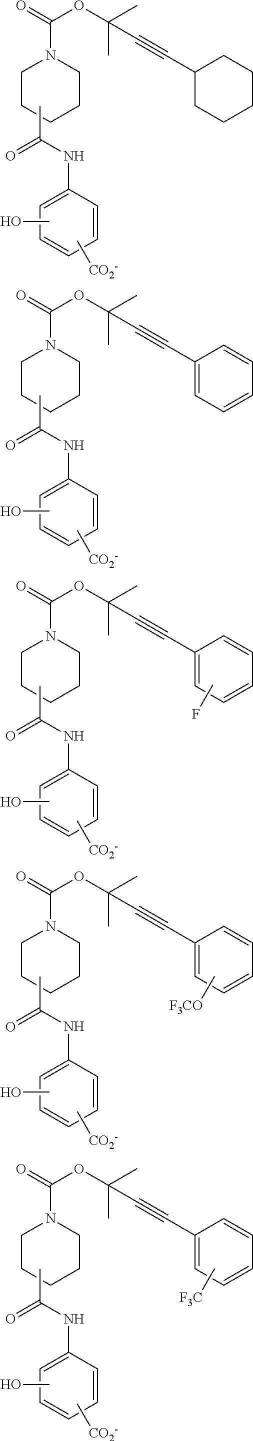

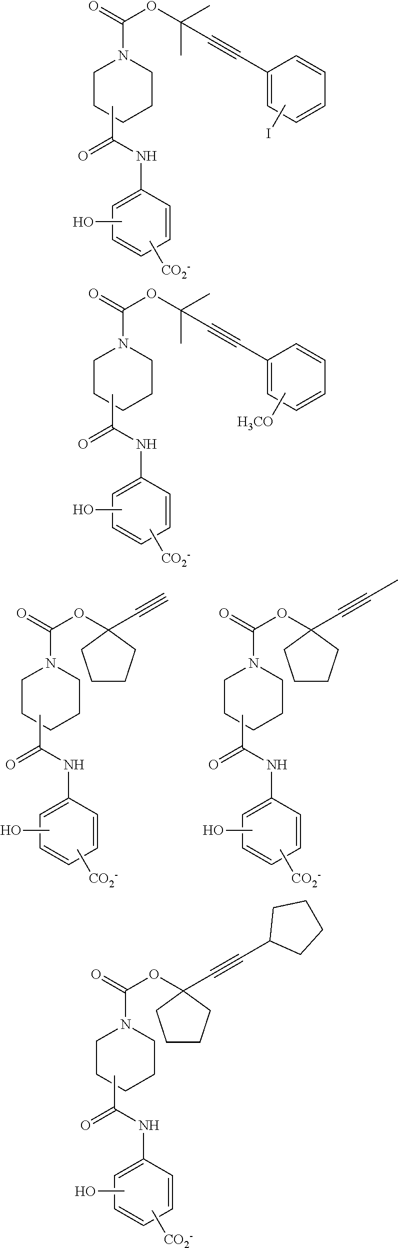

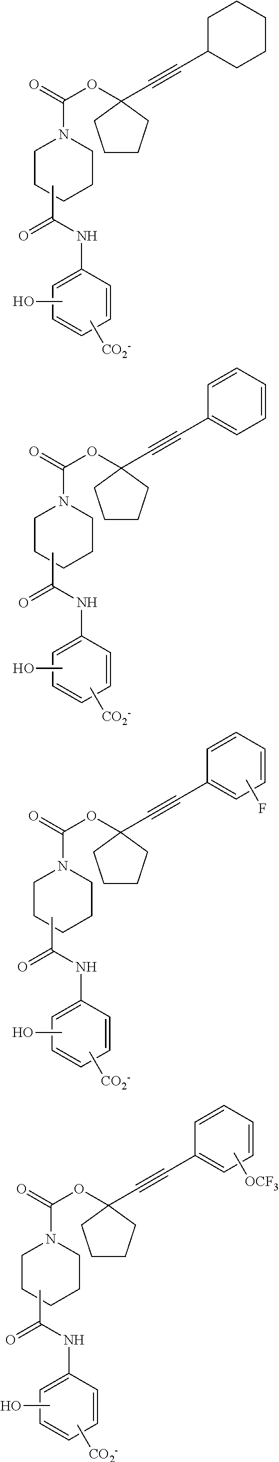

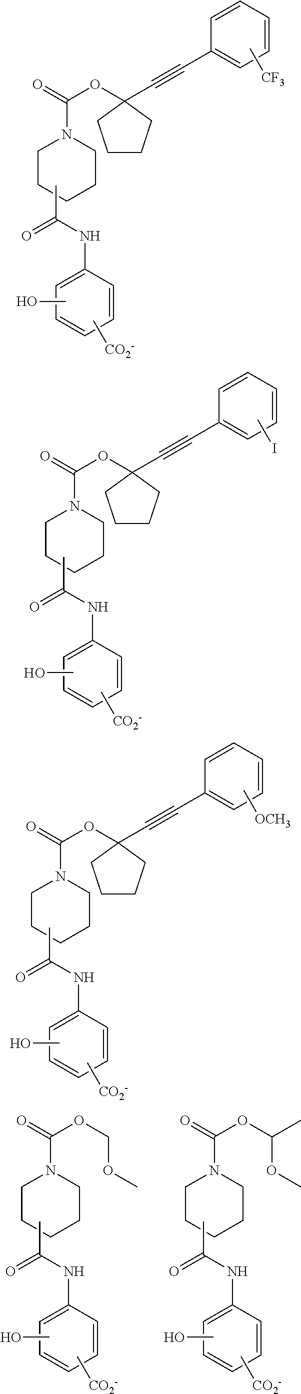

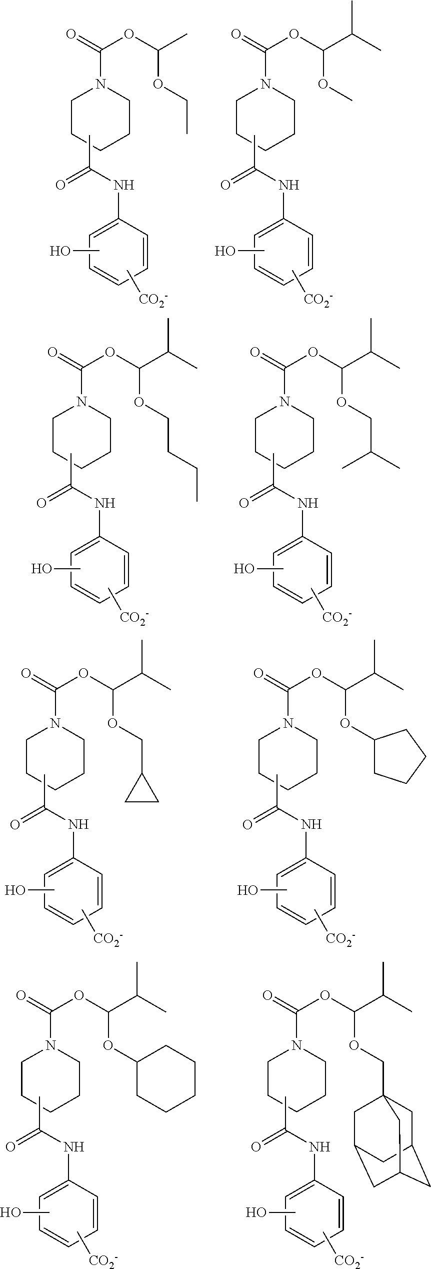

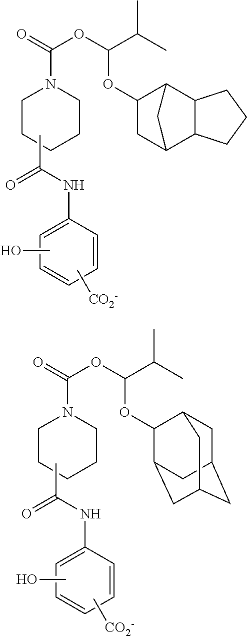

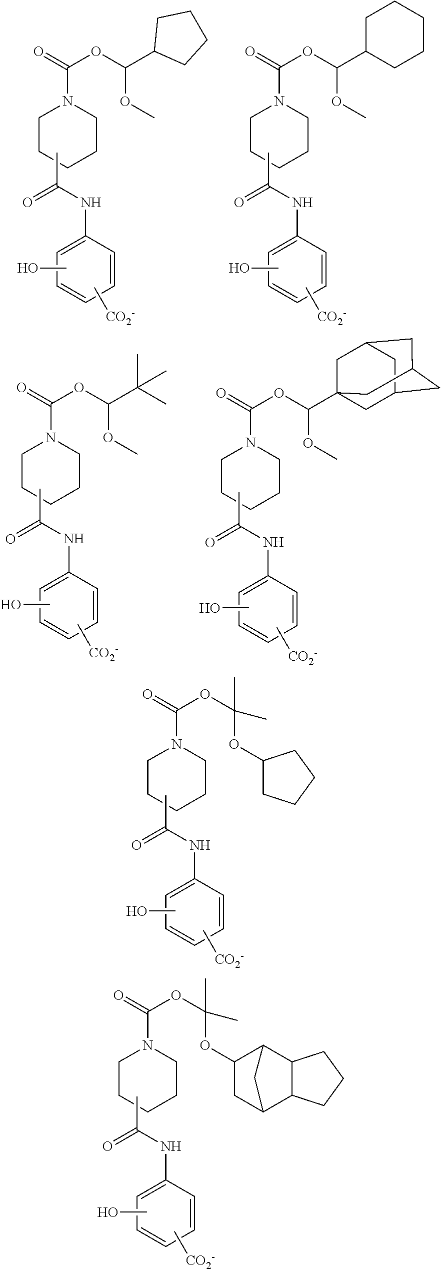

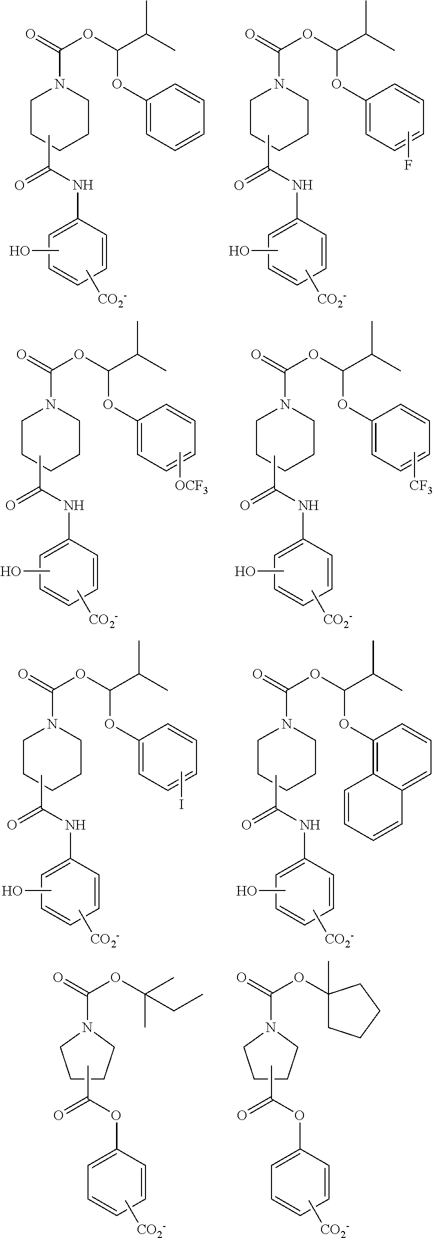

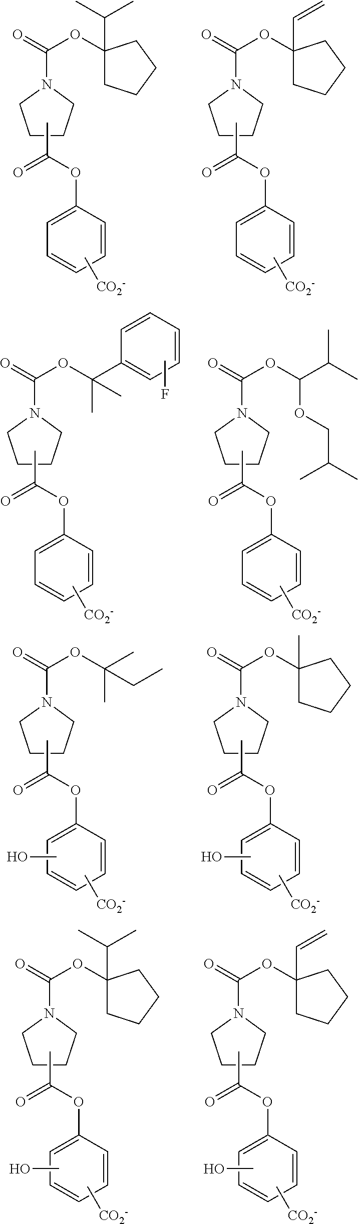

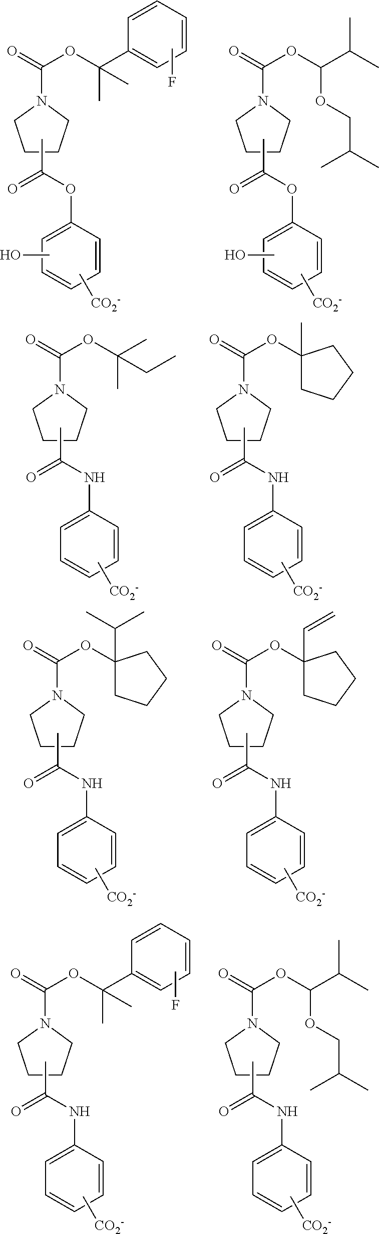

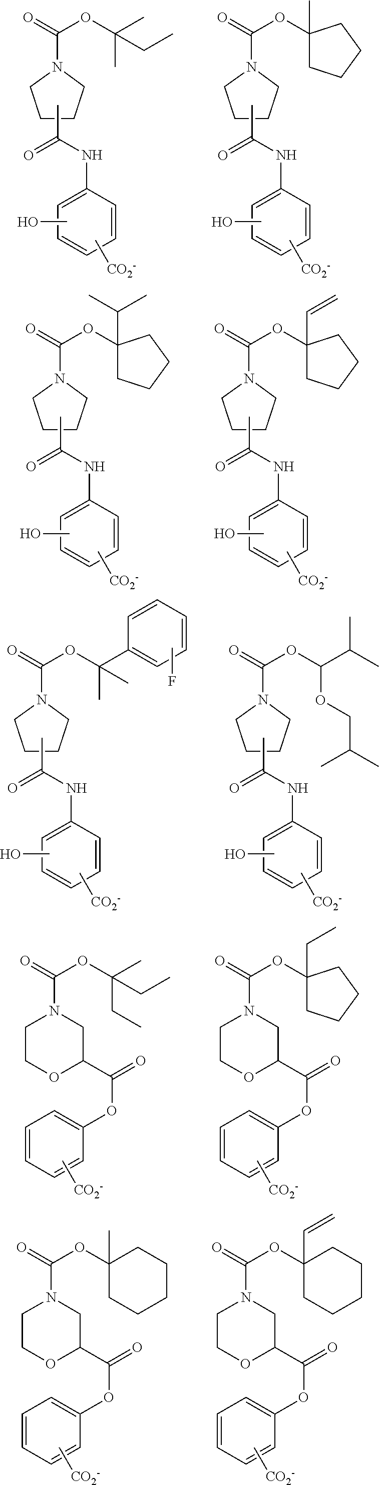

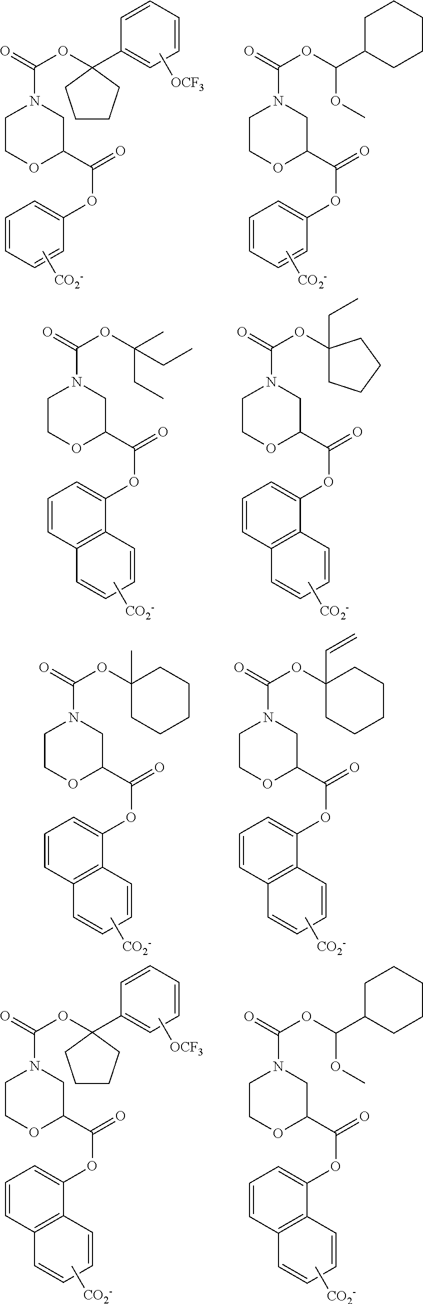

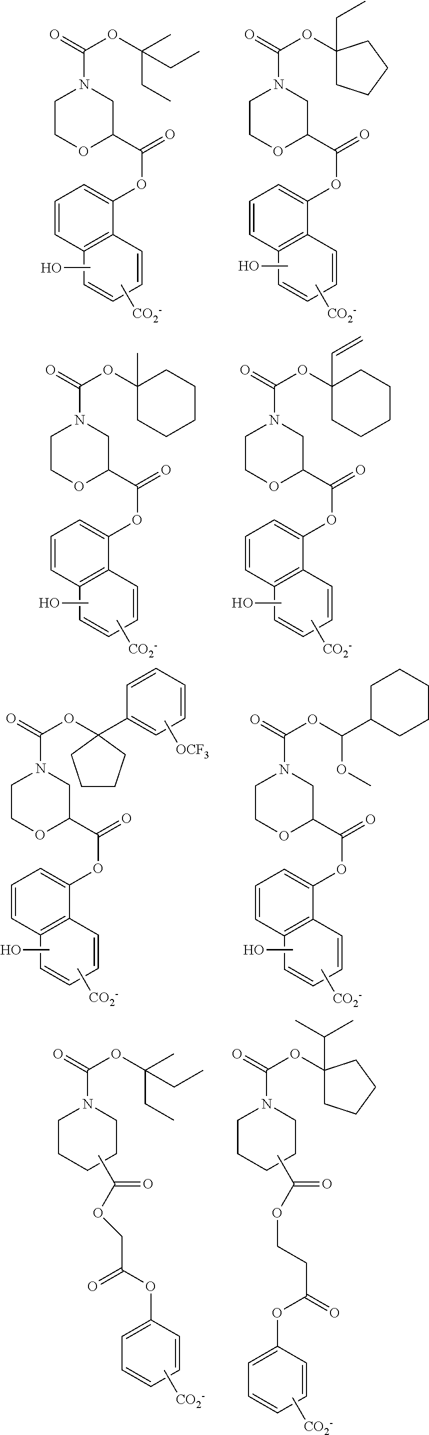

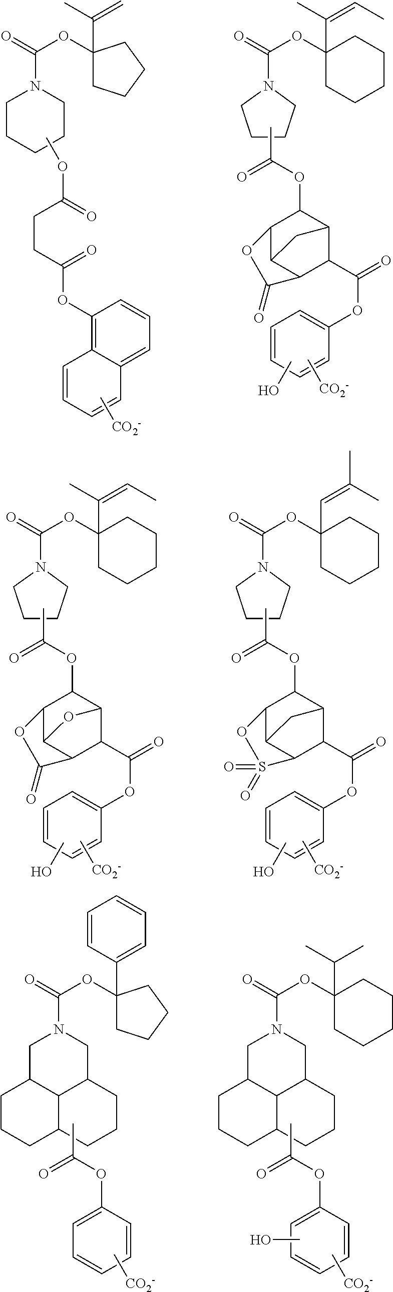

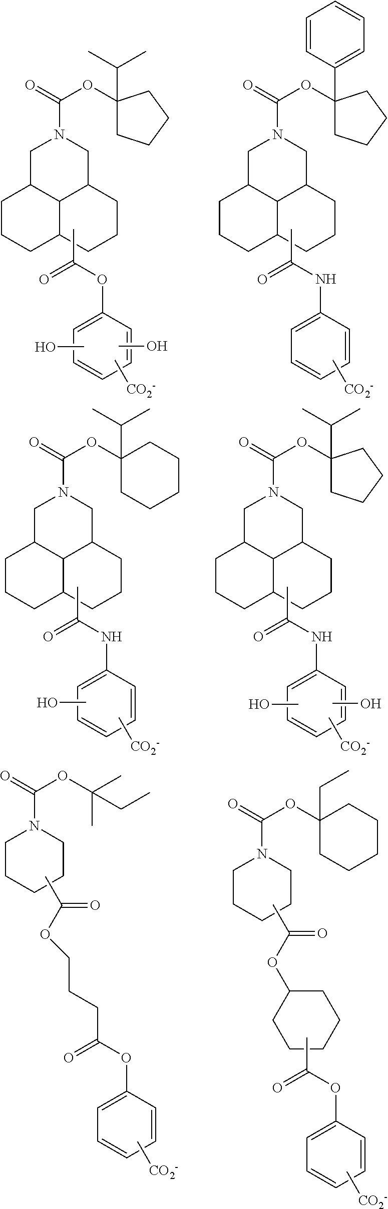

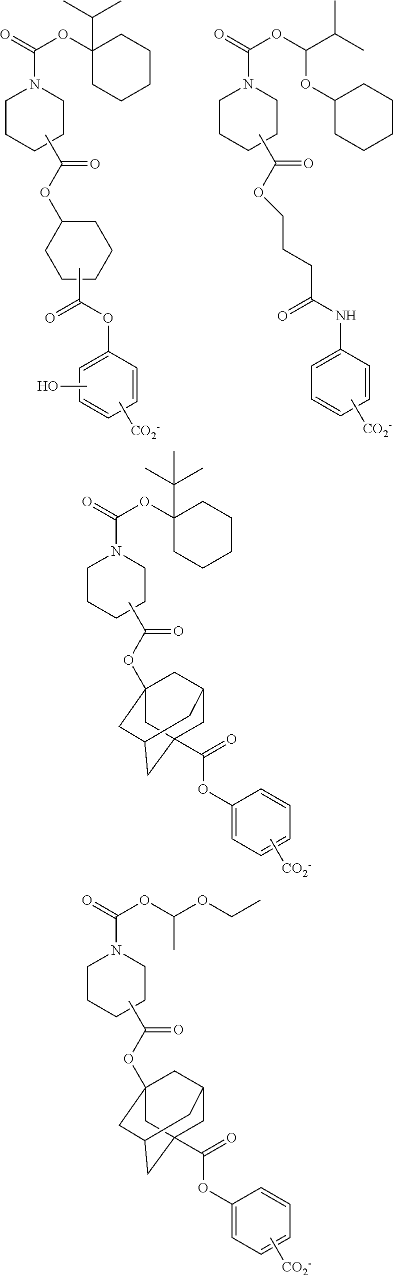

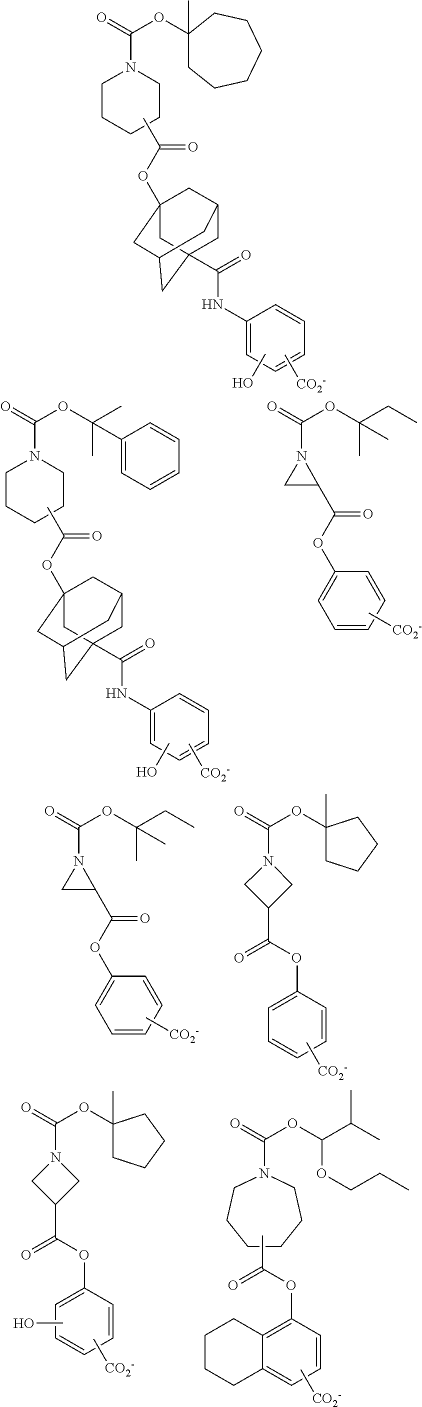

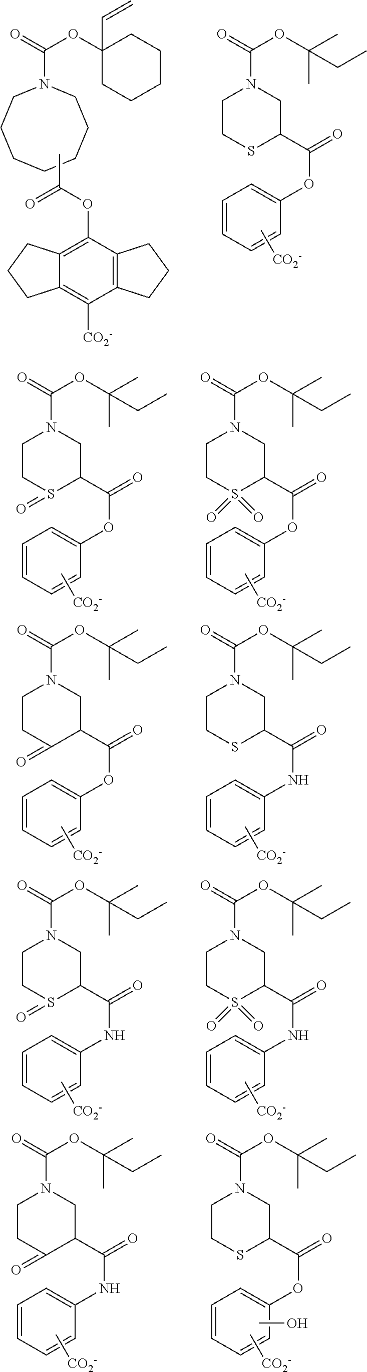

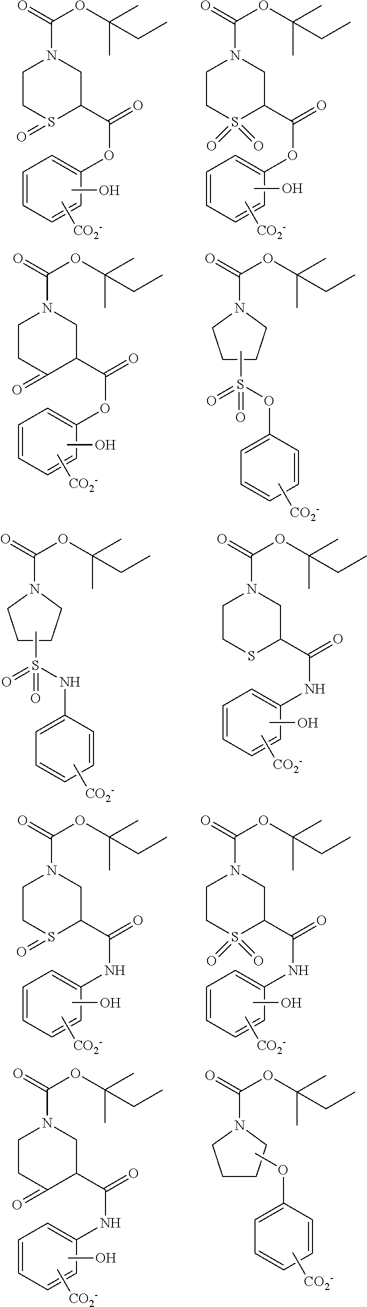

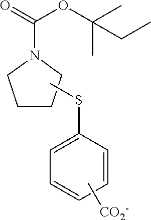

In formula (1), Z⁺ is an onium cation. The preferred onium cation has the formula (cation-1), (cation-2) or (cation-3).

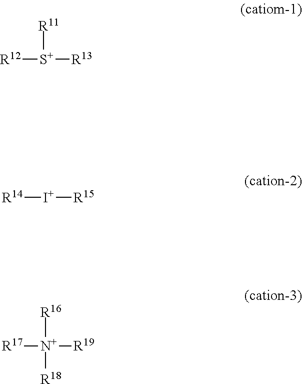

In formulae (cation-1) to (cation-3), R¹¹ to R¹⁹ are each independently a C₁-C₃₀ hydrocarbyl group which may contain a heteroatom. The hydrocarbyl group may be saturated or unsaturated and straight, branched or cyclic. Examples thereof include C₁-C₃₀ alkyl groups such as methyl, ethyl, n-propyl, isopropyl, n-butyl, and tert-butyl; C₃-C₃₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₃₀ alkenyl groups such as vinyl, allyl, propenyl, butenyl, and hexenyl; C₃-C₃₀ cyclic unsaturated hydrocarbyl groups such as cyclohexenyl; C₆-C₃₀ aryl groups such as phenyl, naphthyl and thienyl; C₇-C₃₀ aralkyl groups such as benzyl, 1-phenylethyl and 2-phenylethyl, and combinations thereof. Inter alia, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

Also, R¹¹ and R¹² may bond together to form a ring with the sulfur atom to which they are attached. Examples of the sulfonium cation having formula (cation-1) wherein R¹¹ and R¹² form a ring are shown below.

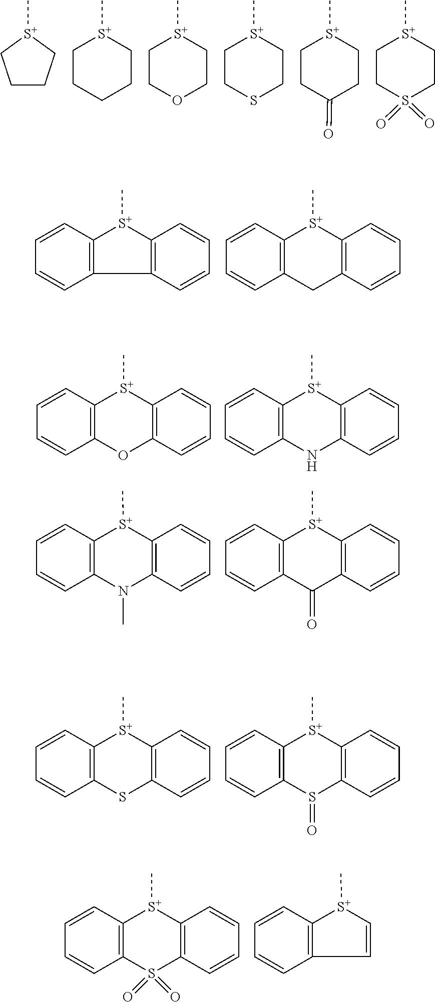

Herein the broken line designates a point of attachment to R¹³.

Examples of the sulfonium cation having formula (cation-1) are shown below, but not limited thereto.

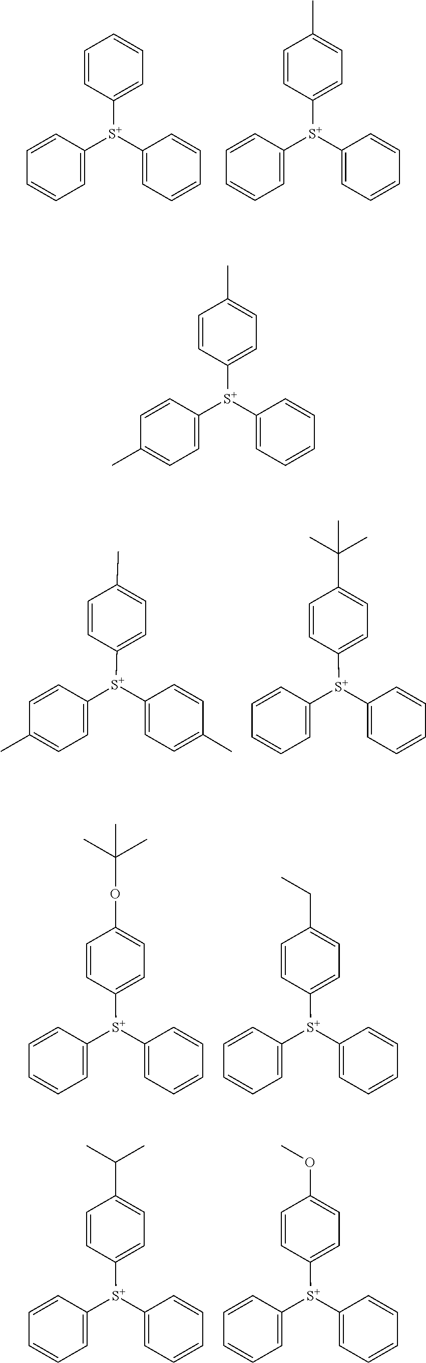

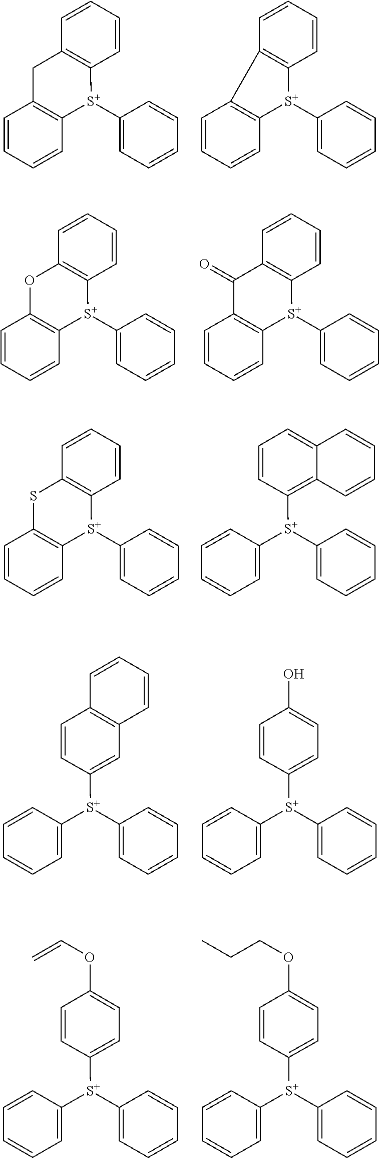

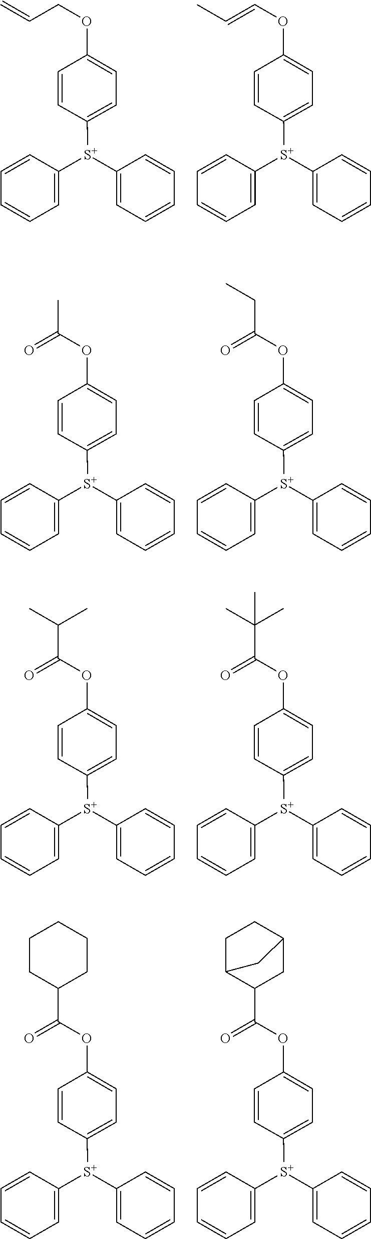

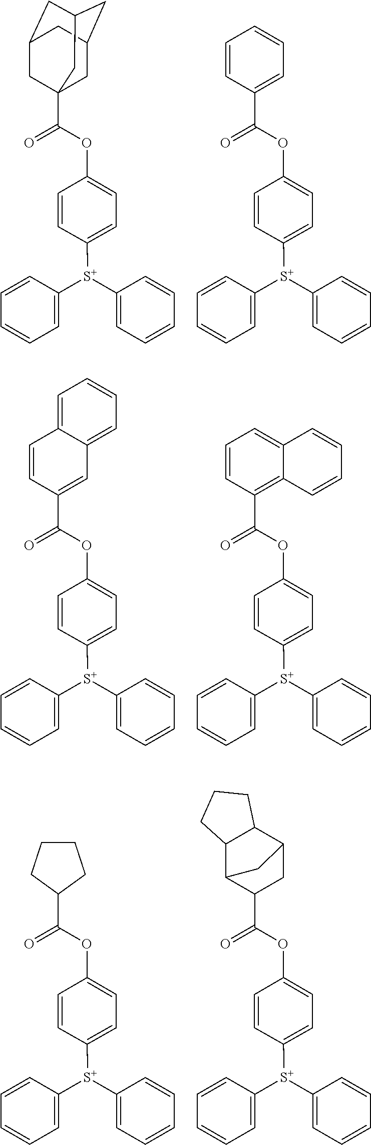 [SMILES: O=C(Oc1ccc([S+](c2ccccc2)c2ccccc2)cc1)C12CC3CC(CC(C3)C1)C2 ; O=C(Oc1ccc([S+](c2ccccc2)c2ccccc2)cc1)c1ccccc1 ; O=C(Oc1ccc([S+](c2ccccc2)c2ccccc2)cc1)c1ccc2ccccc2c1 ; O=C(Oc1ccc([S+](c2ccccc2)c2ccccc2)cc1)c1cccc2ccccc12 ; O=C(Oc1ccc([S+](c2ccccc2)c2ccccc2)cc1)C1CCCC1 ; O=C(Oc1ccc([S+](c2ccccc2)c2ccccc2)cc1)C1CC2CC1C1CCCC21]

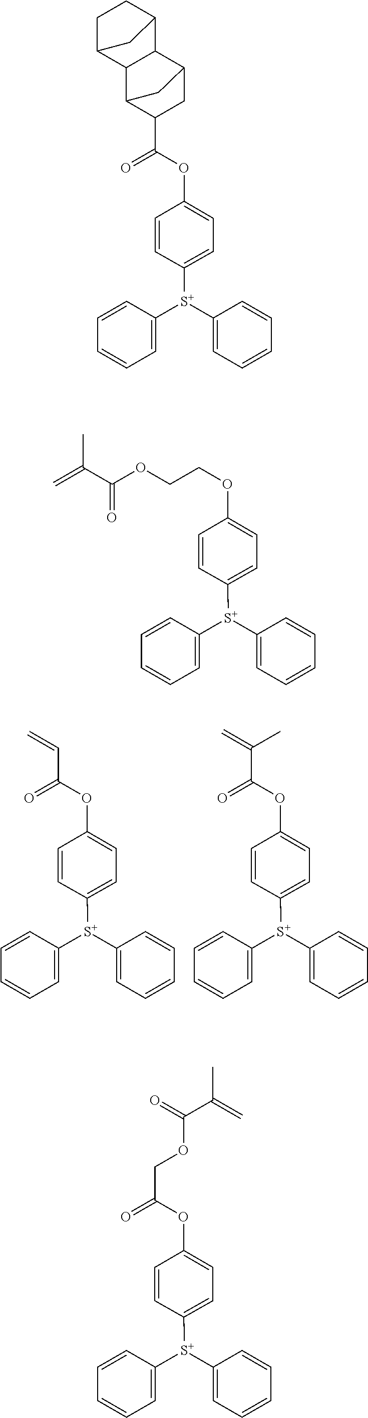 [SMILES: O=C(Oc1ccc([S+](c2ccccc2)c2ccccc2)cc1)C1CC2CC1C1C3CCC(C3)C21 ; C=C(C)C(=O)OCCOc1ccc([S+](c2ccccc2)c2ccccc2)cc1 ; C=CC(=O)Oc1ccc([S+](c2ccccc2)c2ccccc2)cc1 ; C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1]

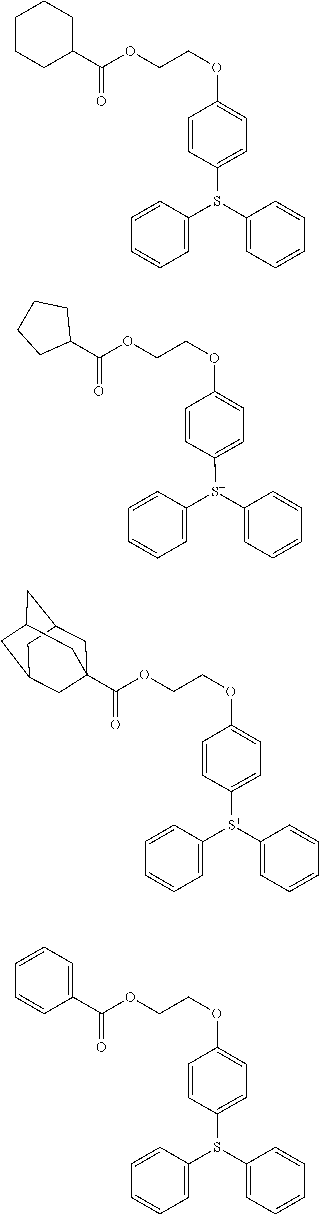 [SMILES: O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1]

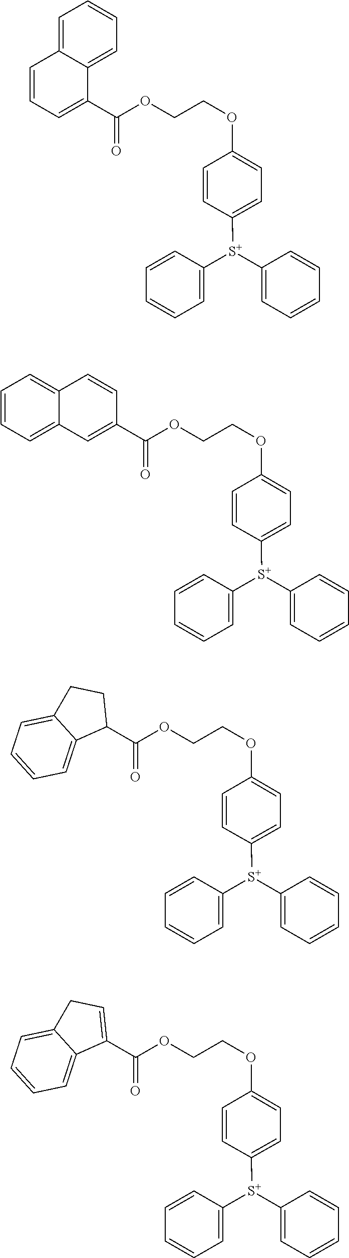 [SMILES: O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

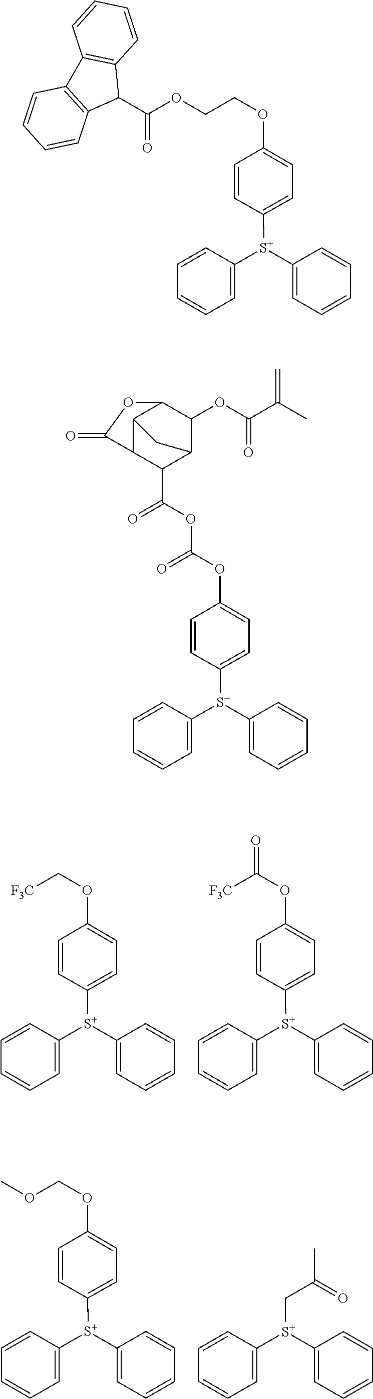 [SMILES: O=C(OCCOc1ccc([S+](c2ccccc2)c2ccccc2)cc1)C1c2ccccc2-c2ccccc21 ; C=C(C)C(=O)OC1C2CC3C1OC(=O)C3C2C(=O)OC(=O)Oc1ccc([S+](c2ccccc2)c2ccccc2)cc1 ; FC(F)(F)COc1ccc([S+](c2ccccc2)c2ccccc2)cc1 ; O=C(Oc1ccc([S+](c2ccccc2)c2ccccc2)cc1)C(F)(F)F ; COCOc1ccc([S+](c2ccccc2)c2ccccc2)cc1 ; CC(=O)C[S+](c1ccccc1)c1ccccc1]

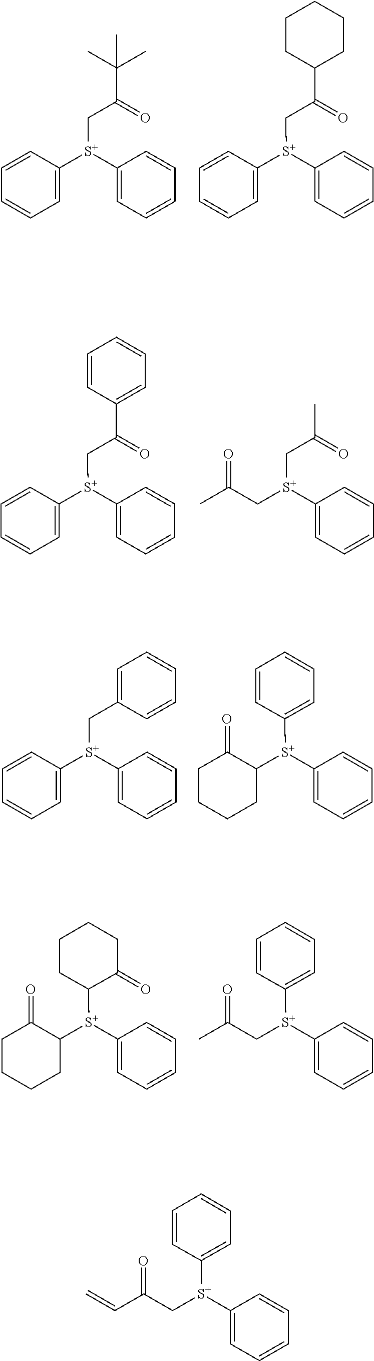

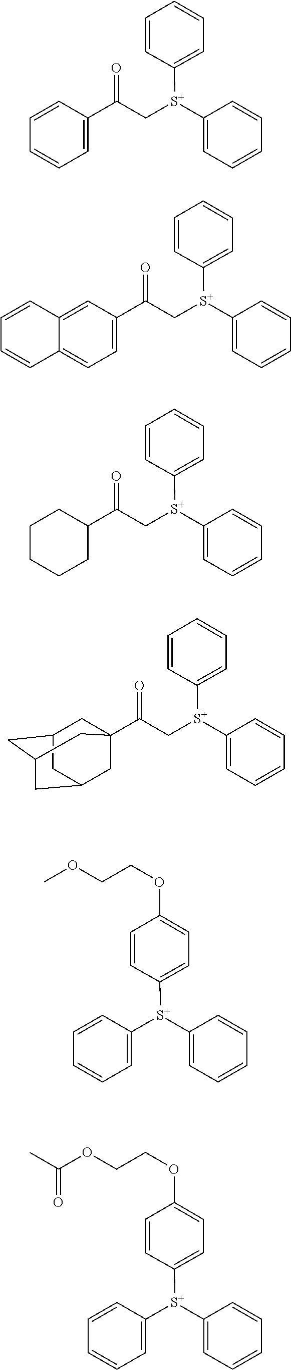 [SMILES: O=C(C[S+](c1ccccc1)c1ccccc1)c1ccccc1 ; O=C(C[S+](c1ccccc1)c1ccccc1)c1ccc2ccccc2c1 ; O=C(C[S+](c1ccccc1)c1ccccc1)C1CCCCC1 ; O=C(C[S+](c1ccccc1)c1ccccc1)C12CC3CC(CC(C3)C1)C2 ; COCCOc1ccc([S+](c2ccccc2)c2ccccc2)cc1 ; CC(=O)OCCOc1ccc([S+](c2ccccc2)c2ccccc2)cc1]

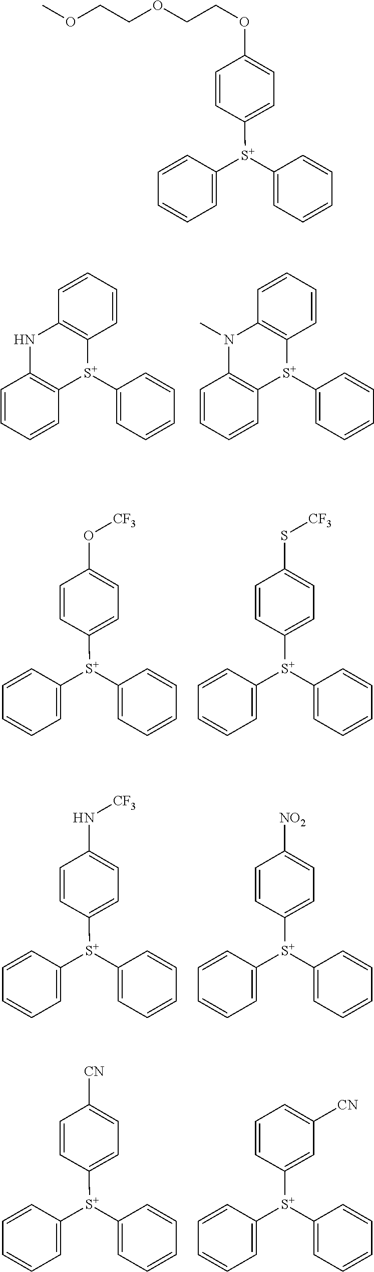

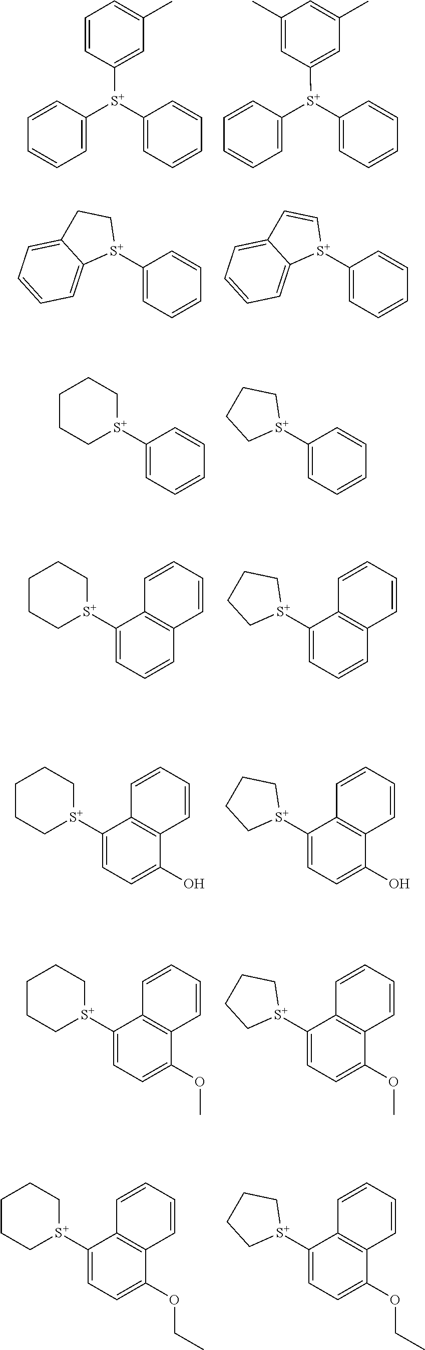

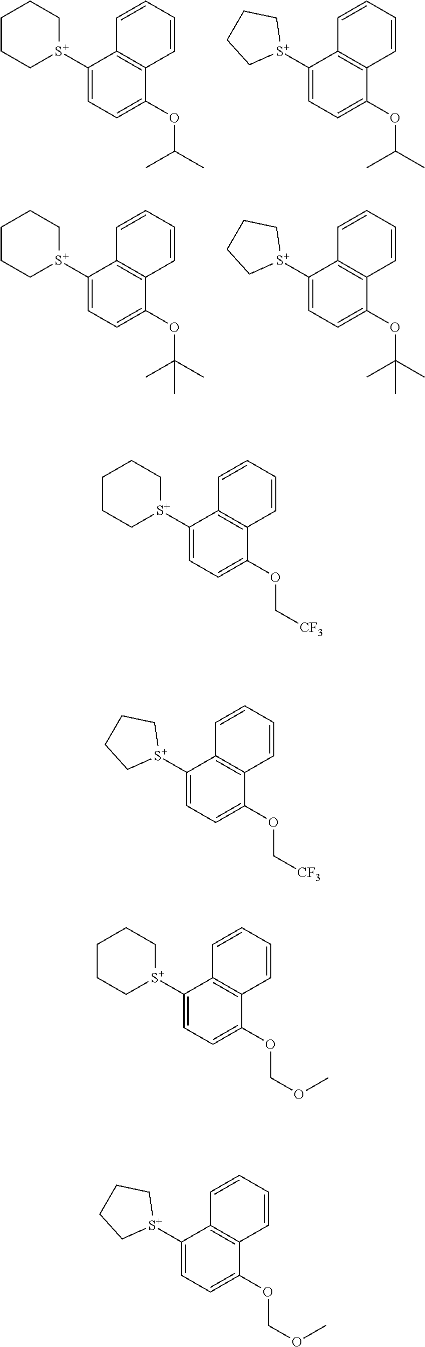

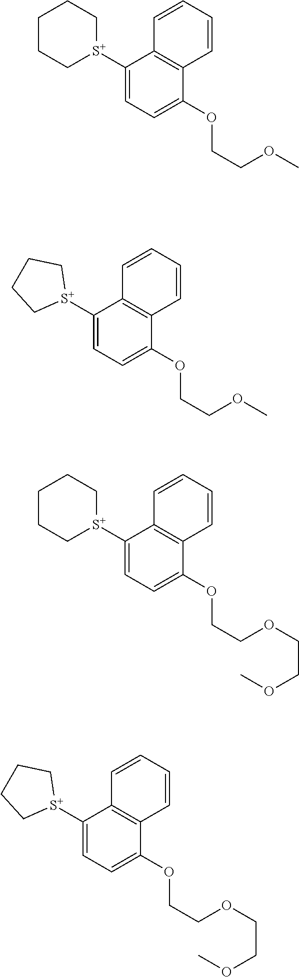 [SMILES: COCCOc1ccc([S+]2CCCCC2)c2ccccc12 ; COCCOc1ccc([S+]2CCCC2)c2ccccc12 ; COCCOCCOc1ccc([S+]2CCCCC2)c2ccccc12 ; COCCOCCOc1ccc([S+]2CCCC2)c2ccccc12]

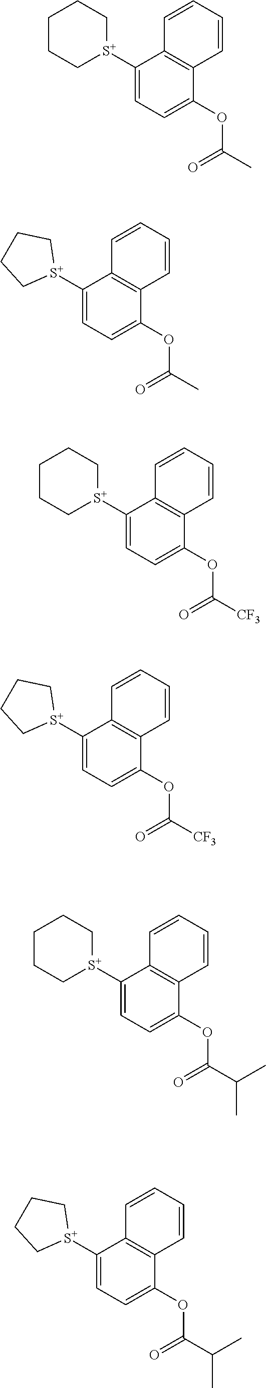 [SMILES: CC(=O)Oc1ccc([S+]2CCCCC2)c2ccccc12 ; CC(=O)Oc1ccc([S+]2CCCC2)c2ccccc12 ; O=C(Oc1ccc([S+]2CCCCC2)c2ccccc12)C(F)(F)F ; O=C(Oc1ccc([S+]2CCCC2)c2ccccc12)C(F)(F)F ; CC(C)C(=O)Oc1ccc([S+]2CCCCC2)c2ccccc12 ; CC(C)C(=O)Oc1ccc([S+]2CCCC2)c2ccccc12]

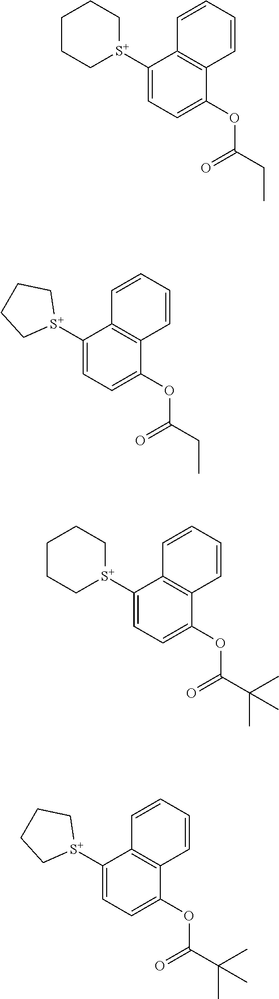 [SMILES: CCC(=O)Oc1ccc([S+]2CCCCC2)c2ccccc12 ; CCC(=O)Oc1ccc([S+]2CCCC2)c2ccccc12 ; CC(C)(C)C(=O)Oc1ccc([S+]2CCCCC2)c2ccccc12 ; CC(C)(C)C(=O)Oc1ccc([S+]2CCCC2)c2ccccc12]

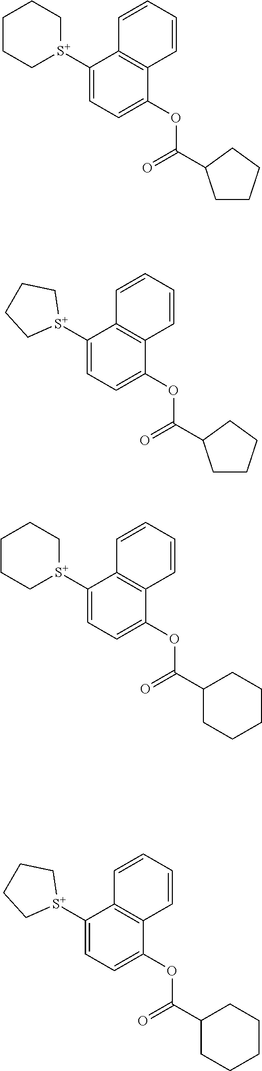 [SMILES: O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1]

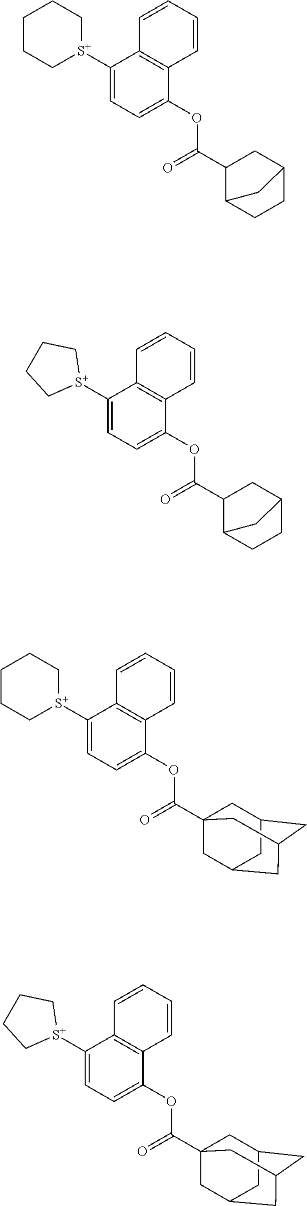 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2]

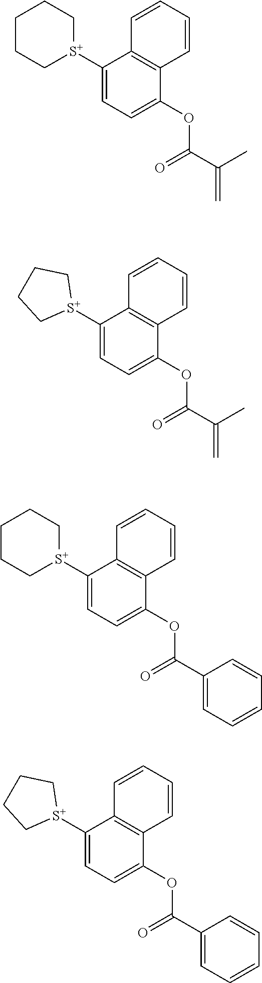 [SMILES: C=C(C)C(=O)Oc1ccc([S+]2CCCCC2)c2ccccc12 ; C=C(C)C(=O)Oc1ccc([S+]2CCCC2)c2ccccc12 ; O=C(Oc1ccc([S+]2CCCCC2)c2ccccc12)c1ccccc1 ; O=C(Oc1ccc([S+]2CCCC2)c2ccccc12)c1ccccc1]

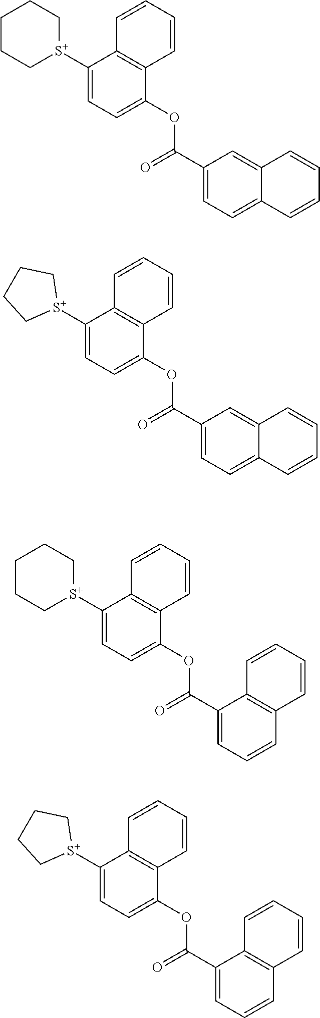 [SMILES: O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12]

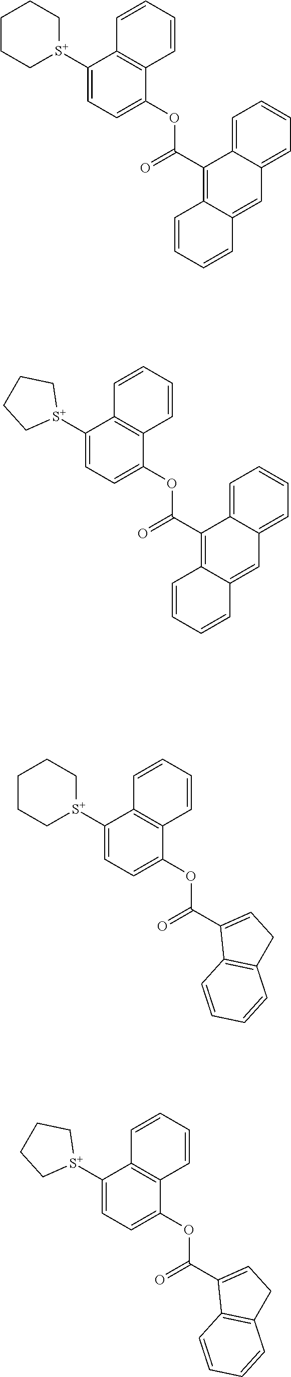 [SMILES: O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21]

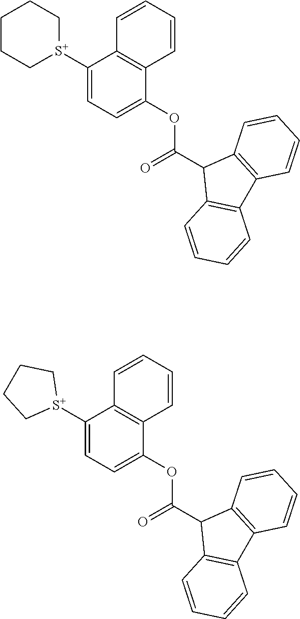 [SMILES: O=C(Oc1ccc([S+]2CCCCC2)c2ccccc12)C1c2ccccc2-c2ccccc21 ; O=C(Oc1ccc([S+]2CCCC2)c2ccccc12)C1c2ccccc2-c2ccccc21]

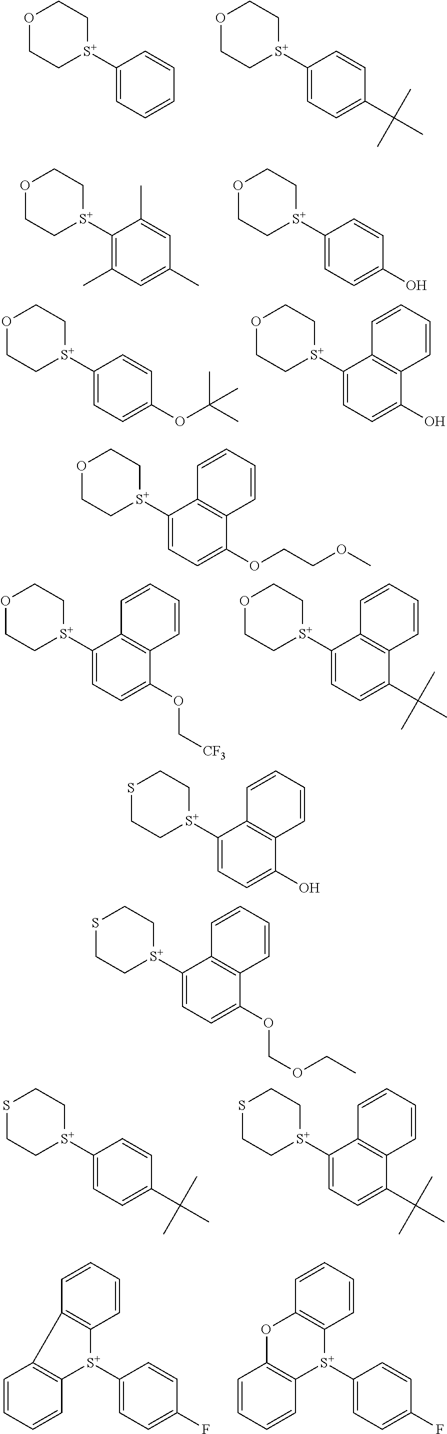

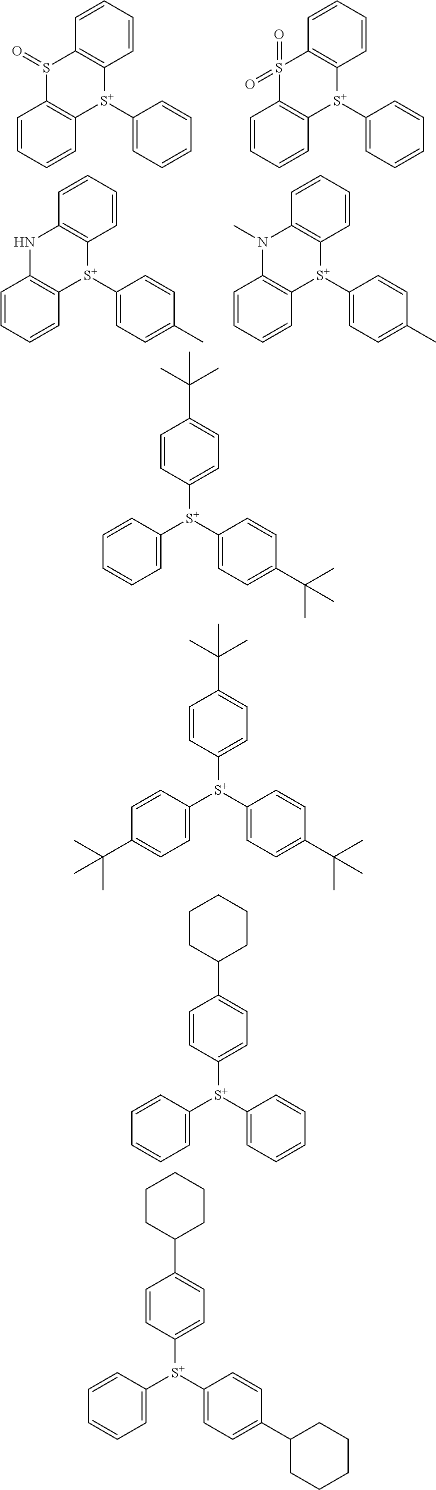

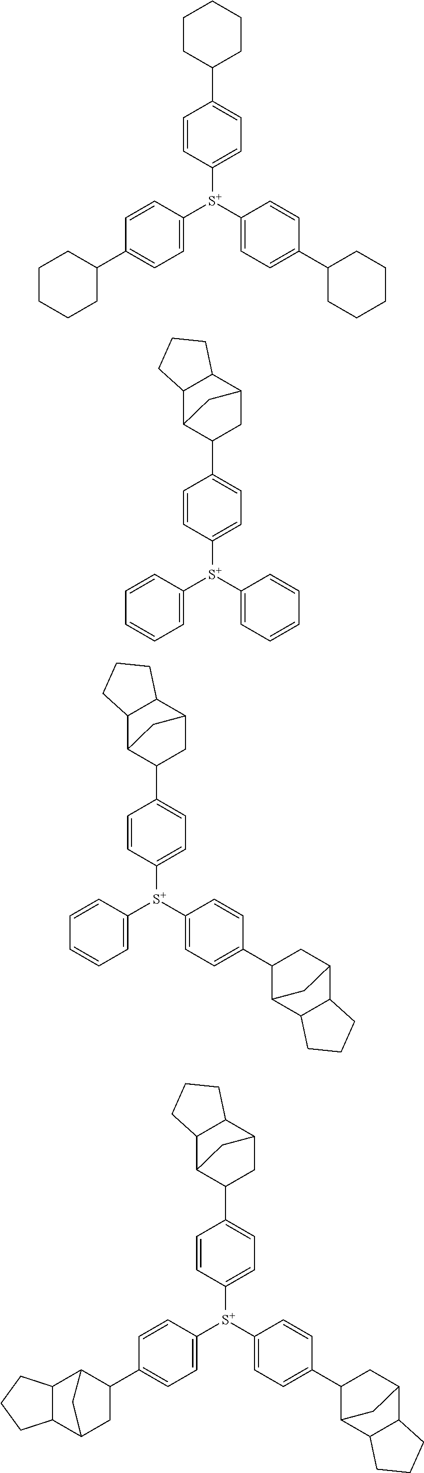 [SMILES: c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

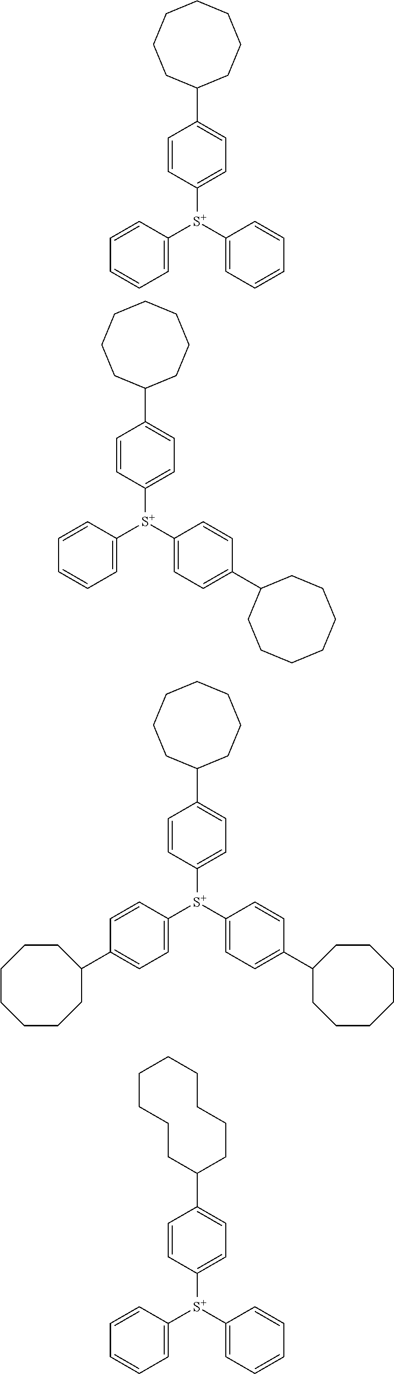 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1]

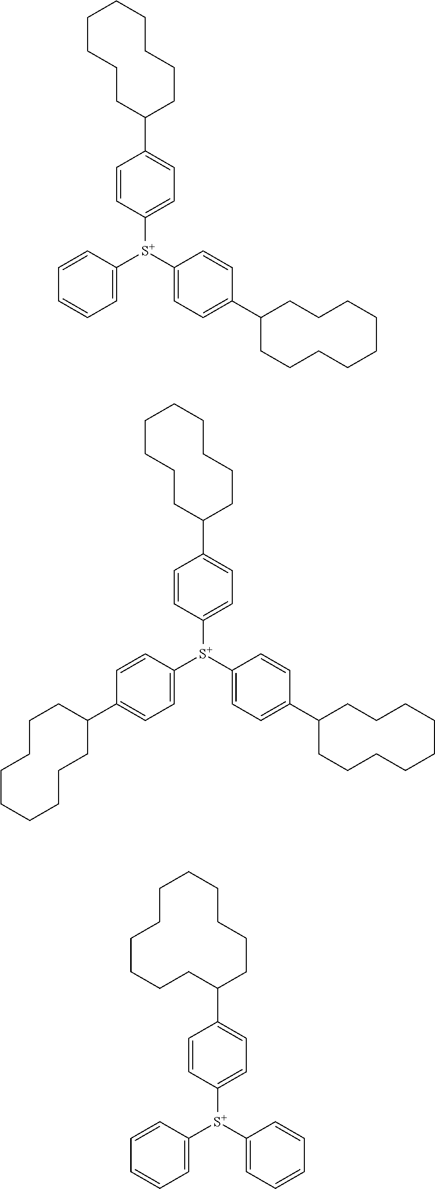 [SMILES: c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1]

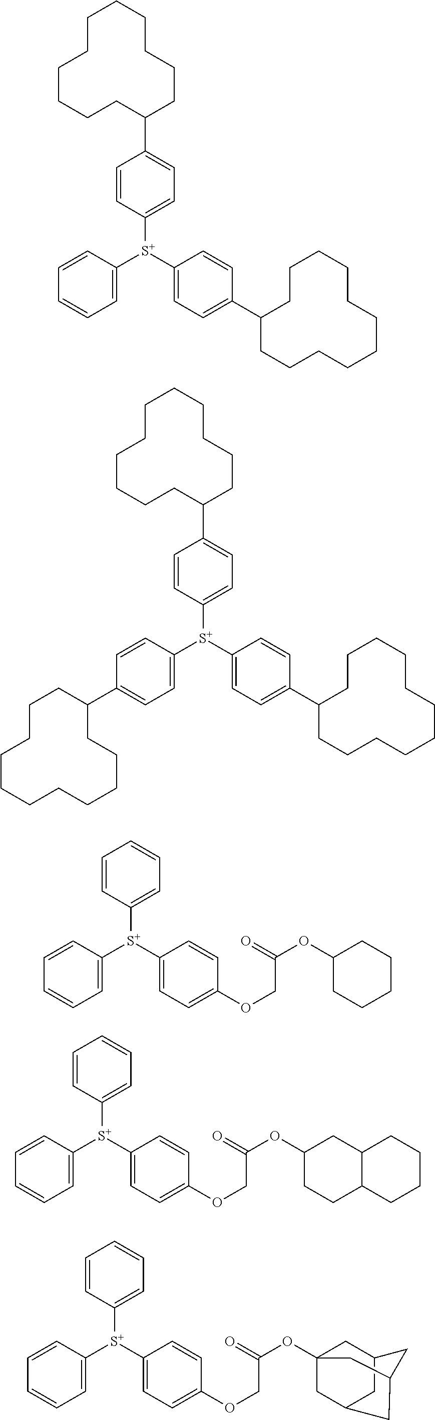 [SMILES: c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1 ; O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1 ; O=C(COc1ccc([S+](c2ccccc2)c2ccccc2)cc1)OC12CC3CC(CC(C3)C1)C2]

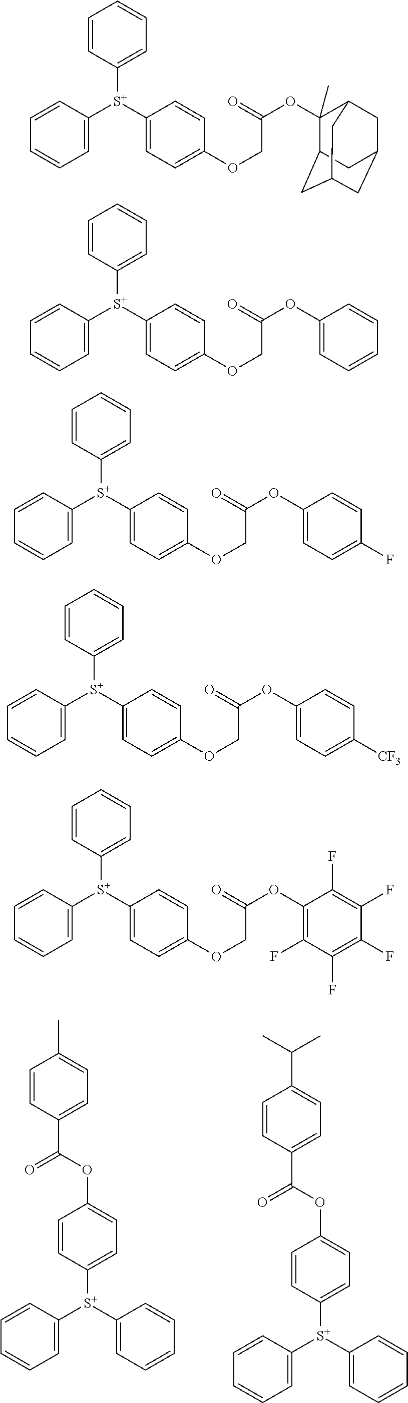

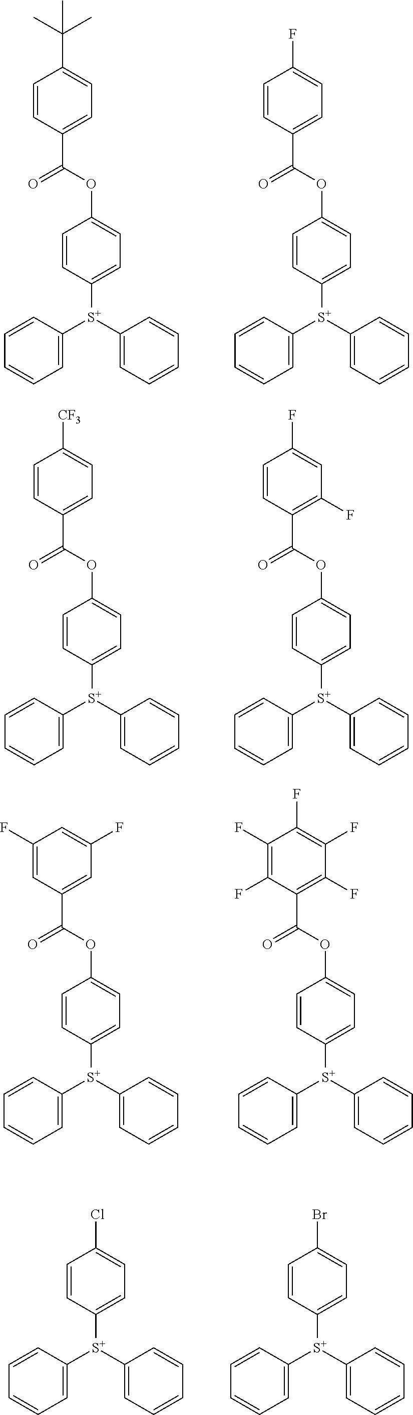

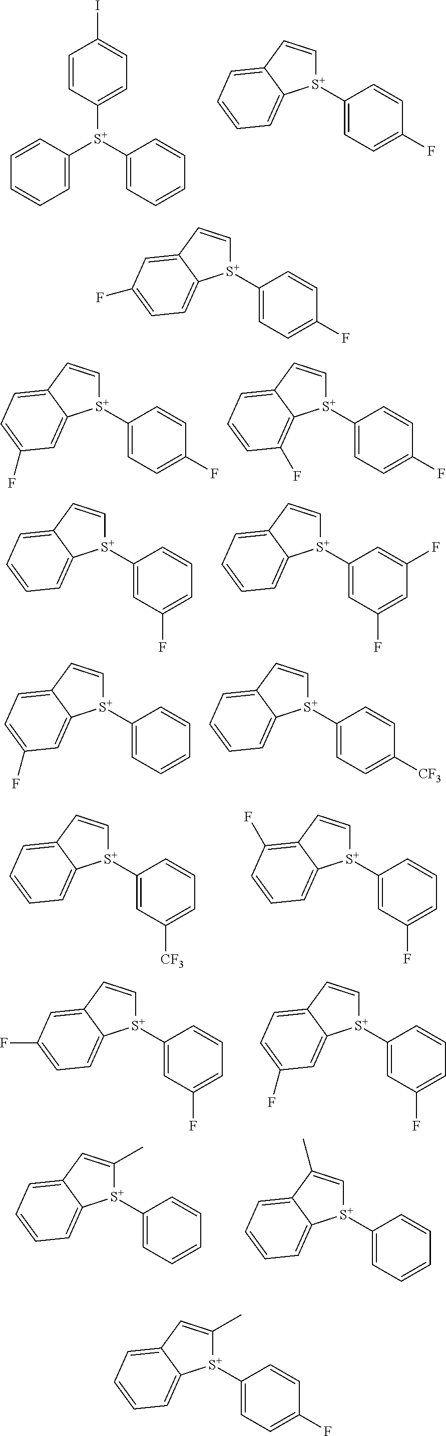

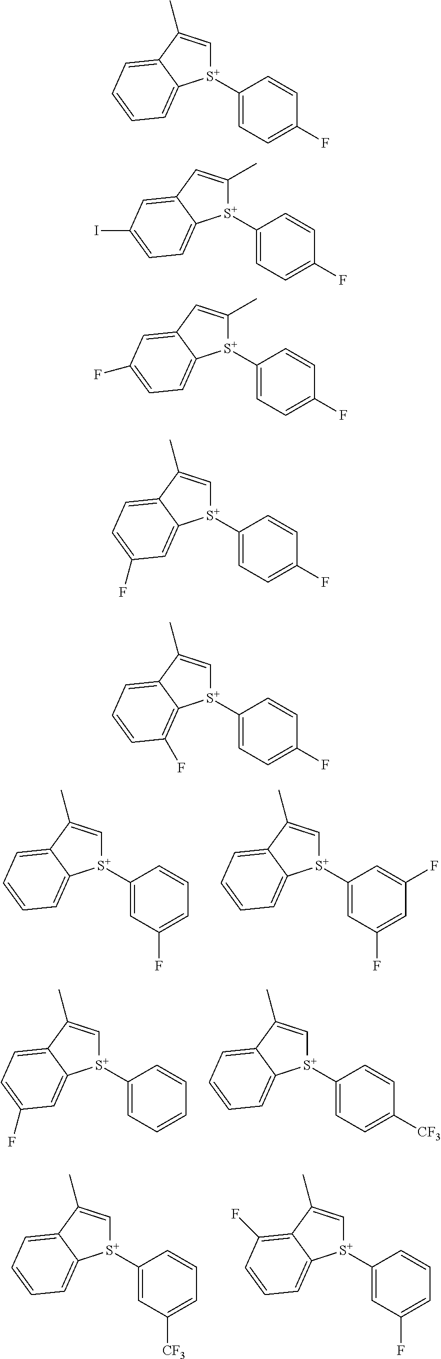

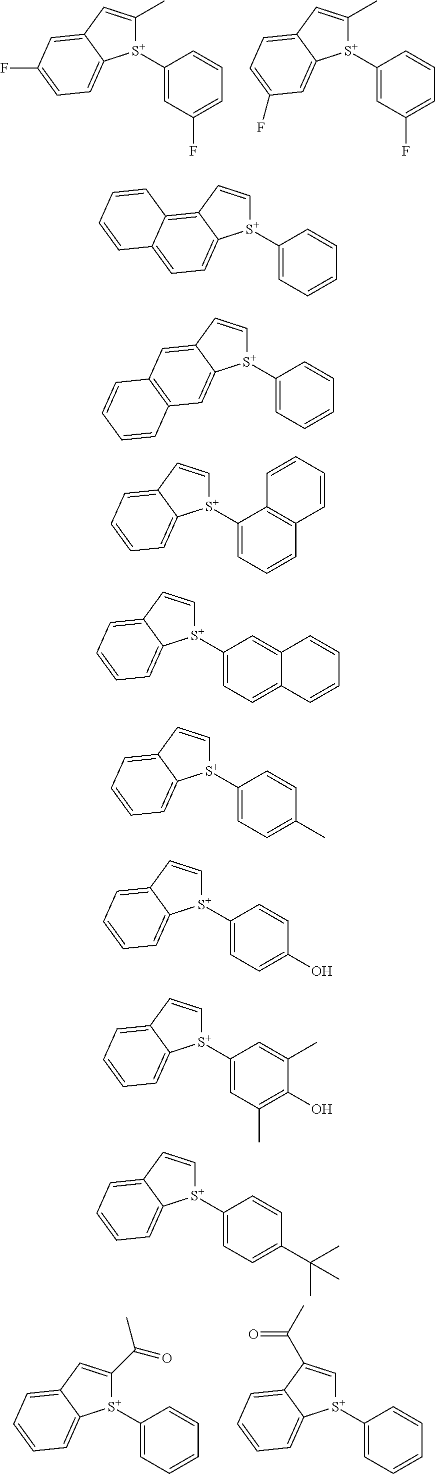

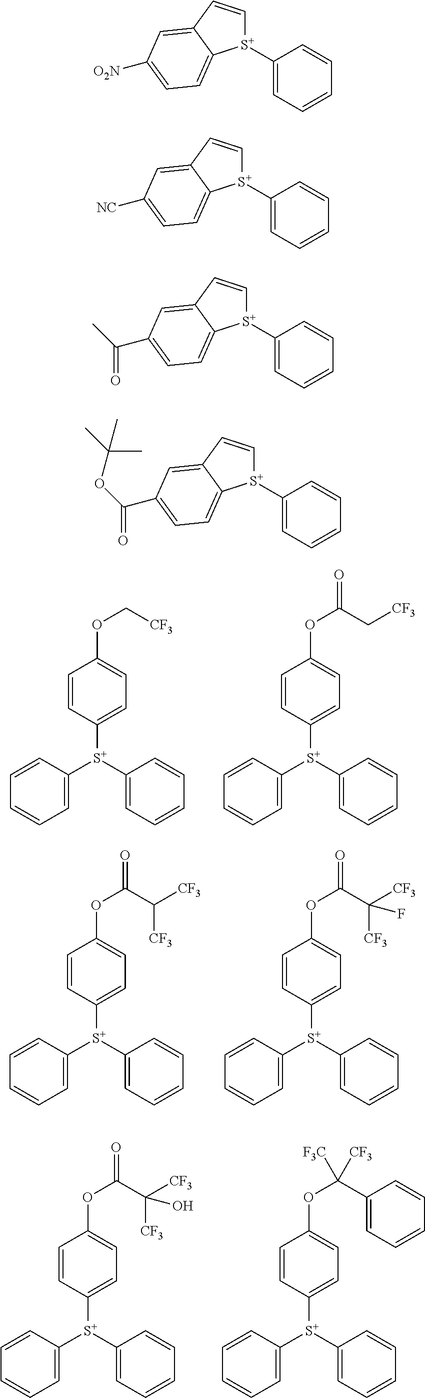

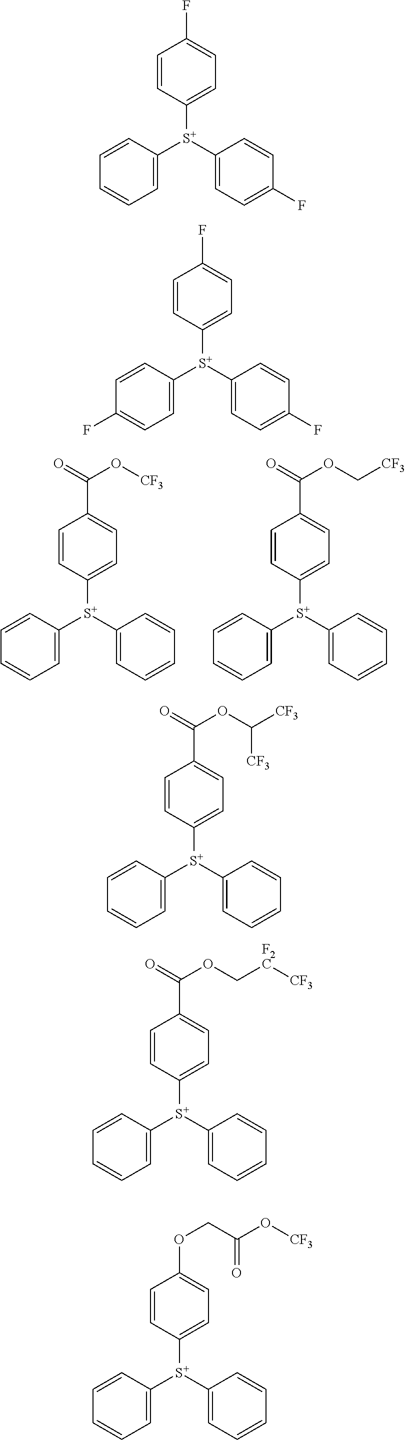

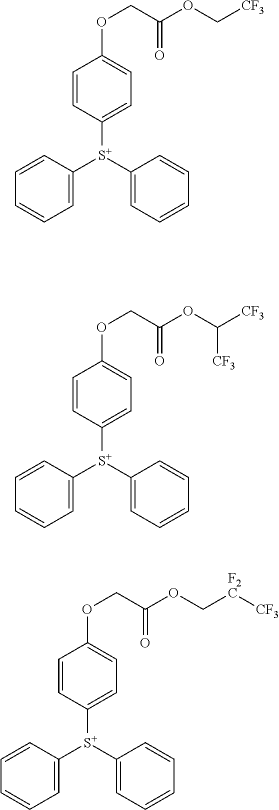 [SMILES: O=C(COc1ccc([S+](c2ccccc2)c2ccccc2)cc1)OCC(F)(F)F ; O=C(COc1ccc([S+](c2ccccc2)c2ccccc2)cc1)OC(C(F)(F)F)C(F)(F)F ; O=C(COc1ccc([S+](c2ccccc2)c2ccccc2)cc1)OCC(F)(F)C(F)(F)F]

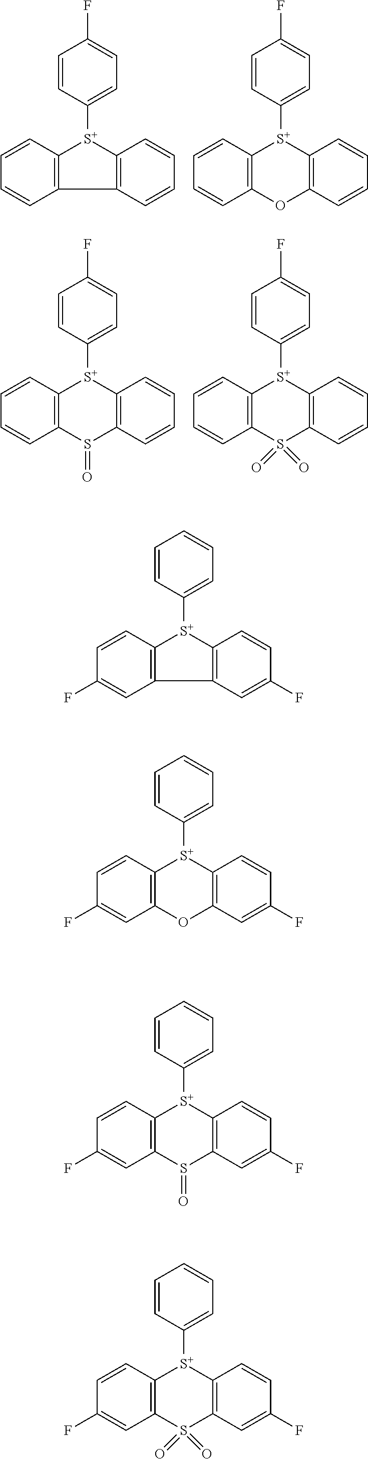

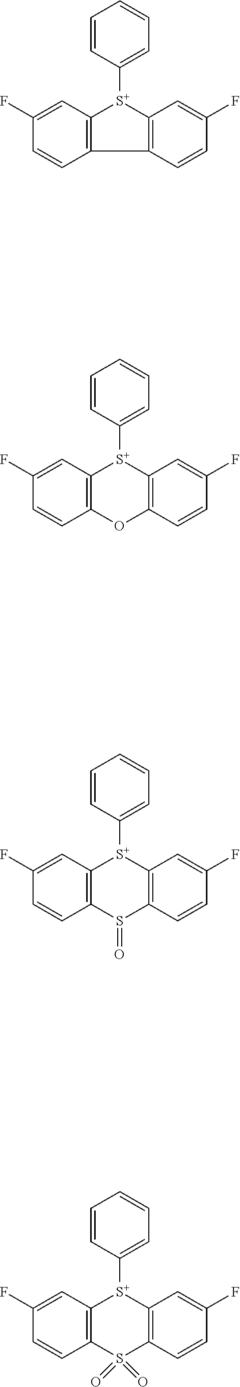 [SMILES: Fc1ccc2c3ccc(F)cc3[s+](-c3ccccc3)c2c1 ; Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21]

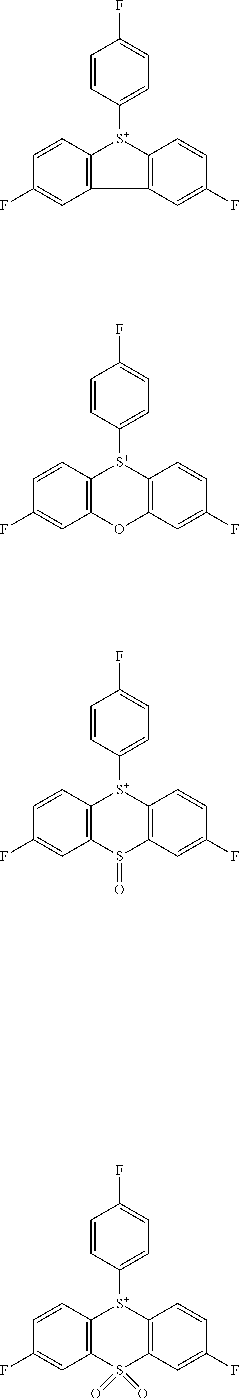 [SMILES: Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21]

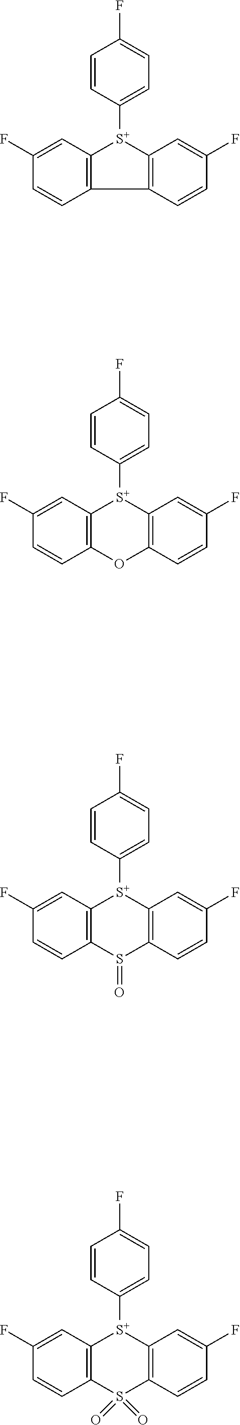 [SMILES: Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21]

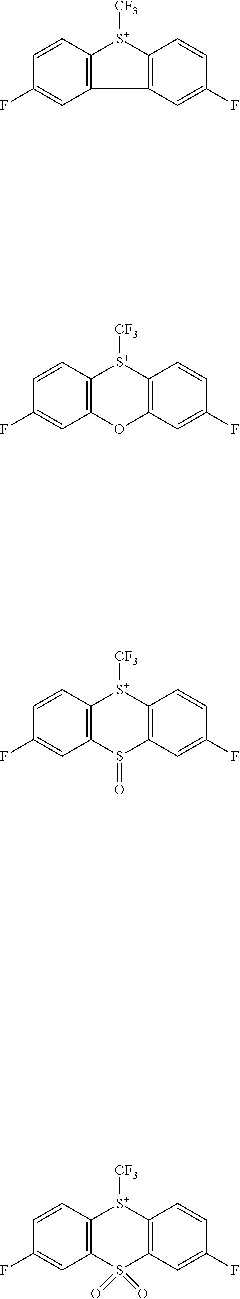 [SMILES: Fc1ccc2c(c1)c1cc(F)ccc1[s+]2C(F)(F)F ; Fc1ccc2c(c1)Oc1cc(F)ccc1[S+]2C(F)(F)F ; O=S1c2cc(F)ccc2[S+](C(F)(F)F)c2ccc(F)cc21 ; O=S1(=O)c2cc(F)ccc2[S+](C(F)(F)F)c2ccc(F)cc21]

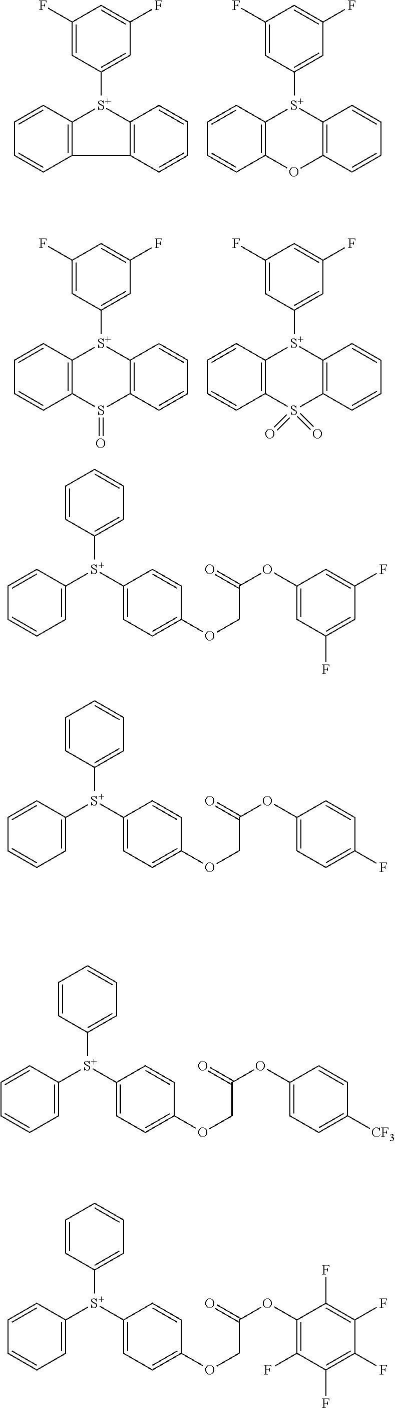

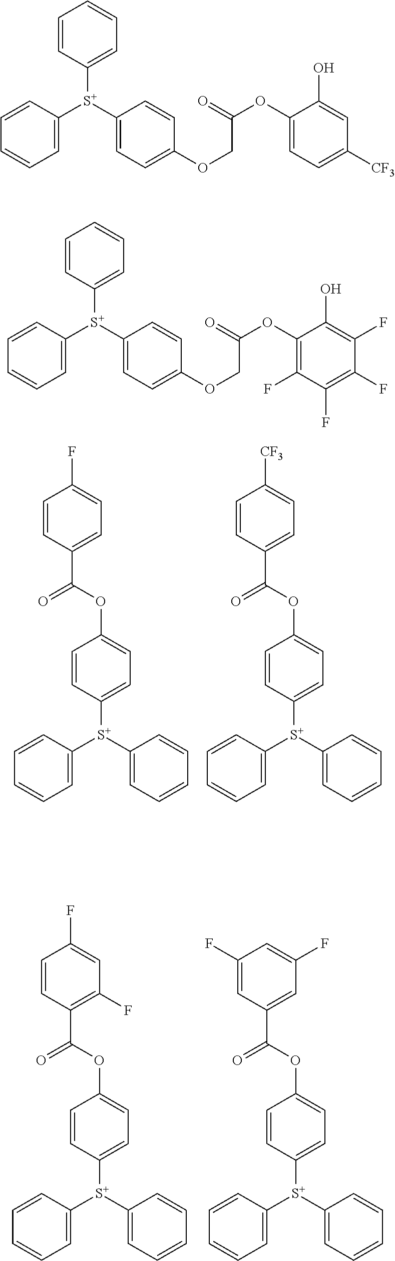 [SMILES: O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F ; O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1]

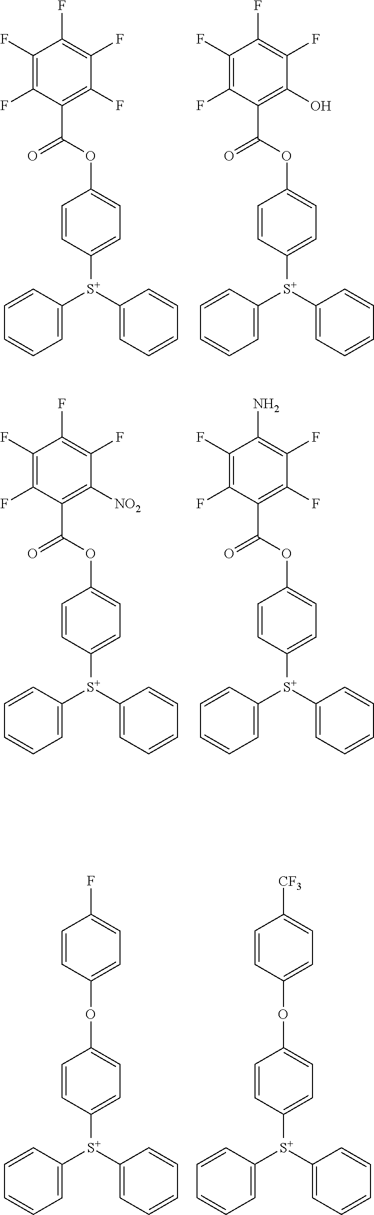 [SMILES: O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F ; O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1]

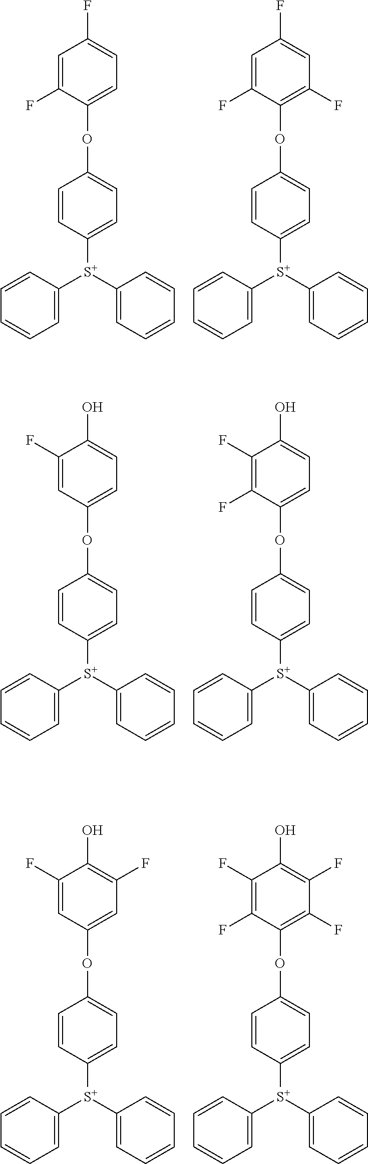 [SMILES: Fc1ccc(Oc2ccc([S+](c3ccccc3)c3ccccc3)cc2)c(F)c1 ; Fc1cc(F)c(Oc2ccc([S+](c3ccccc3)c3ccccc3)cc2)c(F)c1 ; Oc1ccc(Oc2ccc([S+](c3ccccc3)c3ccccc3)cc2)cc1F ; Oc1ccc(Oc2ccc([S+](c3ccccc3)c3ccccc3)cc2)c(F)c1F ; Oc1c(F)cc(Oc2ccc([S+](c3ccccc3)c3ccccc3)cc2)cc1F ; Oc1c(F)c(F)c(Oc2ccc([S+](c3ccccc3)c3ccccc3)cc2)c(F)c1F]

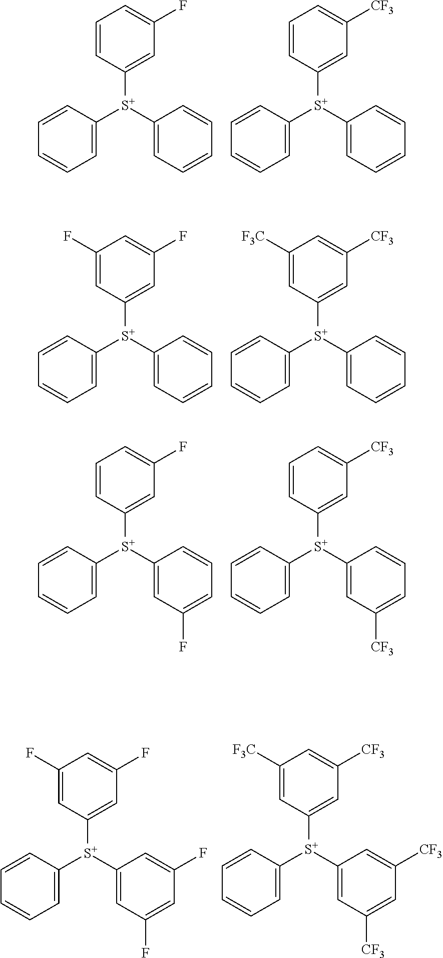

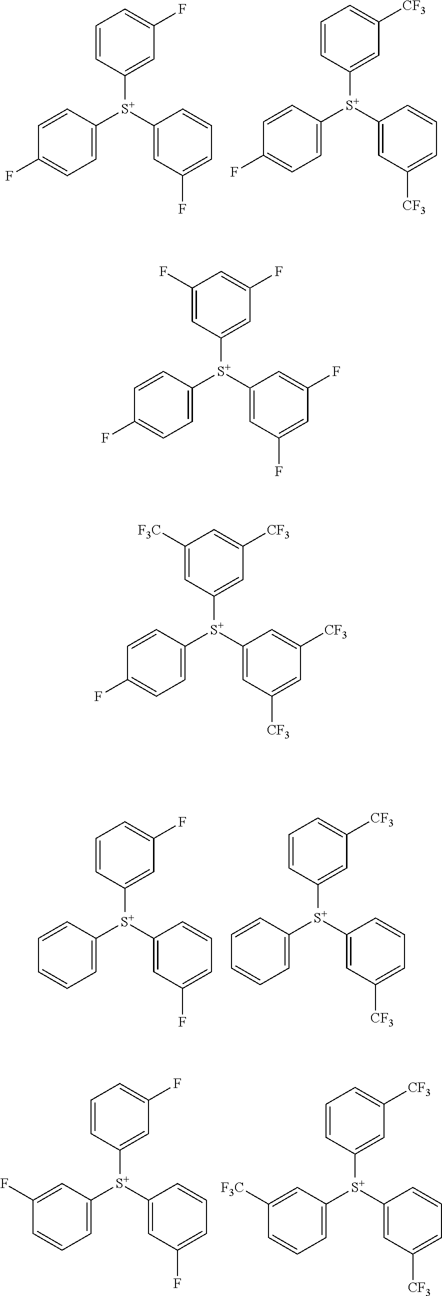

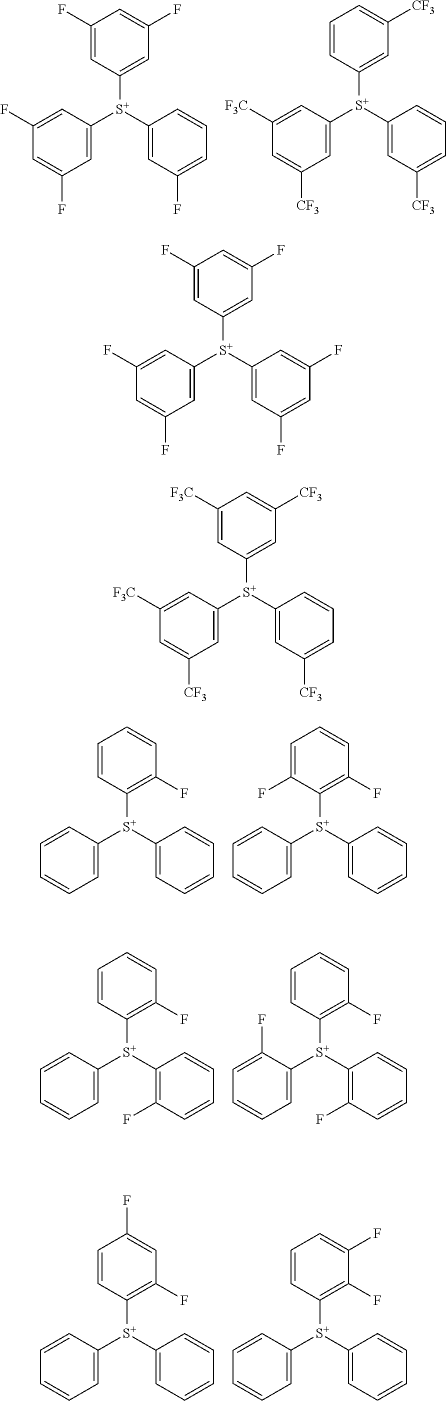

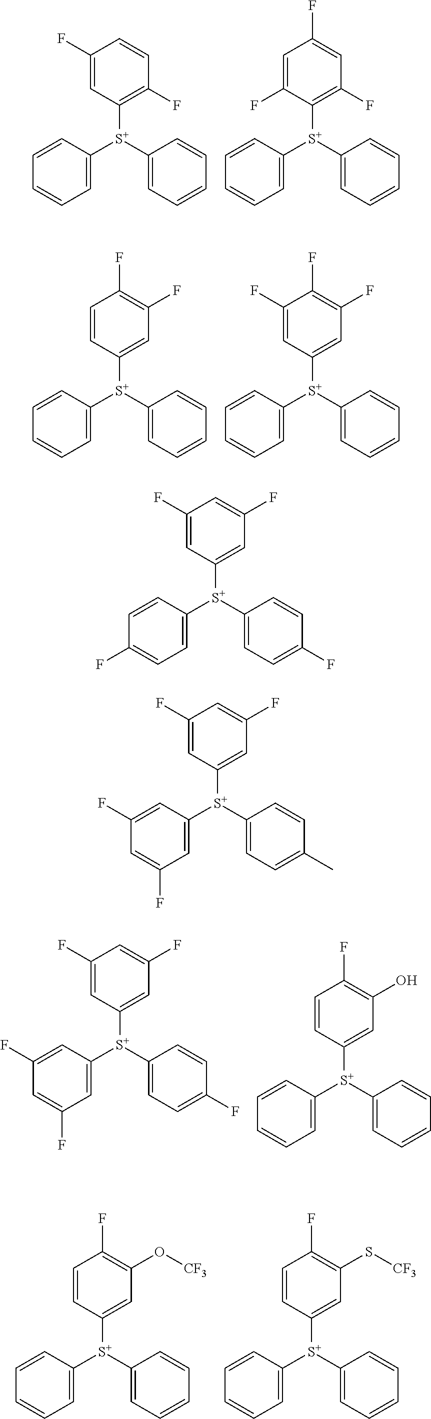

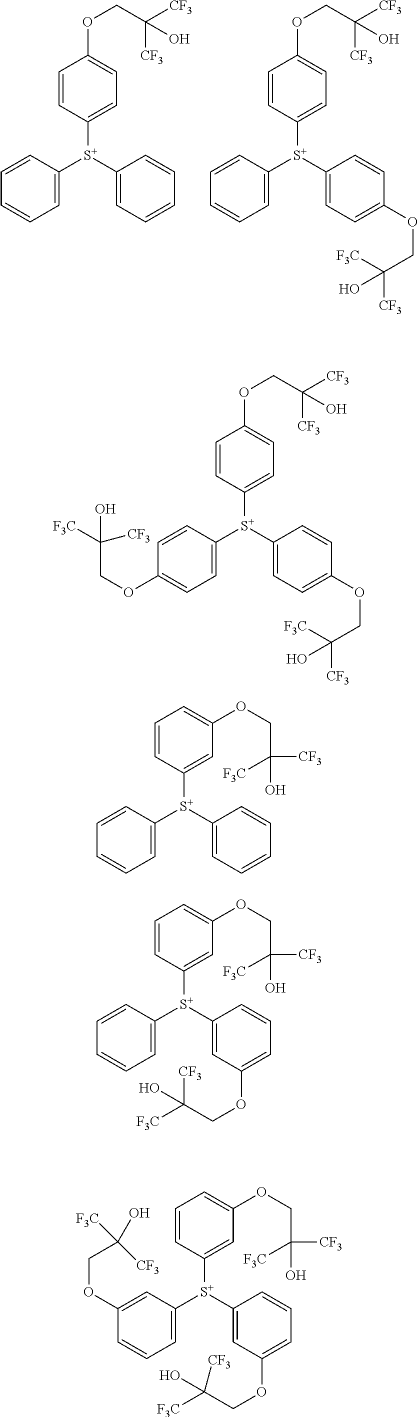

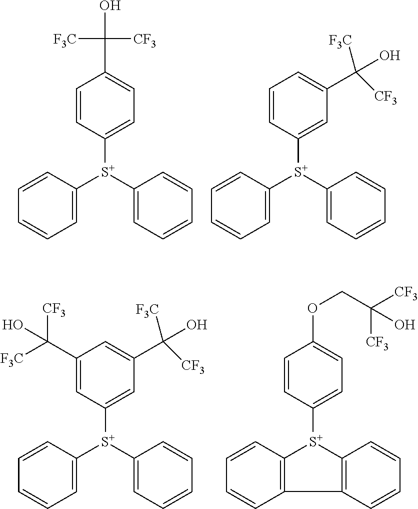 [SMILES: OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F ; OC(COc1ccc(-[s+]2c3ccccc3c3ccccc32)cc1)(C(F)(F)F)C(F)(F)F]

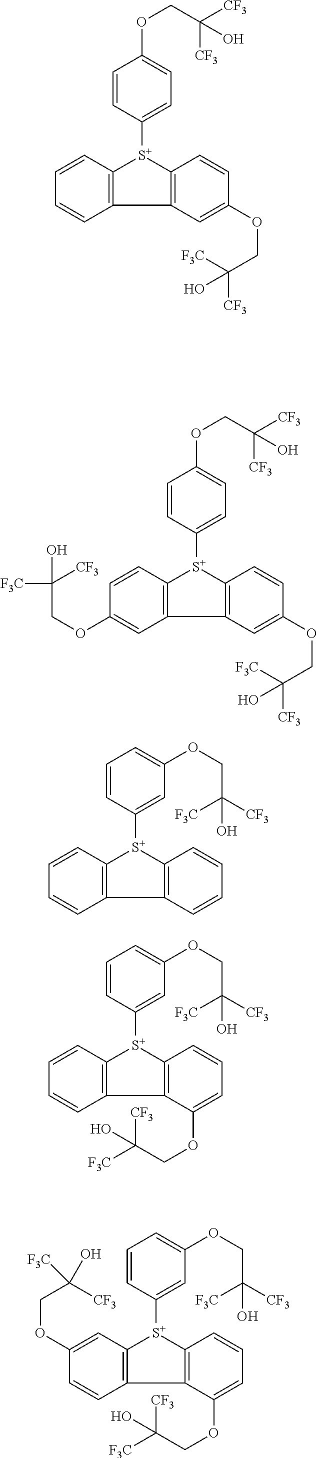 [SMILES: OC(COc1ccc(-[s+]2c3ccccc3c3cc(OCC(O)(C(F)(F)F)C(F)(F)F)ccc32)cc1)(C(F)(F)F)C(F)(F)F ; OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F ; OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F ; OC(COc1cccc(-[s+]2c3cc(OCC(O)(C(F)(F)F)C(F)(F)F)ccc3c3c(OCC(O)(C(F)(F)F)C(F)(F)F)cccc32)c1)(C(F)(F)F)C(F)(F)F]

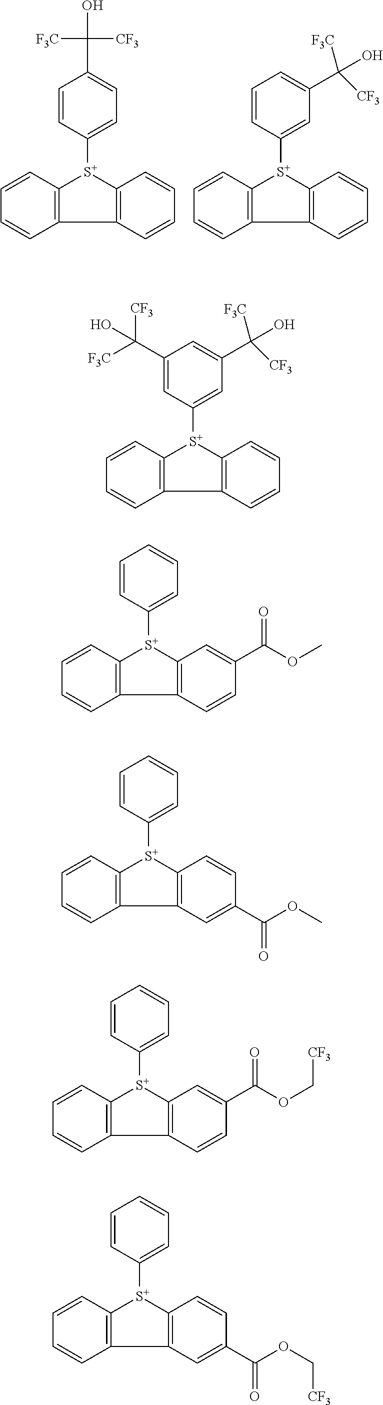

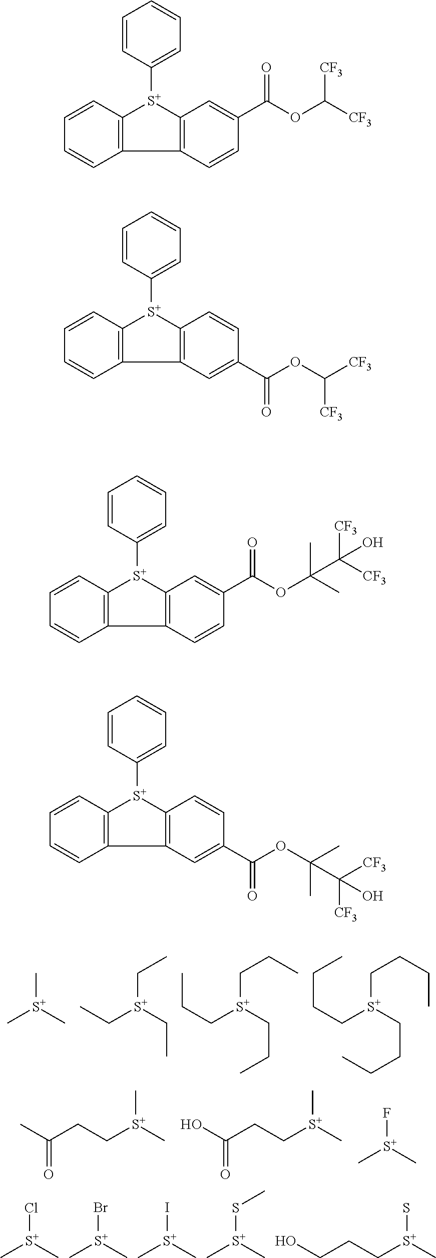

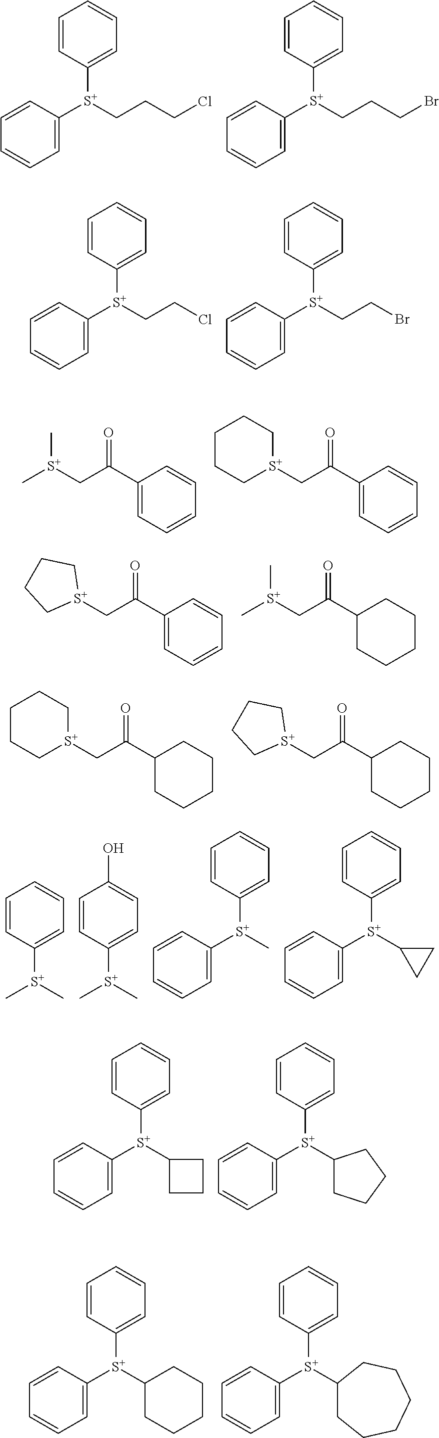

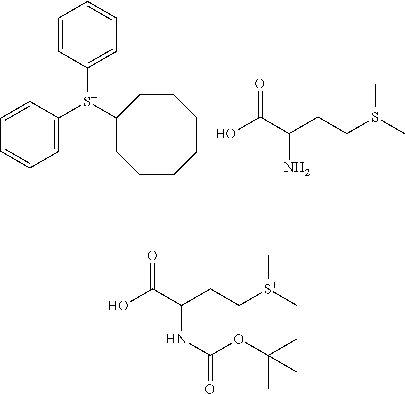

Examples of the iodonium cation having formula (cation-2) are shown below, but not limited thereto.

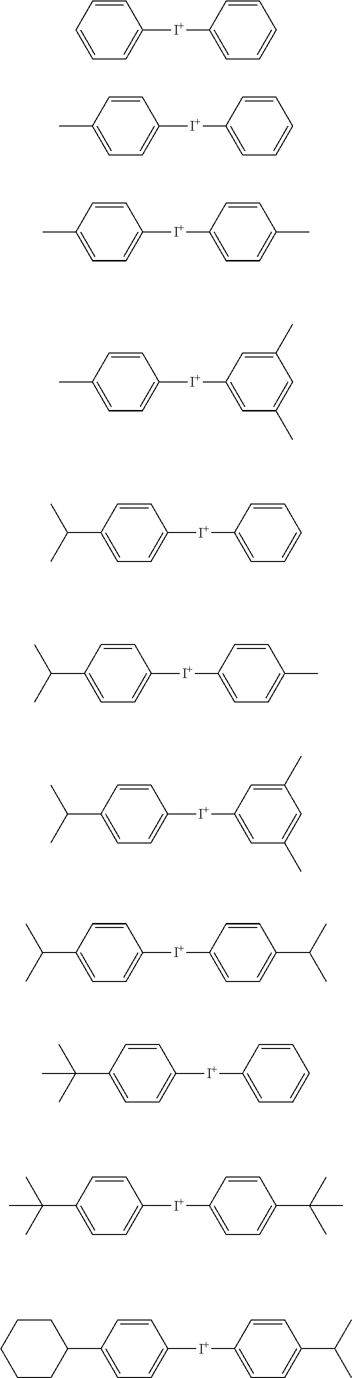

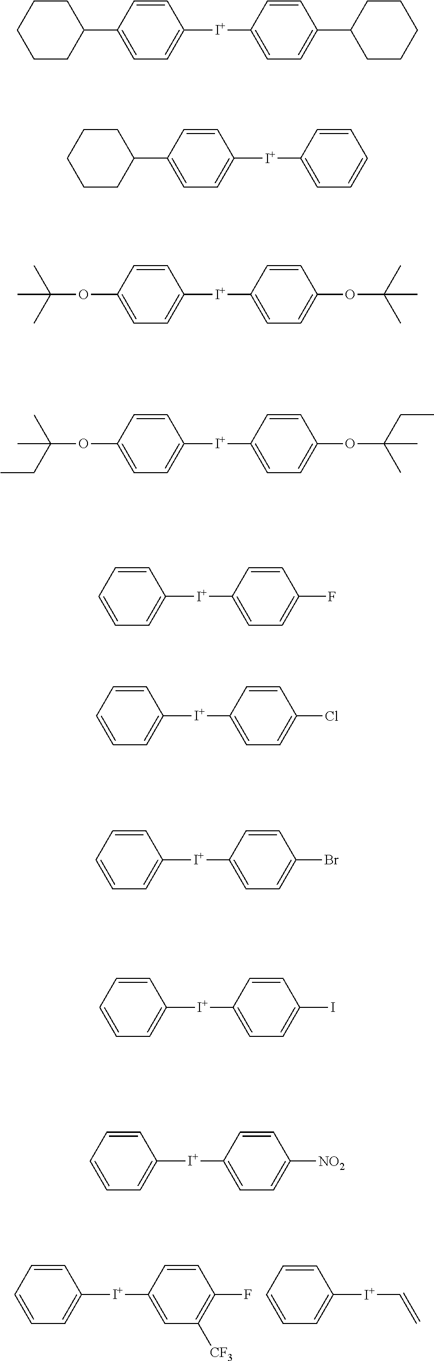

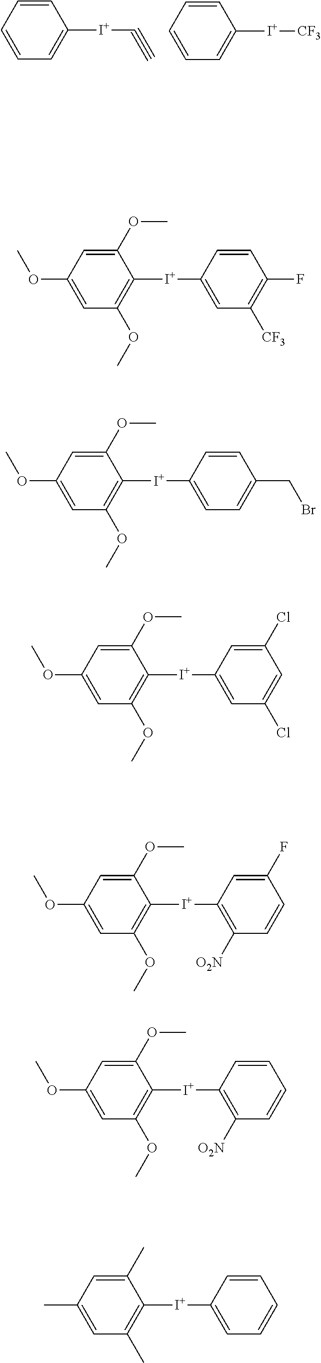

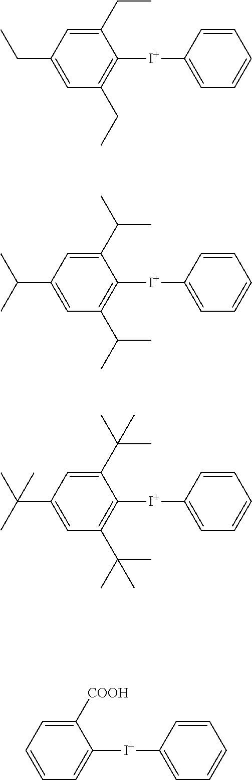 [SMILES: CCc1cc(CC)c([I+]c2ccccc2)c(CC)c1 ; CC(C)c1cc(C(C)C)c([I+]c2ccccc2)c(C(C)C)c1 ; CC(C)(C)c1cc(C(C)(C)C)c([I+]c2ccccc2)c(C(C)(C)C)c1 ; O=C(O)c1ccccc1[I+]c1ccccc1]

Examples of the ammonium cation having formula (cation-3) are shown below, but not limited thereto.

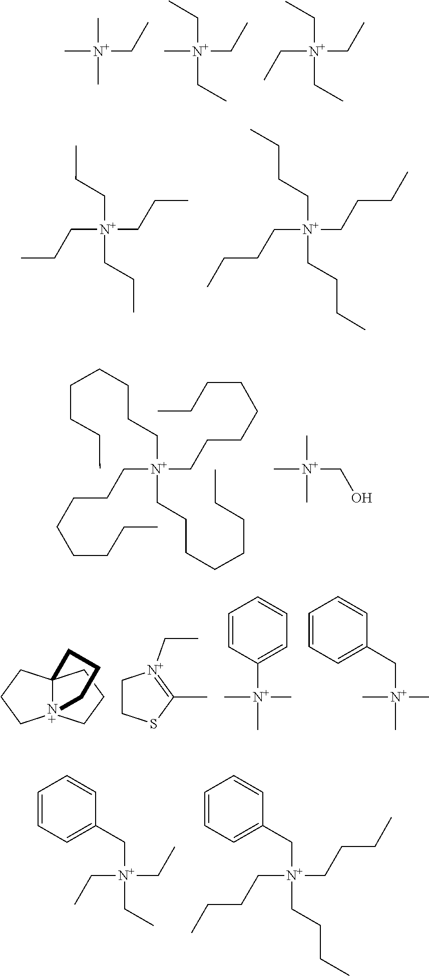

Specific structures of the inventive onium salt include arbitrary combinations of the anion with the cation, both as exemplified above.

The inventive onium salt may be synthesized by any well-known method, for example, according to the following scheme. Reference is now made to the synthesis of onium salt having the formula (NSQ-1-ex).

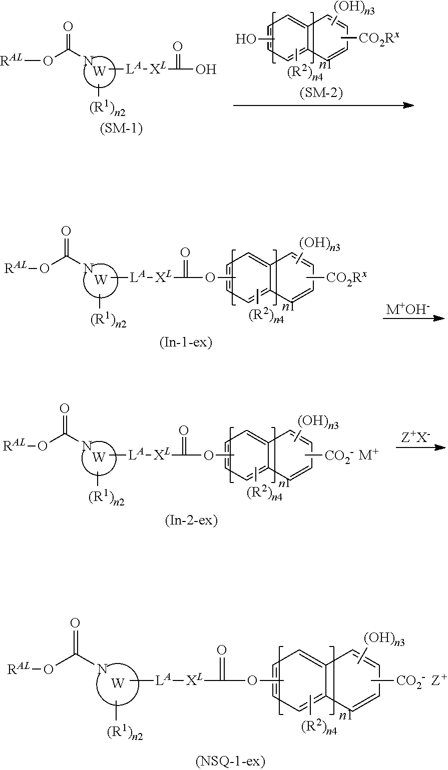

Herein, n1 to n4, W, L^{A}, X^{L}, R¹, R², R^{AL} and Z⁺ are as defined above. RX is a group which forms a primary or secondary ester with the adjacent —CO₂—. M⁺ is a counter cation. X⁻ is a counter anion.

The first step is to react reactant SM-1 with reactant SM-2, which are commercially available or can be synthesized by the standard method, to form an intermediate In-1-ex. Any condensing agents may be used when an ester bond is formed directly from the carboxy group on reactant SM-1 and the hydroxy group on reactant SM-2. Examples of the condensing agent include N,N′-dicyclohexylcarbodiimide, N,N′-diisopropylcarbodiimide, 1-[3-(dimethylamino)propyl]-3-ethylcarbodiimide, and 1-ethyl-3-(3-dimethylaminopropyl)carbodiimide hydrochloride. From the standpoint of ease of removal of urea compounds formed as by-products after reaction, it is desirable to use 1-ethyl-3-(3-dimethylaminopropyl)carbodiimide hydrochloride. The reaction is carried out by dissolving reactants SM-1 and SM-2 in a halogenated solvent such as methylene chloride and adding a condensing agent. The reaction rate can be increased by adding 4-dimethylaminopyridine as a catalyst. While it is desirable in view of yield to monitor the reaction by silica gel thin layer chromatography (TLC) until the reaction is complete, the reaction time is typically about 12 to 24 hours. After the reaction is terminated, urea compounds formed by side reactions are removed by filtration or water-washing if necessary. The reaction solution is subjected to standard aqueous work-up, obtaining Intermediate In-1-ex. If necessary, the intermediate is purified by a standard technique such as distillation, chromatography or recrystallization.

The second step is alkaline hydrolysis of Intermediate In-1-ex to Intermediate In-2-ex. Specifically, the carboxylic ester in Intermediate In-1-ex is subjected to alkaline hydrolysis using an alkali metal hydroxide salt or a hydroxide salt of organic cation, obtaining Intermediate In-2-ex or carboxylic acid salt. Examples of the alkali metal hydroxide salt used herein include lithium hydroxide, sodium hydroxide, and potassium hydroxide. Examples of the hydroxide salt of organic cation include tetramethylammonium hydroxide and benzyltrimethylammonium hydroxide. The reaction is carried out by dissolving Intermediate In-1-ex in an ether solvent such as tetrahydrofuran or 1,4-dioxane and adding an aqueous solution of the hydroxide salt. While it is desirable in view of yield to monitor the reaction by silica gel TLC until the reaction is complete, the reaction time is typically about 12 to 24 hours. The reaction mixture is subjected to standard aqueous work-up, obtaining Intermediate In-2-ex. If necessary, the intermediate is purified by a standard technique such as chromatography or recrystallization.

The third step is a salt exchange between Intermediate In-2-ex and an onium salt: Z⁺X⁻ to synthesize an onium salt NSQ-1-ex. X⁻ is preferably a chloride ion, bromide ion, iodide ion or methylsulfate anion because the exchange reaction readily takes place in a quantitative way. It is desirable in view of yield to monitor the reaction process by silica gel TLC. The reaction mixture is subjected to standard aqueous work-up, obtaining an onium salt NSQ-1-ex. If necessary, the onium salt is purified by a standard technique such as chromatography or recrystallization.

In the above-illustrated scheme, the third step of ion exchange may be readily carried out by any well-known procedure, for example, with reference to JP-A 2007-145797.

It is noted that the preparation method according to the above scheme is merely exemplary and the method of preparing the inventive onium salt is not limited thereto.

### Quencher

The inventive onium salt is useful as a quencher. As used herein, the term “quencher” refers to a compound capable of trapping the acid, which is generated by the acid generator in the resist composition upon light exposure, to prevent the acid from diffusing to the unexposed region and to assist in forming the desired pattern.

In a system where the inventive onium salt and an onium salt capable of generating strong acid such as α-fluorinated sulfonic acid, imide acid or methide acid are co-present, a corresponding carboxylic acid and strong acid generate upon light exposure. On the other hand, in the region receiving a reduced dose of exposure, much onium salt remains undecomposed. The strong acid functions as a catalyst for inducing deprotection reaction of acid labile groups on the base polymer whereas the inventive onium salt induces little deprotection reaction. The strong acid undergoes ion exchange with the residual carboxylic acid sulfonium salt. It is converted to a strong acid onium salt and instead, carboxylic acid is released. Differently stated, through ion exchange, the strong acid is neutralized with the carboxylic acid onium salt. That is, the inventive onium salt functions as a quencher. This onium salt type quencher tends to form a resist pattern with a reduced LWR as compared with the conventional quenchers in the form of amine compounds.

Salt exchange between strong acid and carboxylic acid onium salt is infinitely repeated. The site where strong acid is generated at the end of exposure shifts from the site where the onium salt of strong acid generation type is initially present. It is believed that since the cycle of photo-acid generation and salt exchange is repeated many times, the points of acid generation are averaged, which leads to a resist pattern with reduced LWR after development.

As the compound that exerts a quencher effect by a similar mechanism, Patent Documents 1 to 6 describe carboxylic acid onium salts, alkanesulfonic acid onium salts, arenesulfonic acid onium salts, and α,α-difluorocarboxylic acid onium salts. With respect to the type of onium salt, sulfonium, iodonium and ammonium salts are included. However, on use of an alkanesulfonic acid onium salt or arenesulfonic acid onium salt, the generated acid has a certain acid strength so that part thereof may induce deprotection reaction as the acid generator rather than as the quencher, leading to a lowering of resolution and an increase of acid diffusion, which invite losses of resist performance factors like exposure latitude (EL) and mask error factor (MEF). Also, the α,α-difluorocarboxylic acid onium salt as described in Patent Document 6, despite a carboxylic acid onium salt, has a possibility to provoke deprotection reaction depending on a choice of acid labile group on the base polymer, for the reason that the generated acid has a relatively high acidity like the sulfonic acid onium salt, due to the inclusion of fluorine at α-position of the carboxylate anion. Fluorocarboxylic acid onium salts obtained by simply extending the straight chain similarly allow for substantial acid diffusion and undergo salt exchange with strong acid in the unexposed region, probably leading to losses of resolution, EL and MEF. Further, the alkanecarboxylic acid onium salt is highly hydrophilic though it functions as a quencher. The fluoroalkanecarboxylic acid onium salt as described in Patent Document 3 has a somewhat controlled level of hydrophilicity as compared with the non-fluorinated type, but the control of hydrophilicity is insufficient when the carbon count is small. Although some onium salts of perfluoroalkanecarboxylic acid having a larger carbon count are known, they are deemed incompatible with resist compositions because the carboxylic acids have surfactant-like physical properties. Incompatibility with resist compositions can cause defect formation. Additionally, perfluoroalkanecarboxylic acids are unfavorable from the biotic and environmental aspects.

Patent Documents 7 to 9 describe carboxylic acid onium salts having an indole, indoline or piperidine carboxylic acid structure which is a nitrogen-containing heterocyclic compound. Patent Document 10 describes a carboxylic acid onium salt having an aminobenzoic acid structure. Patent Document 11 describes a carboxylic acid onium salt having an amide bond. Although these compounds act as the quencher, some problems remain. Since the aromatic amine and amide bond are not highly basic, the acid diffusion controlling ability is not sufficient. Since the piperidine carboxylic acid is highly water soluble, it promotes the penetration of an alkaline developer into a resist film in unexposed areas, causing the resist pattern to collapse or peel from the substrate.

The inventive onium salt solves the outstanding problem. The onium salt having a nitrogen-containing aliphatic heterocycle and aromatic carboxylic acid structure in the anion functions as a quencher. That is, the aromatic carboxylic acid anion site is effective for trapping the strong acid generated from the acid generator. The acid labile group with which the nitrogen-containing aliphatic heterocycle site is protected undergoes deprotection reaction under the action of strong acid, whereupon a nitrogen-containing aliphatic heterocyclic compound which is highly basic is formed. The highly basic nitrogen-containing aliphatic heterocycle site restrains excessive diffusion of acid to unexposed areas while the carboxylic acid anion site continuously repeats proton exchange with the strong acid. Due to the synergy of these effects, the dissolution contrast between exposed and unexposed areas is enhanced, and the diffusion of strong acid is adequately controlled. Thus satisfactory lithographic performance is achievable in the formation of small-size patterns. The aromatic carboxylic acid structure and the acid labile group with which the nitrogen-containing aliphatic heterocycle site is protected have an adequate number of carbon atoms, which leads to an improvement in organic solvent solubility. This effectively restrains the penetration of an alkaline developer into a resist film in unexposed areas and hence, the collapse or peeling of resist patterns.

### Resist Composition

Another embodiment of the invention is a resist composition comprising (A) a quencher in the form of the onium salt having formula (1) as an essential component.

In the resist composition, the quencher (A) is preferably used in an amount of 0.1 to 40 parts by weight, more preferably 1 to 20 parts by weight per 80 parts by weight of the base polymer (C) described below. An amount of quencher (A) in the range ensures a satisfactory quenching function, eliminating the risk of lowering sensitivity or leaving foreign particles due to shortage of solubility.

### (B) Organic Solvent

The resist composition may comprise (B) an organic solvent. The organic solvent used herein is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone (GBL), and mixtures thereof.

Of the foregoing organic solvents, it is recommended to use PGME, PGMEA, cyclohexanone, GBL, DAA, ethyl lactate, and mixtures thereof because the base polymer (C) is most soluble therein.

The organic solvent (B) is preferably added in an amount of 200 to 5,000 parts by weight, and more preferably 400 to 3,500 parts by weight per 80 parts by weight of the base polymer (C). The organic solvent may be used alone or in admixture.

### (C) Base Polymer

The resist composition may further comprise (C) a base polymer. The base polymer preferably contains repeat units having the formula (a1), which are also referred to as repeat units (a1).

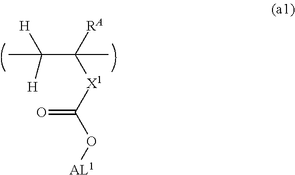

In formula (a1), R^{A} is hydrogen, fluorine, methyl or trifluoromethyl. X¹ is a single bond, phenylene group, naphthylene group, or *—C(═O)—O—X¹¹—. The phenylene and naphthylene groups may be substituted with an optionally fluorinated C₁-C₁₀ alkoxy moiety or halogen. X¹¹ is a C₁-C₁₀ saturated hydrocarbylene group which may contain a hydroxy moiety, ether bond, ester bond or lactone ring, a phenylene group or naphthylene group. The asterisk (*) designates a point of attachment to the carbon atom in the backbone.

In formula (a1), AL¹ is an acid labile group. The acid labile group may be selected from a variety of such groups, for example, those groups described in JP-A 2013-080033 (U.S. Pat. No. 8,574,817) and JP-A 2013-083821 (U.S. Pat. No. 8,846,303).

Typical of the acid labile group are groups of the following formulae (AL-3) to (AL-5).

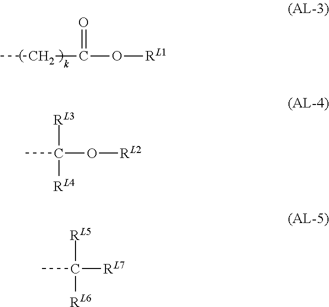

In formulae (AL-3) and (AL-4), R^{AL1} and R^{AL2} are each independently a C₁-C₄₀ saturated hydrocarbyl group which may contain a heteroatom such as oxygen, sulfur, nitrogen or fluorine. The saturated hydrocarbyl group may be straight, branched or cyclic. Inter alia, C₁-C₂₀ saturated hydrocarbyl groups are preferred.

In formula (AL-3), k is an integer of 0 to 10, preferably 1 to 5.

In formula (AL-4), R^{L3} and R^{L4} are each independently hydrogen or a C₁-C₂₀ saturated hydrocarbyl group which may contain a heteroatom such as oxygen, sulfur, nitrogen or fluorine. The saturated hydrocarbyl group may be straight, branched or cyclic. Any two of R^{AL2}, R^{AL3} and R^{L4} may bond together to form a C₃-C₂₀ ring with the carbon atom or carbon and oxygen atoms to which they are attached. The ring preferably contains 4 to 16 carbon atoms and is typically alicyclic.

In formula (AL-5), R^{AL5}, R^{AL6} and R^{L7} are each independently a C₁-C₂₀ saturated hydrocarbyl group which may contain a heteroatom such as oxygen, sulfur, nitrogen or fluorine. The saturated hydrocarbyl group may be straight, branched or cyclic. Any two of R^{AL5}, R^{AL6} and R^{AL7} may bond together to form a C₃-C₂₀ ring with the carbon atom to which they are attached. The ring preferably contains 4 to 16 carbon atoms and is typically alicyclic.

Examples of the repeat unit (a1) are shown below, but not limited thereto. Herein R^{A} and AL¹ are as defined above.
n

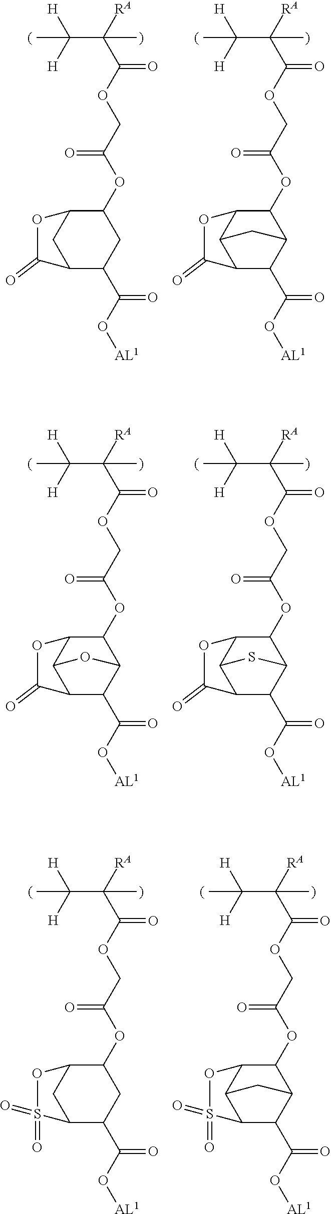

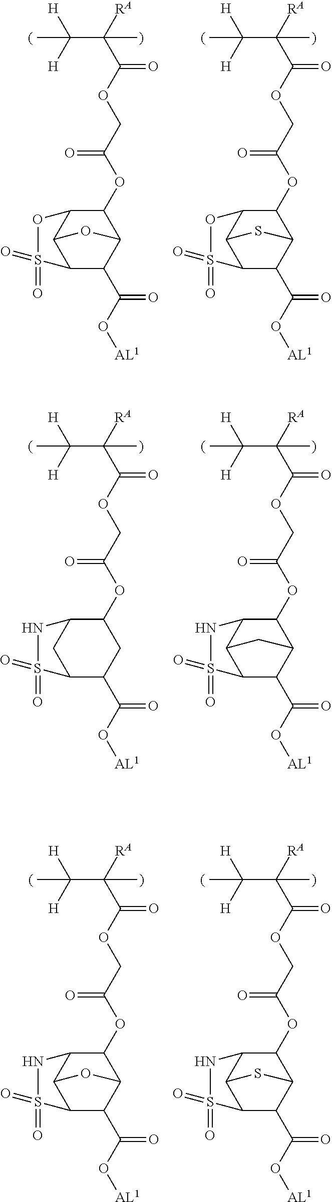

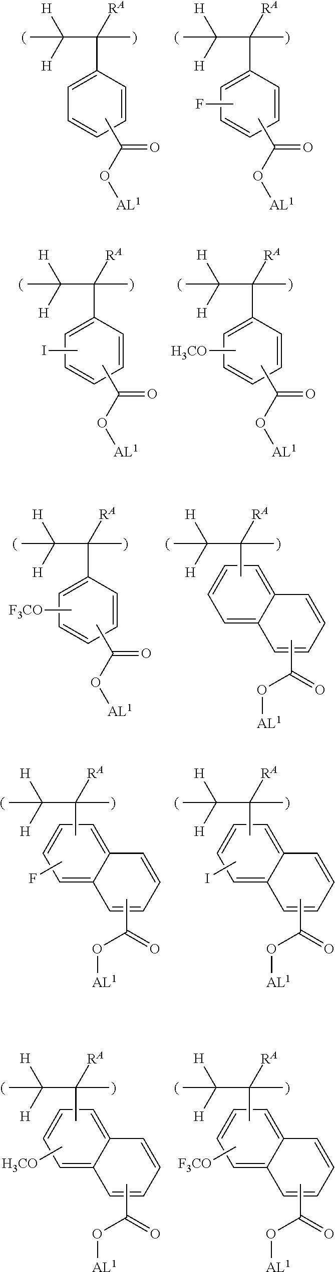

The base polymer may further comprise repeat units having the formula (a2), which are also referred to as repeat units (a2).

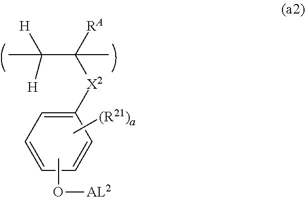

In formula a2, R^{a} is hydrogen, flourine, methyl or trifluoromethyl. X² is a single bond or *—C(═O)—O—. The asterisk (*) designates a point of attachment to the carbon atom in the backbone. R²¹ is halogen, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom. The subscript “a” is an integer of 0 to 4, preferably 0 or 1. AL² is an acid labile group, examples of which are the same as the acid labile group AL¹.

Examples of the repeat unit (a2) are shown below, but not limited thereto. Herein R^{A} and AL² are as defined above.

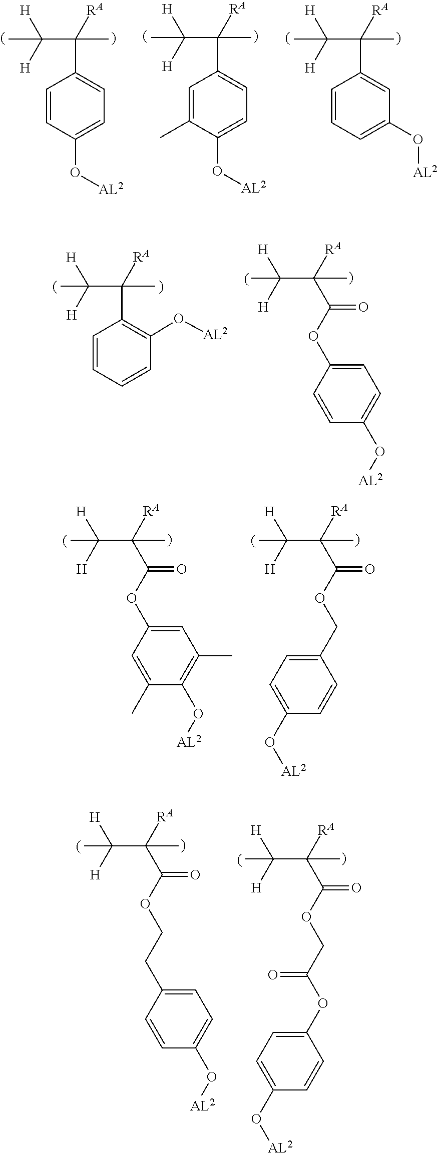

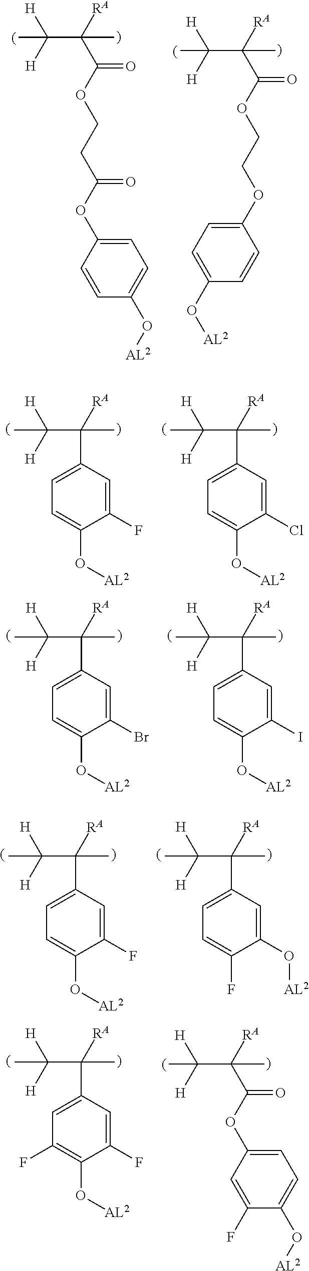

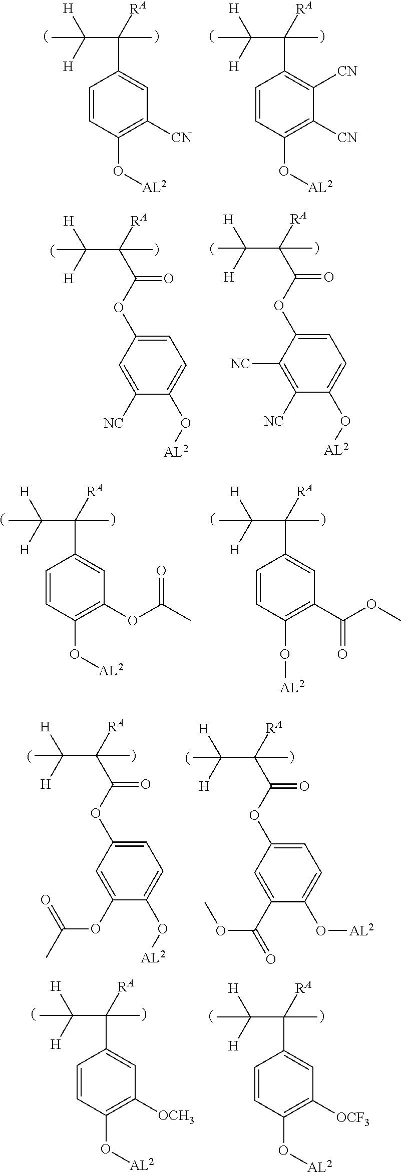

The base polymer may further comprise repeat units having the formula (b1) or repeat units having the formula (b2), which are also referred to as repeat units (b1) or (b2).

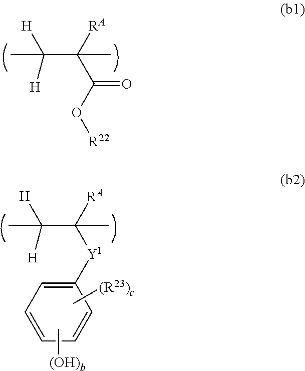

In formulae (b1) and (b2), R is each independently hydrogen, fluorine, methyl or trifluoromethyl. Y¹ is a single bond or *—C(═O)—O— wherein * designates a point of attachment to the carbon atom in the backbone. R²² is hydrogen or a C₁-C₂₀ group containing at least one structure selected from hydroxy other than phenolic hydroxy, cyano, carbonyl, carboxy, ether bond, ester bond, sulfonic ester bond, carbonate bond, lactone ring, sultone ring, and carboxylic anhydride (—C(═O)—O—C(═O)—). R²³ is halogen, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom. The subscript “b” is an integer of 1 to 4, “c” is an integer of 0 to 4, and the sum of b+c is from 1 to 5.

Examples of the repeat unit (b1) are shown below, but not limited thereto. Herein R^{A} is as defined above.

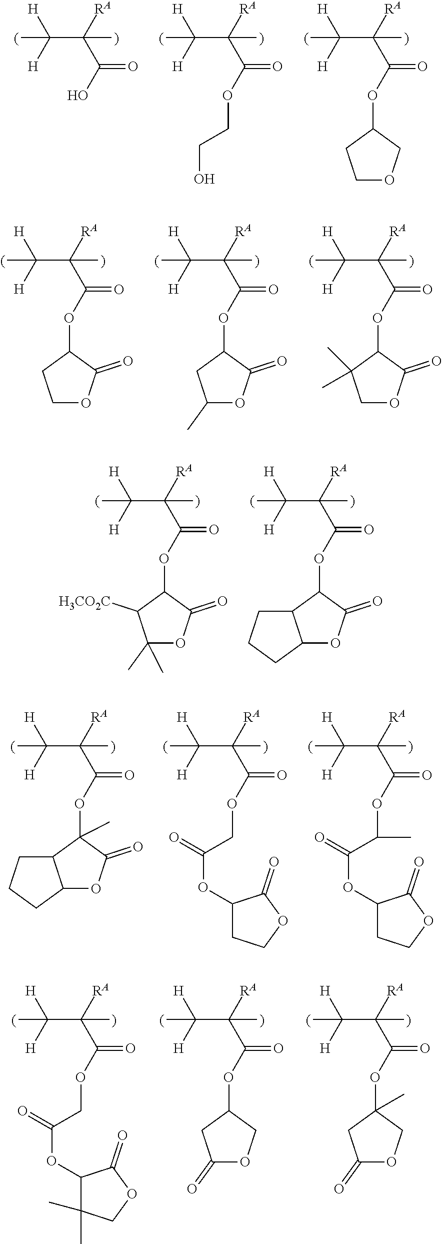

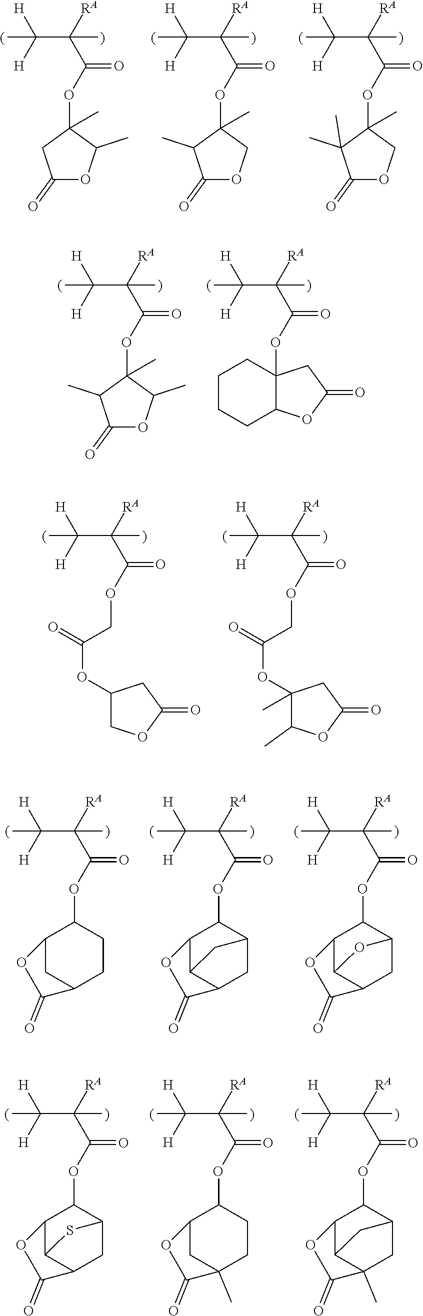

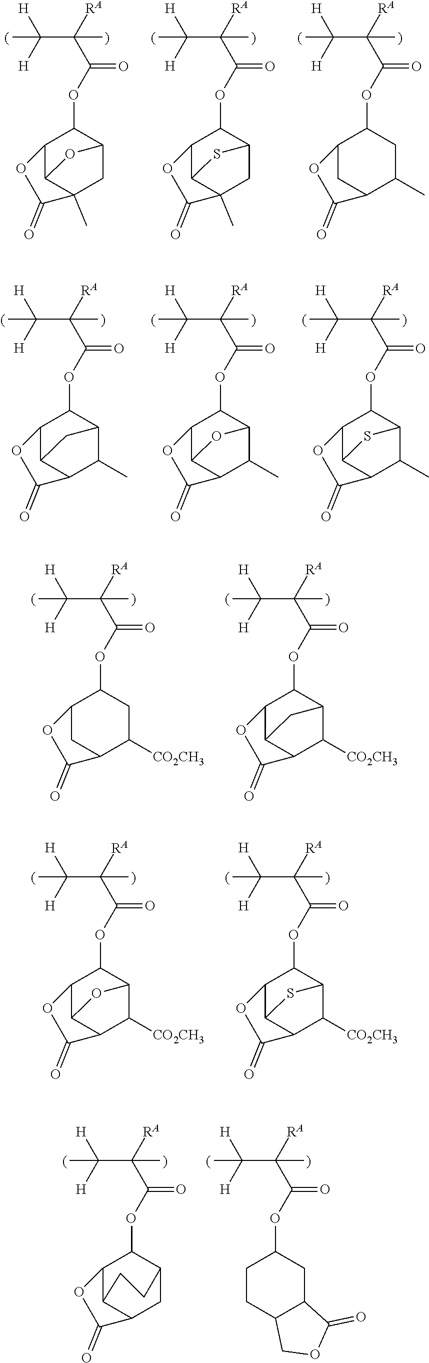

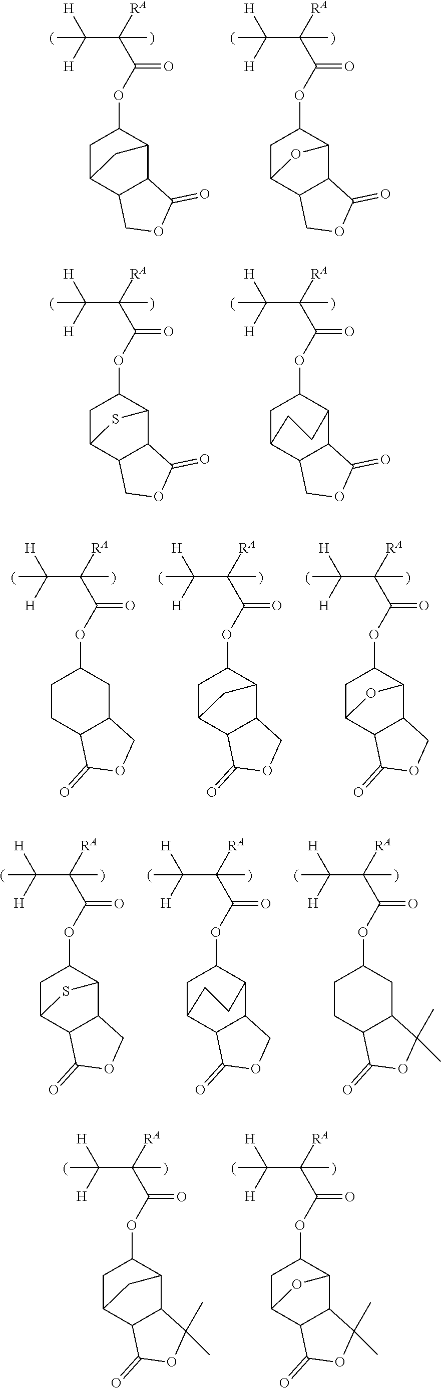

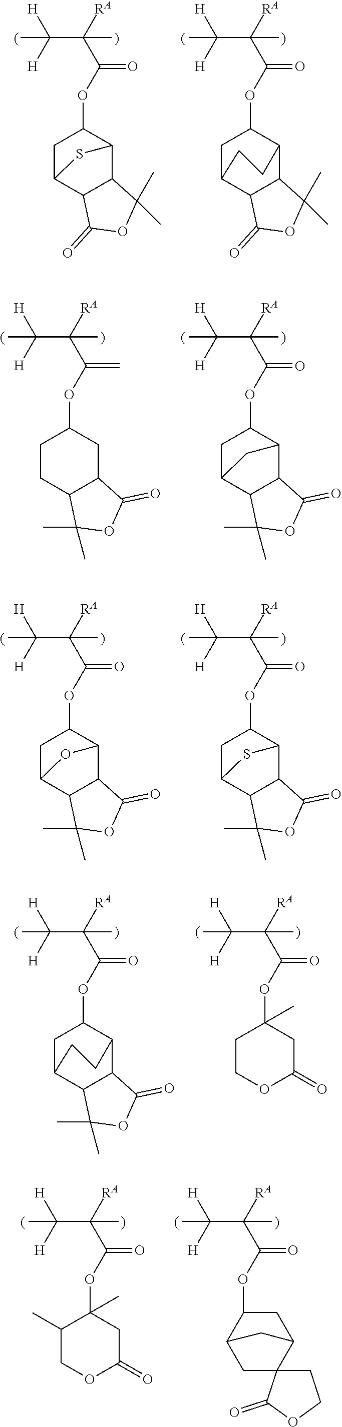

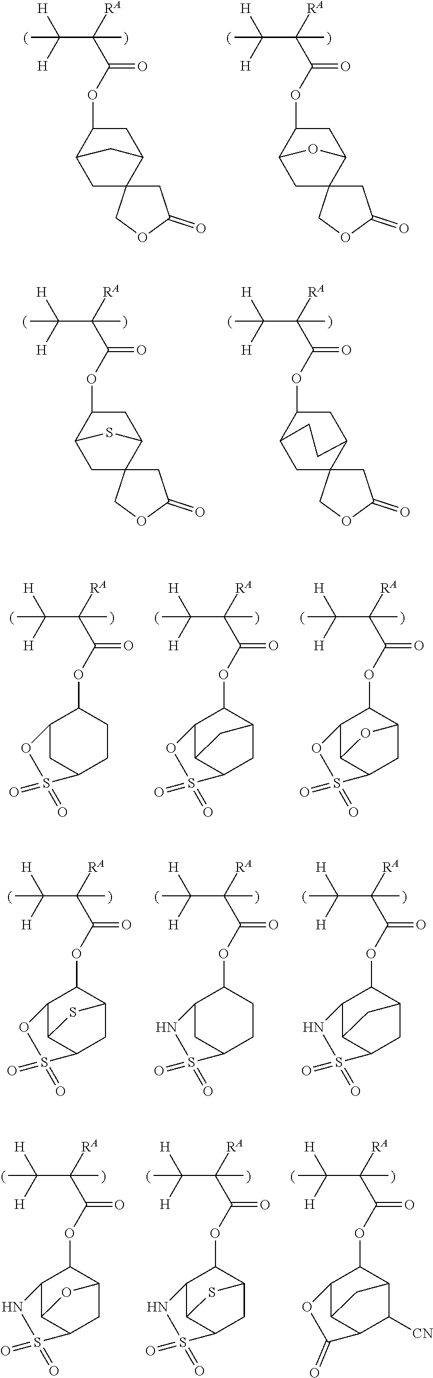

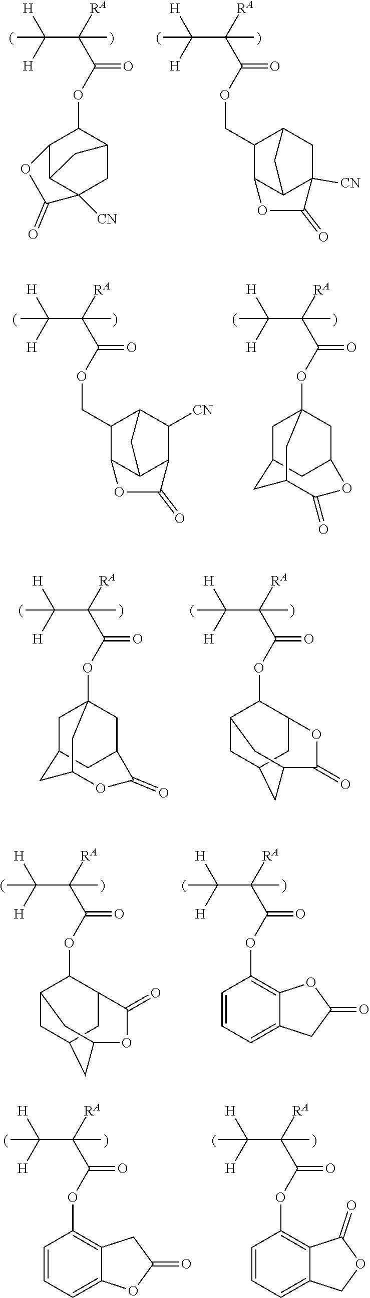

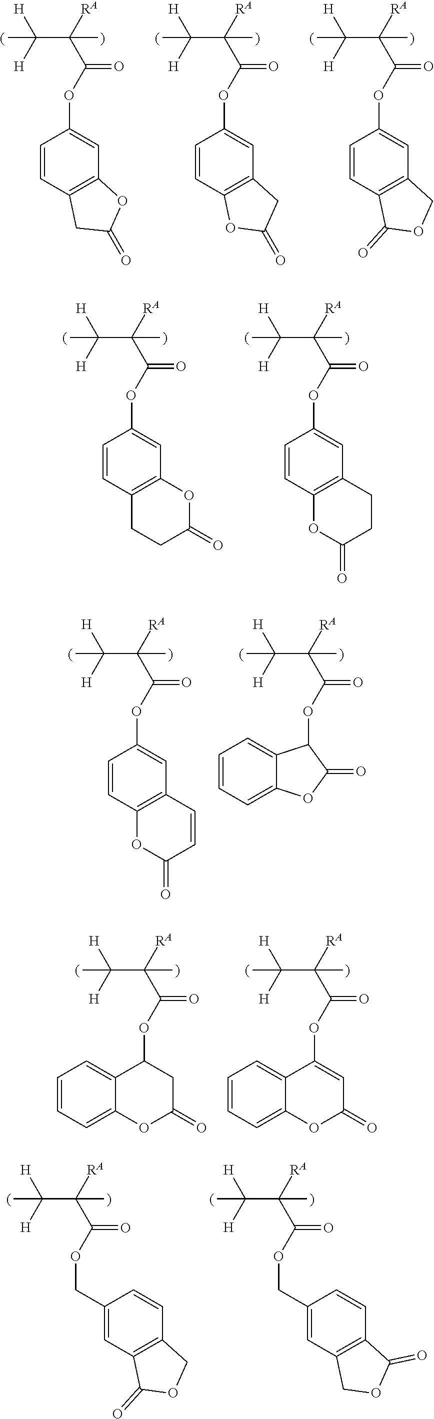

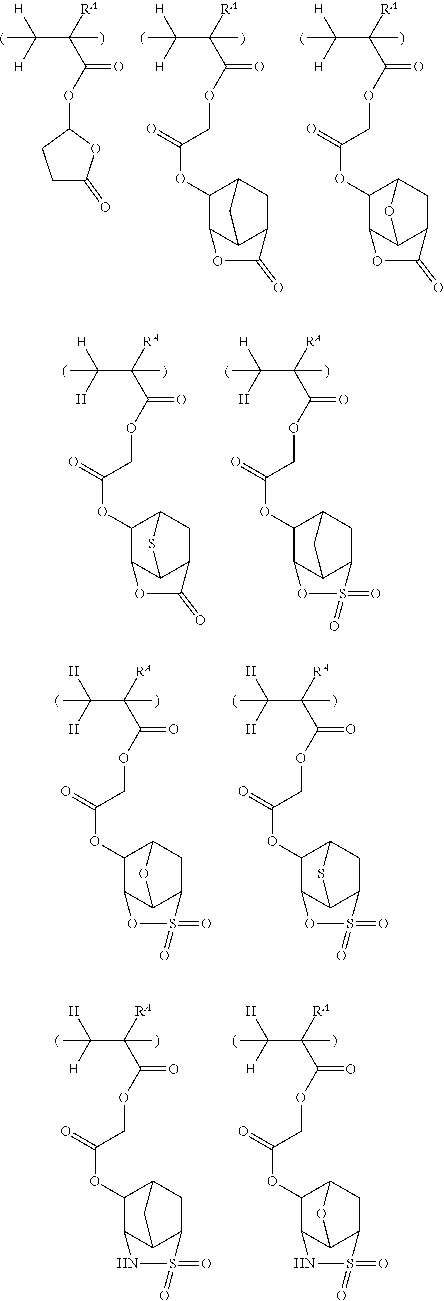

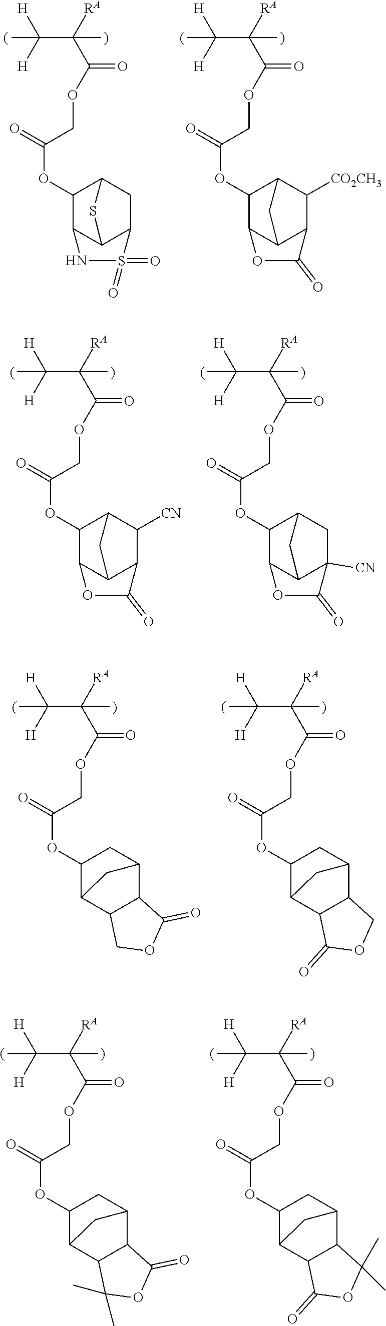

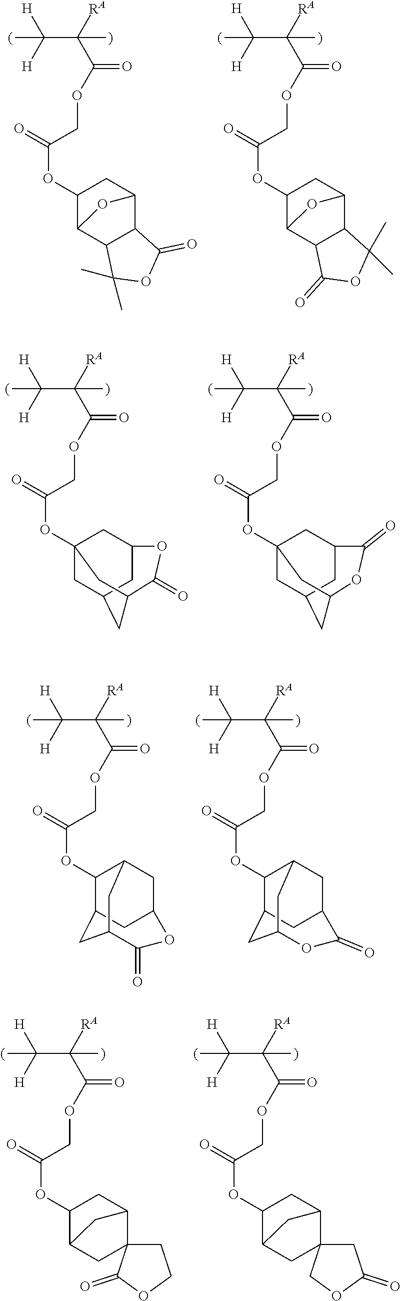

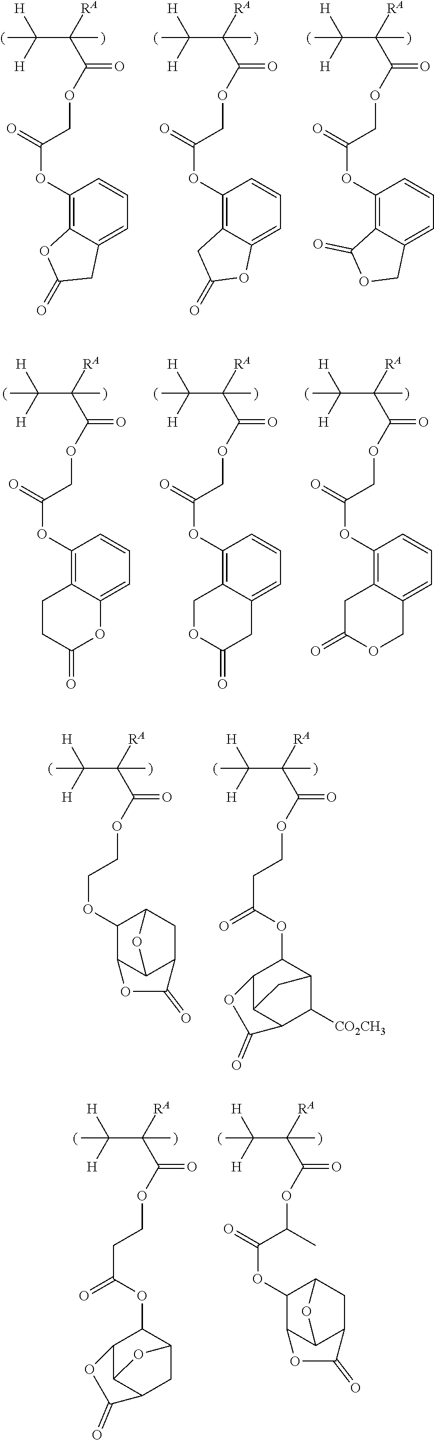

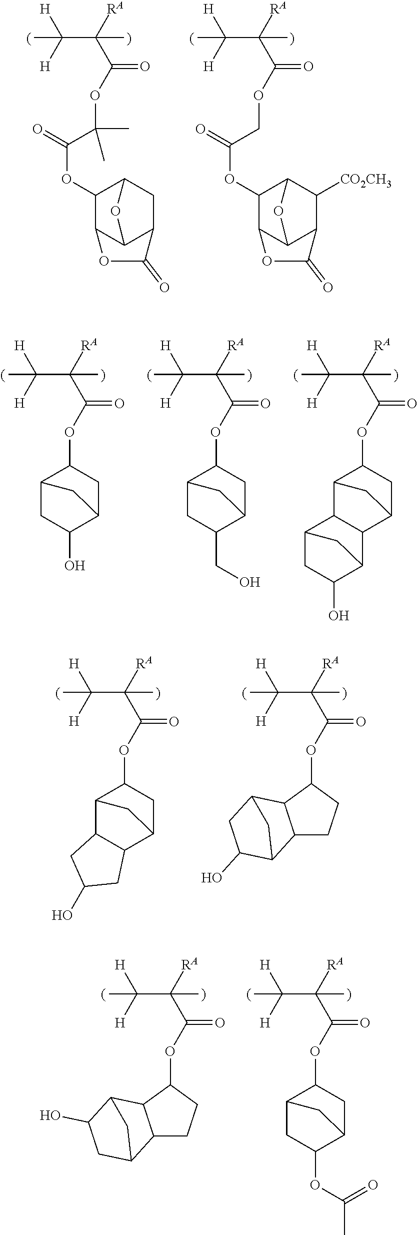

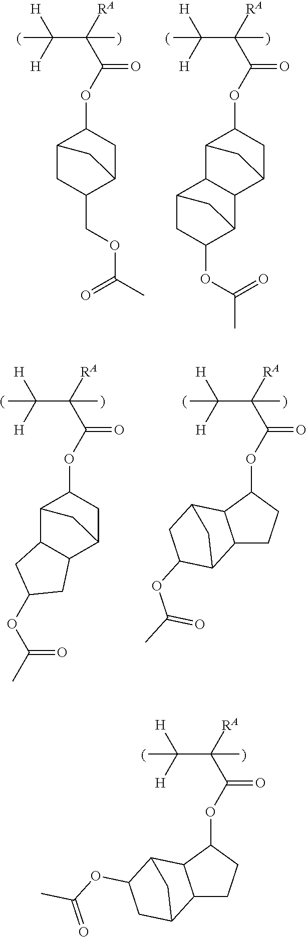

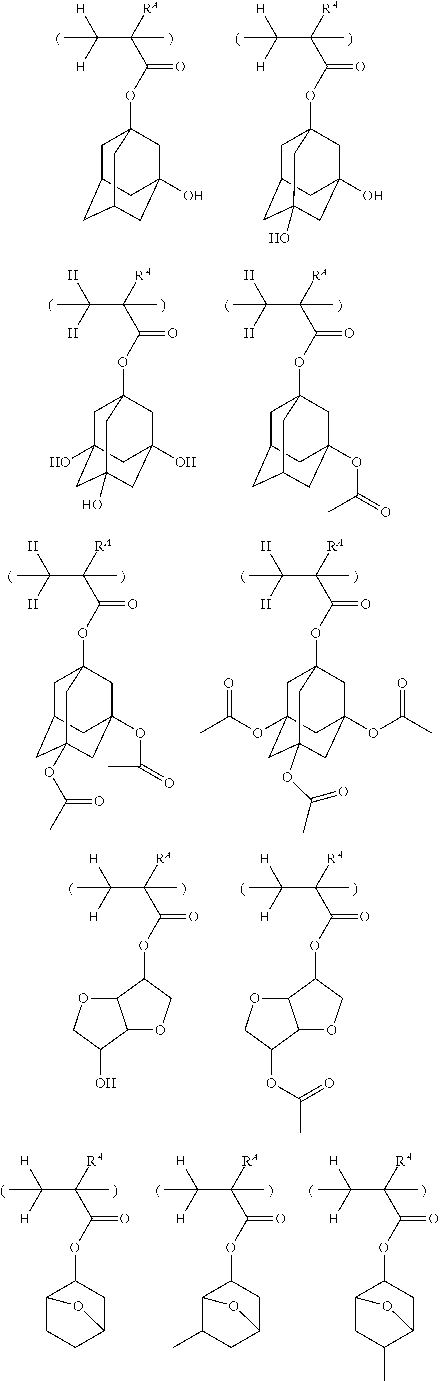

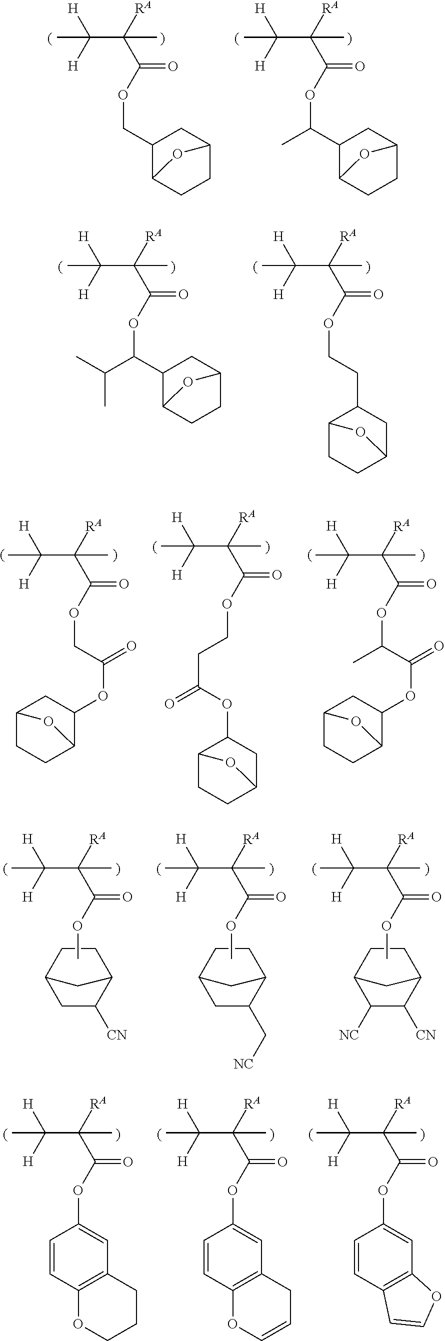

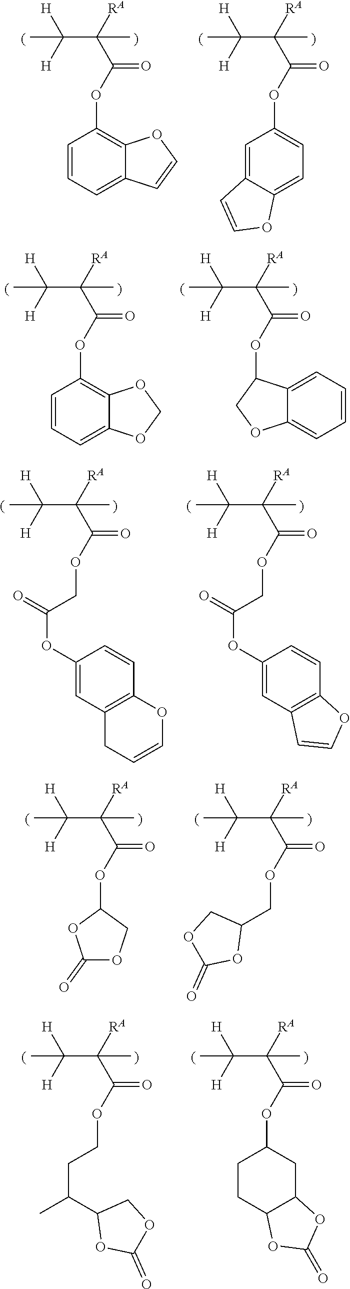

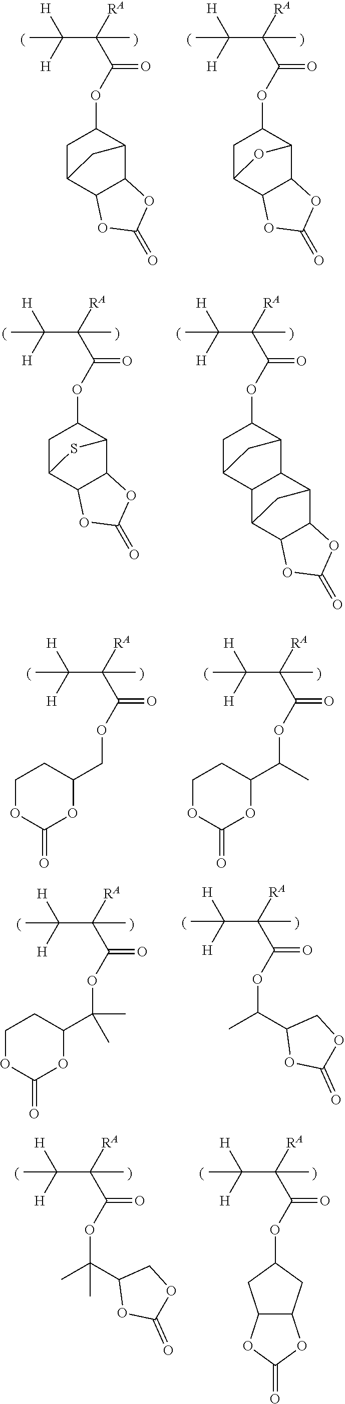

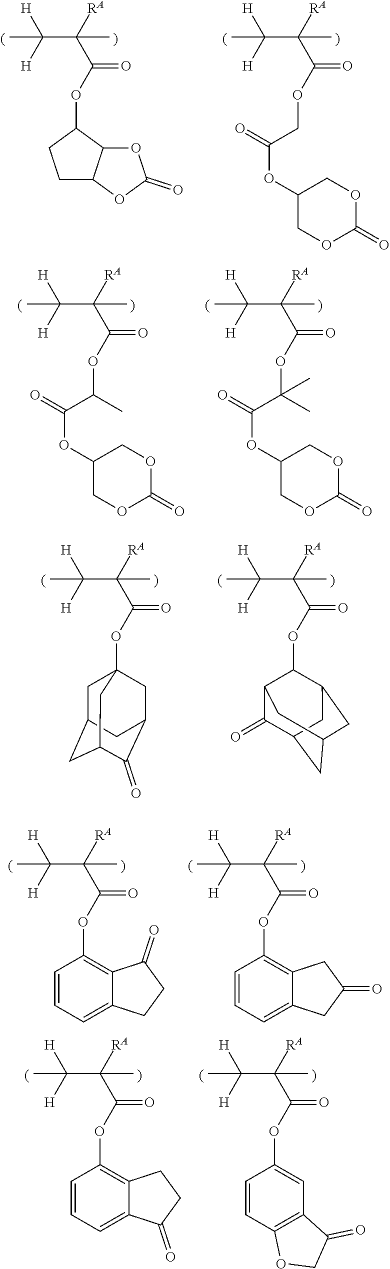

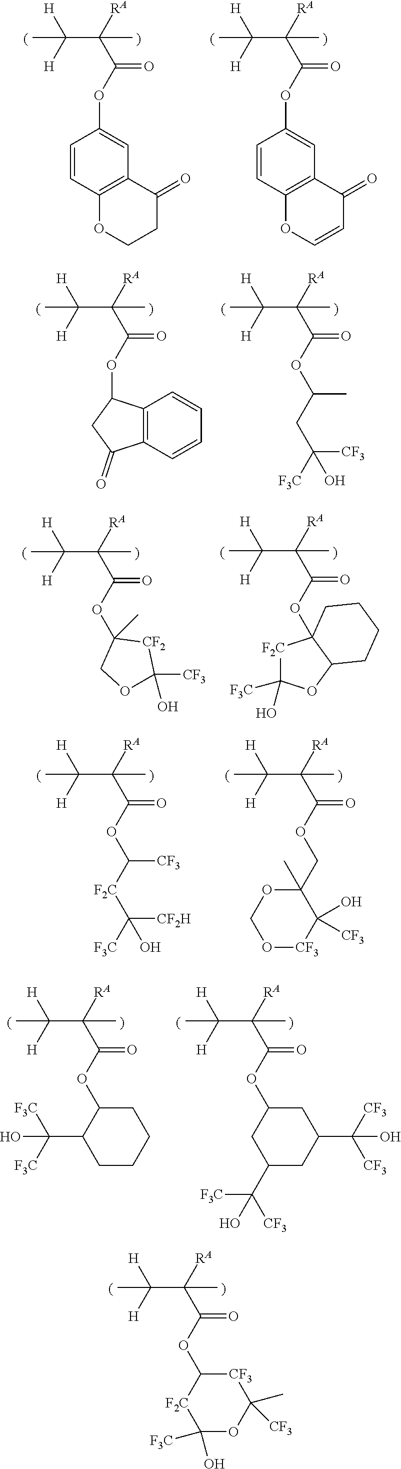

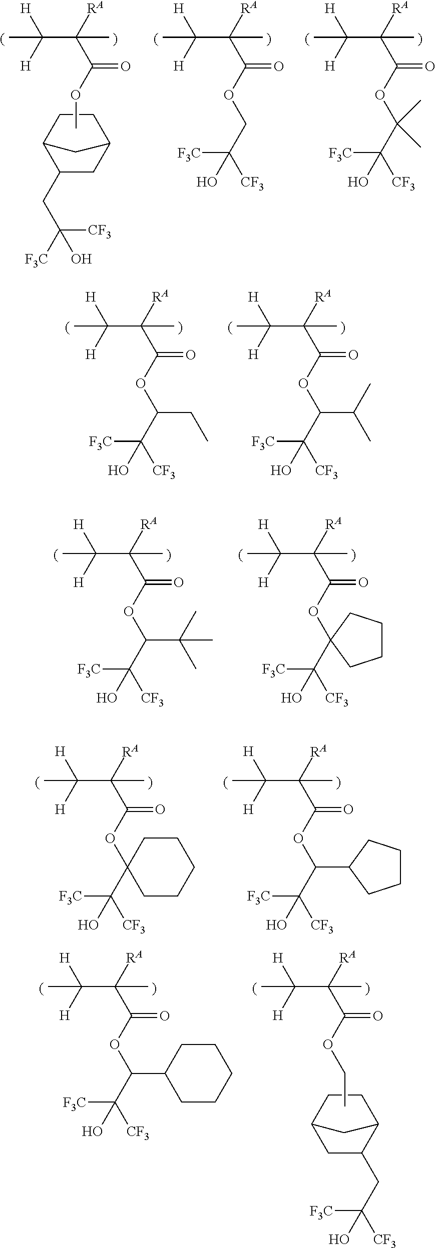

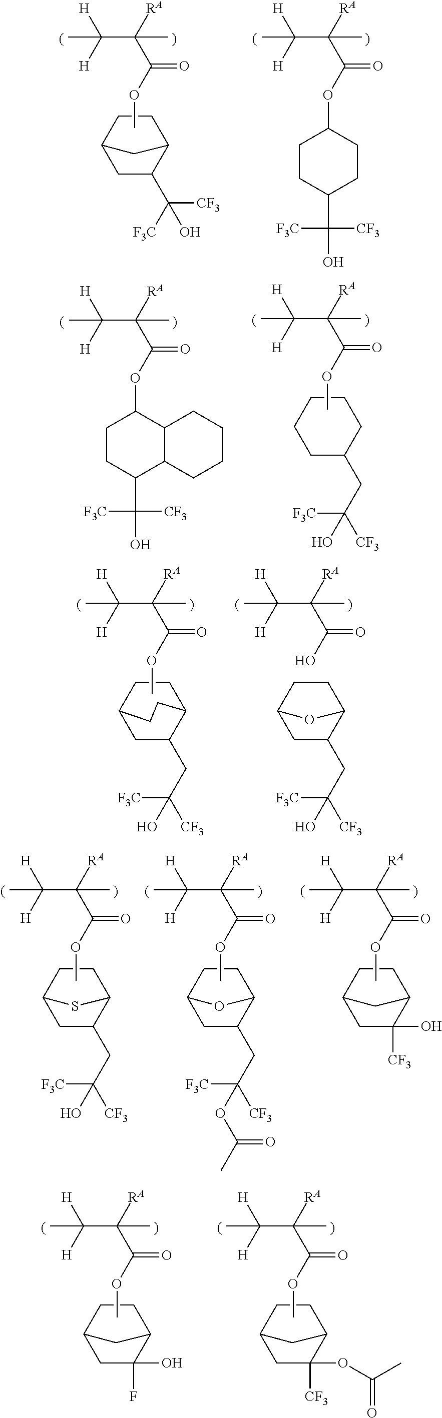

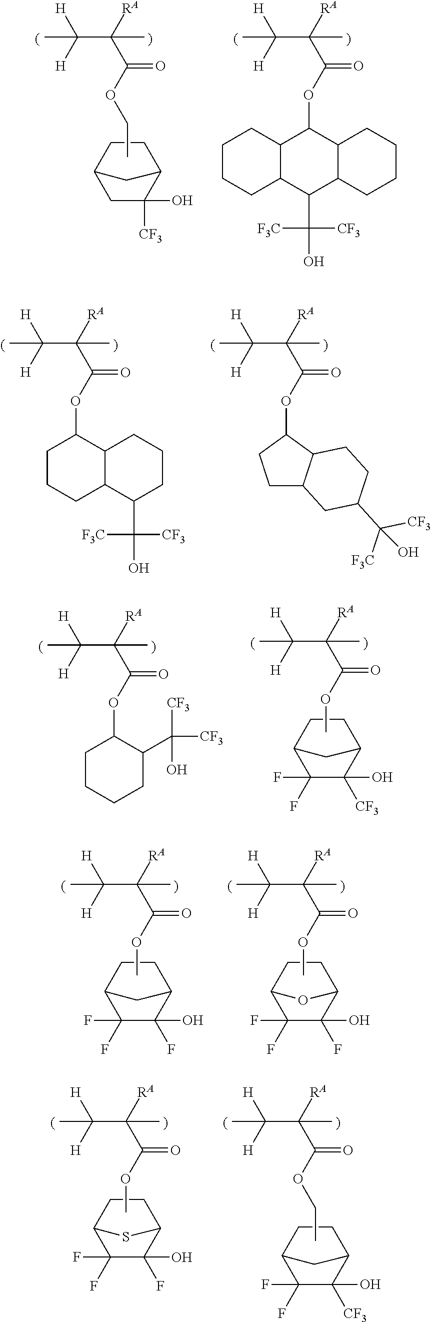

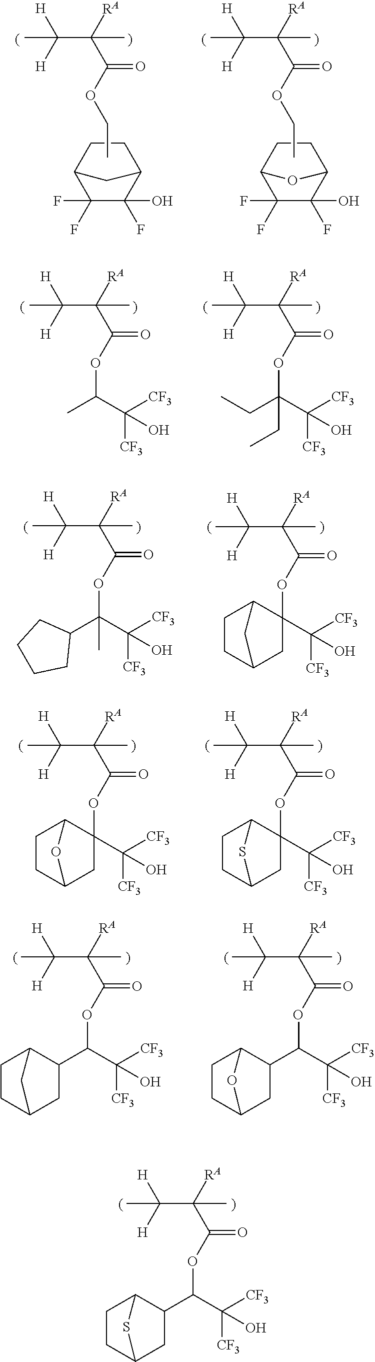

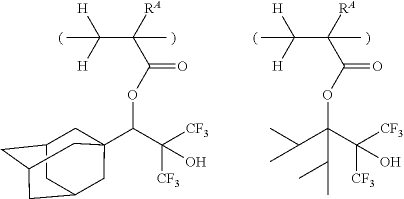

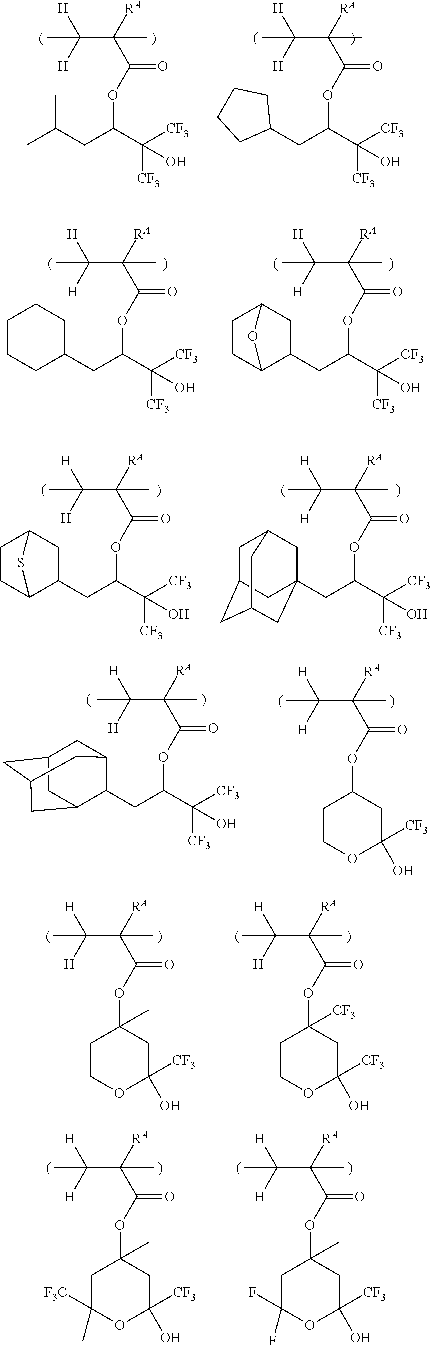

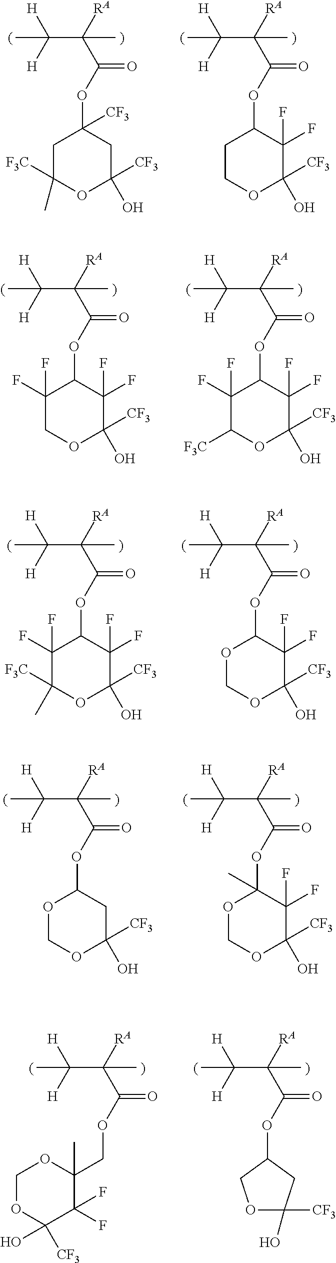

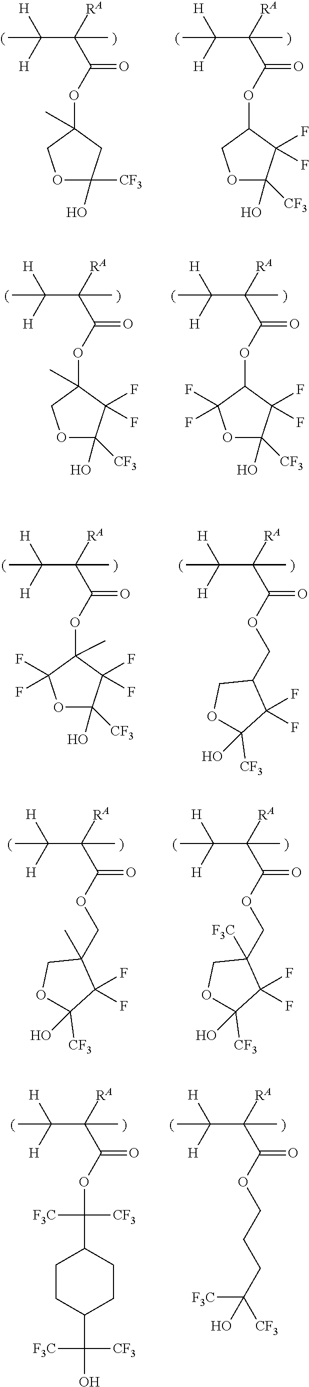

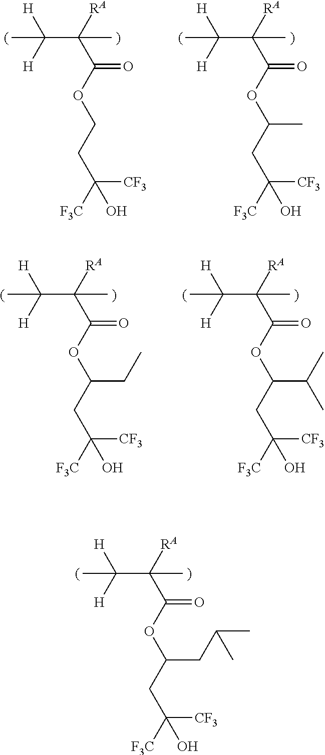

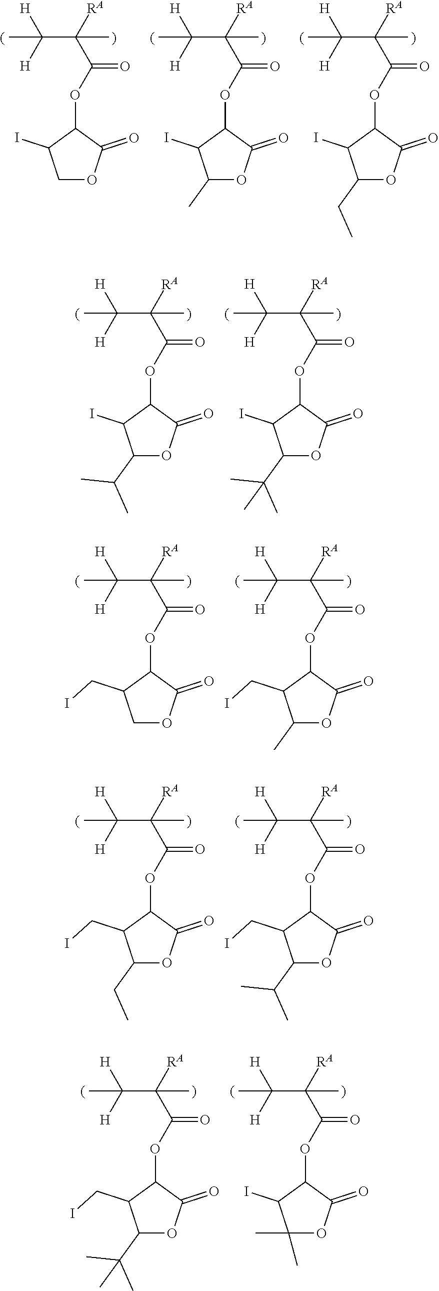

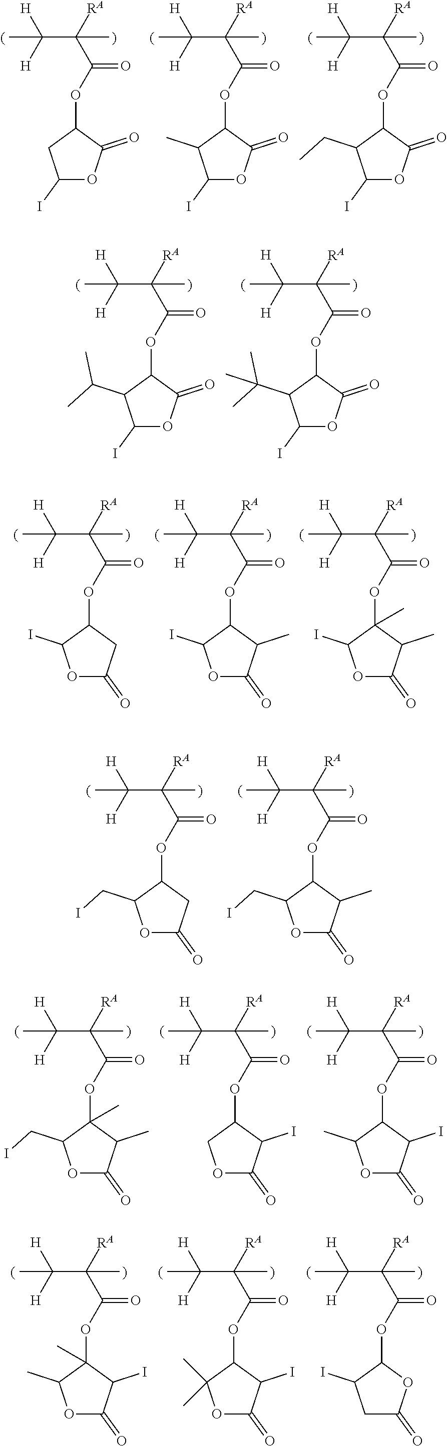

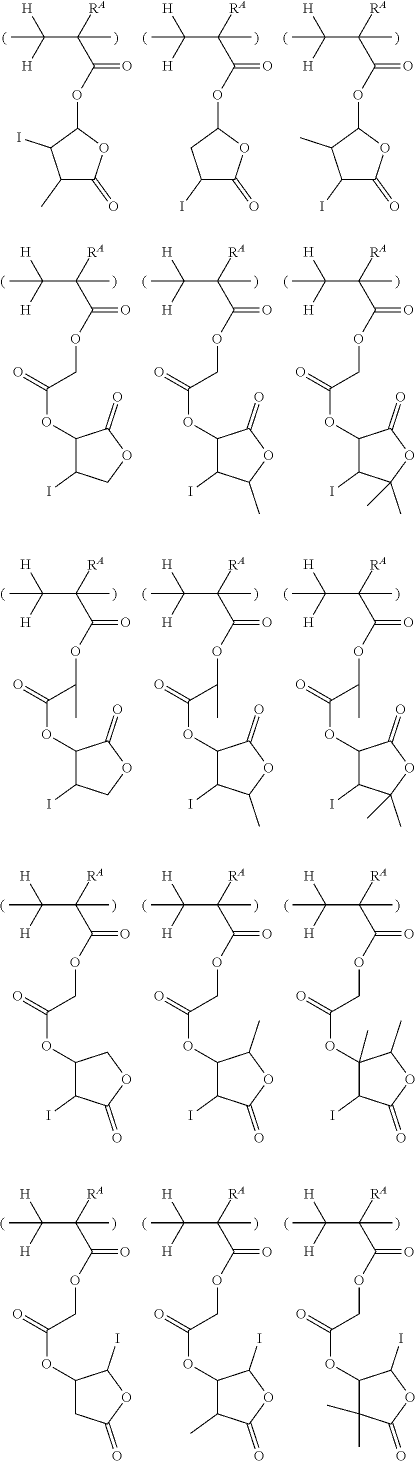

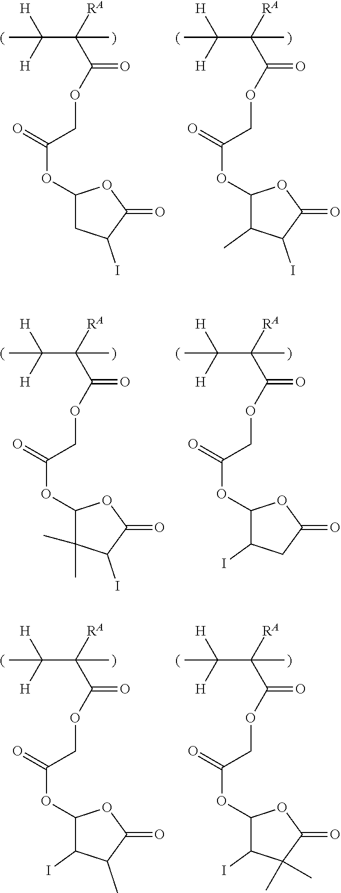

Examples of the repeat unit (b2) are shown below, but not limited thereto. Herein R^{A} is as defined above.

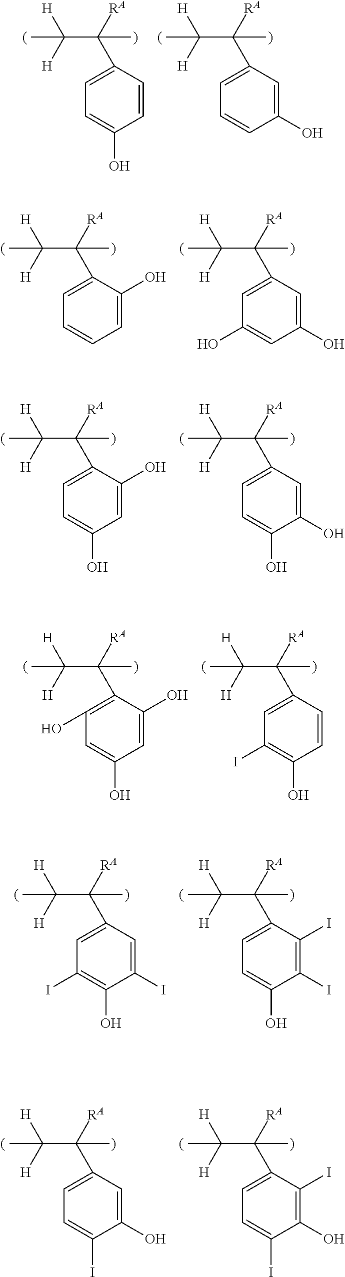

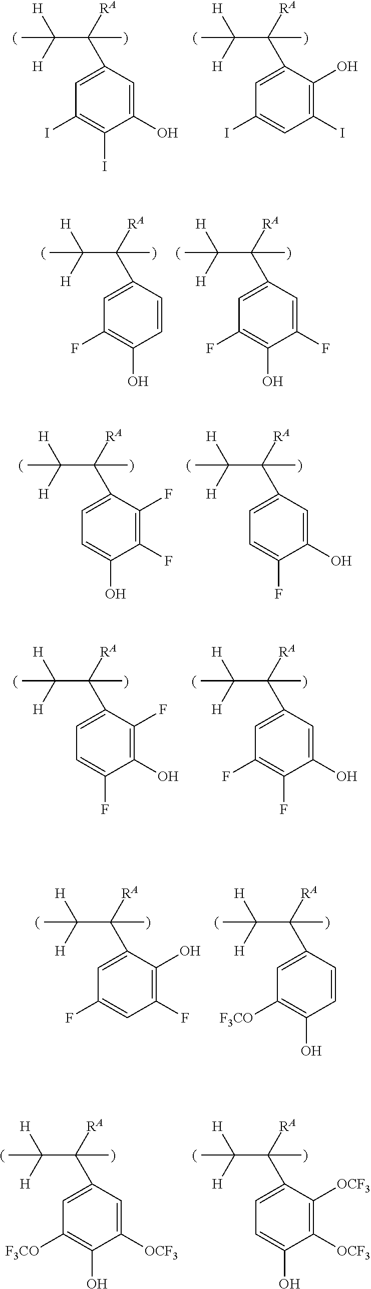

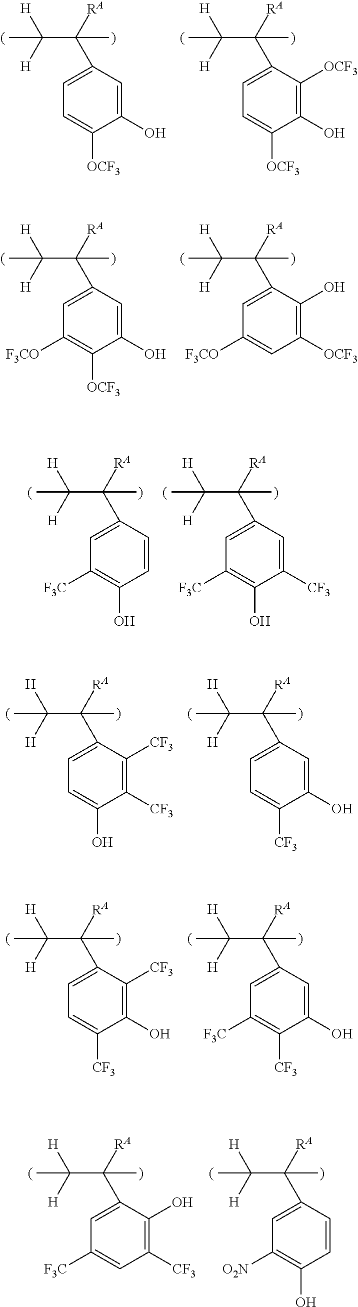

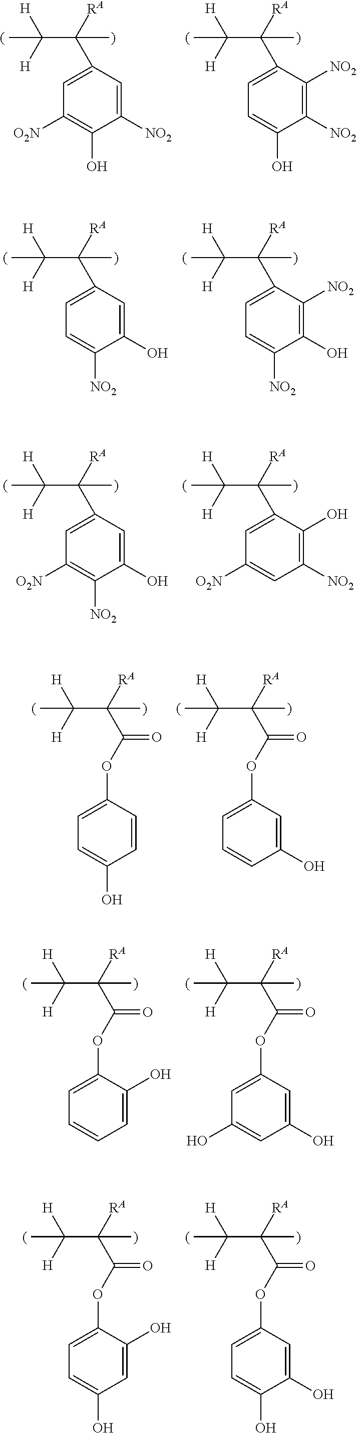

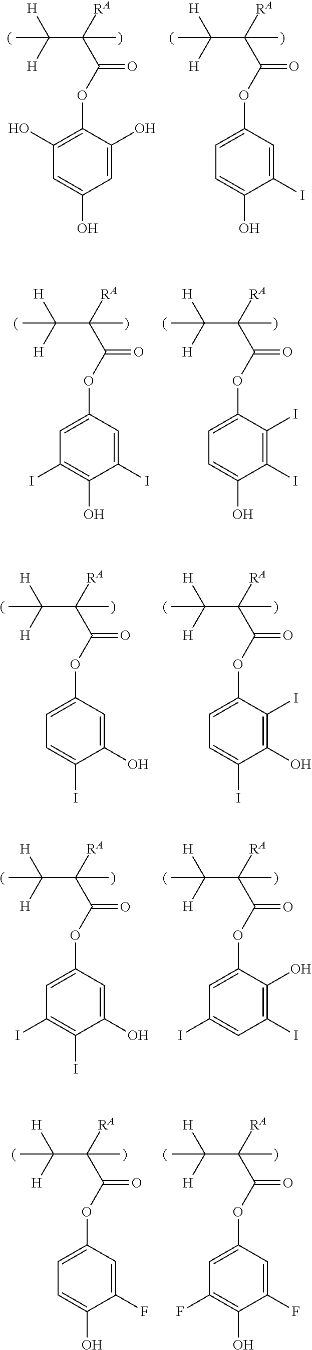

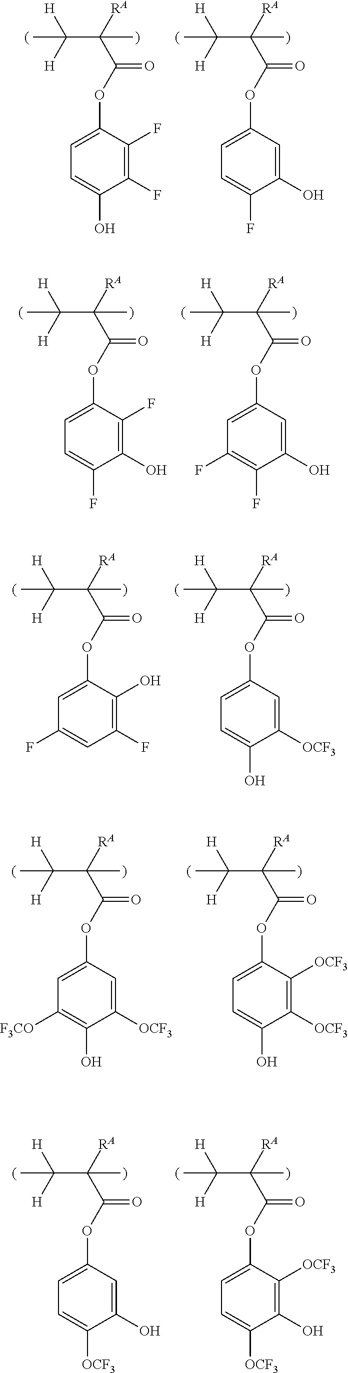

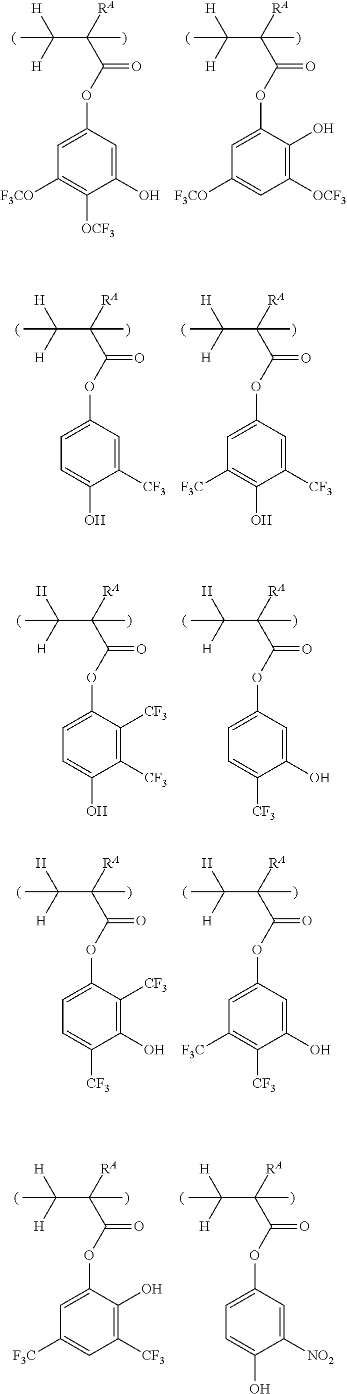

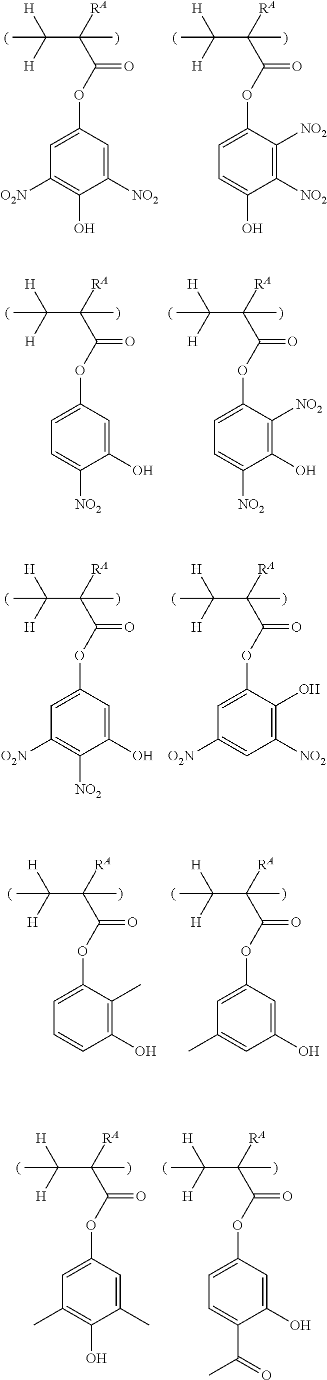

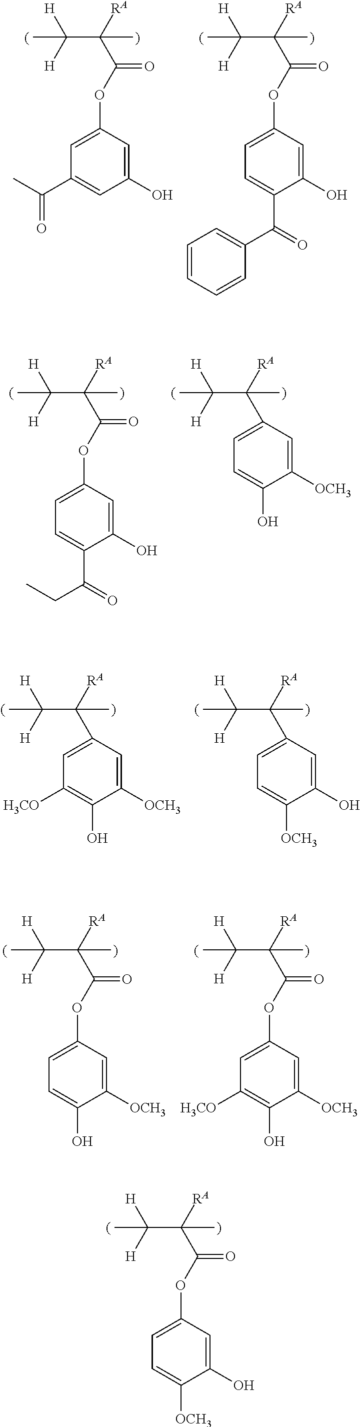

Of the repeat units (b1) and (b2), those units having a lactone ring as the polar group are preferred in the case of ArF lithography, and those units having a phenol site as the polar group are preferred in the case of KrF, EB or EUV lithography.

In a preferred embodiment, the base polymer further comprises repeat units of at least one type selected from repeat units having the formulae (c1) to (c4), which are also referred to as repeat units (c1) to (c4).

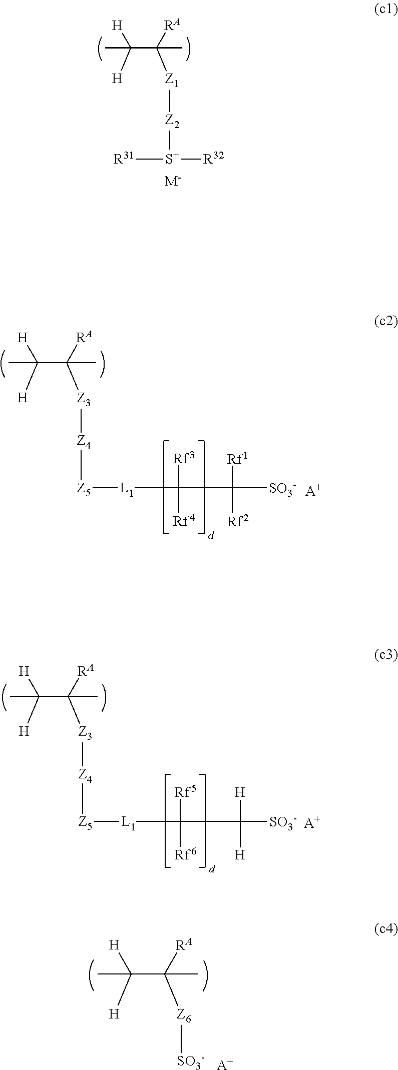

In formulae (c1) to (c4), R^{A} is each independently hydrogen, fluorine, methyl or trifluoromethyl. Z¹ is a single bond or phenylene group. Z² is *—C(═O)—O—Z²¹—, *—C(═O)—NH—Z²¹—, or *—O—Z²¹—, wherein Z²¹ is a C₁-C₆ aliphatic hydrocarbylene group, phenylene, or divalent group obtained by combining the foregoing, which may contain a carbonyl moiety, ester bond, ether bond or hydroxy moiety. Z³ is each independently a single bond, phenylene group, naphthylene group or *—C(═O)—O—Z³¹—, wherein Z³¹ is a C₁-C₁₀ aliphatic hydrocarbylene group which may contain a hydroxy moiety, ether bond, ester bond or lactone ring, or a phenylene group or naphthylene group. Z⁴ is each independently a single bond, *—Z⁴¹—C(═O)—O—, *—C(═O)—NH—Z⁴¹— or *—O—Z⁴¹—, wherein Z⁴¹ is a C₁-C₂₀ hydrocarbylene group which may contain a heteroatom. Z⁵ is each independently a single bond, *—Z⁵—C(═O)—O—, *—C(═O)—NH—Z⁵— or *—O—Z⁵—, wherein Z⁵¹ is a C₁-C₂₀ hydrocarbylene group which may contain a heteroatom. Z⁶ is a single bond, methylene, ethylene, phenylene, fluorinated phenylene, trifluoromethyl-substituted phenylene, *—C(═O)—O—Z⁶¹—, *—C(═O)—N(H)—Z⁶¹— or *—O—Z⁶¹—, wherein Z⁶¹ is a C₁-C₆ aliphatic hydrocarbylene group, phenylene, fluorinated phenylene or trifluoromethyl-substituted phenylene group, which may contain a carbonyl moiety, ester bond, ether bond or hydroxy moiety. The asterisk (*) designates a point of attachment to the carbon atom in the backbone.

The aliphatic hydrocarbylene group represented by Z²¹, Z³¹ and Z⁶¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl, and cyclohexanediyl; and combinations thereof.

The hydrocarbylene group represented by Z⁴¹ and Z⁵¹ may be saturated or unsaturated and straight, branched or cyclic. Examples thereof are shown below, but not limited thereto.

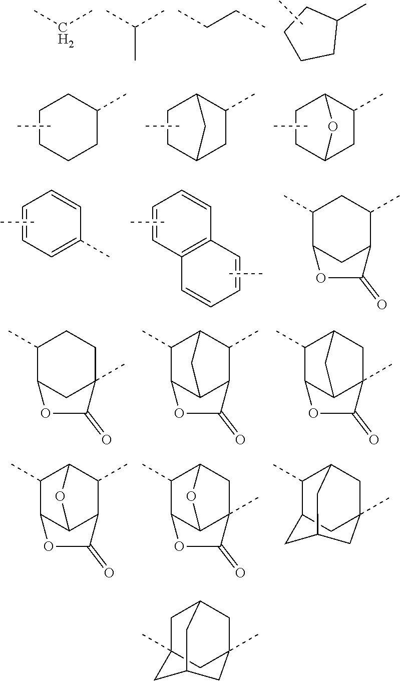

In formula (c1), R³¹ and R³² are each independently a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, propenyl, butenyl, and hexenyl; C₃-C₂₀ cyclic unsaturated hydrocarbyl groups such as cyclohexenyl; C₆-C₂₀ aryl groups such as phenyl, naphthyl, and thienyl; C₇-C₂₀ aralkyl groups such as benzyl, 1-phenylethyl and 2-phenylethyl; and combinations thereof. Inter alia, aryl groups are preferred. In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety.

Also, R³¹ and R³² may bond together to form a ring with the sulfur atom to which they are attached. Examples of the ring are as exemplified above for the ring that R¹¹ and R¹² in formula (cation-1), taken together, form with the sulfur atom to which they are attached.

Examples of the cation in repeat unit (c1) are shown below, but not limited thereto. Herein R^{A} is as defined above.

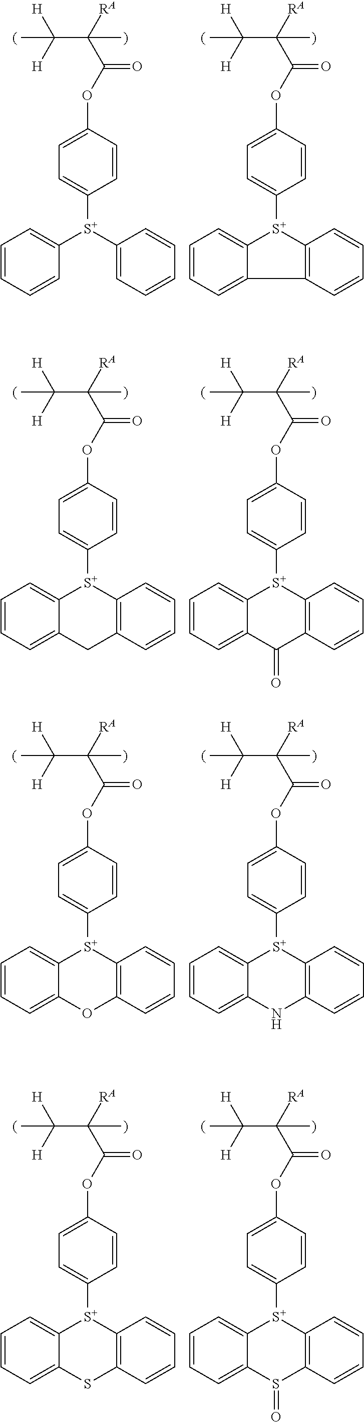

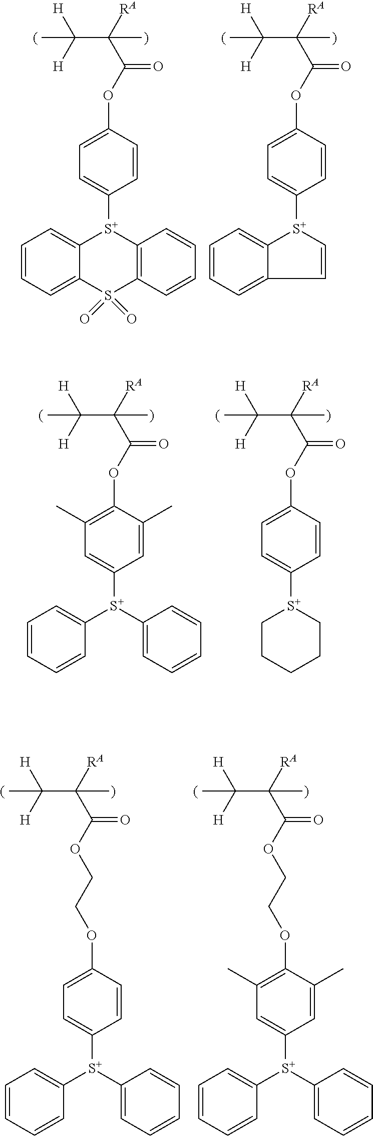

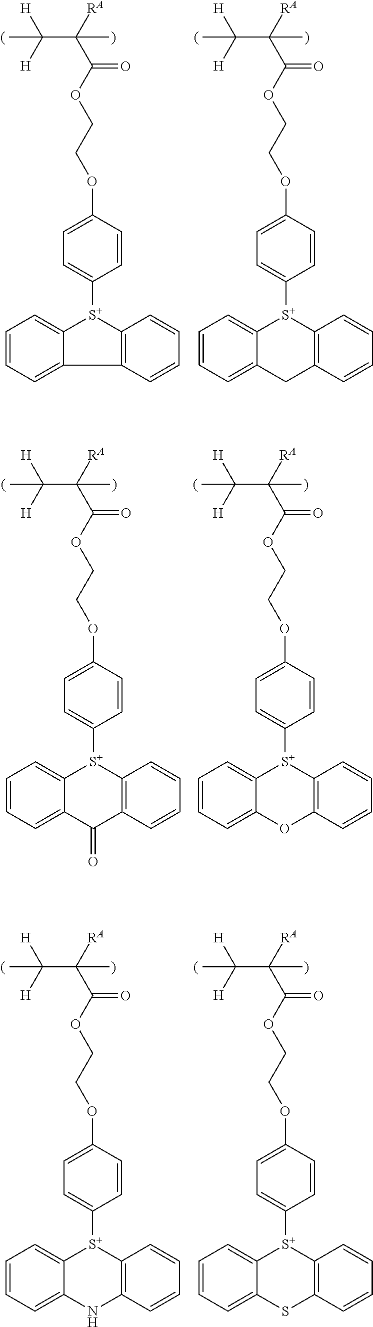

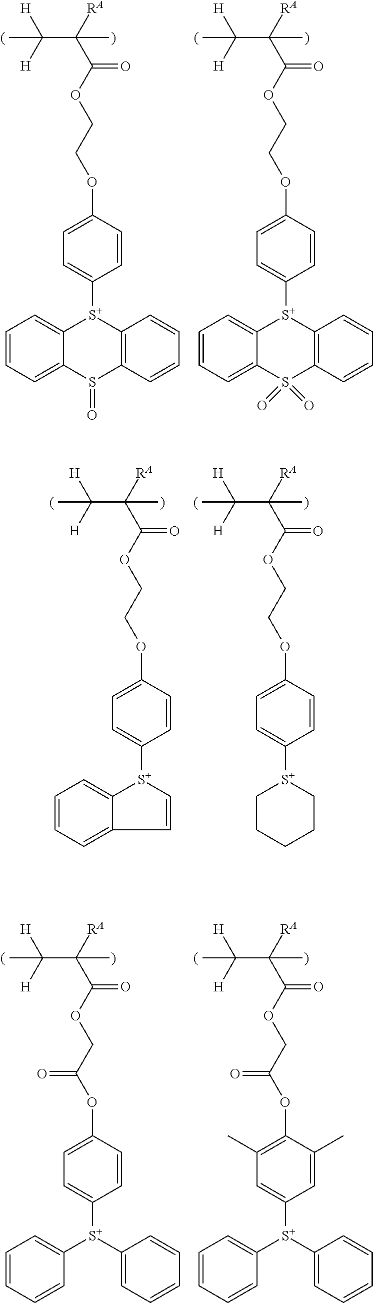

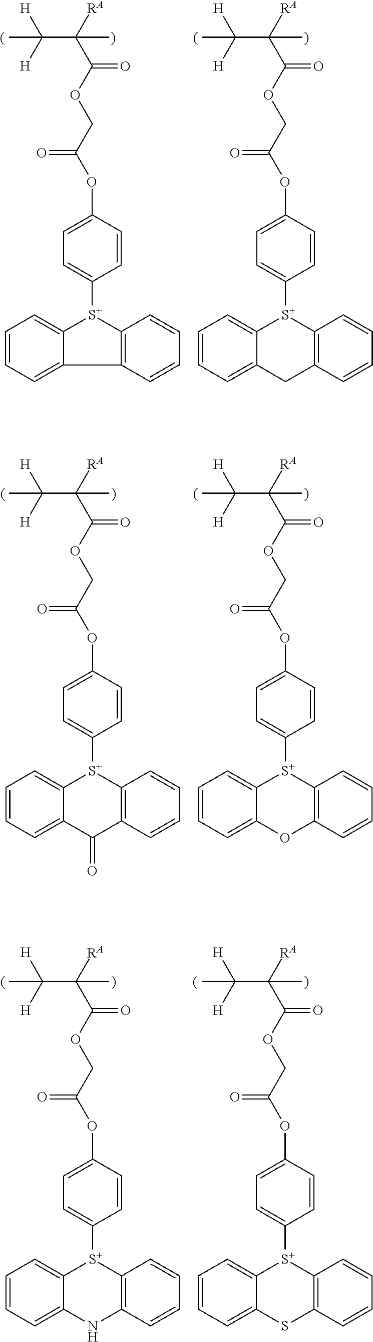

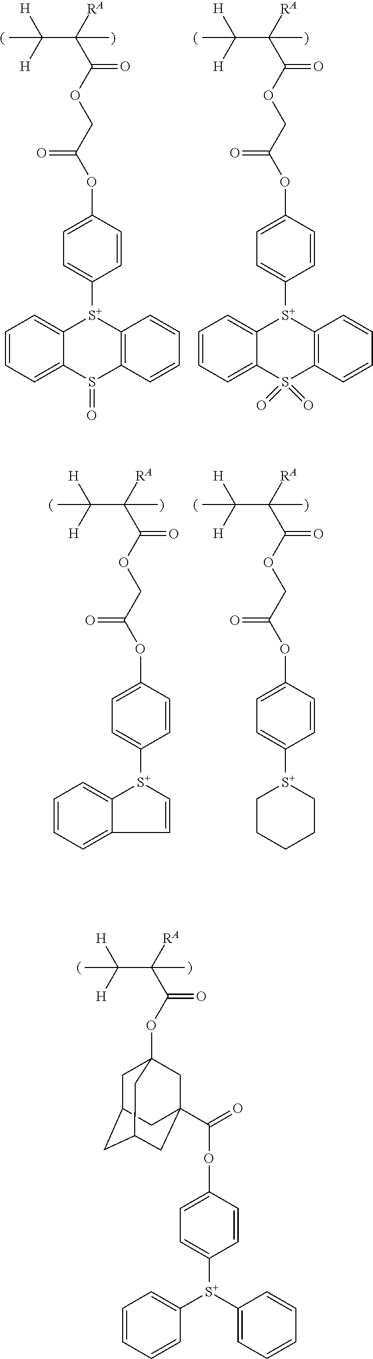

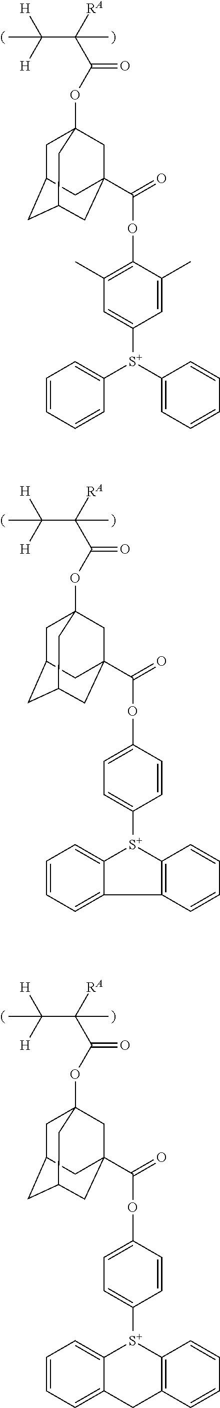

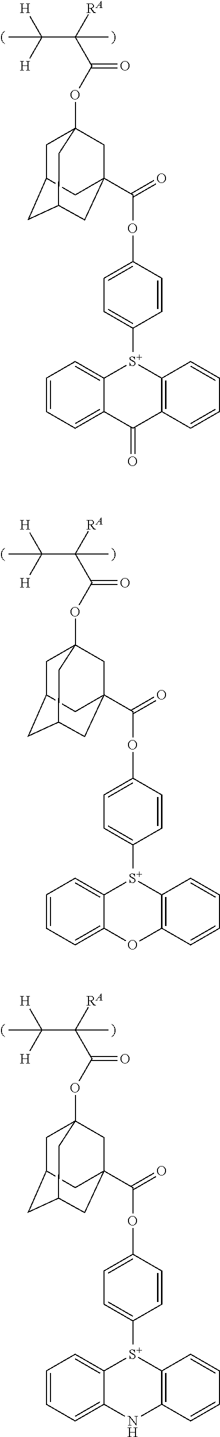

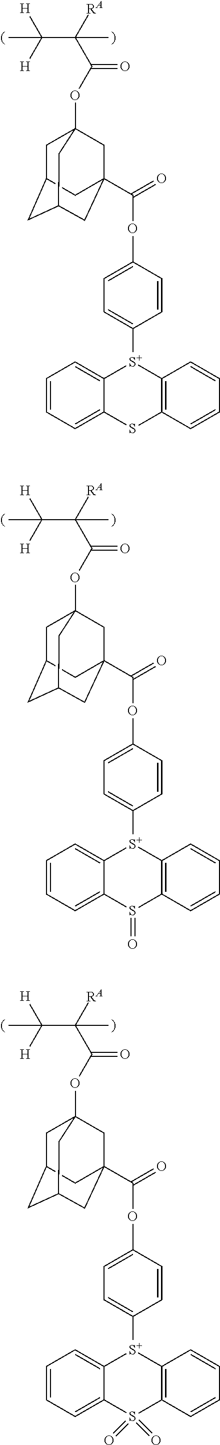

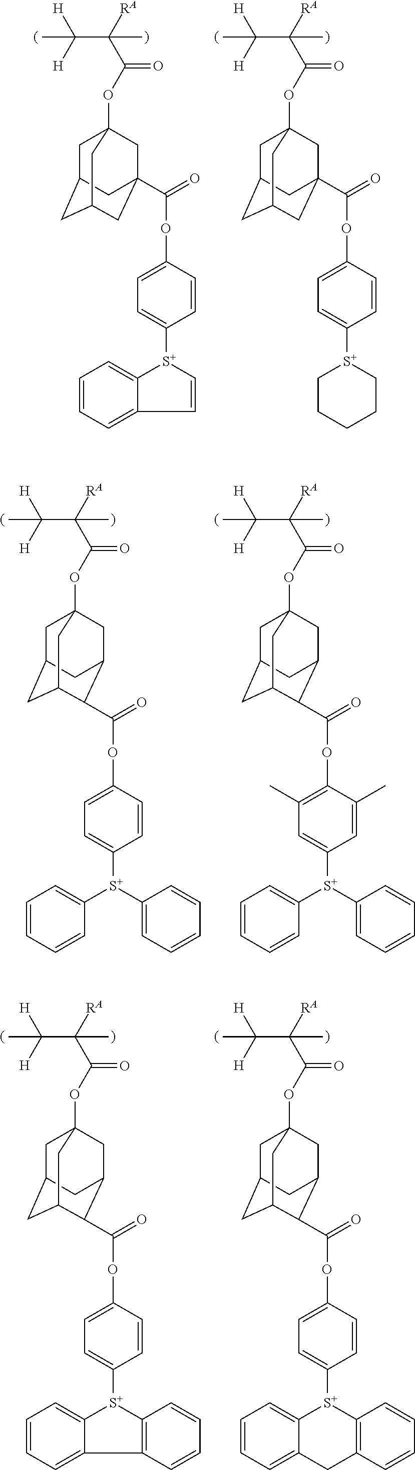

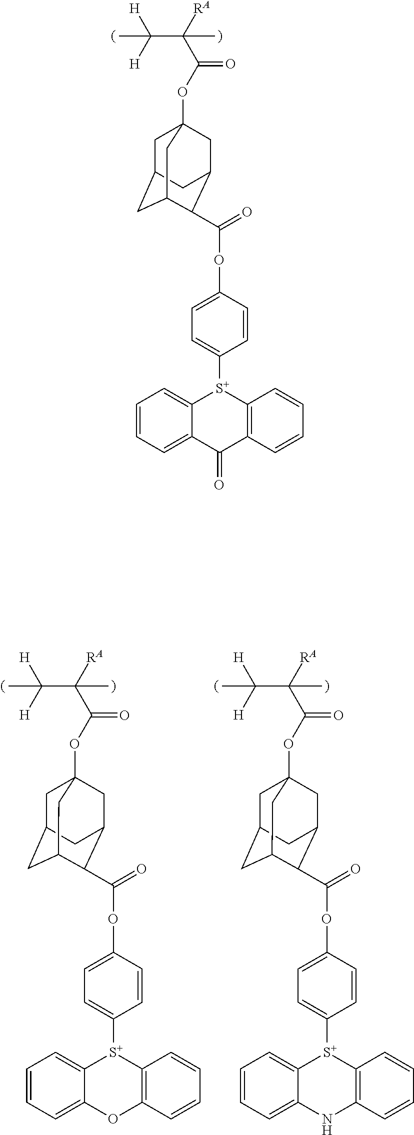

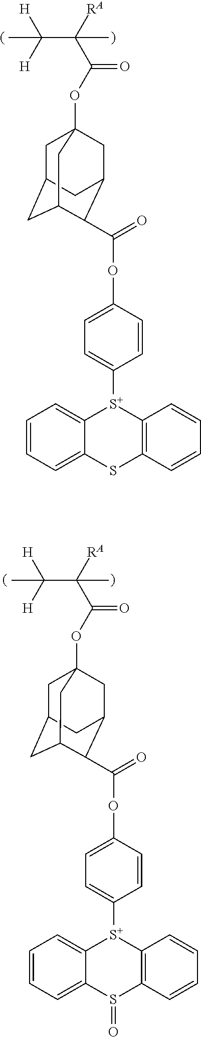

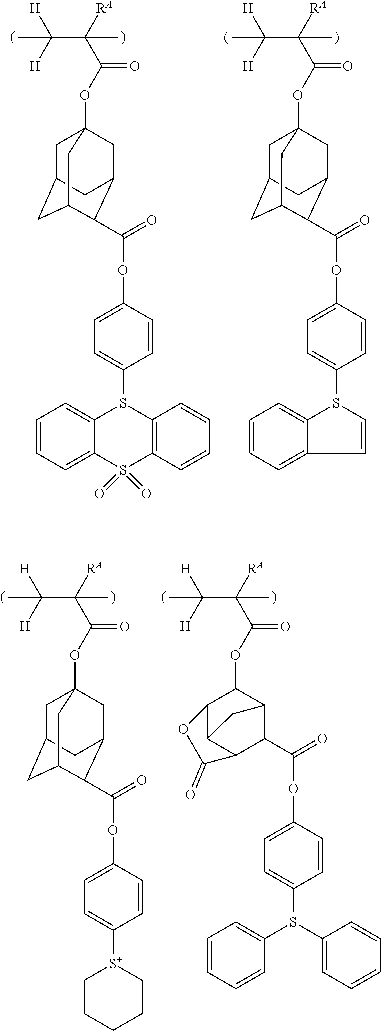

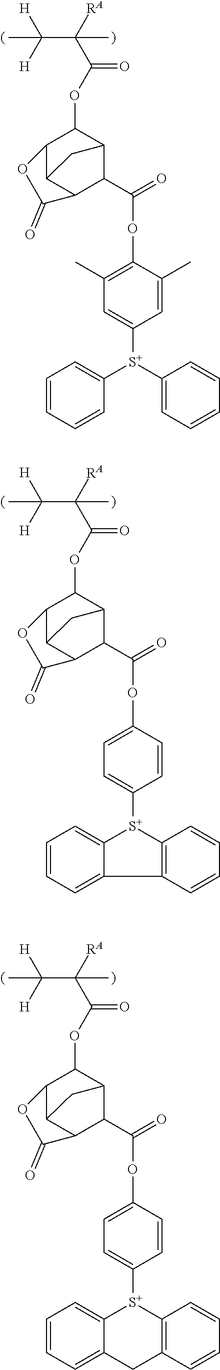

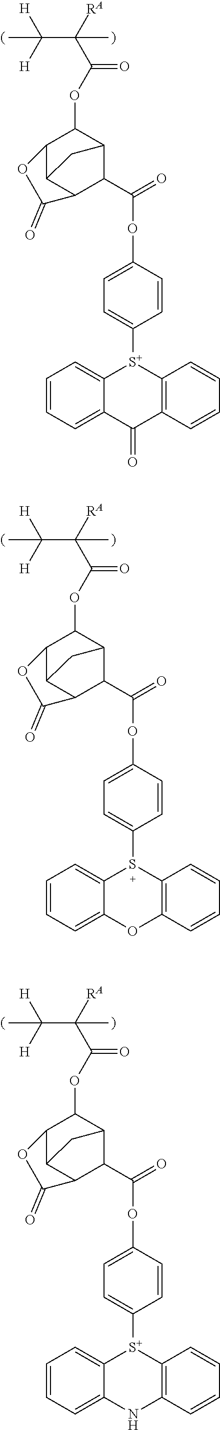

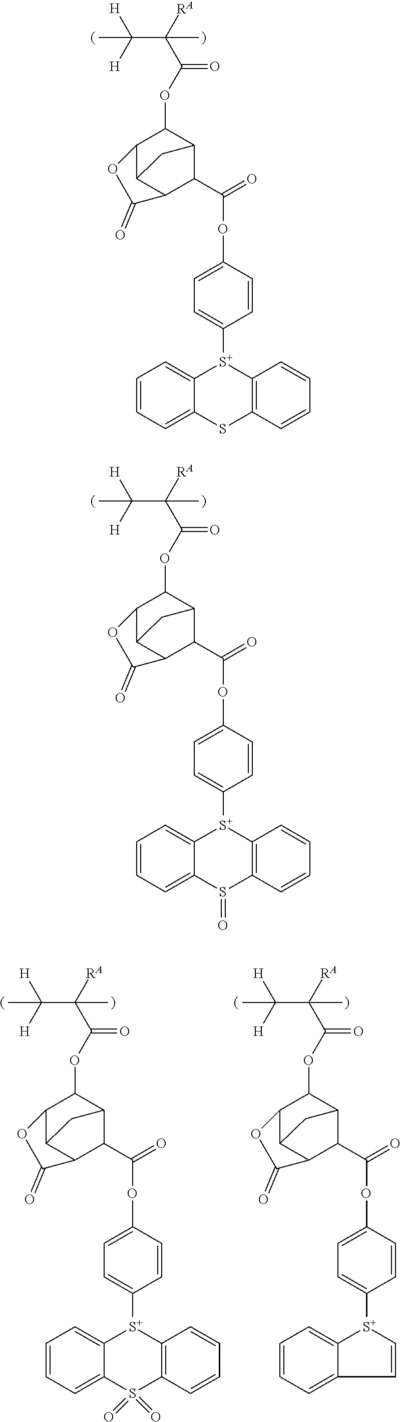

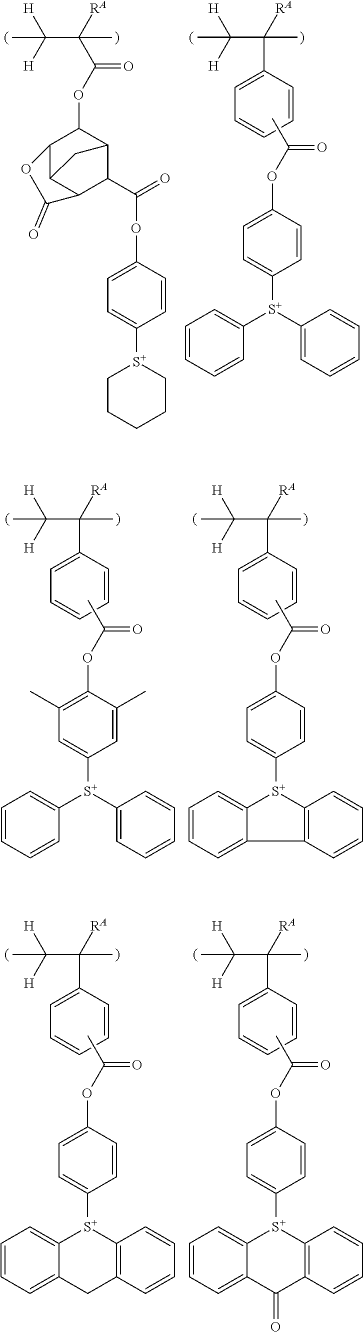

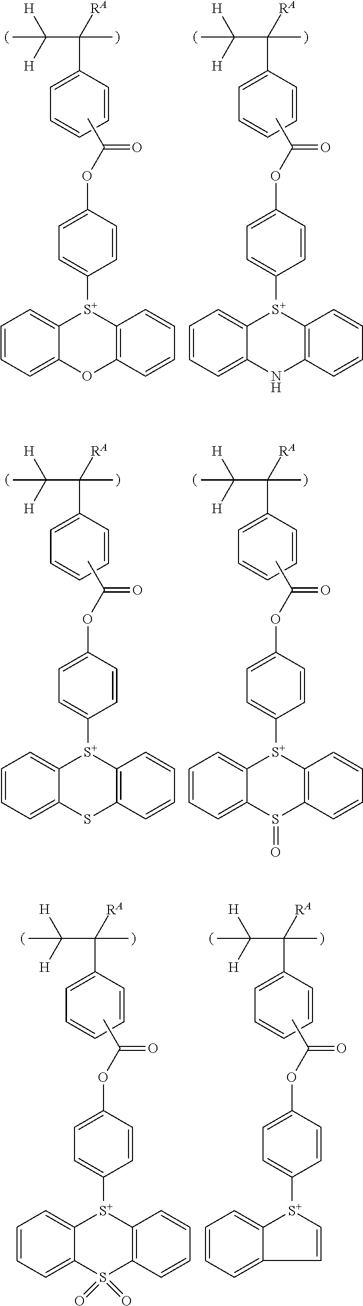

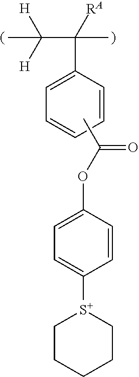

In formula (c1), M⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; methide ions such as tris(trifluoromethylsulfonyl)methide and tris(perfluoroethylsulfonyl)methide.

Also included are sulfonate anions having fluorine substituted at α-position as represented by the formula (c1-1) and sulfonate anions having fluorine substituted at α-position and trifluoromethyl at β-position as represented by the formula (c1-2).

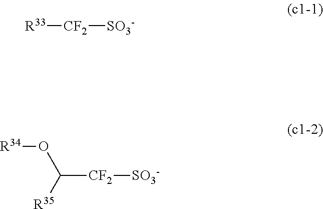

In formula (c1-1), R³³ is hydrogen, a C₁-C₃₀ hydrocarbyl group, C₂-C₃₀ hydrocarbylcarbonyloxy group, or C₂-C₃₀ hydrocarbyloxycarbonyl group. The hydrocarbyl group may contain halogen, ether bond, ester bond, carbonyl moiety, or lactone ring. The hydrocarbyl group and hydrocarbyl moiety in the hydrocarbylcarbonyloxy and hydrocarbyloxycarbonyl groups may be saturated or unsaturated and straight, branched or cyclic. Examples of the hydrocarbyl group are as will be exemplified later for R^{fa1} in formula (2A′).

In formula (c1-2), R³⁴ is hydrogen, or a C₁-C₃₀ hydrocarbyl group or C₂-C₃₀ hydrocarbylcarbonyl group, which may contain halogen, ether bond, ester bond, carbonyl moiety or lactone ring. R³⁵ is hydrogen, fluorine, or C₁-C₆ fluorinated alkyl group. The hydrocarbyl group and hydrocarbyl moiety in the hydrocarbylcarbonyl group may be saturated or unsaturated and straight, branched or cyclic. Examples of the hydrocarbyl group are as will be exemplified later for R^{fa1} in formula (2A′). R³⁵ is preferably trifluoromethyl.

Examples of the sulfonate anion having formula (c1-1) or (c1-2) are shown below, but not limited thereto. R³⁵ is as defined above.

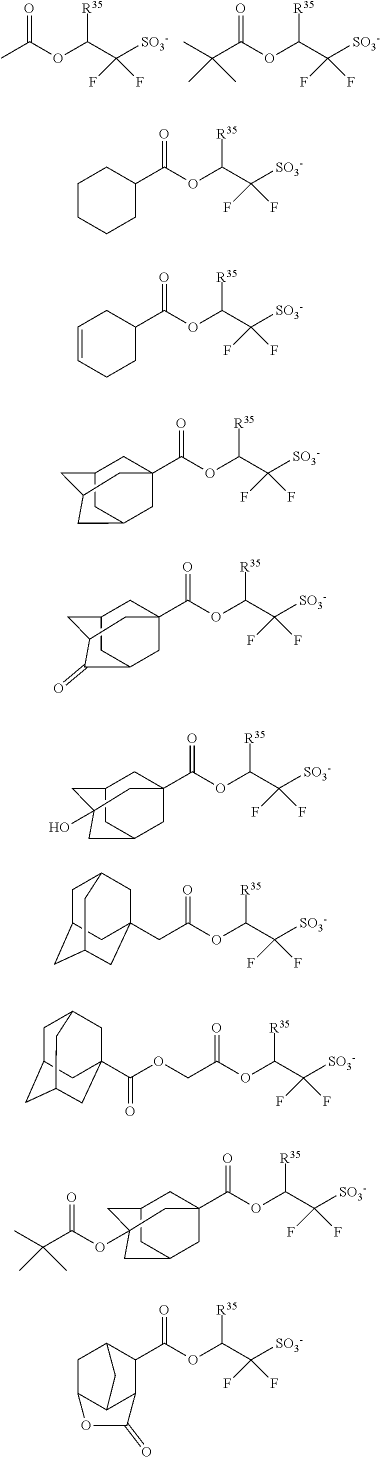

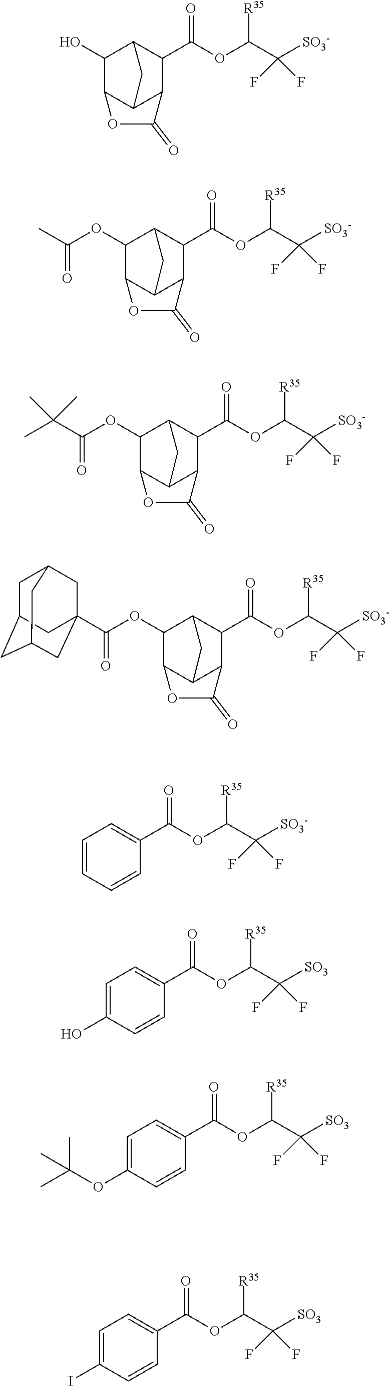

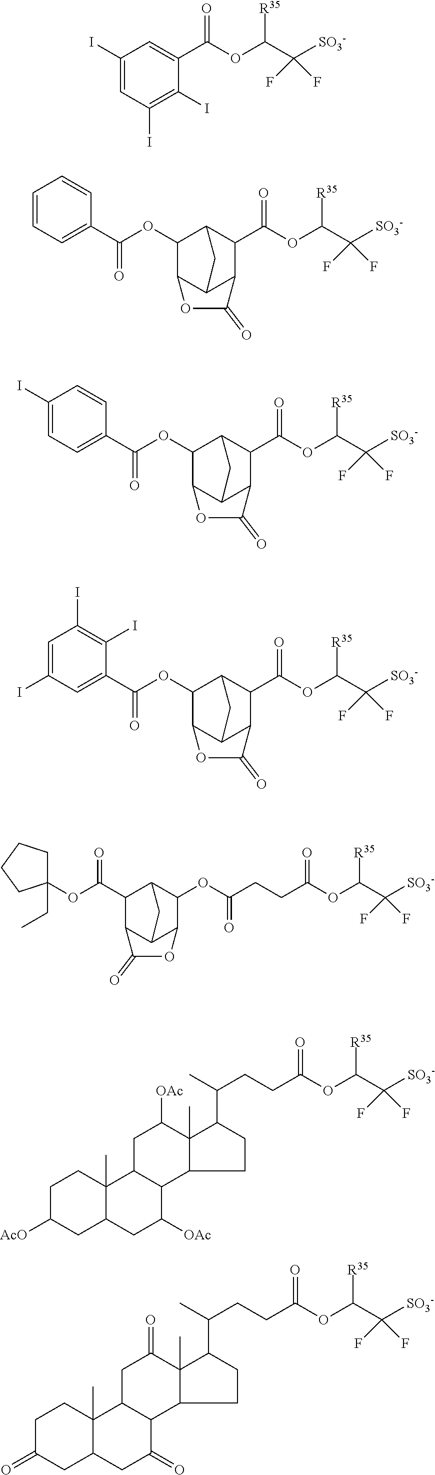

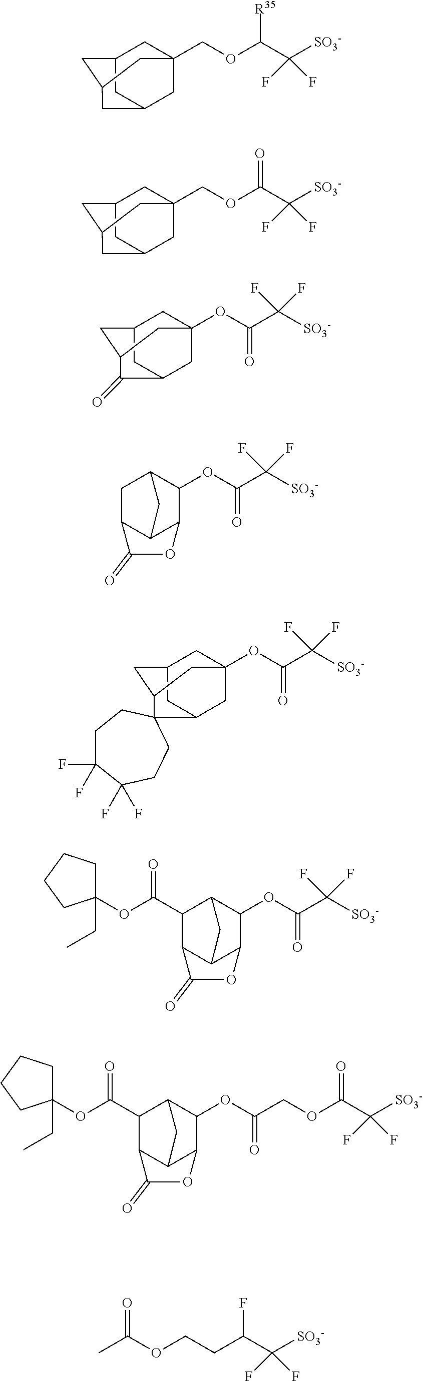

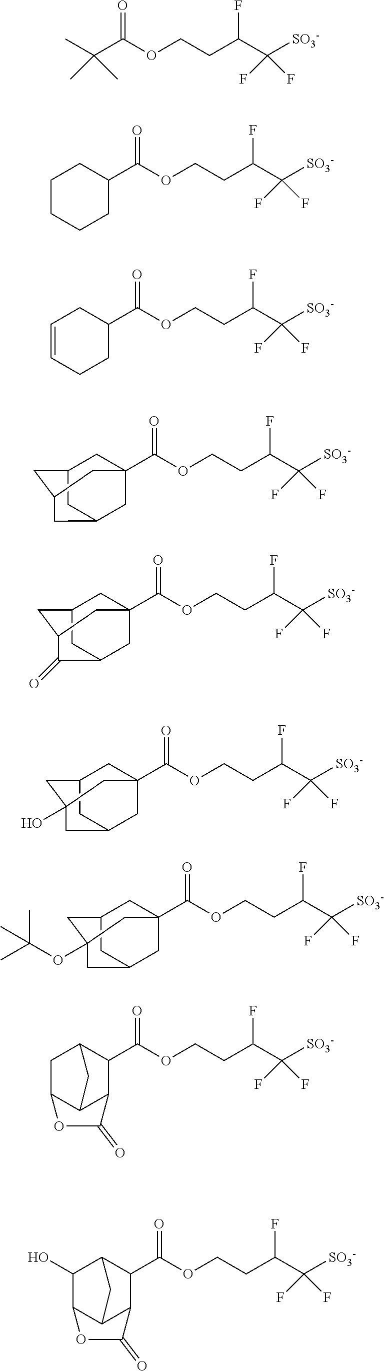

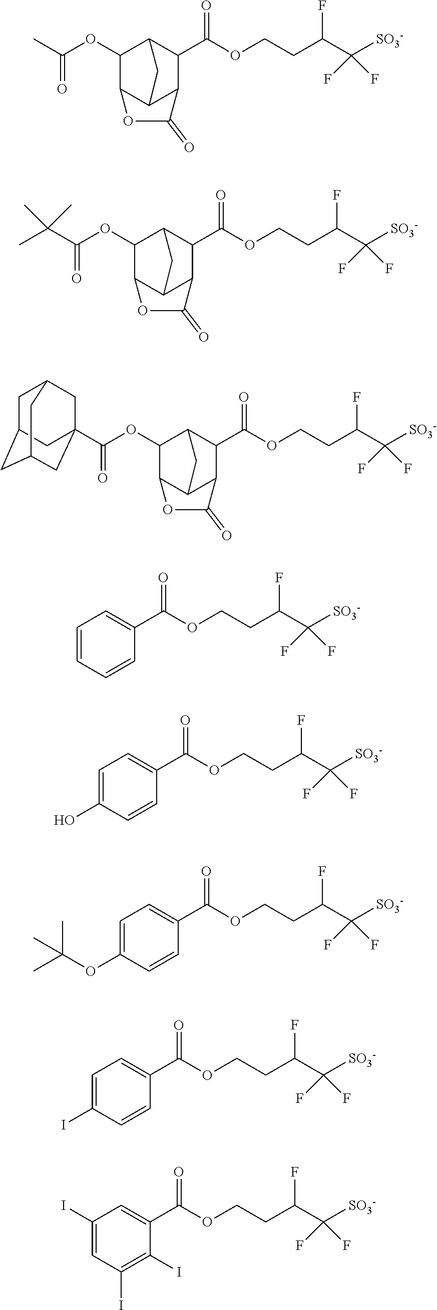

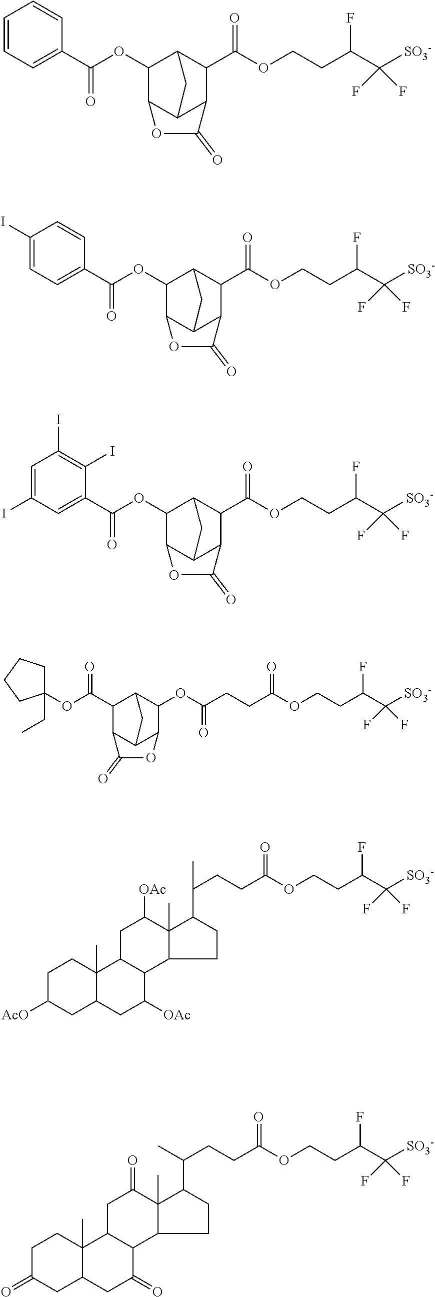

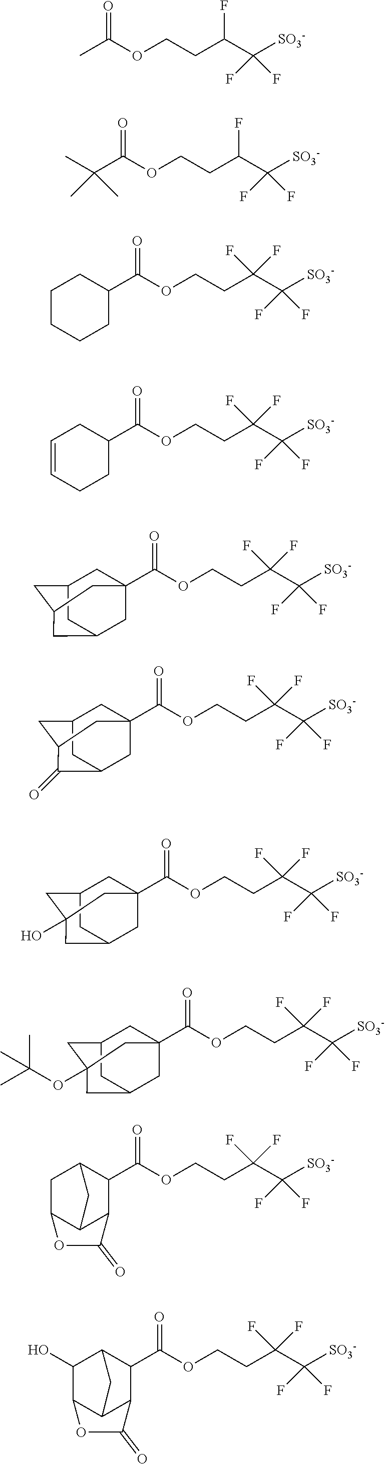

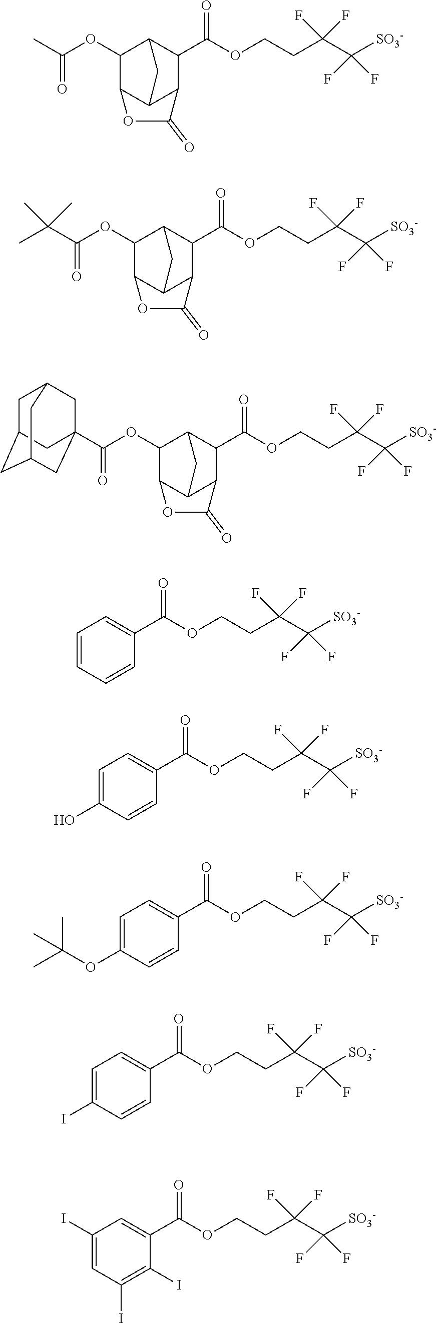

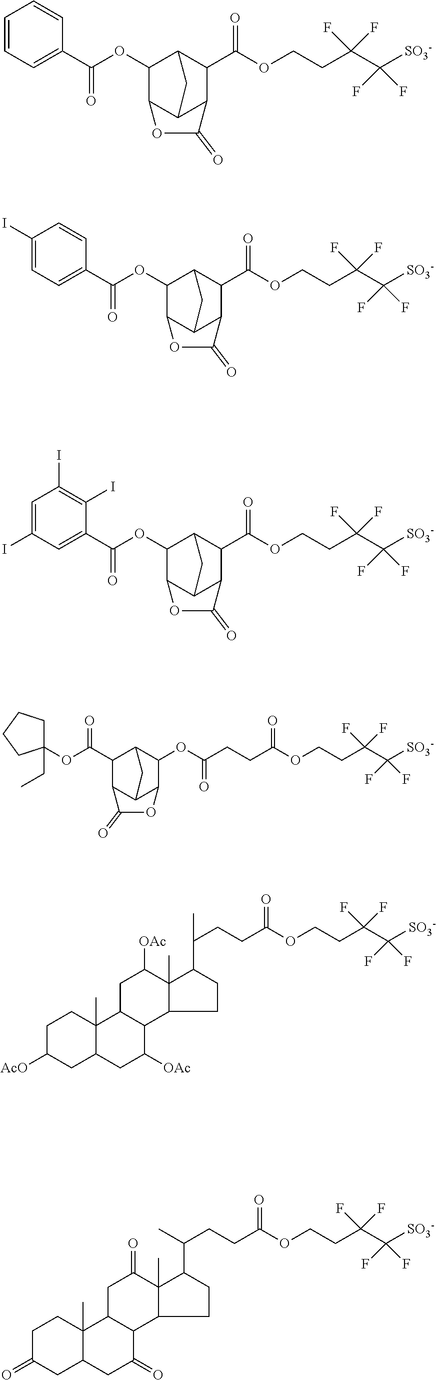

In formulae (c2) and (c3), L¹ is a single bond, ether bond, ester bond, carbonyl group, sulfonic ester bond, carbonate bond or carbamate bond. Of these, an ether bond, ester bond and carbonyl group are preferred from the aspect of synthesis, with an ester bond and carbonyl group being more preferred.

In formula (c2), Rf¹ and Rf² are each independently fluorine or a C₁-C₆ fluorinated saturated hydrocarbyl group. It is preferred for enhancing the acid strength of the generated acid that both Rf¹ and Rf² be fluorine. Rf³ and Rf⁴ are each independently hydrogen, fluorine or a C₁-C₆ fluorinated saturated hydrocarbyl group. It is preferred for increasing solvent solubility that at least one of Rf³ and Rf⁴ be trifluoromethyl.

In formula (c3), Rf^{V} and Rf⁶ are each independently hydrogen, fluorine or a C₁-C₆ fluorinated saturated hydrocarbyl group. It is noted that not all Rf⁵ and Rf⁶ are hydrogen at the same time. It is preferred for increasing solvent solubility that at least one of Rf⁵ and Rf⁶ be trifluoromethyl.

In formulae (c2) and (c3), d is an integer of 0 to 3, preferably 1.

Examples of the anion in repeat unit (c2) are shown below, but not limited thereto. R^{A} is as defined above.

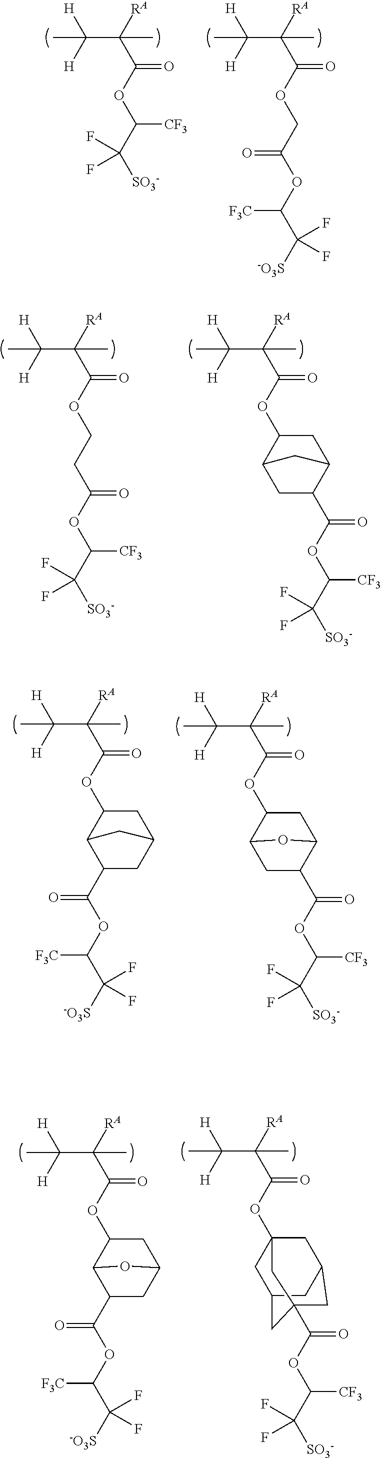

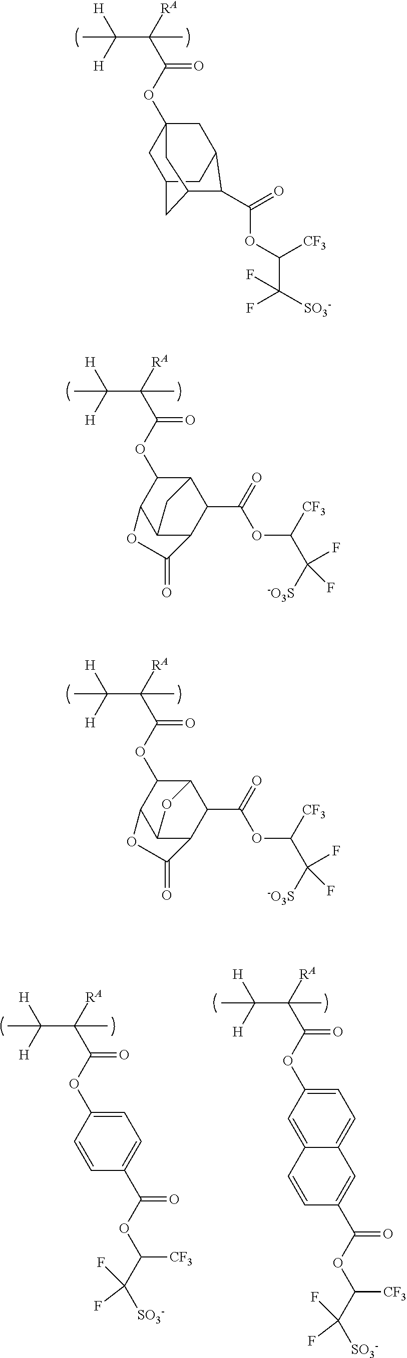

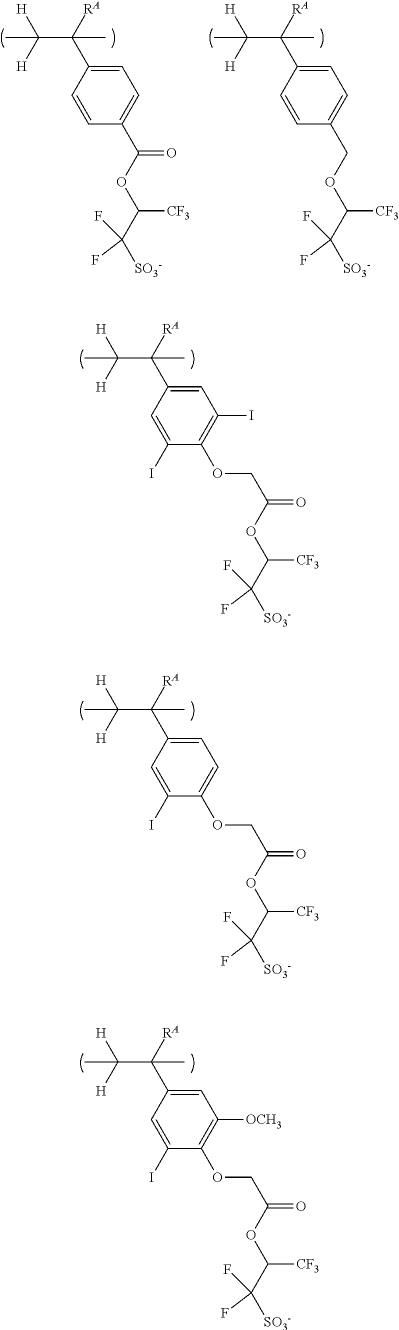

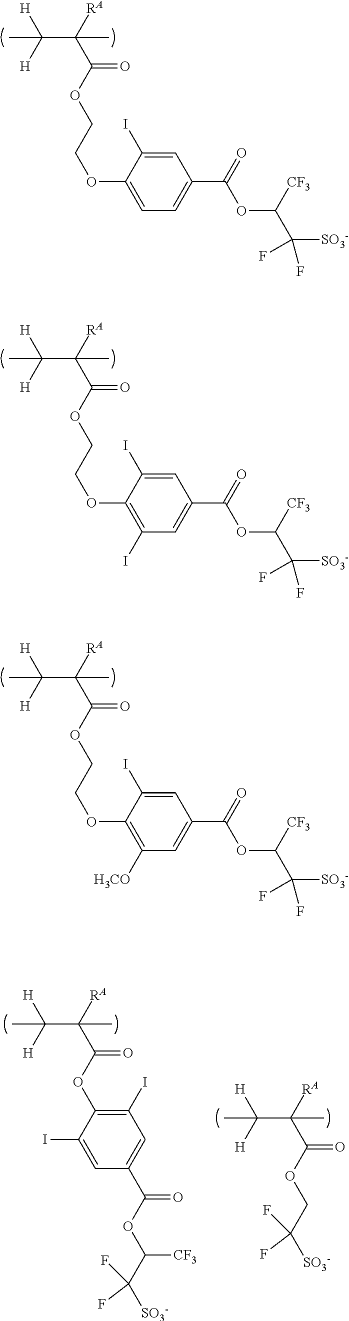

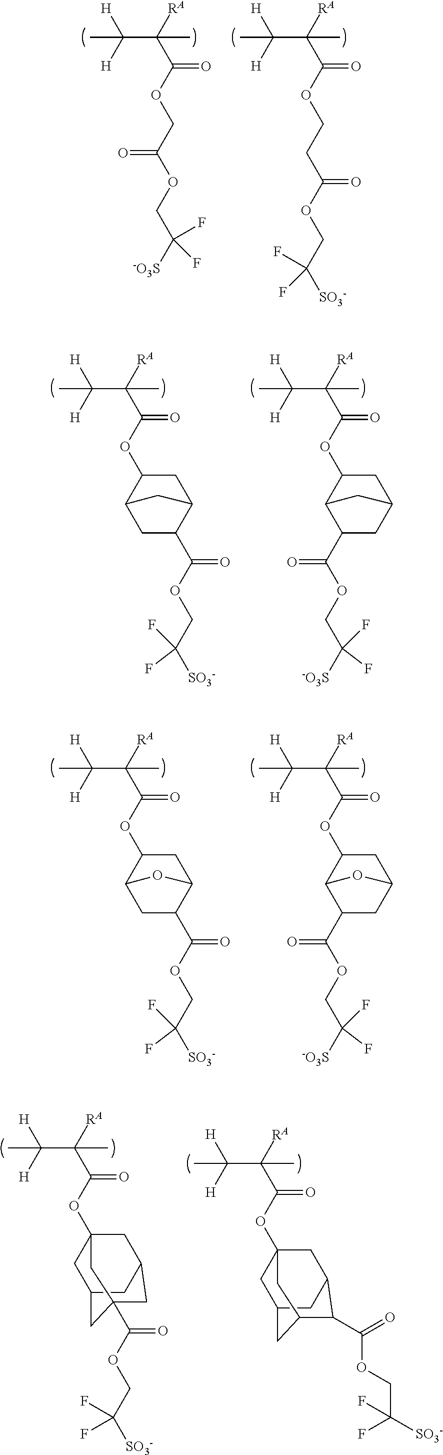

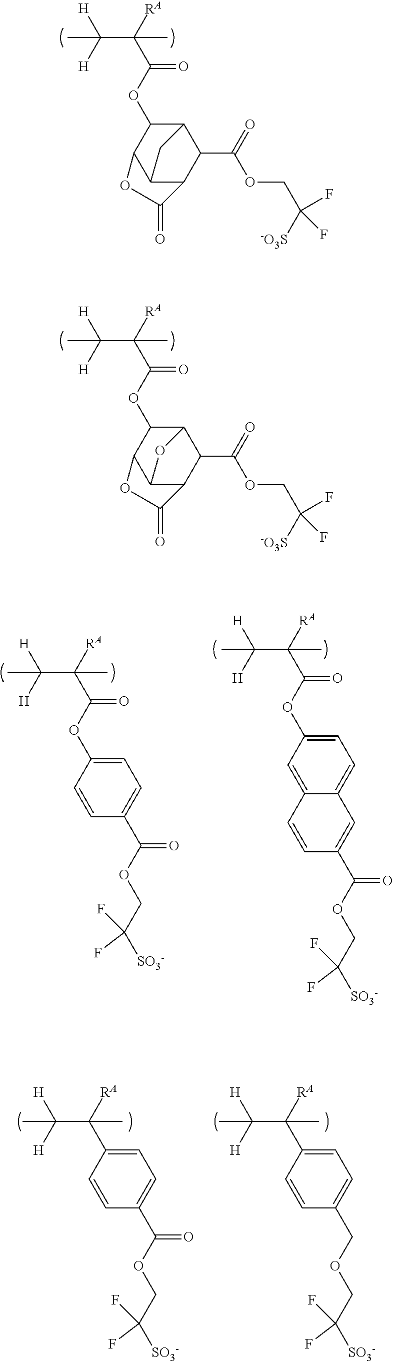

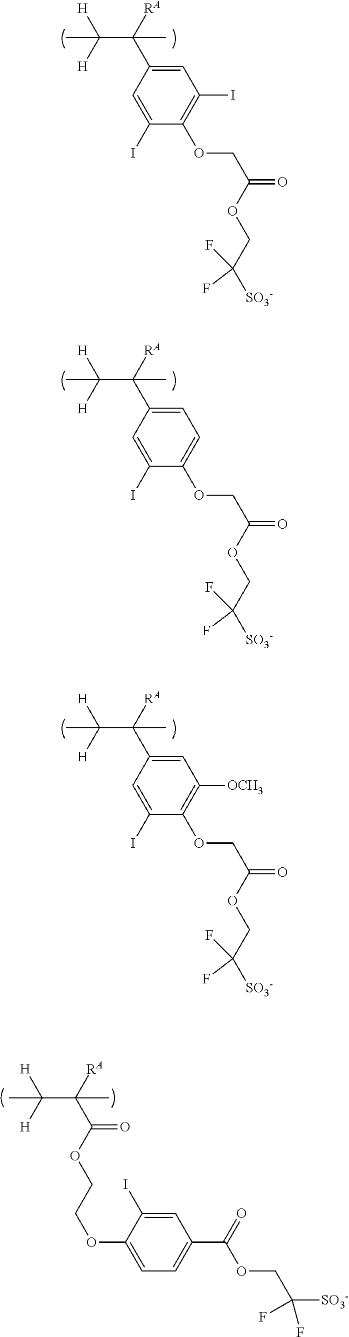

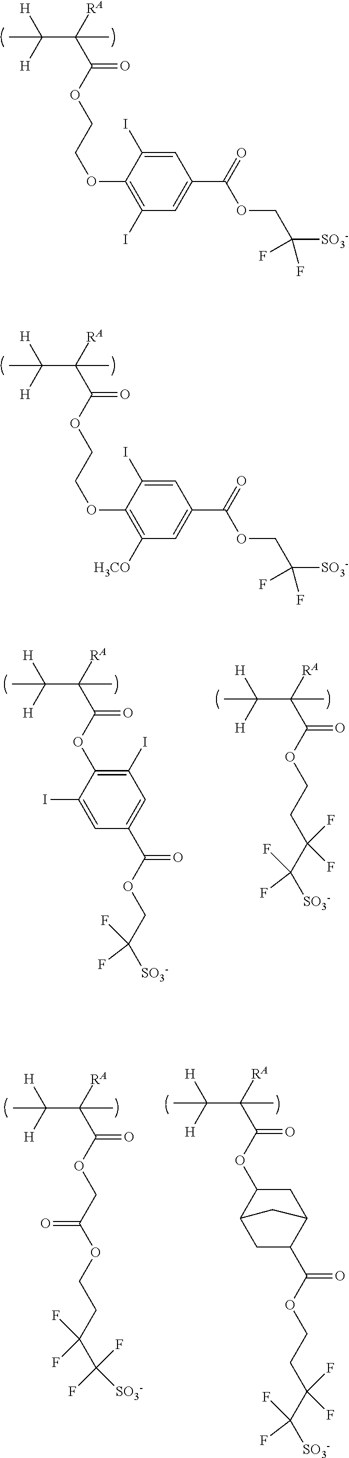

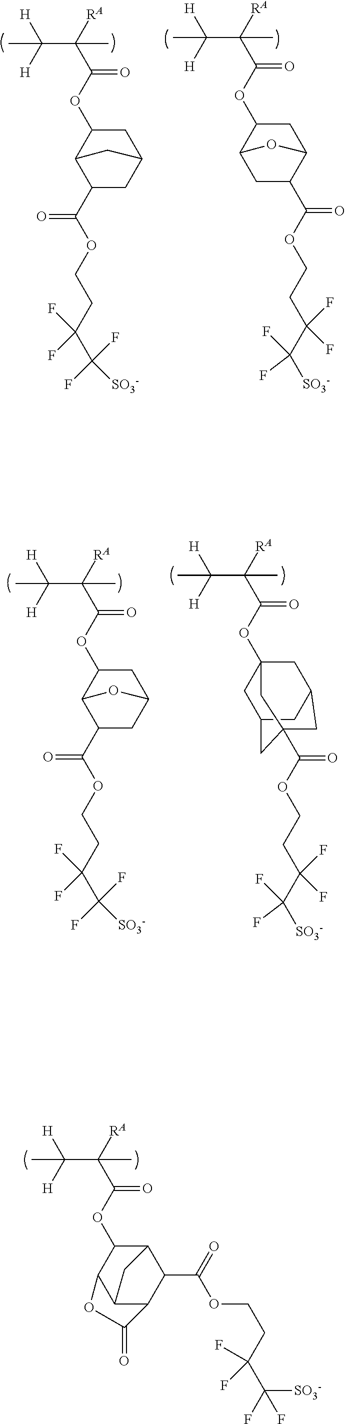

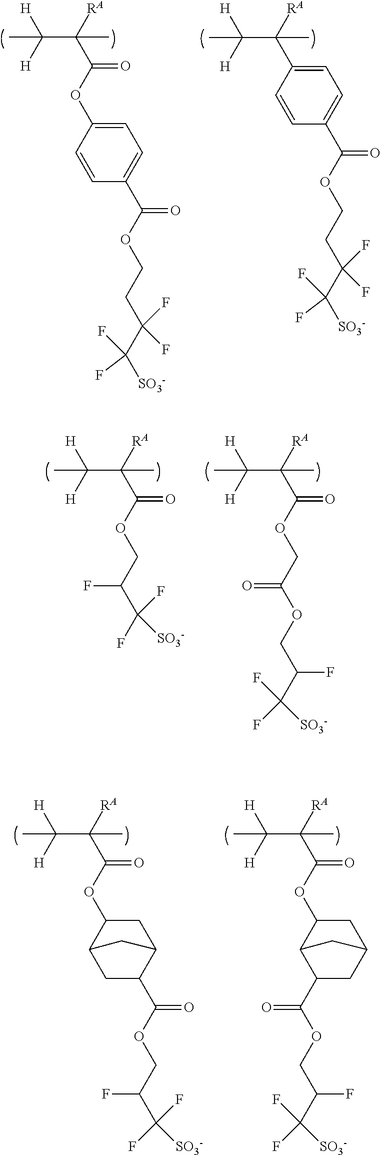

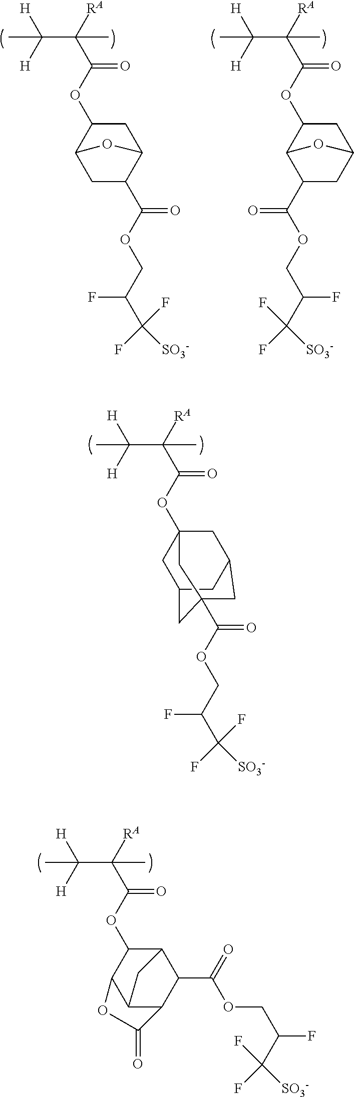

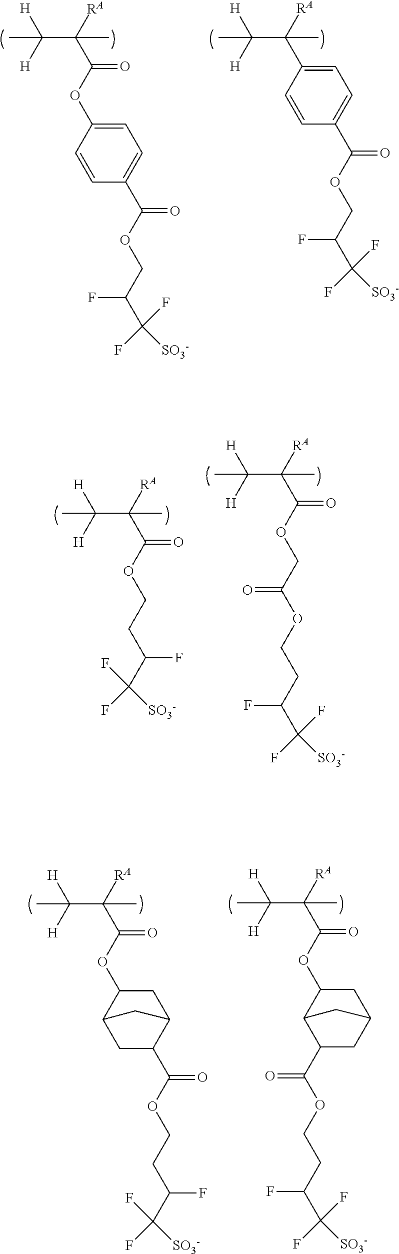

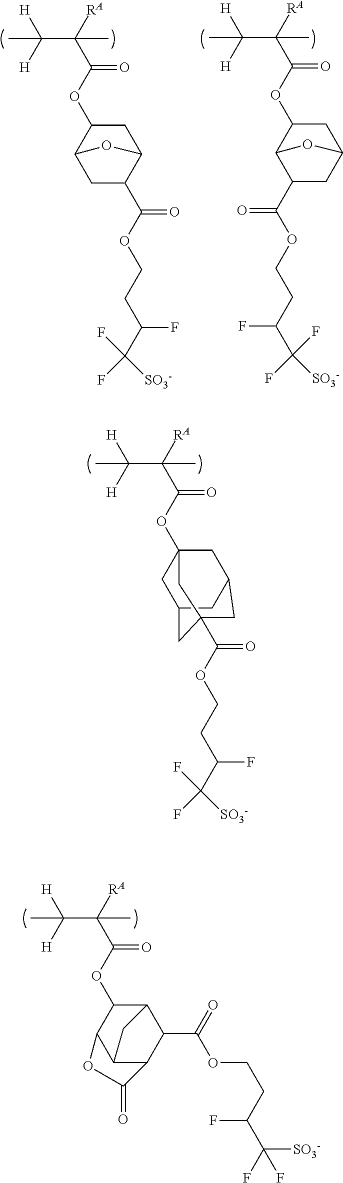

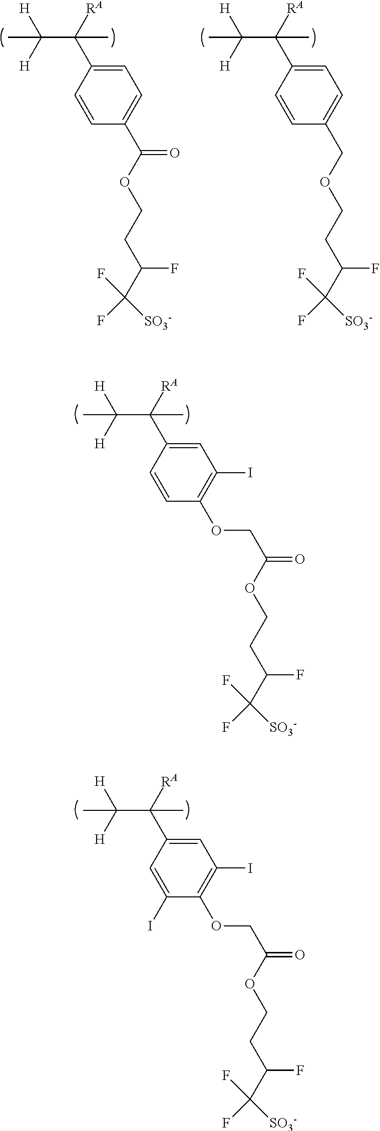

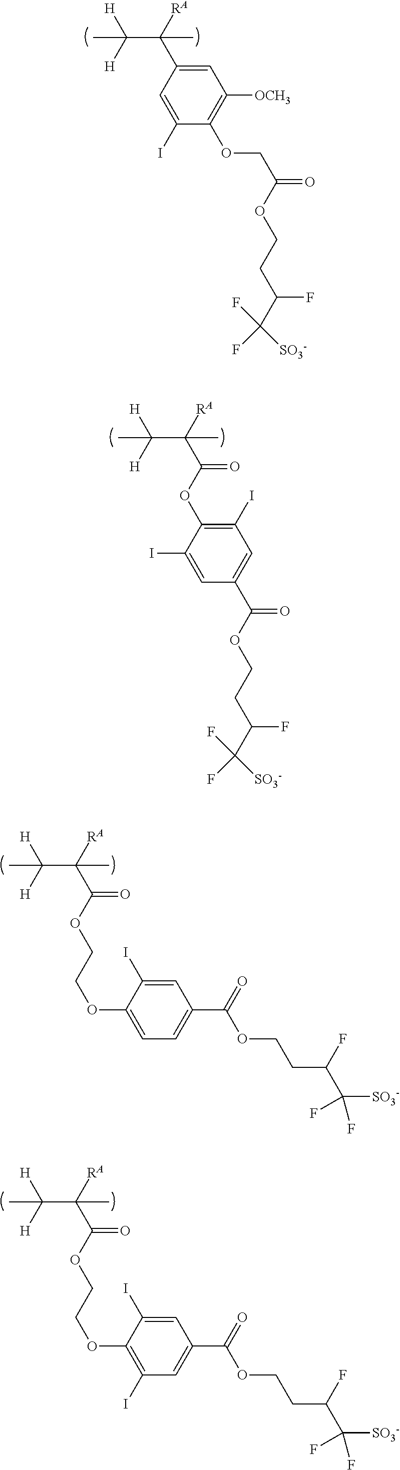

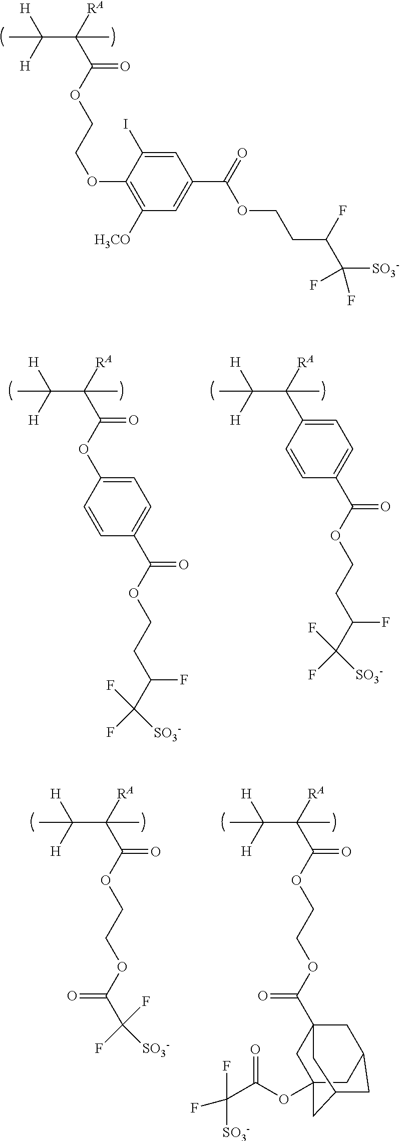

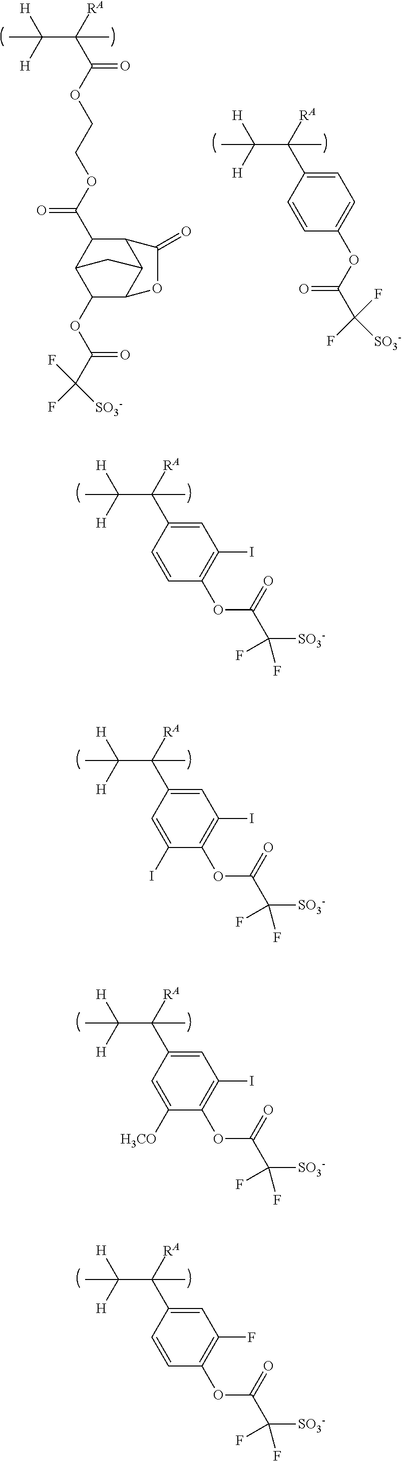

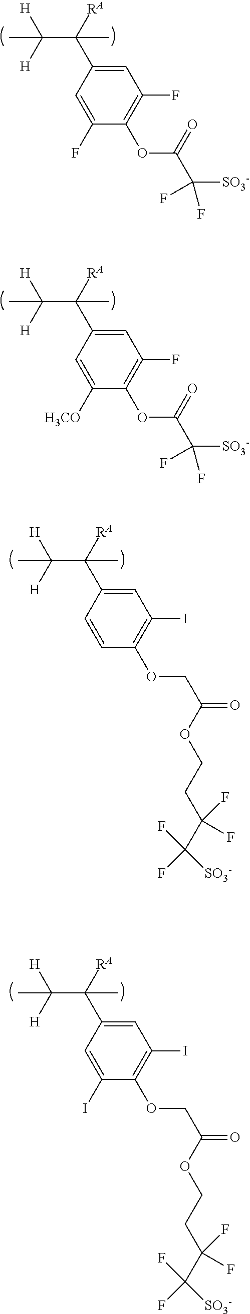

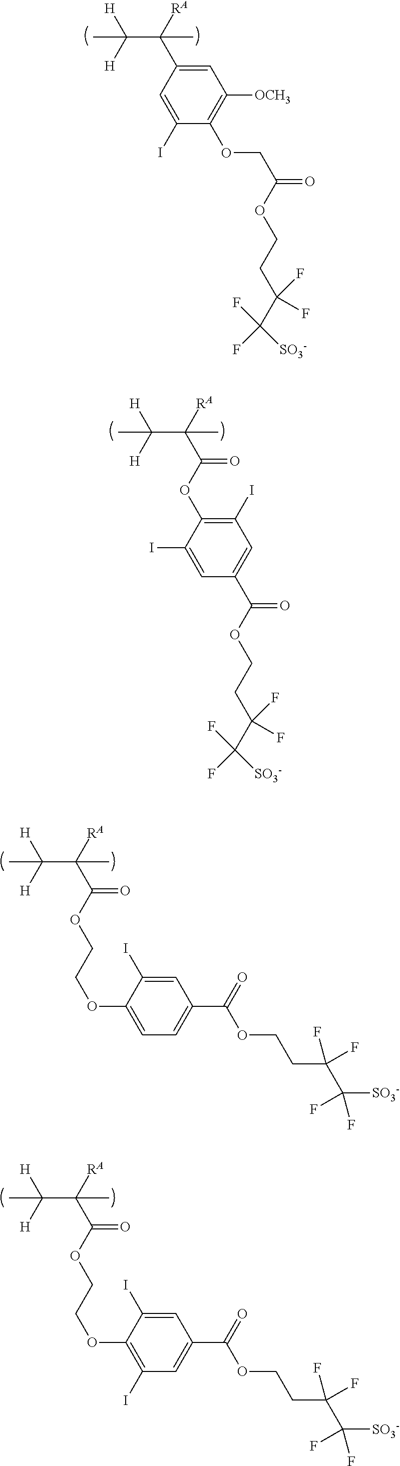

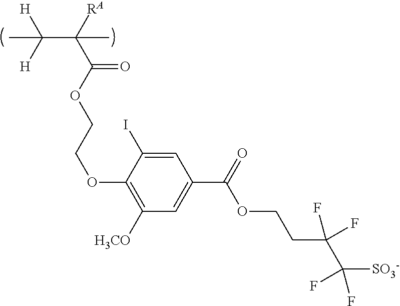

Examples of the anion in repeat unit (6) are shown below, but not limited thereto. R^{A} is as defined above.

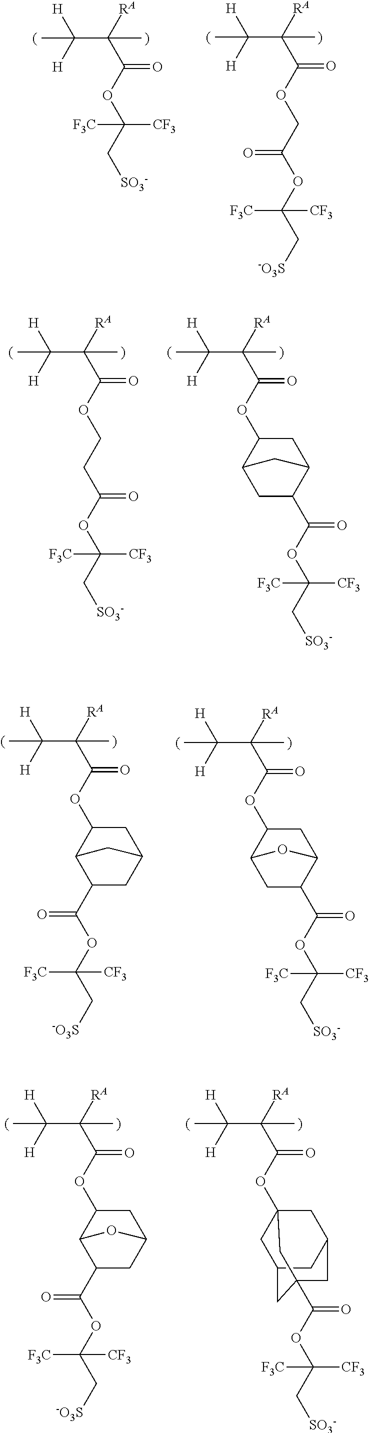

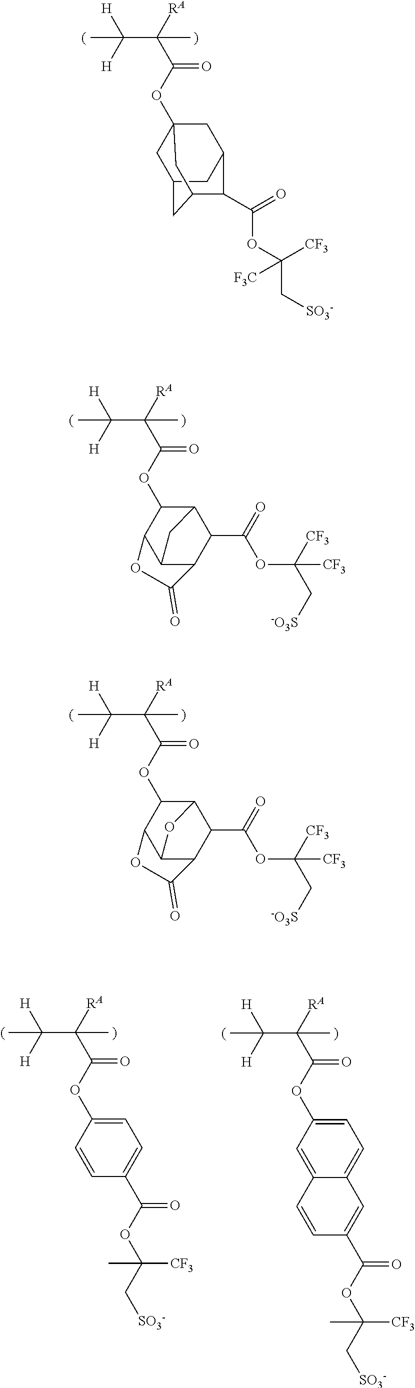

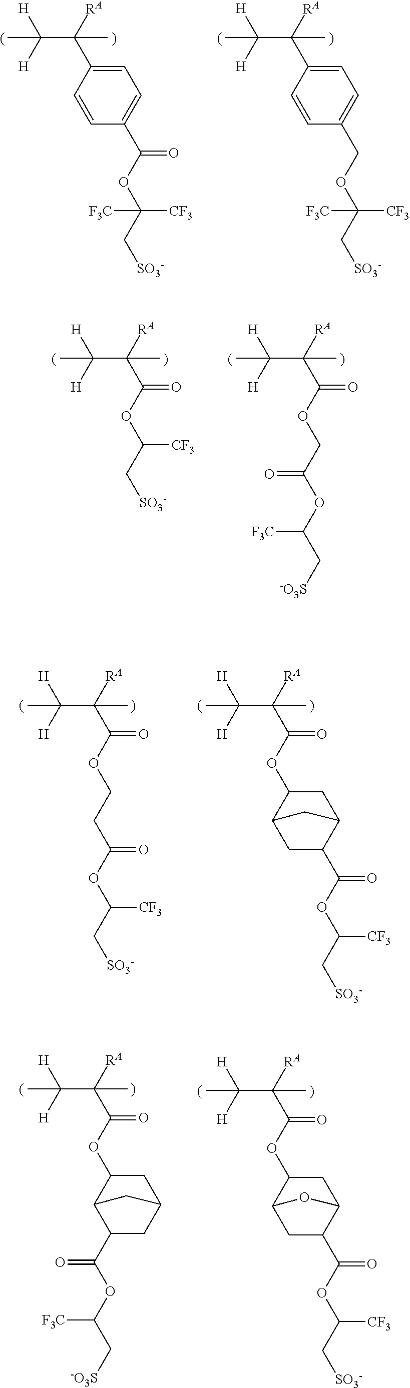

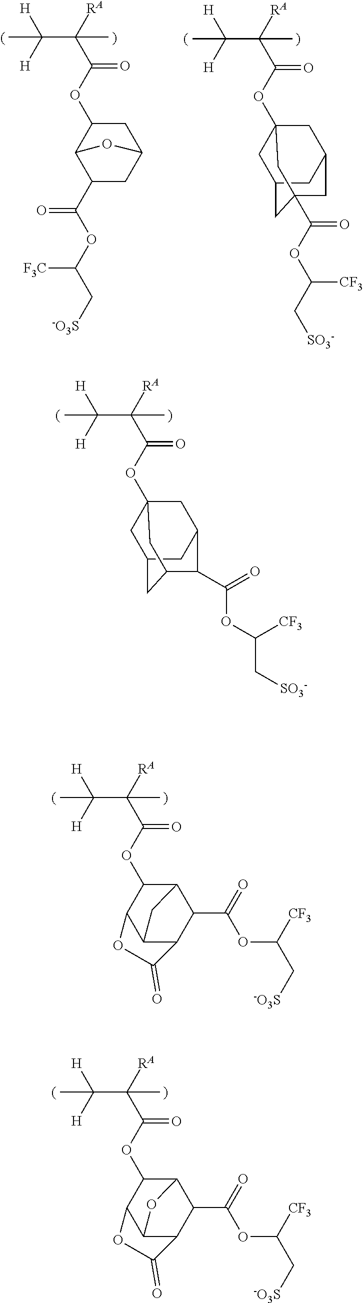

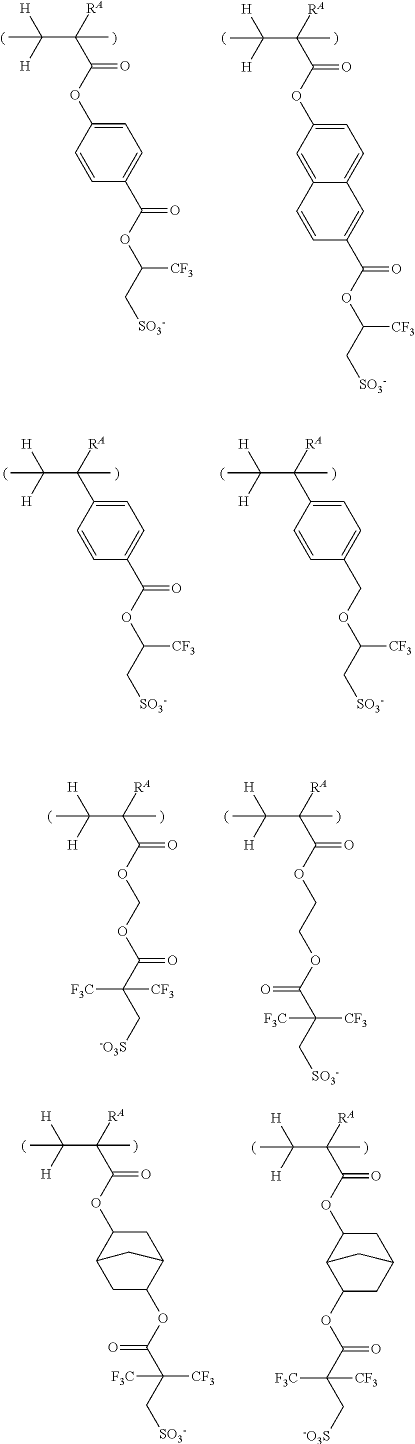

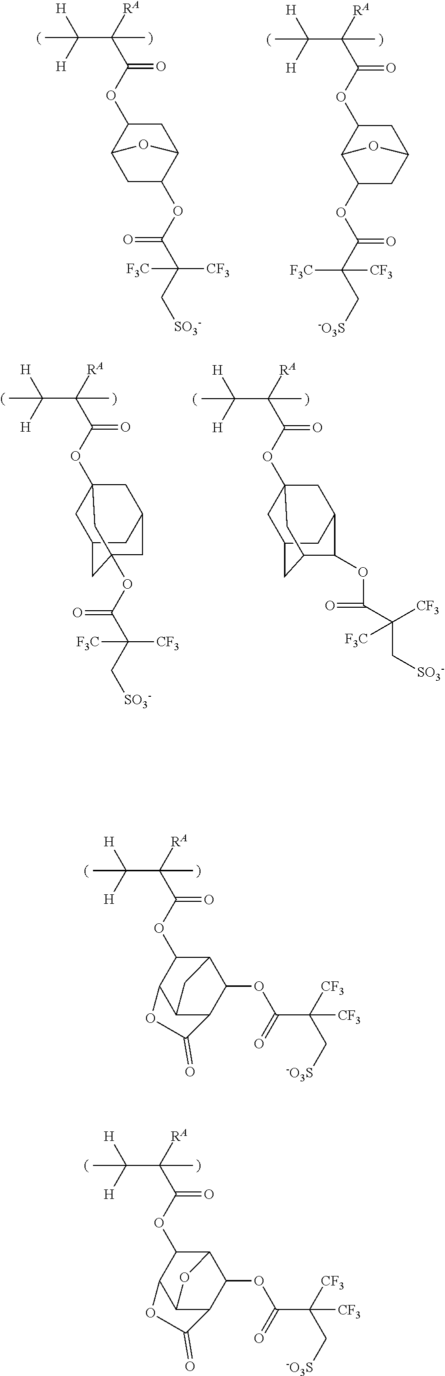

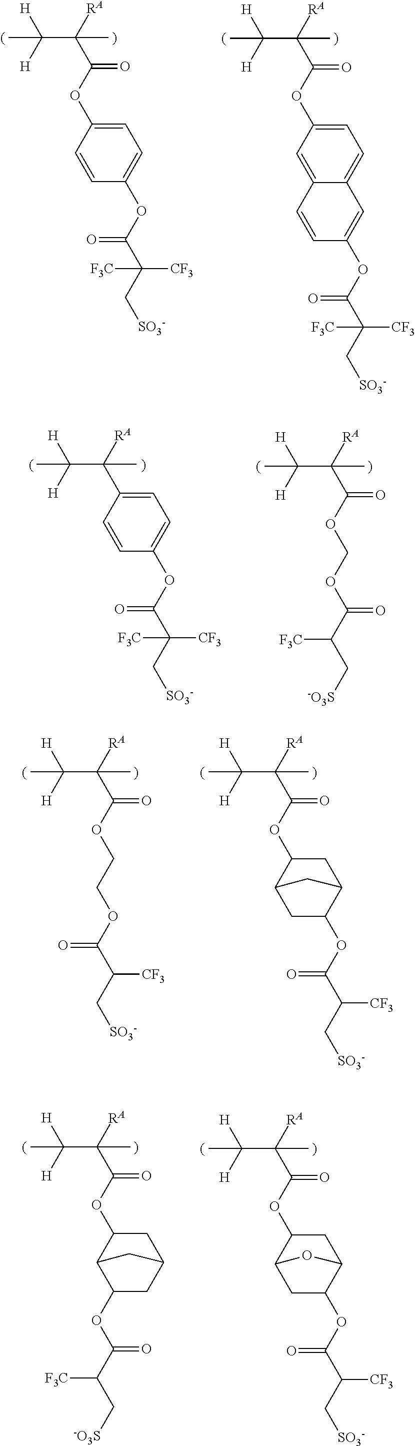

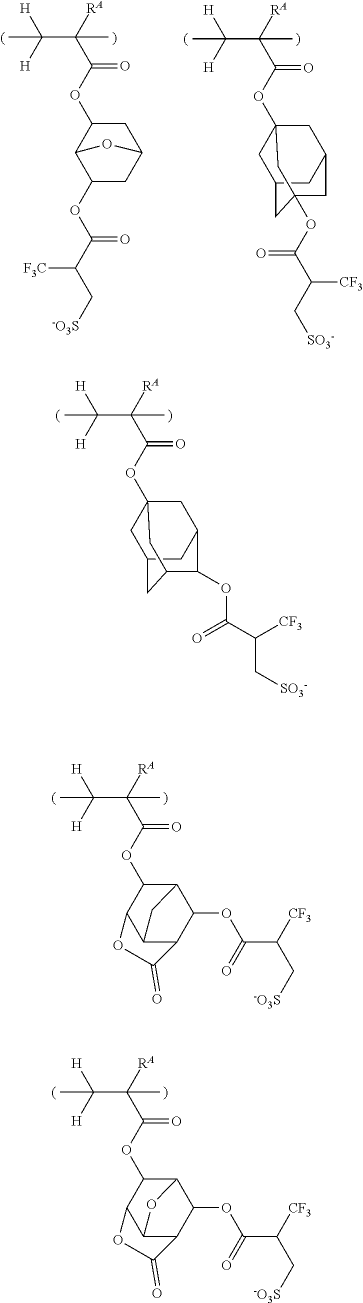

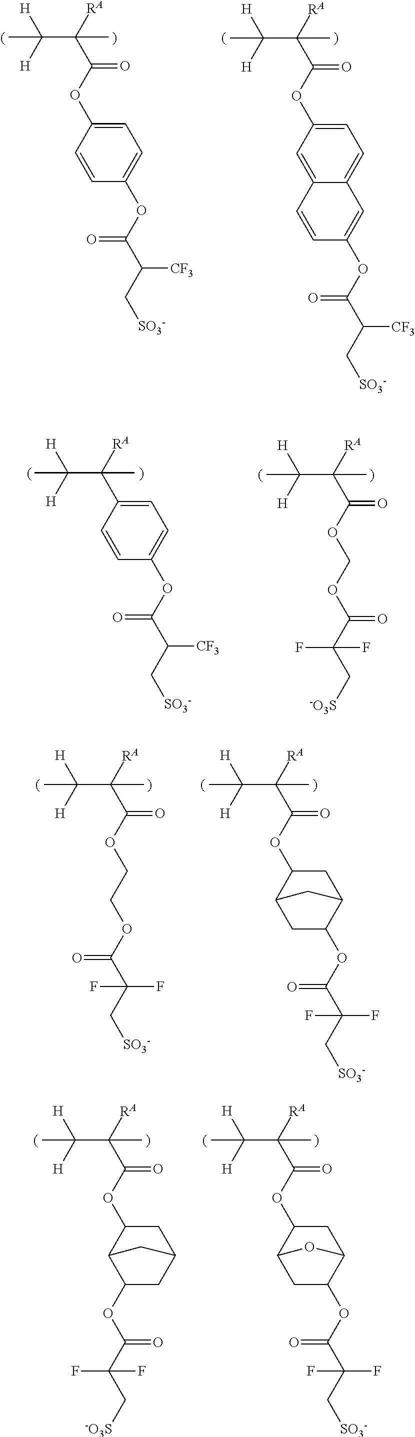

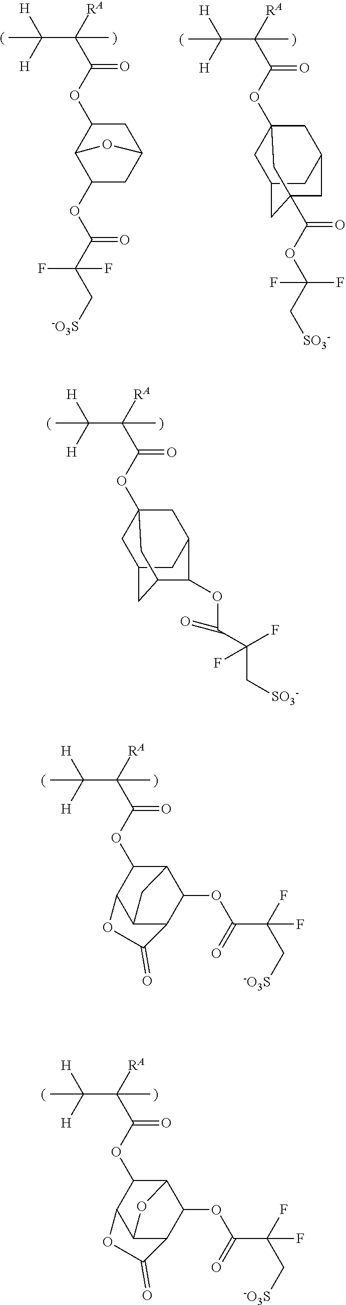

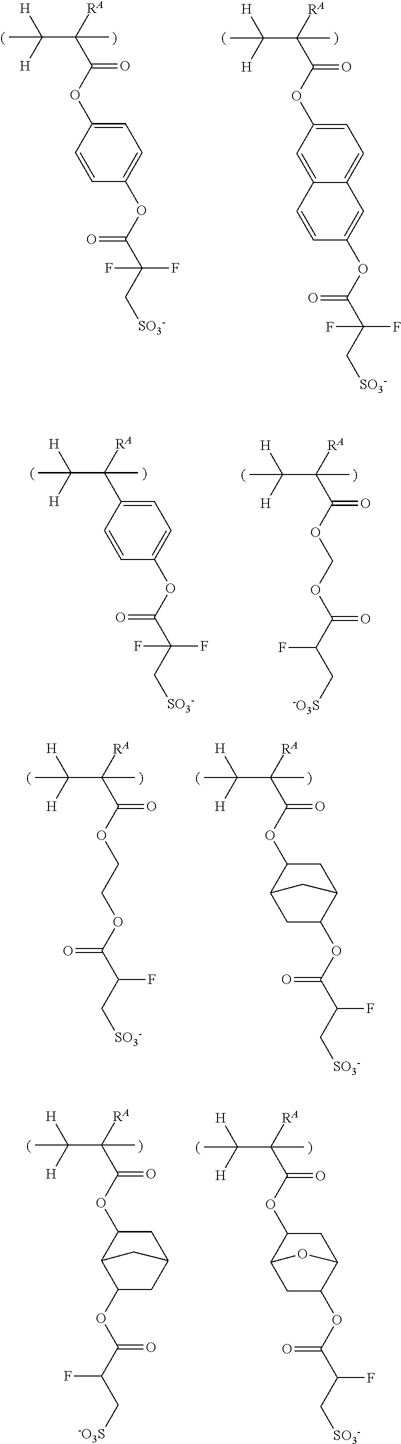

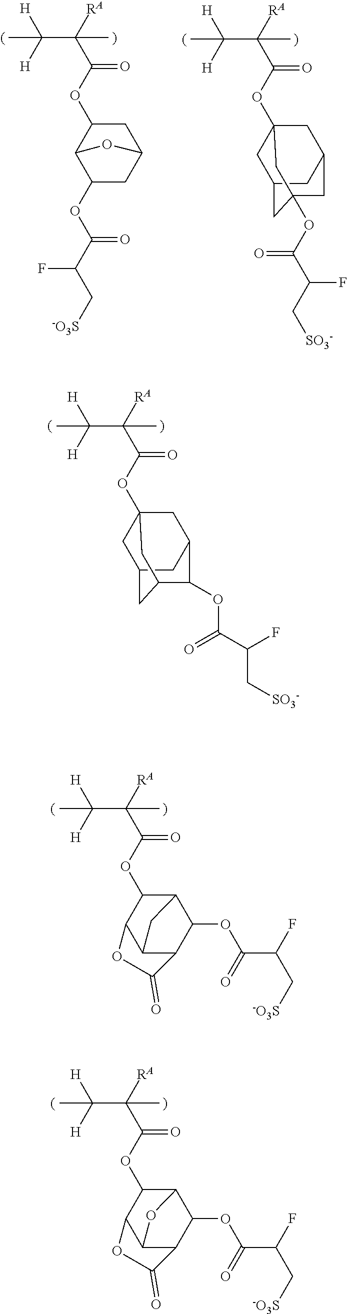

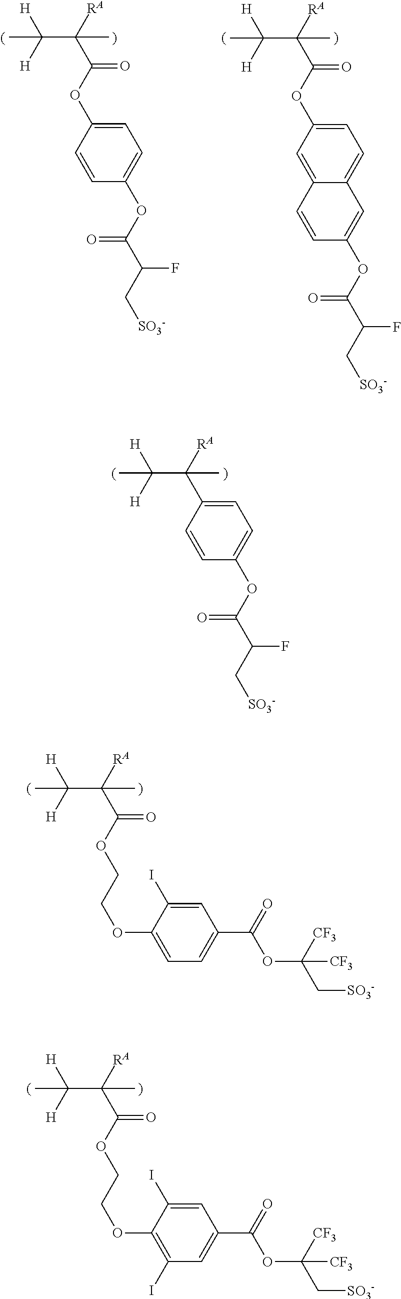

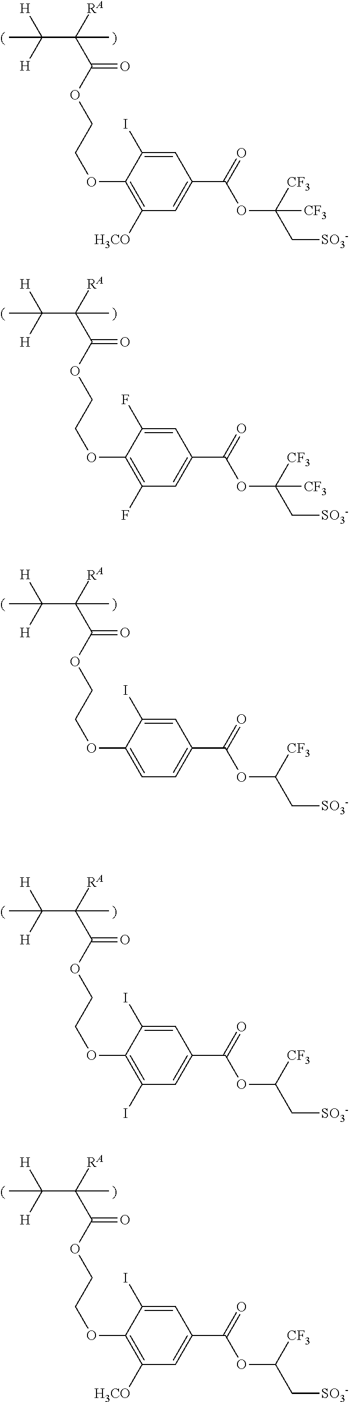

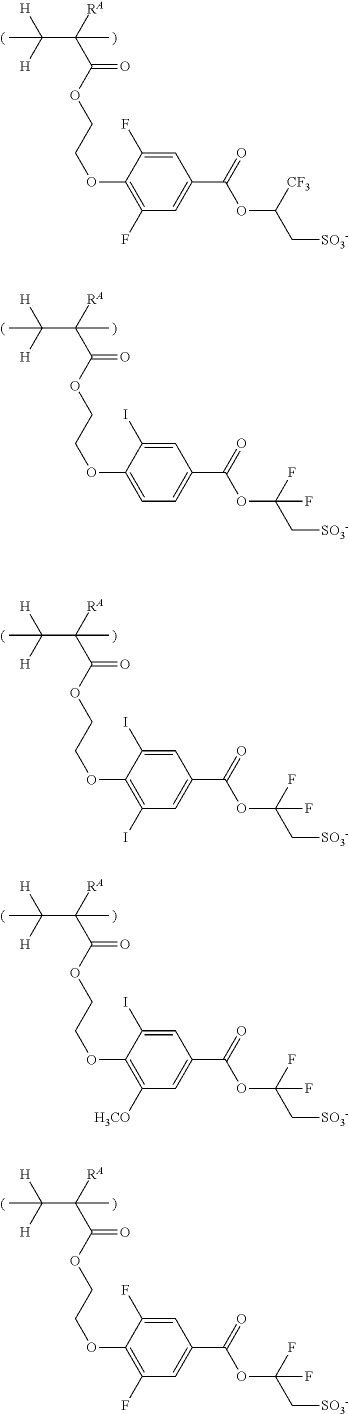

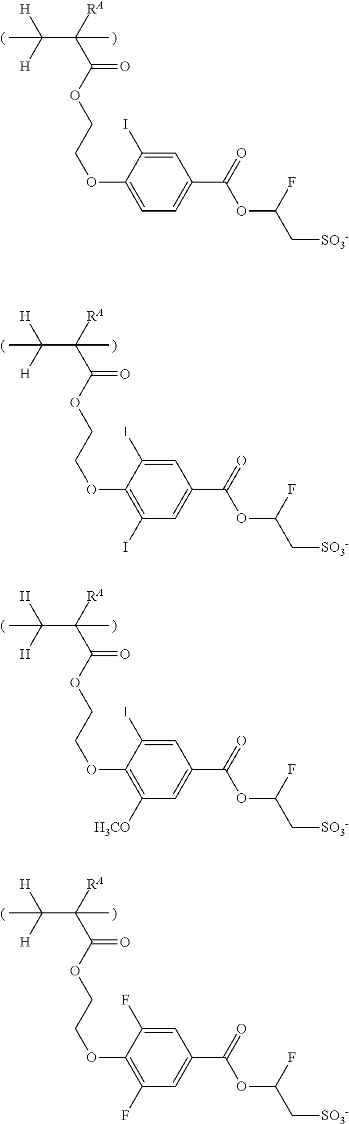

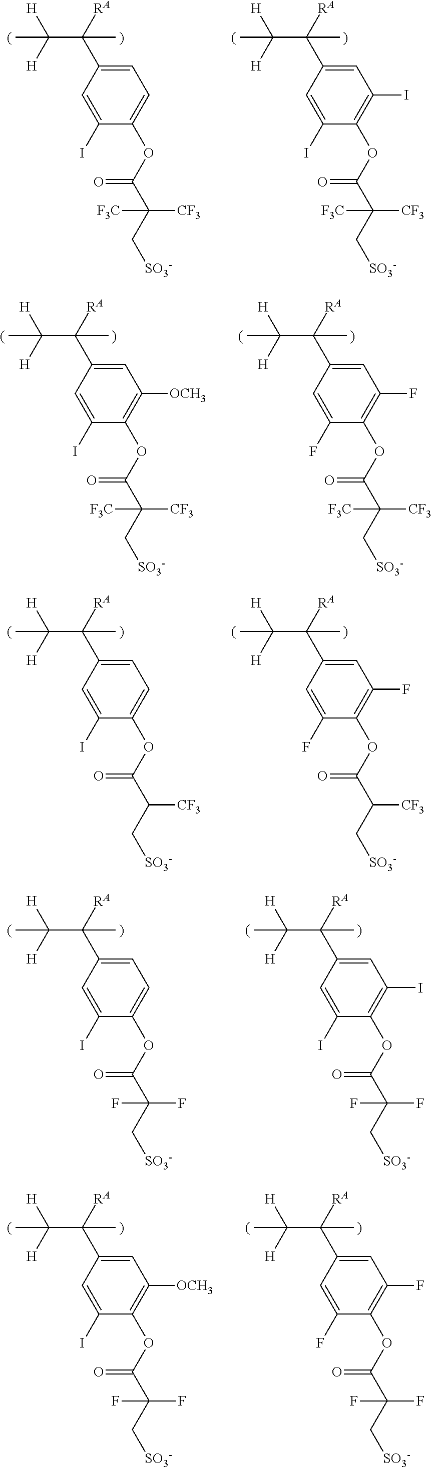

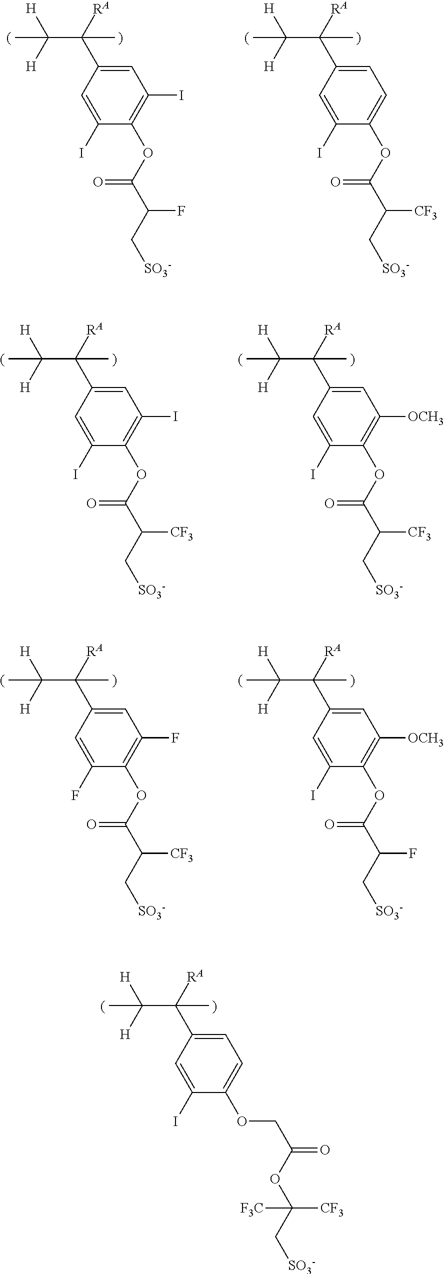

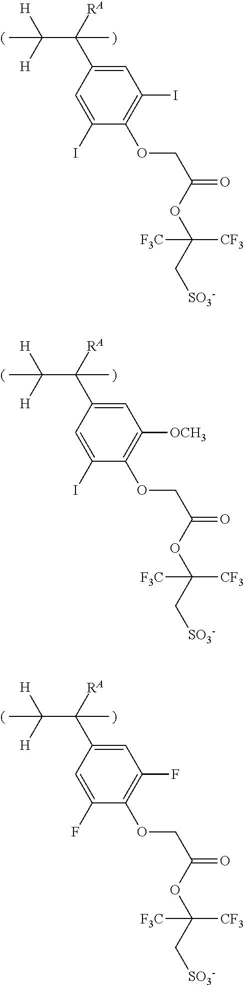

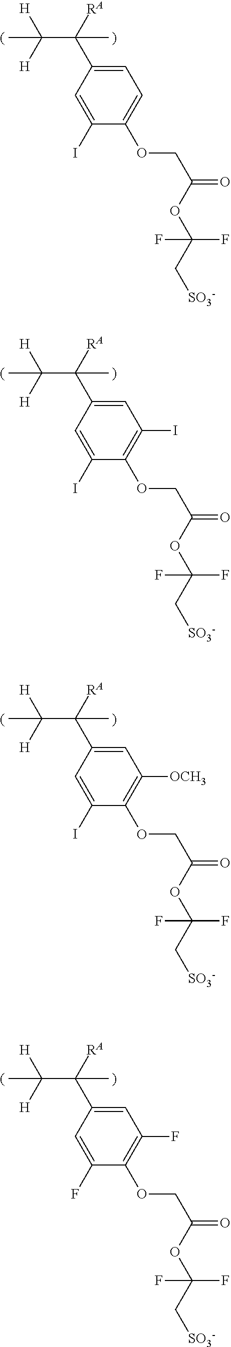

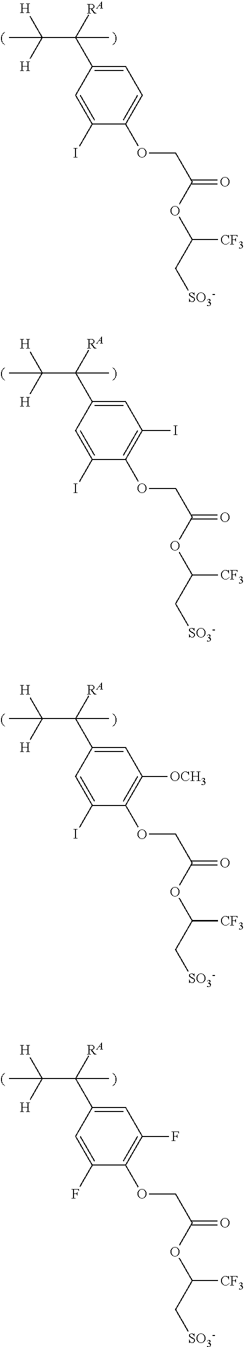

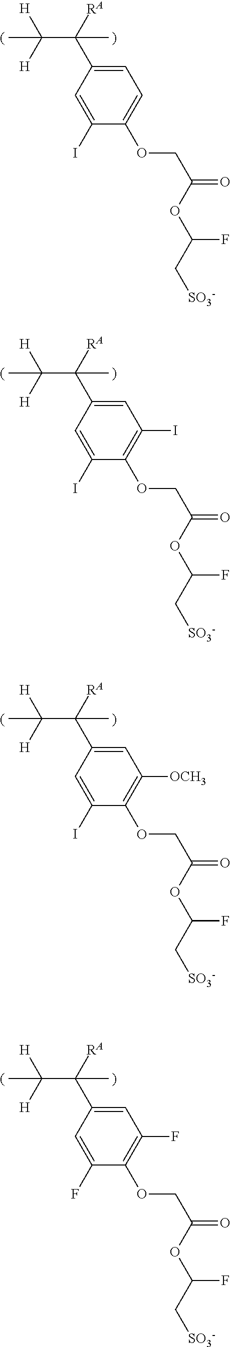

Examples of the anion in repeat unit (c4) are shown below, but not limited thereto. R^{A} is as defined above.

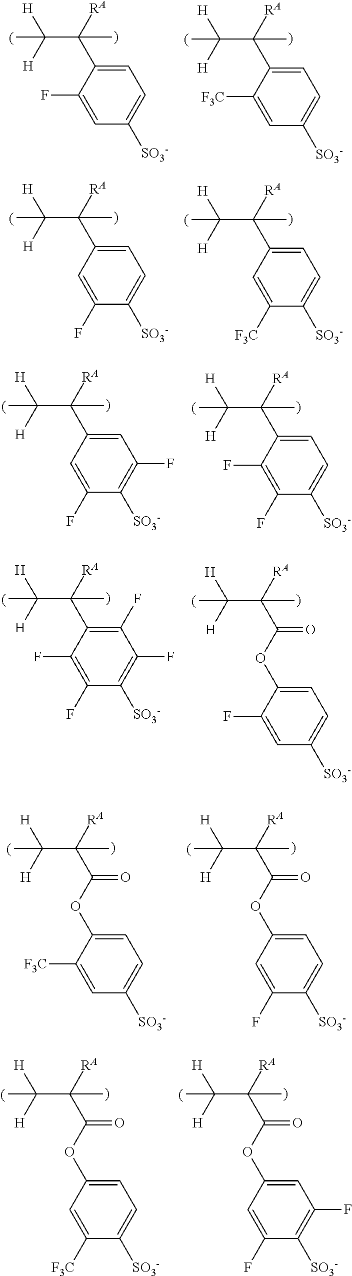

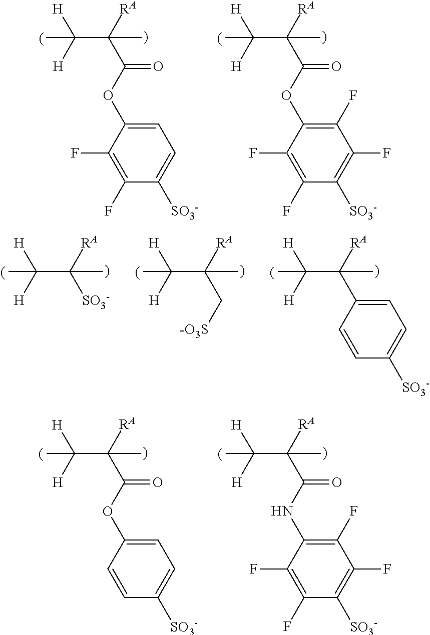

In formulae (c2) to (c4), A⁺ is an onium cation. Suitable onium cations include sulfonium, iodonium and ammonium cations, with sulfonium and iodonium cations being preferred. Specific structures thereof are as exemplified above for the cations having formulae (cation-1) to (cation-3).

The repeat units (c1) to (c4) function as a photoacid generator. Where a base polymer containing repeat units (c1) to (c4), i.e., polymer-bound acid generator is used, the resist composition may or may not contain (D) a photoacid generator to be described later.

The base polymer may further comprise repeat units (d) of a structure having a hydroxy group protected with an acid labile group. The repeat unit (d) is not particularly limited as long as the unit includes one or more structures having a hydroxy group protected with a protective group such that the protective group is decomposed to generate a hydroxy group under the action of acid, Repeat-units having the formula (d1) are preferred.

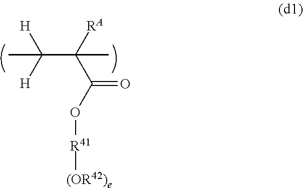

In formula (d1), R^{A} is as defined above. R⁴¹ is a C₁-C₃₀ (e+1)-valent hydrocarbon group which may contain a heteroatom. R⁴² is an acid labile group, and e is an integer of 1 to 4.

In formula (d1), the acid labile group R⁴² is deprotected under the action of acid so that a hydroxy group is generated. The structure of R⁴² is not particularly limited, an acetal structure, ketal structure, alkoxycarbonyl group and alkoxymethyl group having the following formula (d2) are preferred, with the alkoxymethyl group having formula (d2) being more preferred.

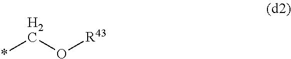

Herein * designates a valence bond and R⁴⁵ is a C₁-C¹⁵ hydrocarbyl group.

Illustrative examples of the acid labile group R⁴², the alkoxymethyl group having formula (d2), and the repeat units (d) are as exemplified for the repeat units (d) in JP-A 2020-111564 (US 20200223796).

In addition to the foregoing units, the base polymer may further comprise repeat units (e) derived from indene, benzofuran, benzothiophene, acenaphthylene, chromone, coumarin, and norbornadiene, or derivatives thereof. Examples of the monomer from which repeat units (e) are derived are shown below, but not limited thereto.

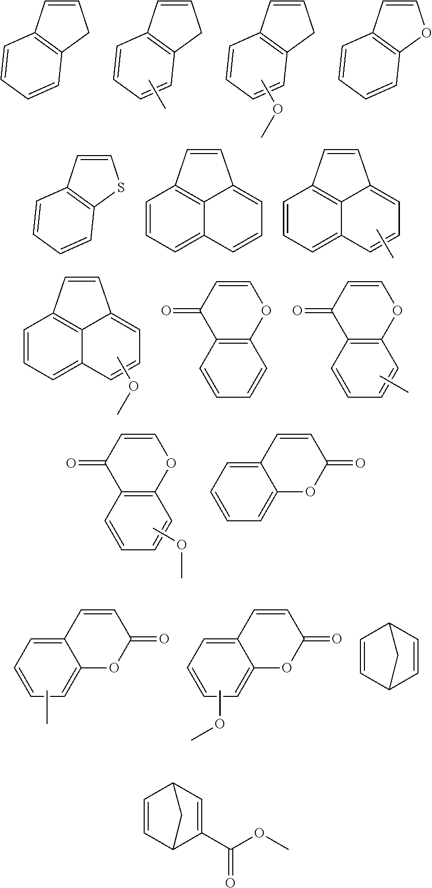

Furthermore, the base polymer may comprise repeat units (f) derived from indane, vinylpyridine, vinylcarbazole, or derivatives thereof.

In the base polymer, a fraction of units (a1), (a2), (b1), (b2), (c1) to (c4), (d), (e), and (f) is: preferably 0<a1≤0.8, 0≤a2≤0.8, 0<a1+a2≤0.8, 0≤b1≤0.6, 0≤b2≤0.6, 0≤b1+b2≤0.6, 0≤c1≤0.4, 0≤c2≤0.4, 0≤c3≤0.4, 0≤c4≤0.4, 0≤c1+c2+c3+c4≤0.4, 0≤d≤0.5, 0≤e≤0.3, and 0≤f≤0.3; more preferably 0<a1≤0.7, 0≤a2≤0.7, 0<a1+a2≤0.7, 0≤b1≤0.5, 0≤b2≤0.5, 0≤b1+b2≤0.5, 0≤c1≤0.3, 0≤c2≤0.3, 0≤c3≤0.3, 0≤c4≤0.3, 0≤c1+c2+c3+c4≤0.3, 0≤d≤0.3, 0≤e≤0.3, and 0≤f≤0.3, with the proviso: a1+a2+b1+b2+c1+c2+c3+c4+d+e+f=1.

The base polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. It is noted that Mw is as measured by GPC versus polystyrene standards using tetrahydrofuran (THF) or N,N-dimethylformamide (DMF) solvent.

Since the influence of dispersity (Mw/Mn) becomes stronger as the pattern rule becomes finer, the base polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight fractions and eliminates the risk of leaving foreign particles on the pattern or degrading the pattern profile after exposure and development.

The base polymer may be synthesized by any desired methods, for example, by dissolving one or more monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, dioxane, cyclohexane, cyclopentane, methyl ethyl ketone (MEK), PGMEA, and GBL. Examples of the polymerization initiator used herein include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, a time of 2 to 12 hours being more preferred in view of production efficiency.

The polymerization initiator may be added to the monomer solution, which is fed to the reactor. Alternatively, a solution of the polymerization initiator is prepared separately from the monomer solution, and the monomer and initiator solutions are independently fed to the reactor. Since there is a possibility that the initiator generates a radical in the standby time, by which polymerization reaction takes place to form a ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The amounts of monomers in the monomer solution may be determined appropriate so as to provide the preferred fractions of repeat units as mentioned above.

It is described how to use the polymer obtained by the above preparation method. The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145] (U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, 1-ethoxy-2-propanol; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as DAA; and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The base polymer (C) may be used alone or as a mixture of two or more polymers which are different in compositional ratio, Mw and/or Mw/Mn. In addition to the polymer defined above, the base polymer (C) may contain a hydrogenated product of ring-opening metathesis polymerization (ROMP) polymer, which is described in JP-A 2003-066612.

### (D) Photoacid Generator

The resist composition may comprise (D) a photoacid generator. The PAG used herein may be any compound capable of generating an acid upon exposure to high-energy radiation. The preferred PAG has the formula (2-1) or (2-2).

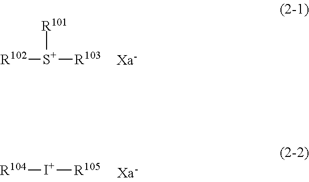

In formulae (2-1) and (2-2), R¹⁰¹ to R¹⁰⁵ are each independently a C₁-C₃₀ hydrocarbyl group which may contain a heteroatom. Any two of R¹⁰¹, R¹⁰² and R¹⁰³ may bond together to form a ring with the sulfur atom to which they are attached.

Examples of the cation in the sulfonium salt having formula (2-1) are as exemplified above for the sulfonium cation having formula (cation-1), but not limited thereto. Examples of the cation in the iodonium salt having formula (2-2) are as exemplified above for the iodonium cation having formula (cation-2), but not limited thereto.

In formulae (2-1) and (2-2), Xa⁻ is an anion of the following formula (2A), (2B), (2C) or (2D).

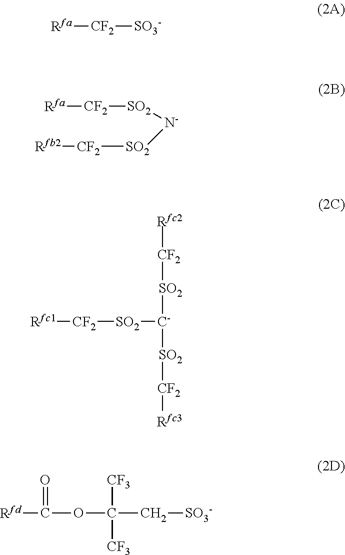

In formula (2A), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified later for hydrocarbyl group R^{fa1} in formula (2A′).

Of the anions of formula (2A), a structure having the formula (2A′) is preferred.

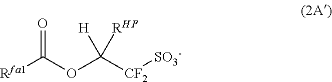

In formula (2A′), R^{HF} is hydrogen or trifluoromethyl, preferably trifluoromethyl.

R^{fa1} is a C₁-C₃₈ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation. The C₁-C₃₈ hydrocarbyl group R^{fa1} may be saturated or unsaturated and straight, branched or cyclic. Suitable hydrocarbyl groups include C₁-C₃₈ alkyl groups such as methyl, ethyl, propyl, isopropyl, butyl, sec-butyl, tert-butyl, pentyl, neopentyl, hexyl, heptyl, octyl, 2-ethylhexyl, nonyl, decyl, undecyl, dodecyl, tridecyl, pentadecyl, heptadecyl, icosanyl; C₃-C₃₈ cyclic saturated hydrocarbyl groups such as cyclopentyl, cyclohexyl, 1-adamantyl, 2-adamantyl, 1-adamantylmethyl, norbornyl, norbornylmethyl, tricyclodecanyl, tetracyclododecanyl, tetracyclododecanylmethyl, dicyclohexylmethyl; C₂-C₃₈ unsaturated aliphatic hydrocarbyl groups such as allyl and 3-cyclohexenyl; C₆-C₃₈ aryl groups such as phenyl, 1-naphthyl, 2-naphthyl; C₇-C₃₈ aralkyl groups such as benzyl and diphenylmethyl; and combinations thereof.

In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, 5-hydroxy-1-adamantyl, 5-tert-butylcarbonyloxy-1-adamantyl, 4-oxatricyclo[4.2.1.0^{3,7}]nonan-S-on-2-yl, and 3-oxocyclohexyl.

With respect to the synthesis of the sulfonium salt having an anion of formula (2A′), reference is made to JP-A 2007-145797, JP-A 2008-106045, JP-A 2009-007327, and JP-A 2009-258695. Also useful are the sulfonium salts described in JP-A 2010-215608, JP-A 2012-041320, JP-A 2012-106986, and JP-A 2012-153644.

Examples of the anion having formula (2A) are as exemplified above for the anions having formulae (c1-1) and (c1-2).

In formula (2B), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (2A′). Preferably R^{b1} and R¹² each are fluorine or a straight C₁-C₄ fluorinated alkyl group. A pair of R^{b1} and R¹² may bond together to form a ring with the linkage (—CF₂—SO₂—N—SO₂—CF₂—) to which they are attached, and the R^{fb1}-R^{fb2} group is preferably a fluorinated ethylene or fluorinated propylene group.

In formula (2C), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (2A′). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C—SO₂—CF₂—) to which they are attached, and the R^{fc1}-R^{fc2} group is preferably a fluorinated ethylene or fluorinated propylene group.

In formula (2D), R^{fd} is a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1}.

With respect to the synthesis of the sulfonium salt having an anion of formula (2D), reference is made to JP-A 2010-215608 and JP-A 2014-133723.

Examples of the anion having formula (2D) are shown below, but not limited thereto.

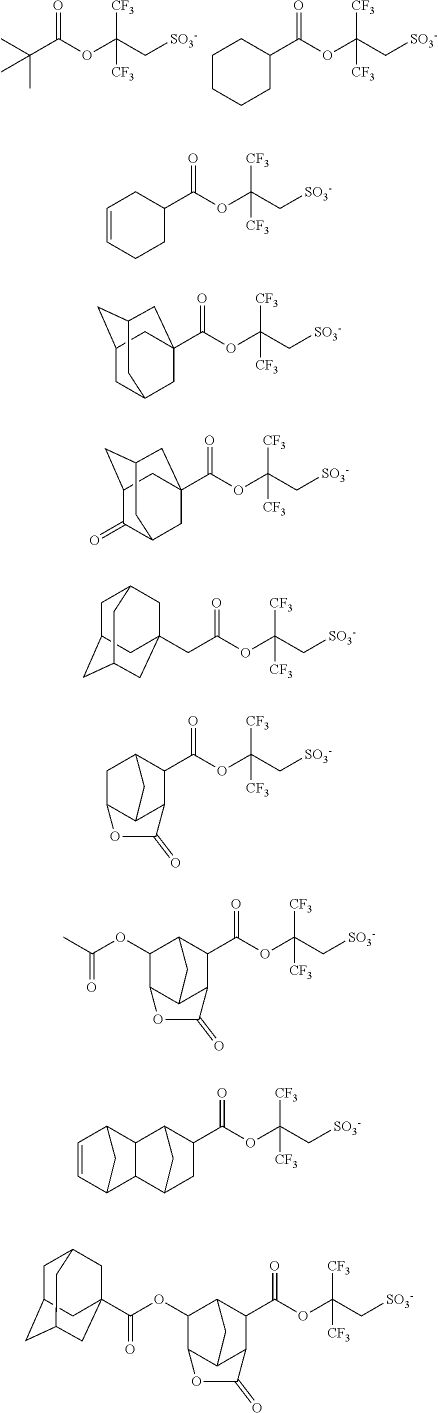

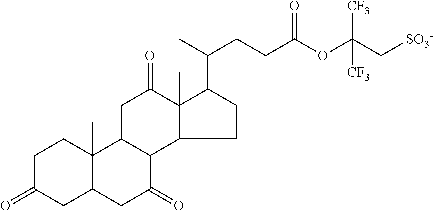

Another example of the non-nucleophilic counter ion is an anion having an iodine or bromine-substituted aromatic ring. The preferred anion has the formula (2E).

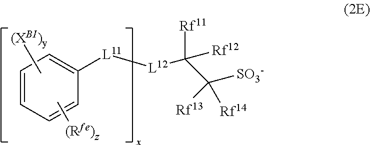

In formula (2E), x is an integer of 1 to 3, y is an integer of 1 to 5, z is an integer of 0 to 3, and y+z is from 1 to 5. Preferably, y is an integer of 1 to 3, more preferably 2 or 3, and z is an integer of 0 to 2.

In formula (2E), X^{BI} is iodine or bromine. When x and/or y is 2 or more, a plurality of X^{BI} may be the same or different.

In formula (2E), L¹¹ is a single bond, ether bond, ester bond, or a C₁-C₆ saturated hydrocarbylene group which may contain an ether bond or ester bond. The saturated hydrocarbylene group may be straight, branched or cyclic.

In formula (2E), L^{fe} is a single bond or a C₁-C₂₀ divalent linking group when x=1, and a C₁-C₂₀ (x+1)-valent linking group which may contain oxygen, sulfur or nitrogen when x=2 or 3.

In formula (2E), R^{fe} is hydroxy, carboxy, fluorine, chlorine, bromine, or amino, or a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group, which may contain fluorine, chlorine, bromine, hydroxy, amino or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{feD} is a C₁-C₁₆ aliphatic hydrocarbyl group, C₆-C₁₂ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy and hydrocarbylsulfonyloxy groups may be straight, branched or cyclic. A plurality of groups R^{fe} may be the same or different when x and/or z is 2 or more. Of these, R^{fe} is preferably hydroxy, —N(R^{feC})—C(═O)—R^{feD}, —N(R^{feC})—C(═O)—O—R^{feD} fluorine, chlorine, bromine, methyl or methoxy.

In formula (2E), Rf¹¹ to Rf¹⁴ are each independently hydrogen, fluorine or trifluoromethyl, at least one of Rf¹¹ to Rf¹⁴ is fluorine or trifluoromethyl. Rf¹¹ and Rf¹², taken together, may form a carbonyl group. Preferably, both Rf¹³ and Rf¹⁴ are fluorine.

Examples of the anion in the onium salt having formula (2E) are shown below, but not limited thereto. Herein X^{BI} is as defined above.

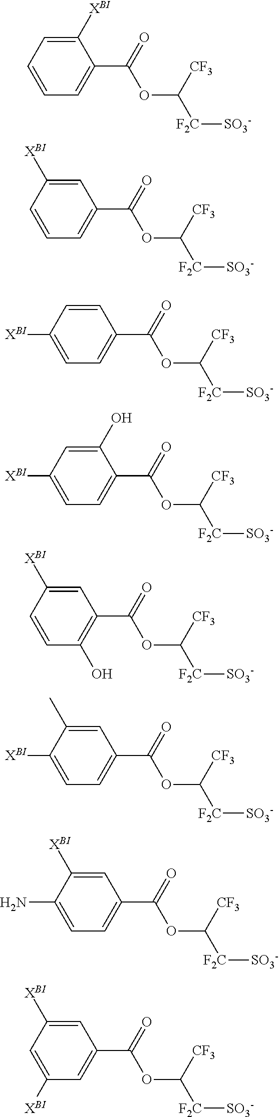

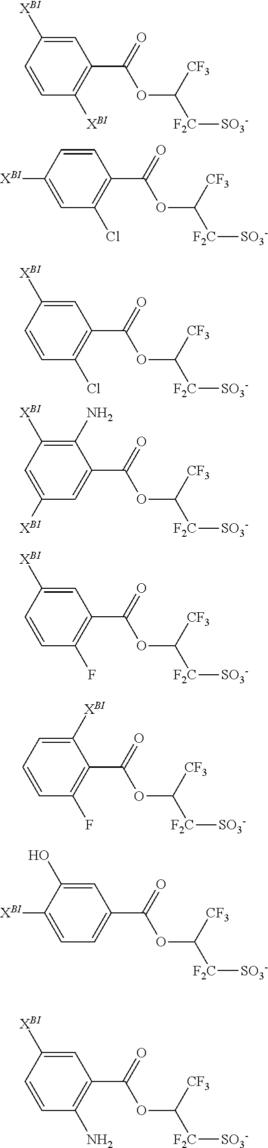

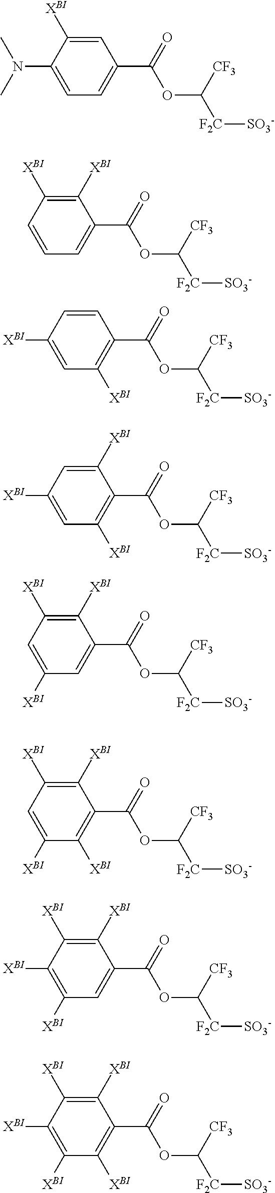

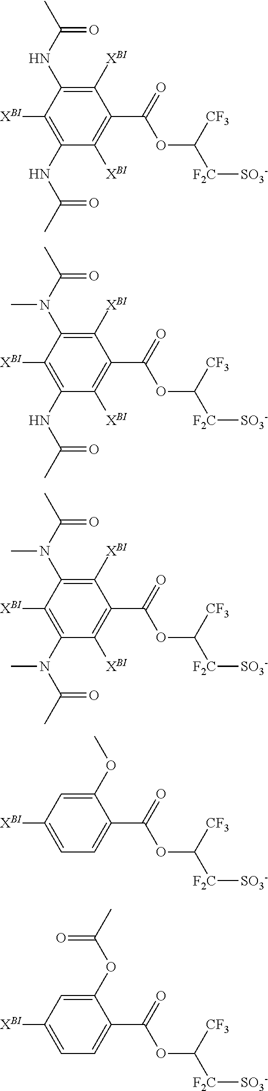

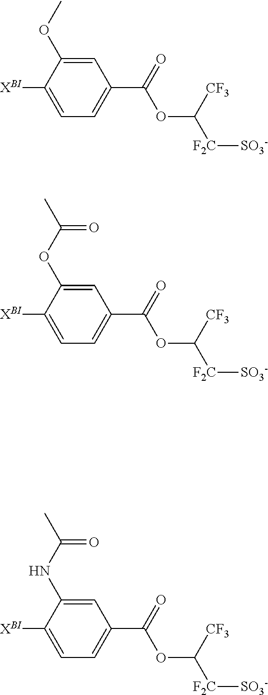

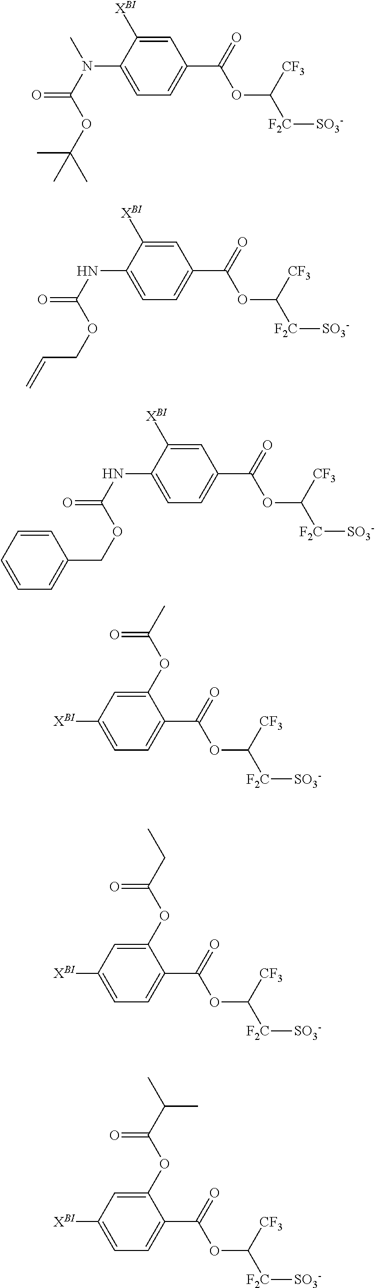

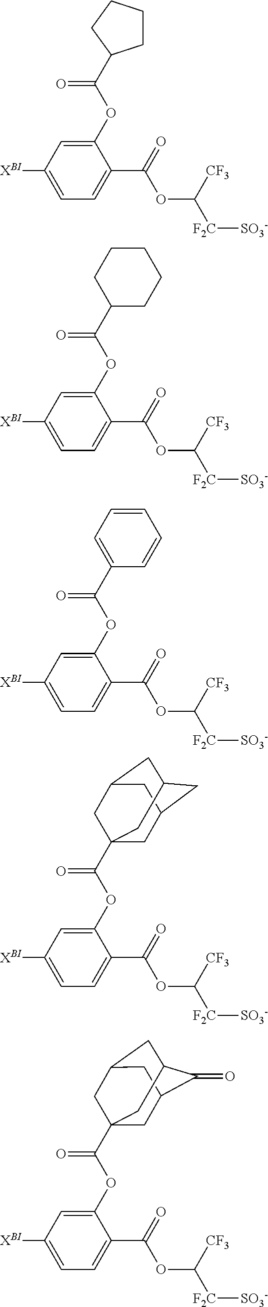

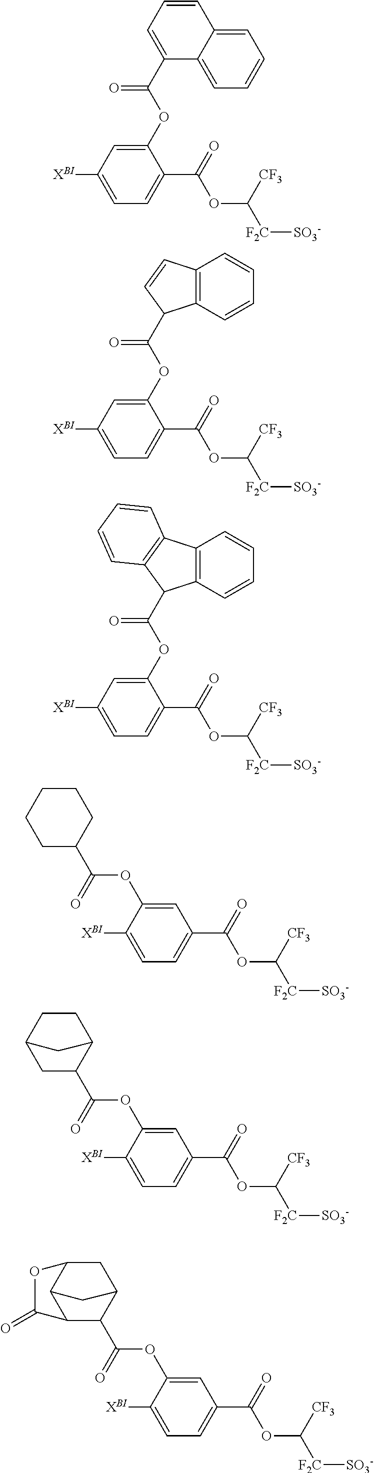

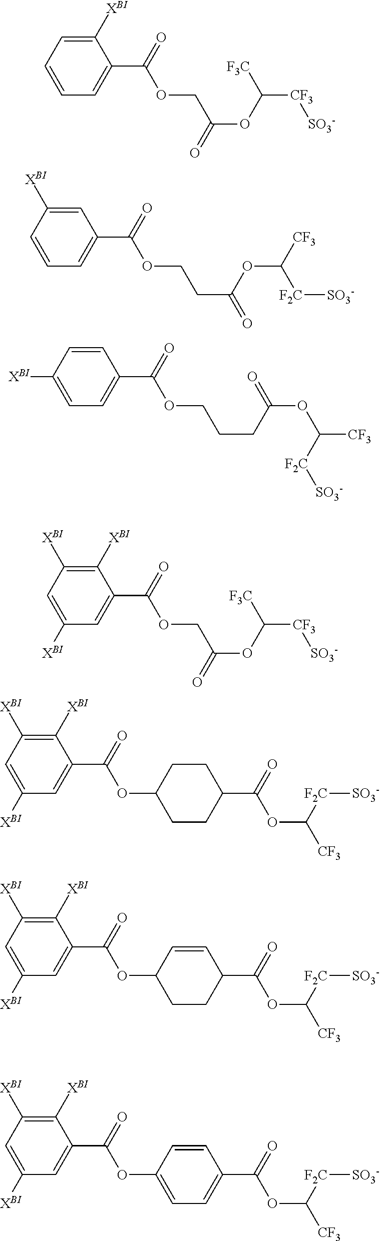

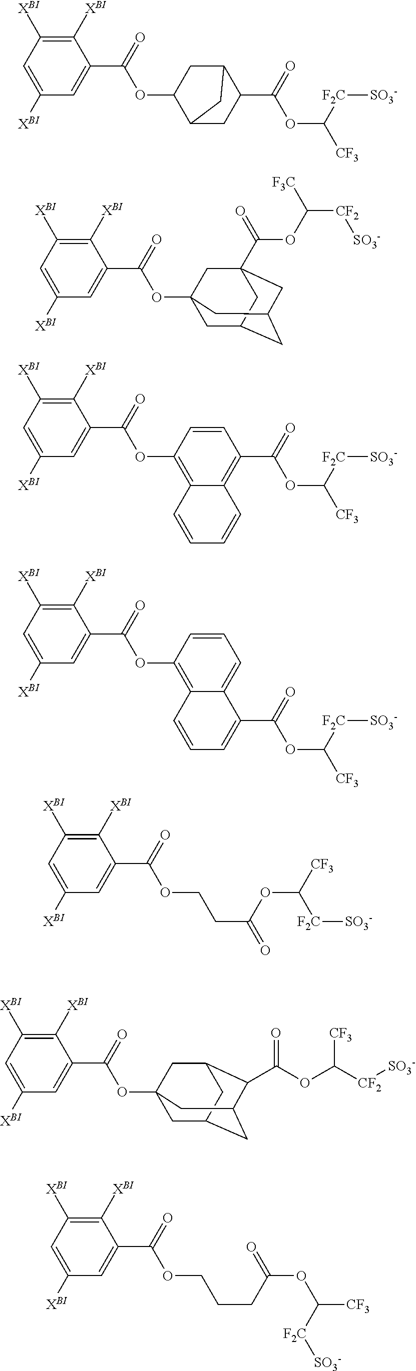

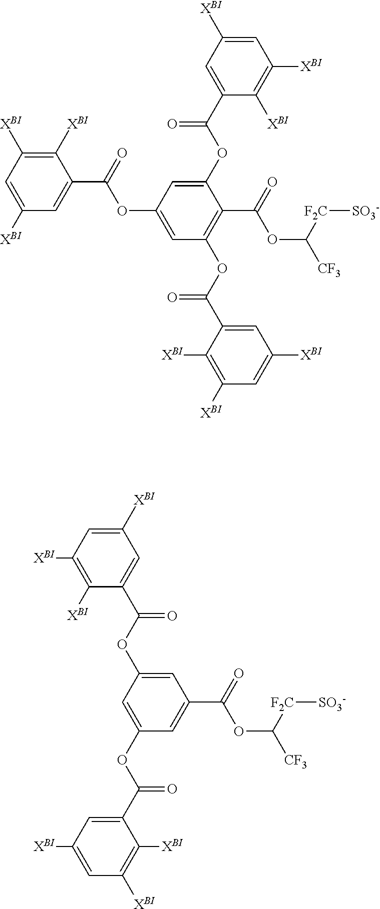

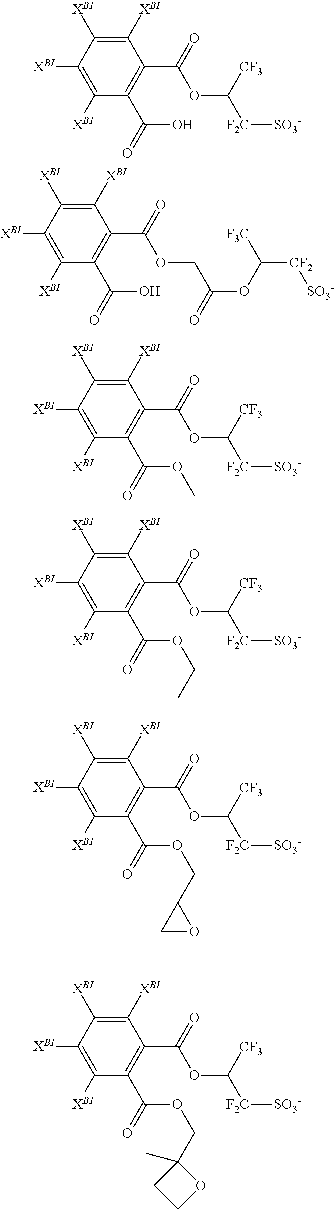

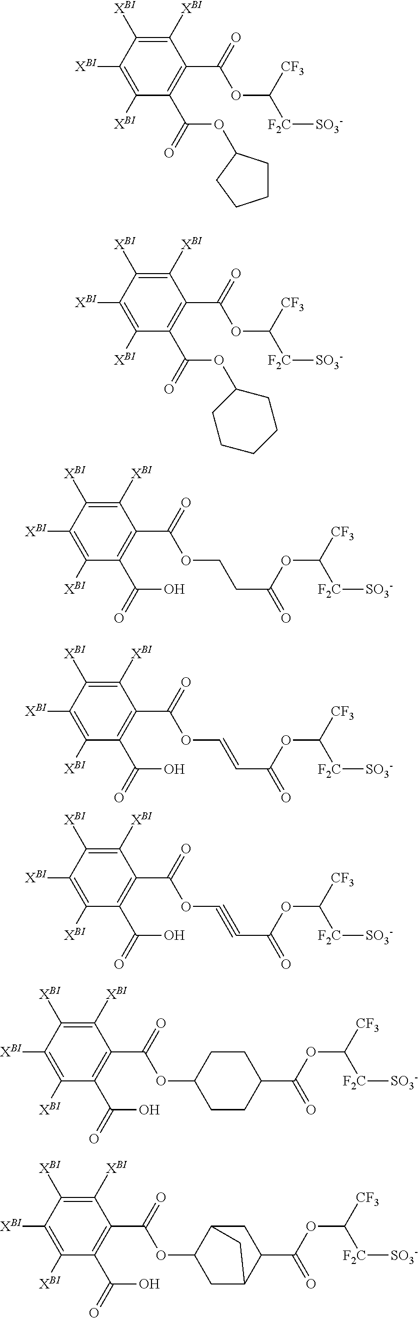

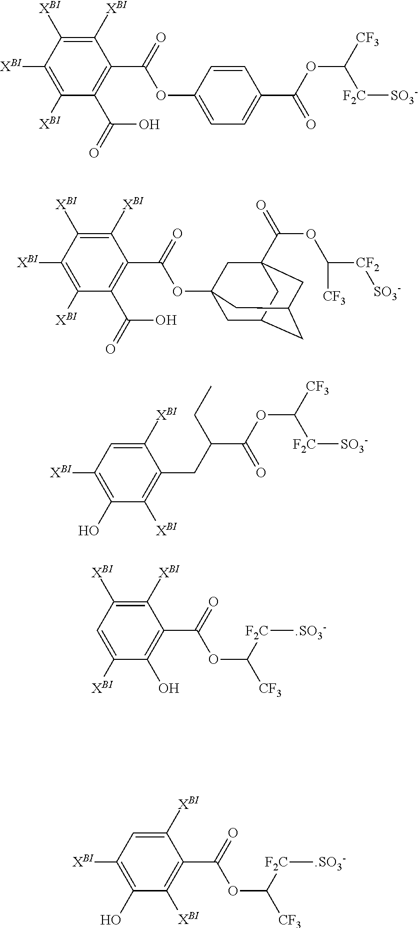

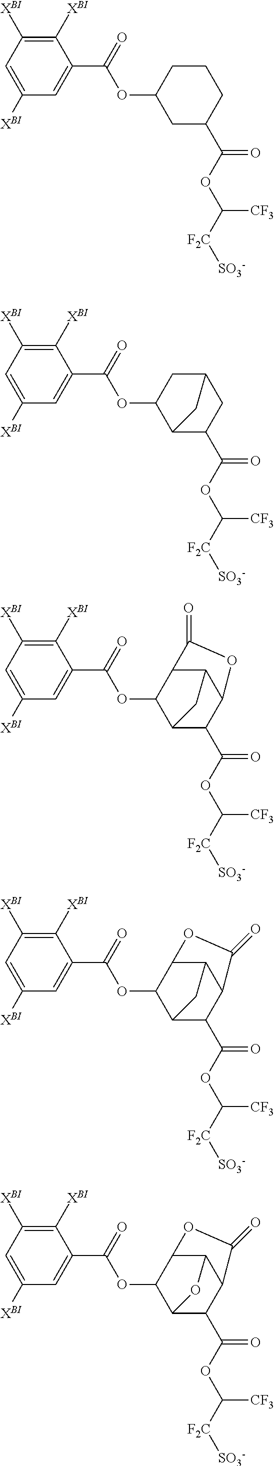

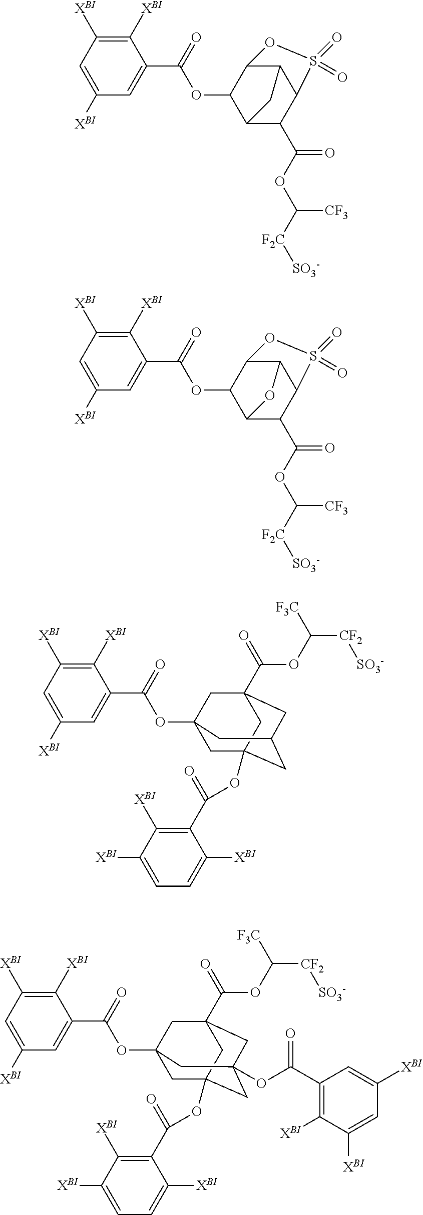

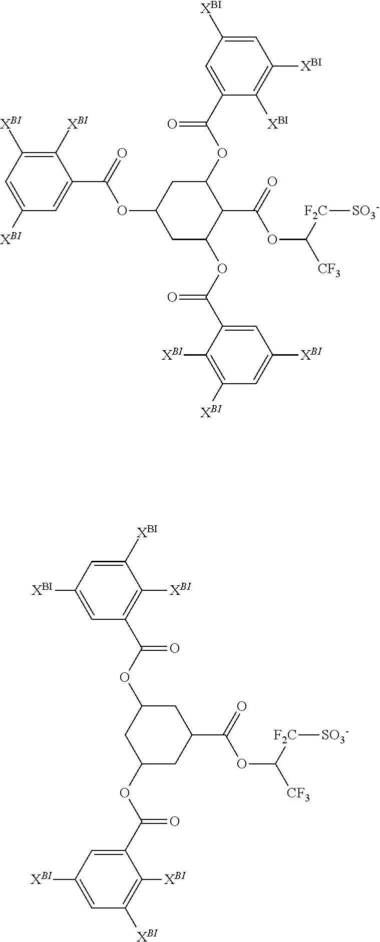

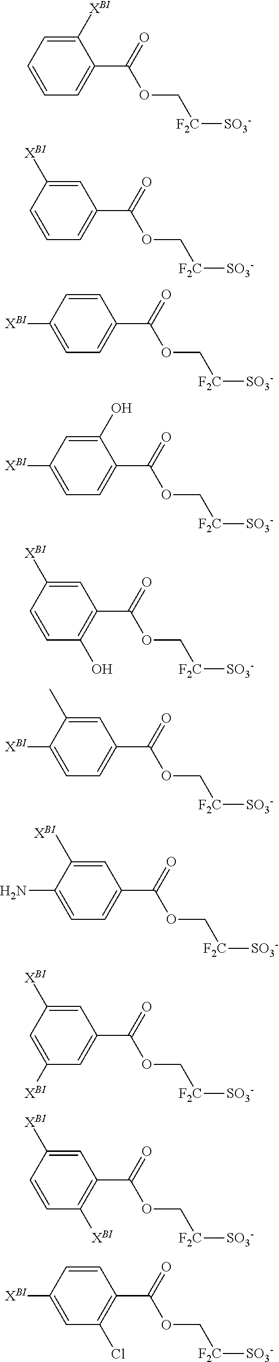

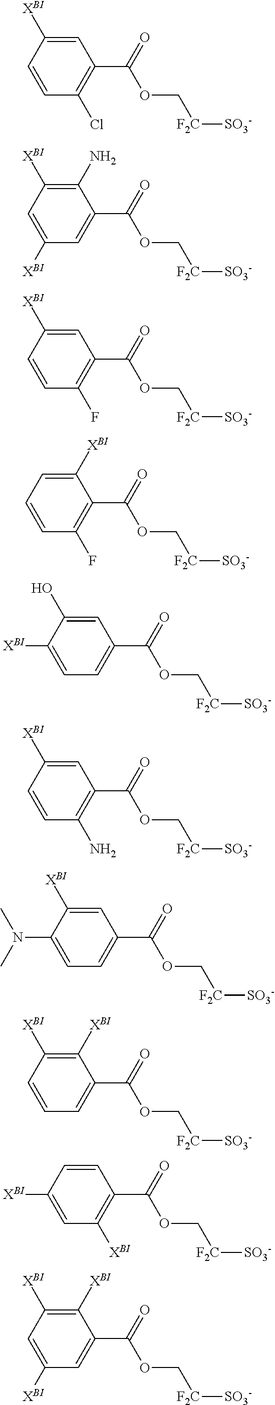

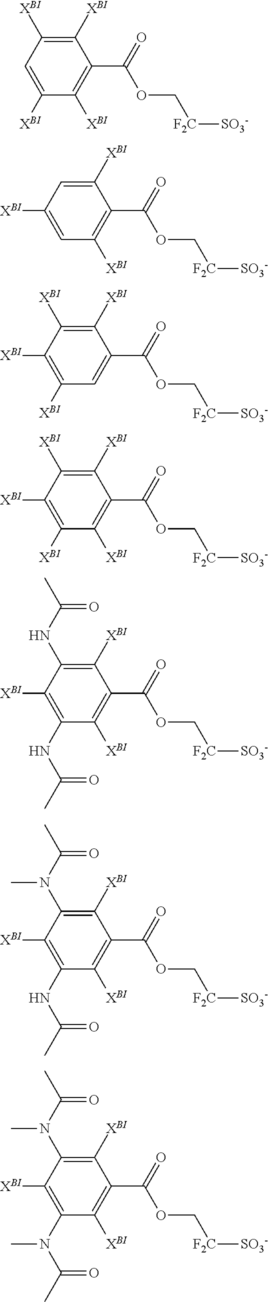

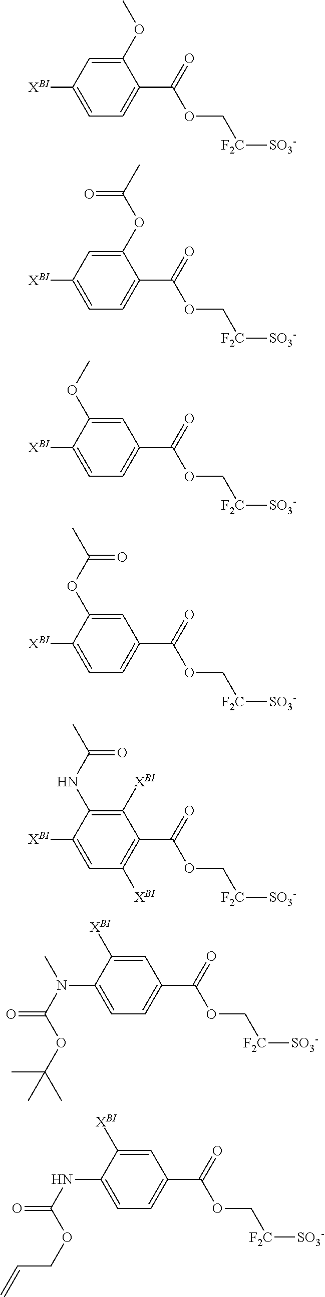

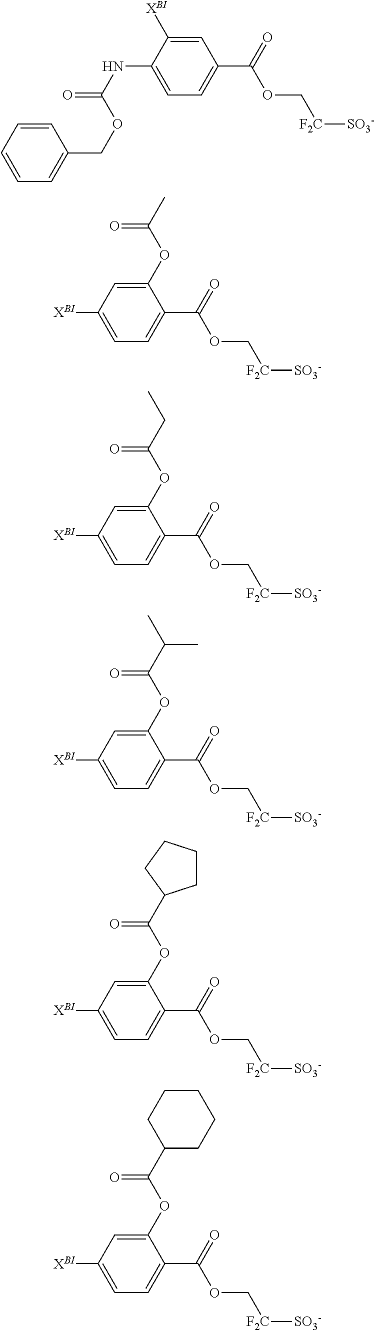

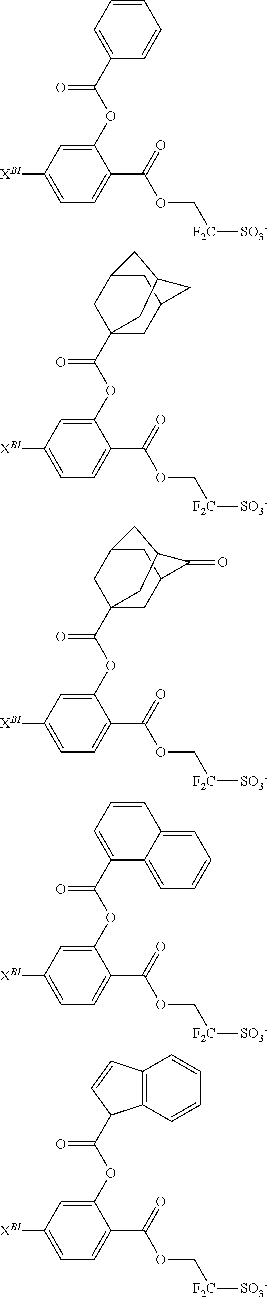

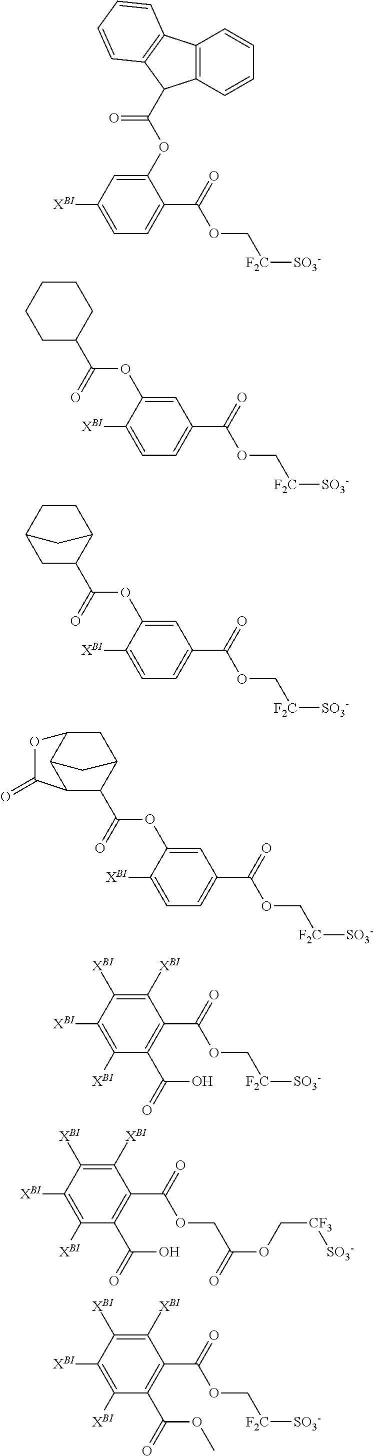

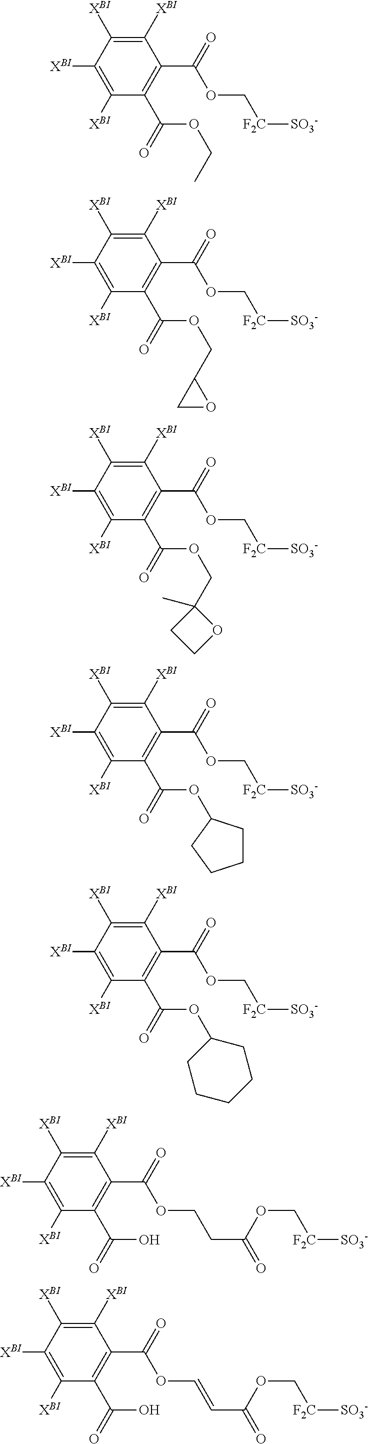

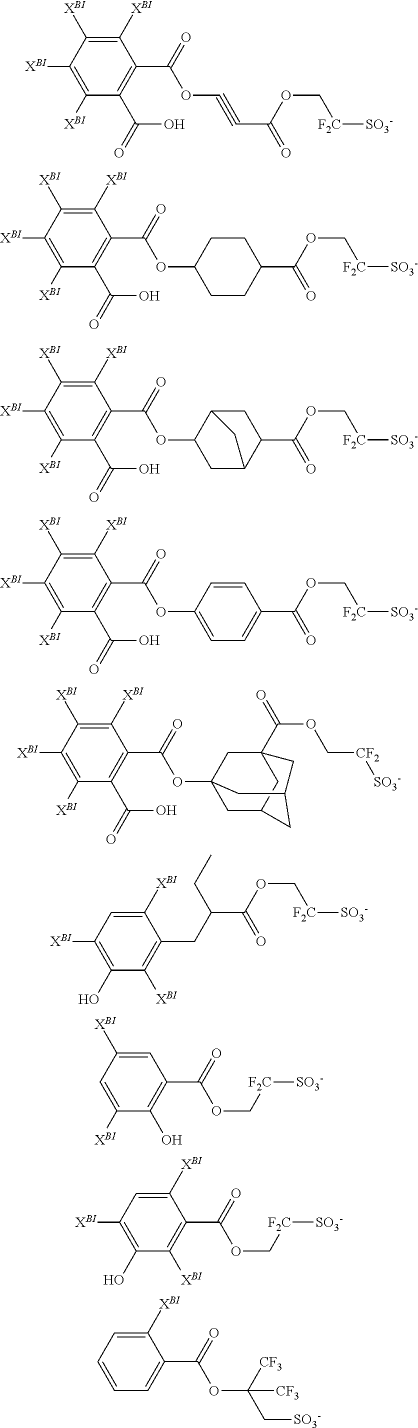

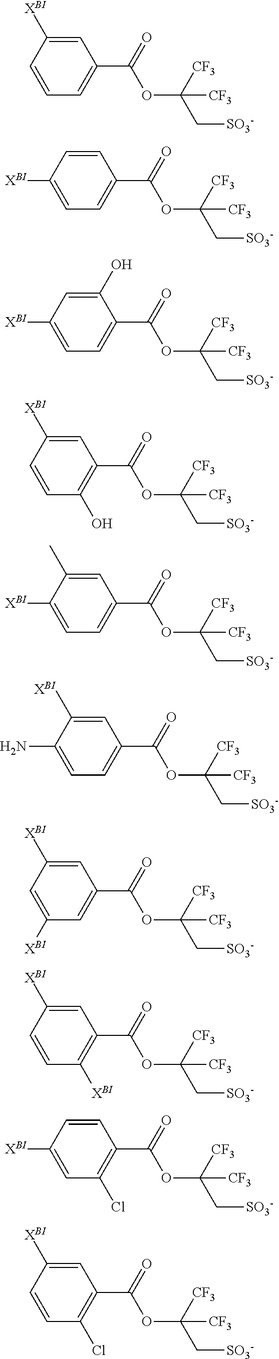

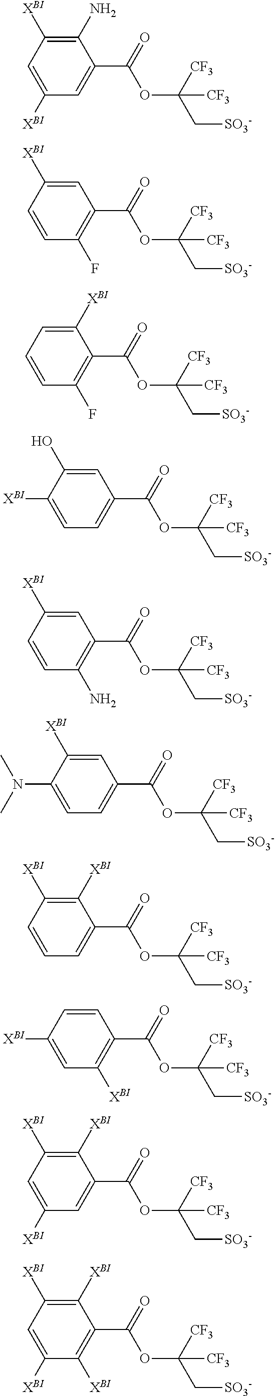

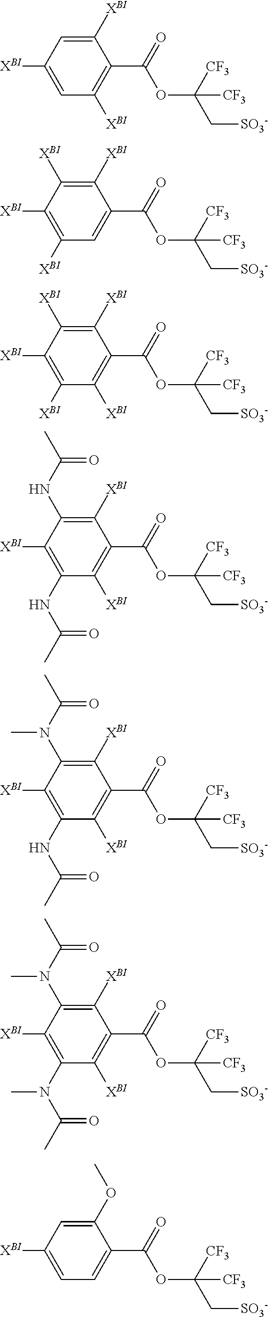

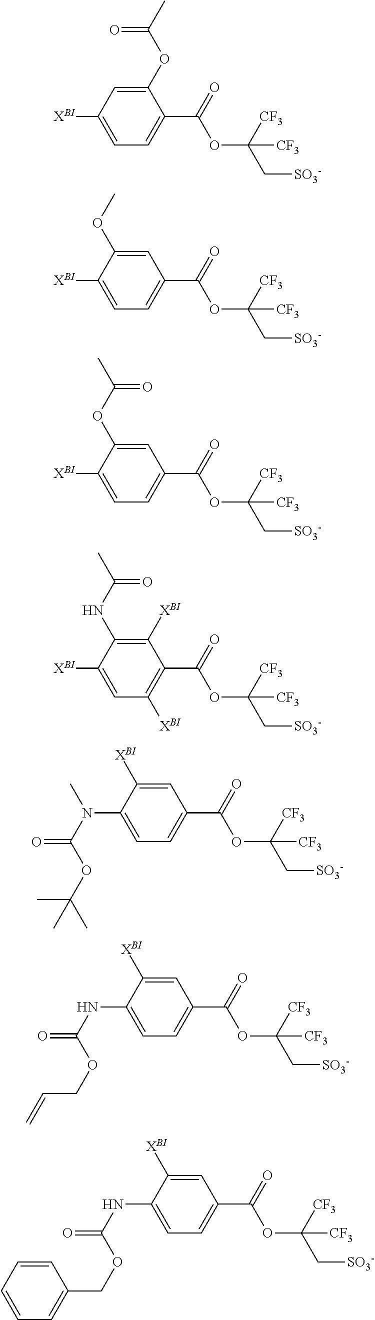

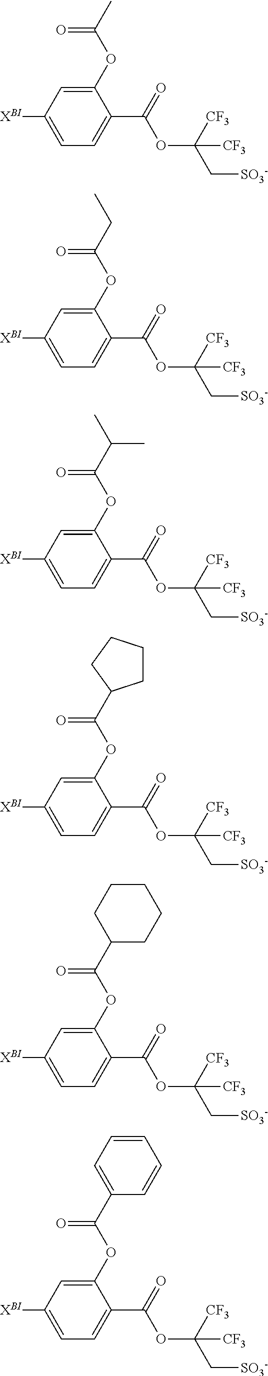

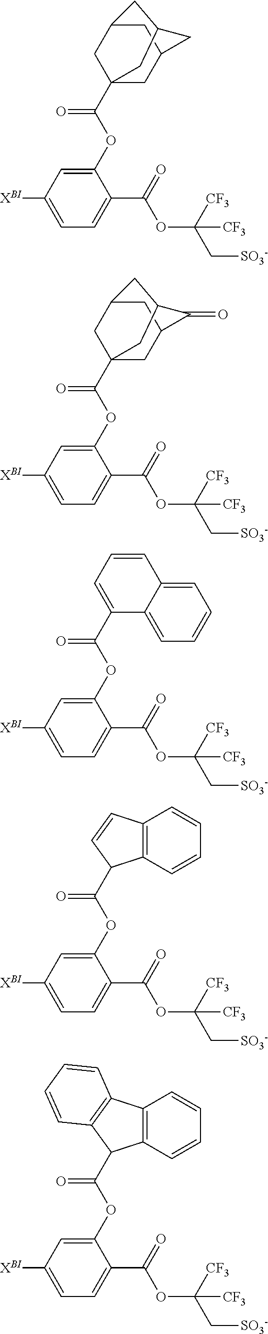

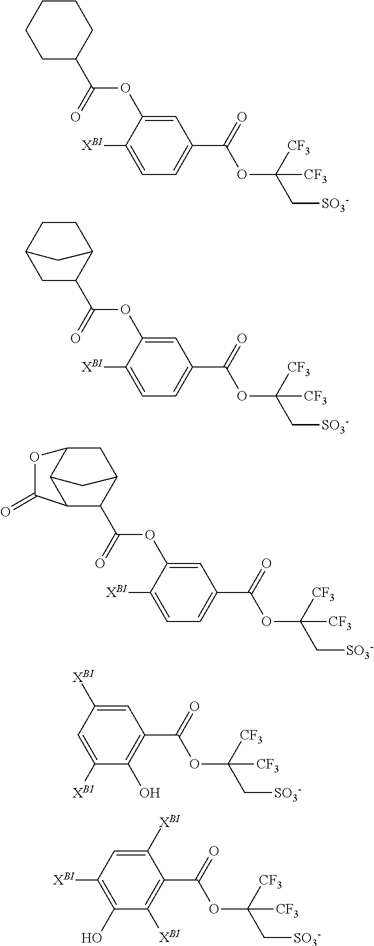

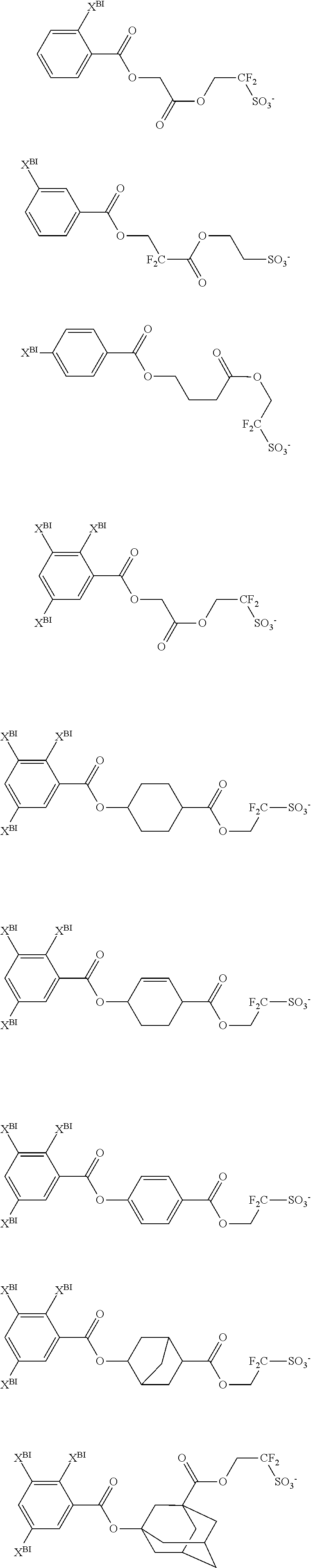

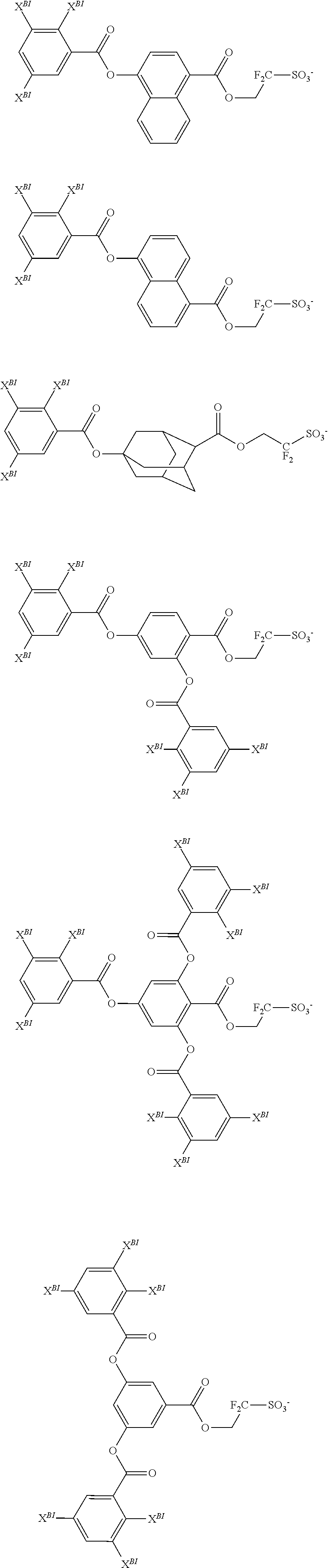

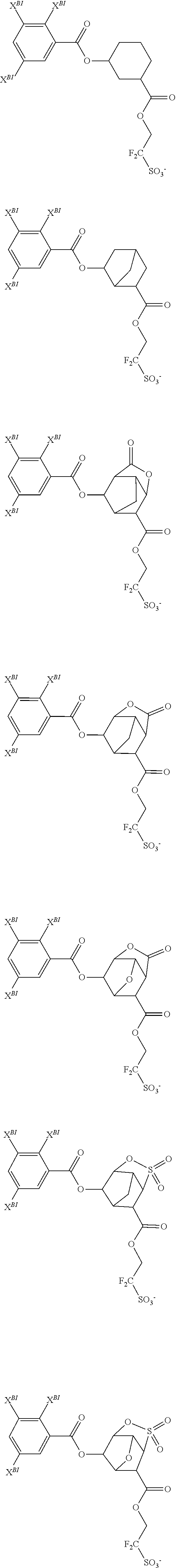

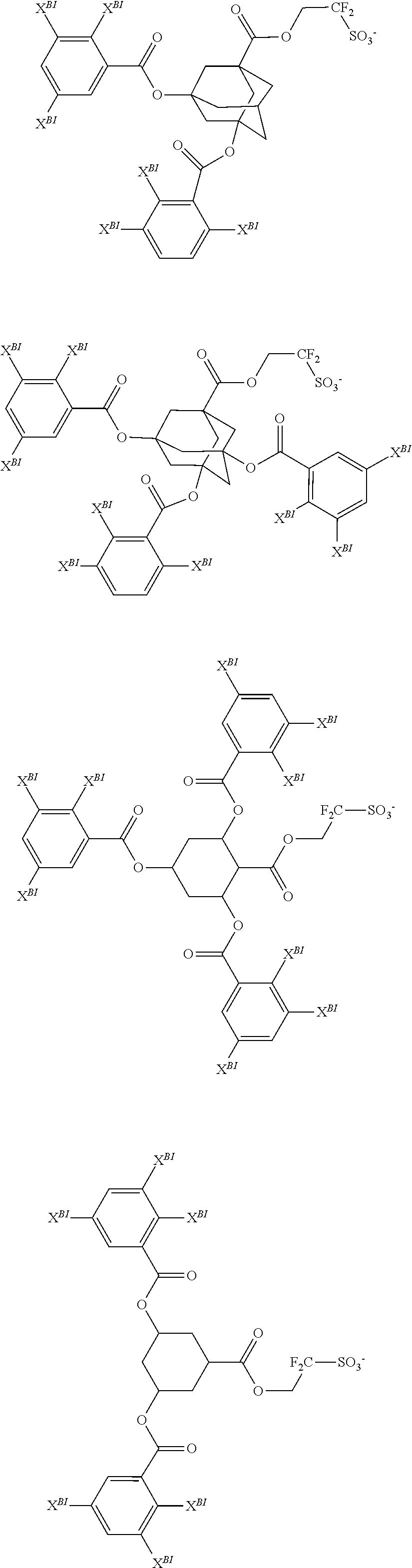

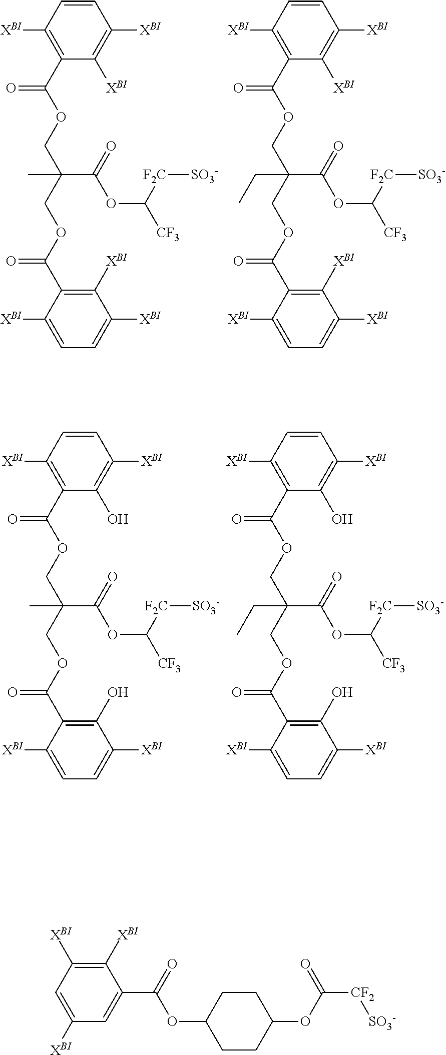

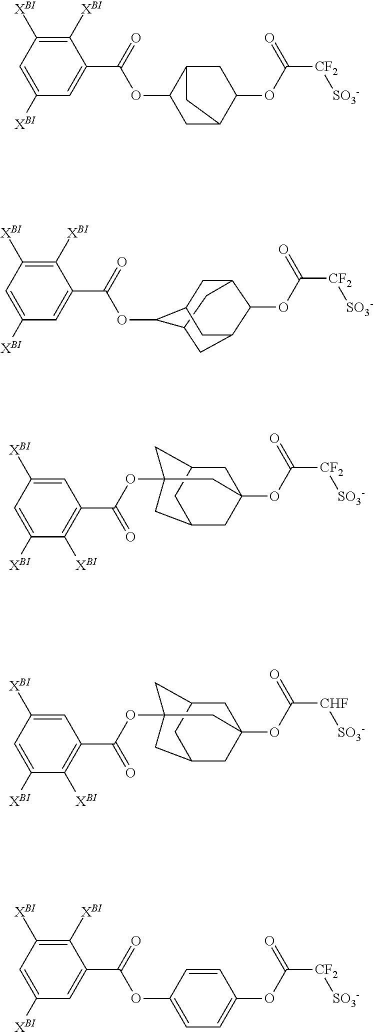

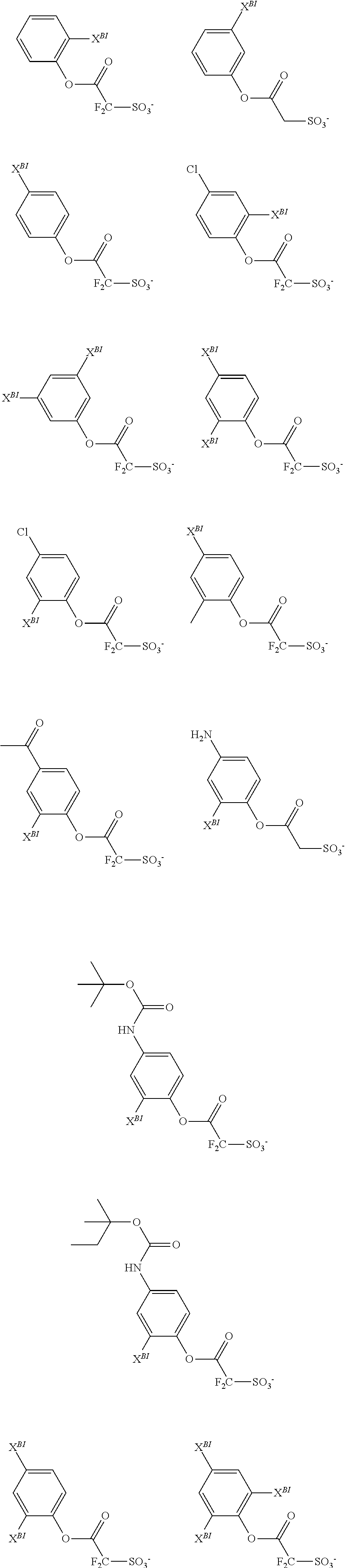

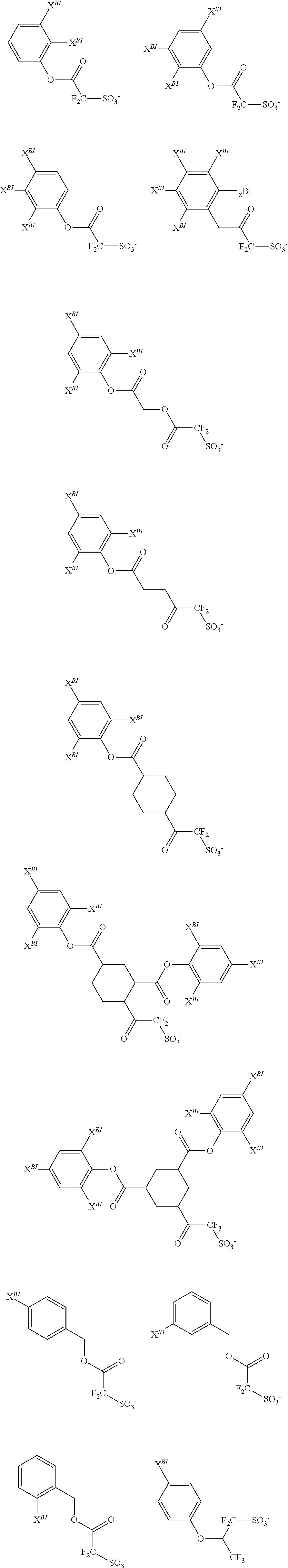

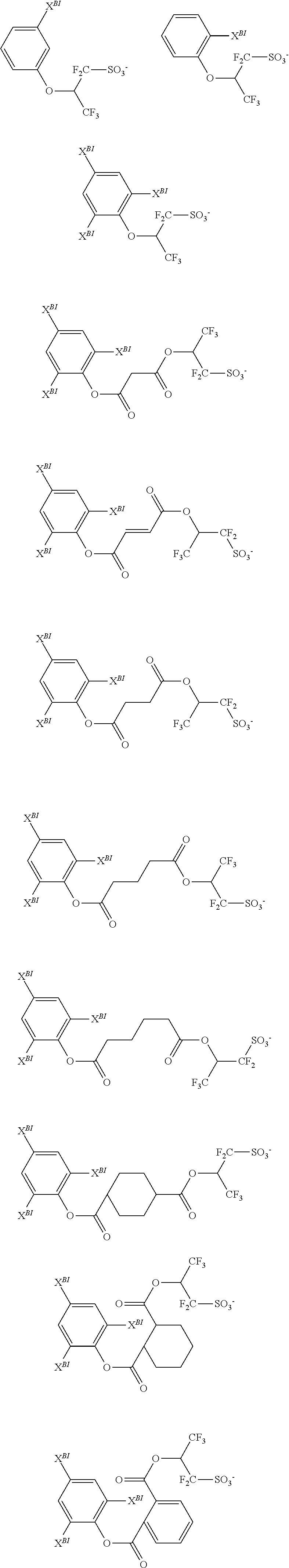

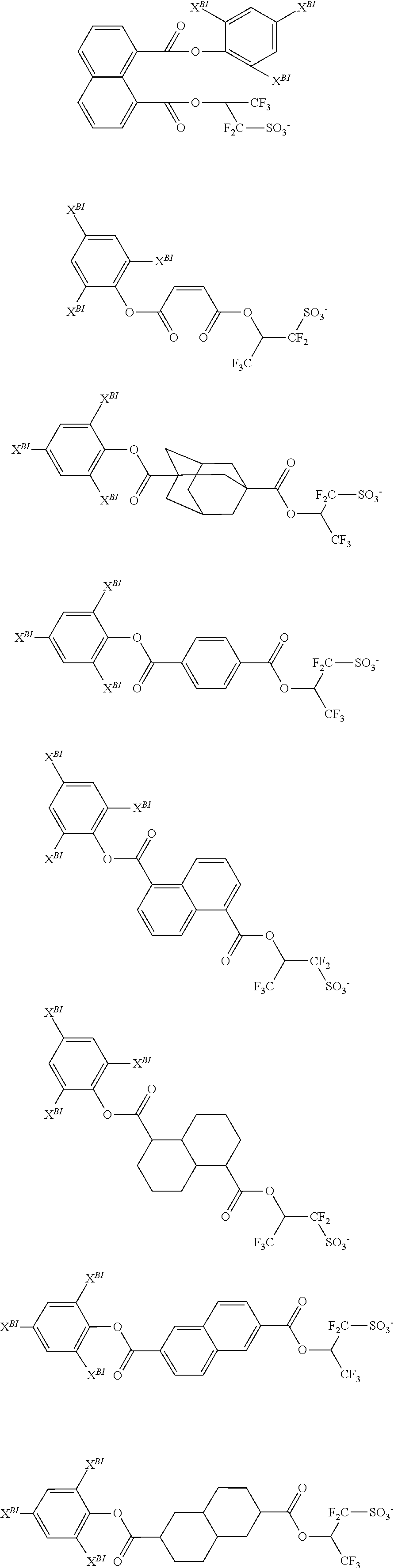

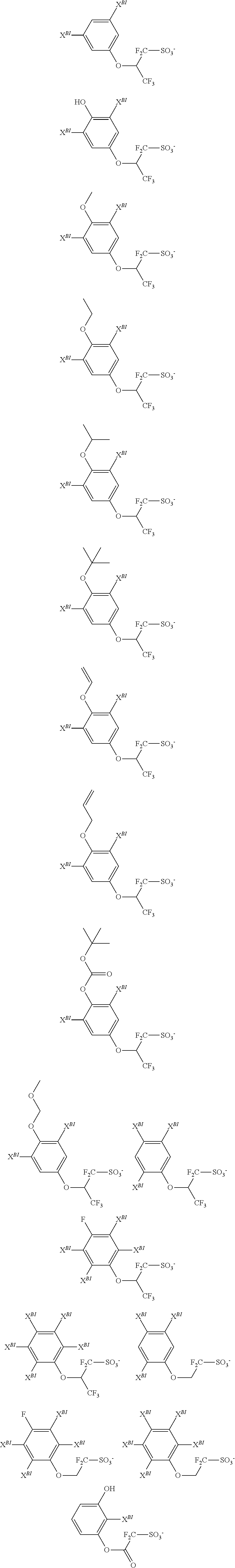

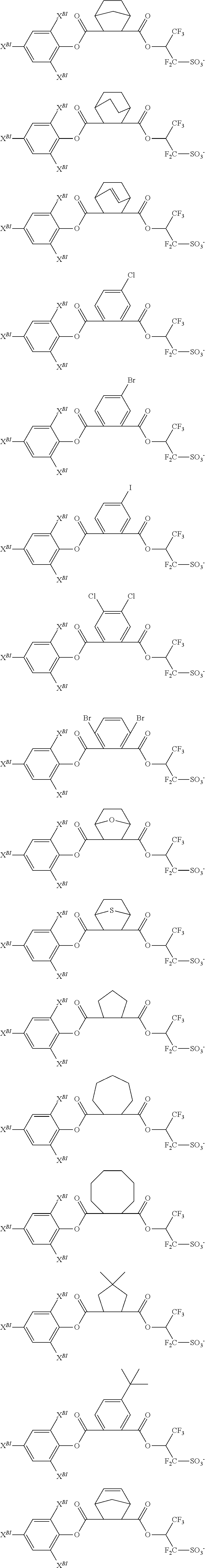

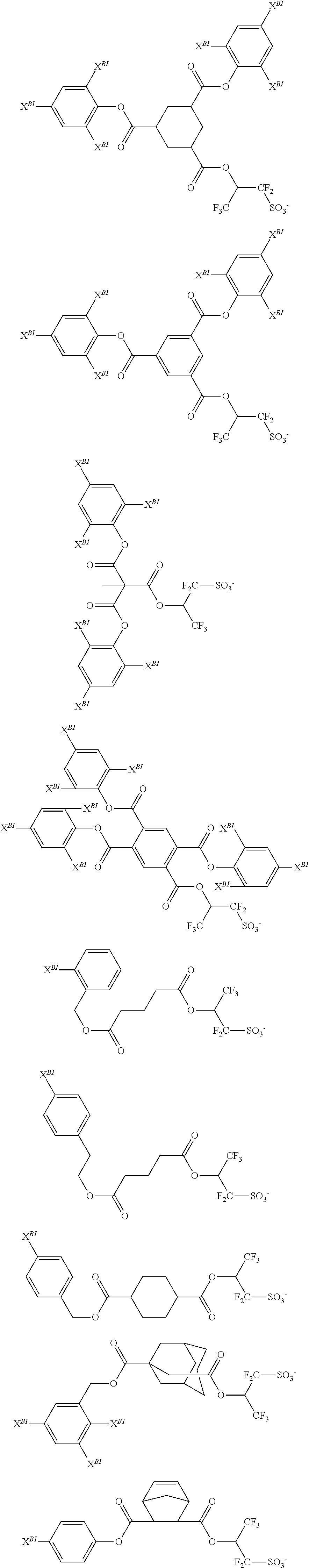

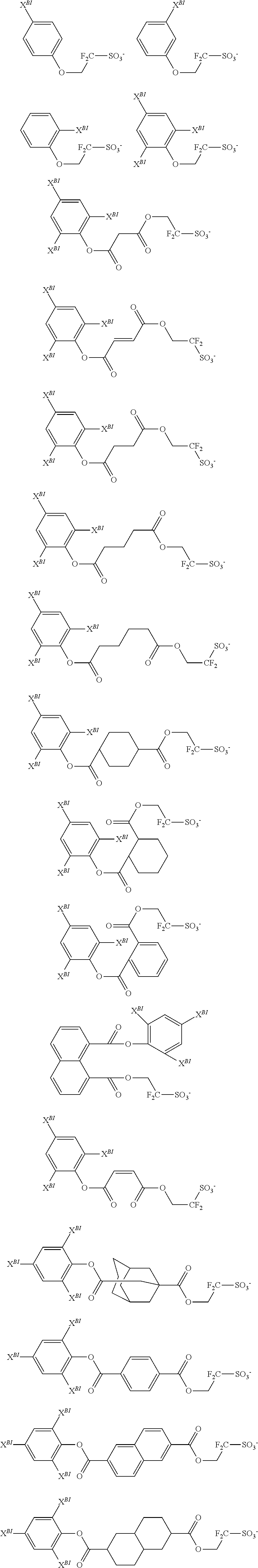

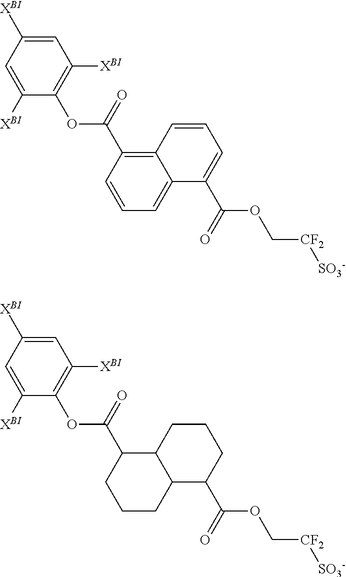

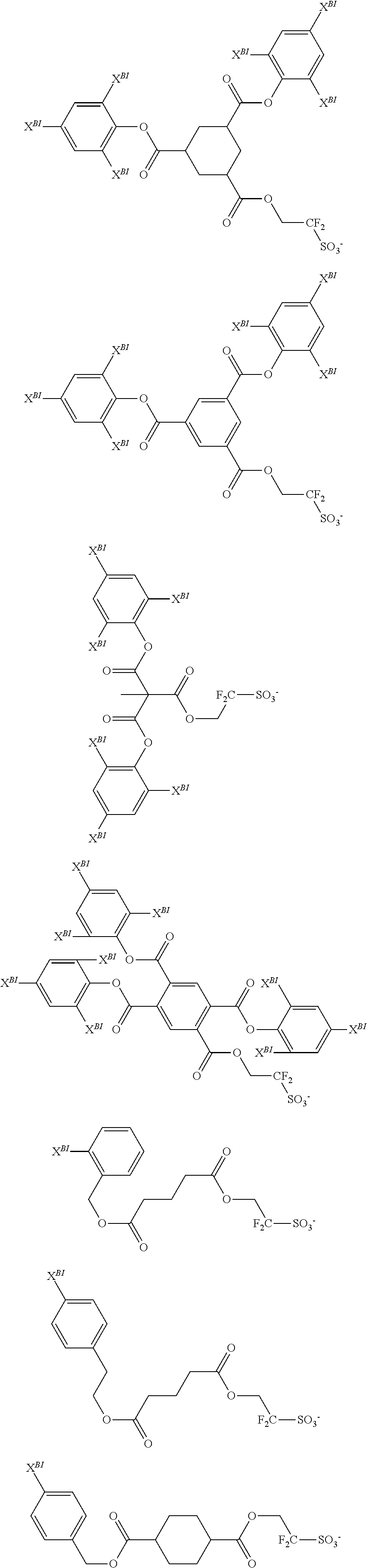

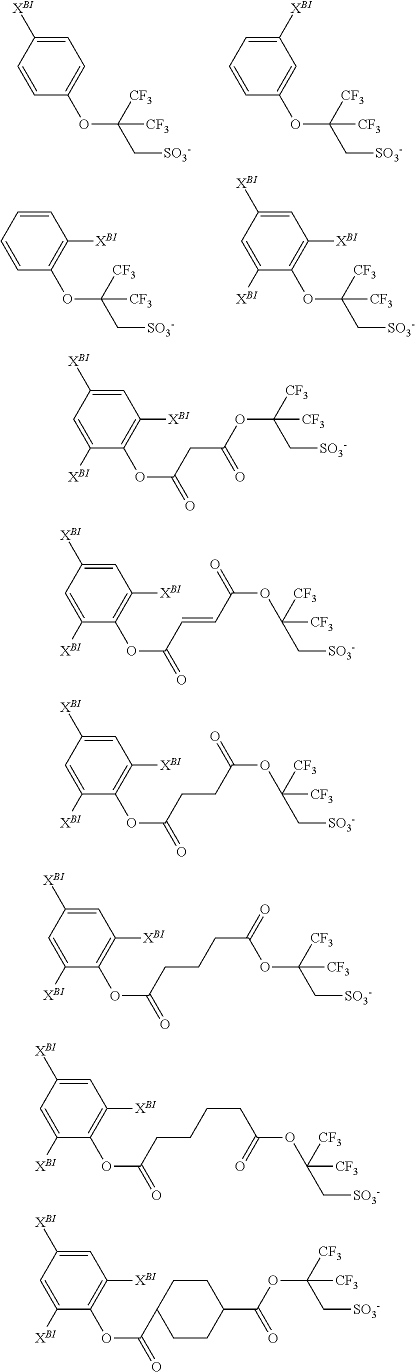

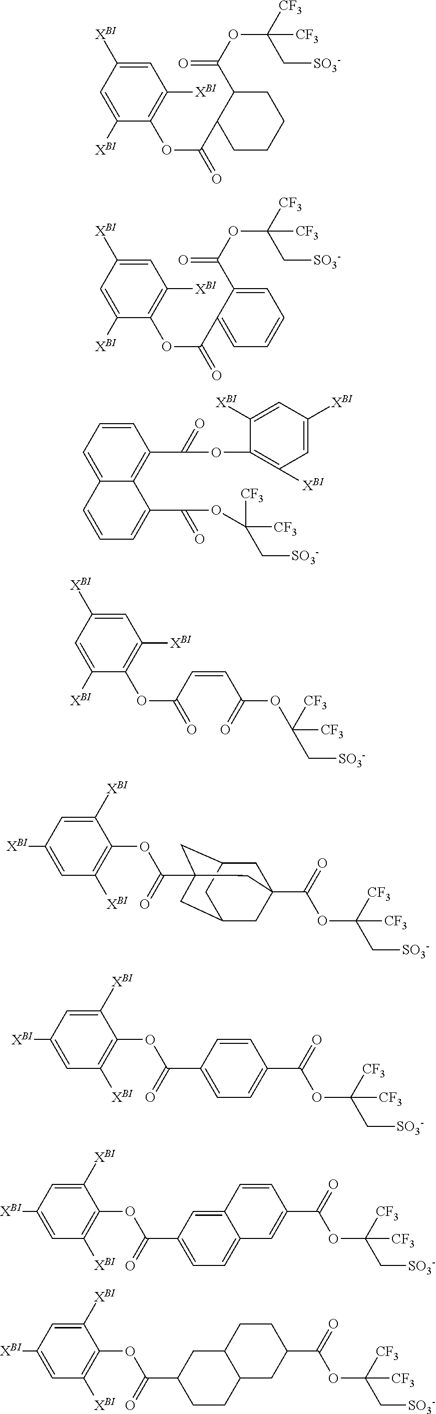

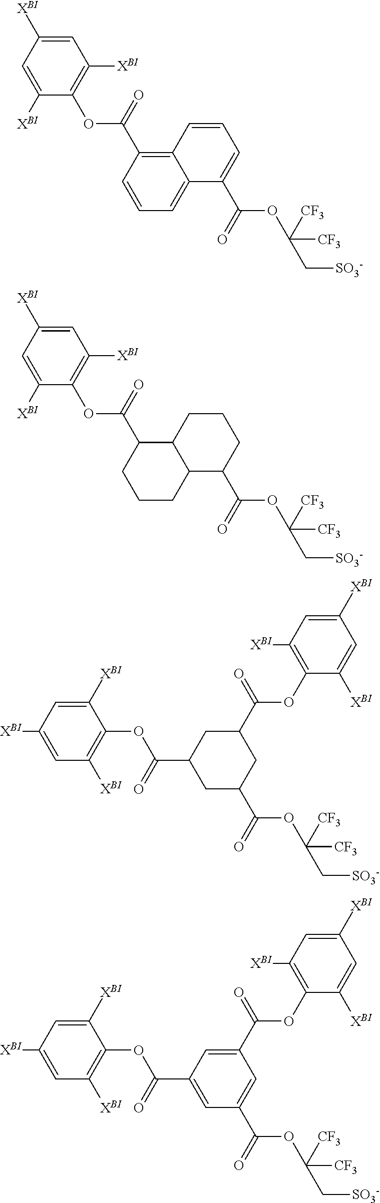

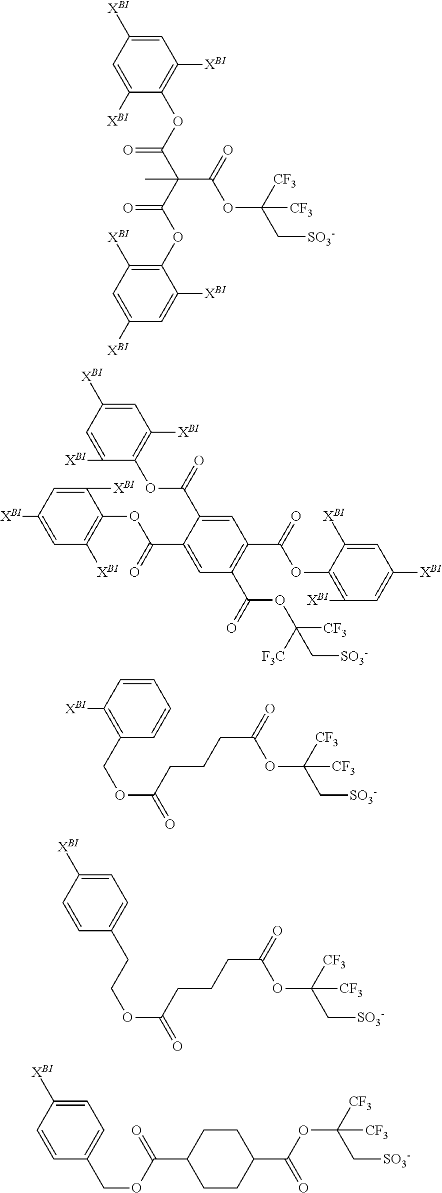

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159.

Further useful examples of the non-nucleophilic counter ion include bulky fluorine-free benzenesulfonic acid anions as described in JP-A 2006-276759, JP-A 2015-117200, JP-A 2016-065016, and JP-A 2019-202974; fluorine-free benzenesulfonic acid or alkylsulfonic acid anions having an iodized aromatic group bonded thereto as described in JP 6645464.

Also useful are bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989.

Also compounds having the formula (3) are preferred as the PAG.

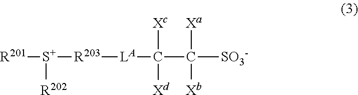

In formula (3), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The C₁-C₃₀ hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decanyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In these hydrocarbyl groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene; and combinations thereof. In these hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred.

In formula (3), L^{A} is a single bond, ether bond or a C₁-C₂₀ hydrocarbylene group which may contain a heteroatom. The hydrocarbylene group may be saturated or unsaturated and straight, branched or cyclic. Examples thereof are as exemplified above for R²⁰³.

In formula (3), X^{a}, X^{b}, X^{c} and X^{d} are each independently hydrogen, fluorine or trifluoromethyl, with the proviso that at least one of X^{a}, X^{b}, X^{c} and X^{d} is fluorine or trifluoromethyl.

Of the PAGs having formula (3), those having the formula (3′) are preferred.

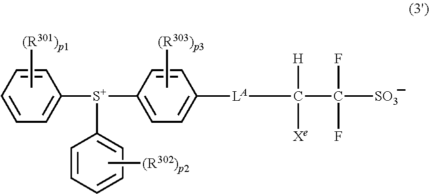

In formula (3′), L^{A} is as defined above. X^{e} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (2A′). The subscripts p1 and p2 are each independently an integer of 0 to 5, and p3 is an integer of 0 to 4.

Examples of the PAG having formula (3) are as exemplified for the PAG having formula (2) in JP-A 2017-026980.

Of the foregoing PAGs, those having an anion of formula (2A′) or (2D) are especially preferred because of reduced acid diffusion and high solubility in the resist solvent. Also those having formula (3′) are especially preferred because of extremely reduced acid diffusion.

When the resist composition contains the PAG (D), it is preferably used in an amount of 0.1 to 40 parts, and more preferably 0.5 to 20 parts by weight per 80 parts by weight of the base polymer (C). An amount of the PAG (D) in the range ensures good resolution and eliminates the risk of leaving foreign particles after development or during separation of resist film. The PAG (D) may be used alone or in admixture of two or more. When the base polymer contains repeat units (c1) to (c4) and/or the resist composition contains the PAG (D), the resist composition functions as a chemically amplified resist composition.

### (E) Nitrogen-Containing Compound

While the resist composition essentially contains the quencher (A), it may further contain a nitrogen-containing compound as another quencher. Suitable nitrogen-containing compounds include primary, secondary and tertiary amine compounds, specifically amine compounds having a hydroxy group, ether bond, ester bond, lactone ring, cyano group, or sulfonic ester bond, as described in JP-A 2008-111103, paragraphs [0146]-[0164], and primary and secondary amine compounds protected with a carbamate group, as described in JP 3790649.

Also a sulfonium salt of sulfonic acid having a nitrogen-containing substituent may be used as the nitrogen-containing compound (E). This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and 2012-046501, for example.

When the resist composition contains the nitrogen-containing compound (E), it is preferably used in an amount of 0.001 to 12 parts by weight, more preferably 0.01 to 8 parts by weight per 80 parts by weight of the base polymer (C). The nitrogen-containing compound may be used alone or in admixture.

### (F) Surfactant

The resist composition may further comprise (F) a surfactant. It is typically a surfactant which is insoluble or substantially insoluble in water and alkaline developer, or a surfactant which is insoluble or substantially insoluble in water and soluble in alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are fluorochemical surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also Useful.

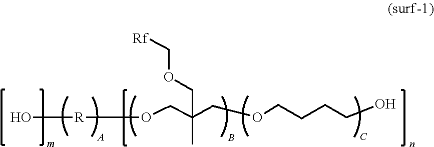

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

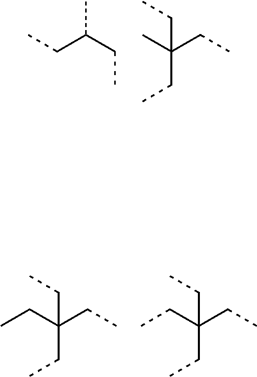

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferred.

Rf is trifluoromethyl or pentafluoroethyl, and preferably trifluoromethyl. The subscript m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the surface of a resist film for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (4A) to (4E).

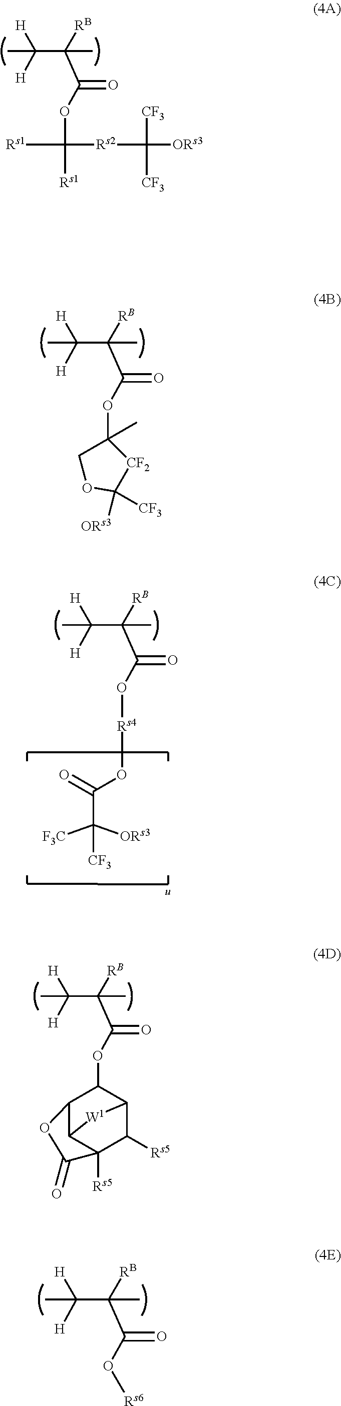

In formulae (4A) to (4E), R^{B} is hydrogen, fluorine, methyl or trifluoromethyl. W¹ is —CH₂—, —CH₂CH₂—, —O—, or two separate —H. R^{s1} is each independently hydrogen or a C₁-C₁-hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is an integer of 1 to 3. R^{s5} is each independently hydrogen or a group: —C(═O)—O—R^{sa} wherein R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group R^{s1} is preferably saturated while it may be straight, branched or cyclic. Examples thereof include alkyl groups such as methyl, ethyl, n-propyl, isopropyl, n-butyl, isobutyl, sec-butyl, tert-butyl, n-pentyl, n-hexyl, n-heptyl, n-octyl, n-nonyl and n-decyl, and cyclic saturated hydrocarbyl groups such as cyclopropyl, cyclobutyl, cyclopentyl, cyclohexyl, adamantyl and norbornyl. Inter alia, C₁-C₆ groups are preferred.

The hydrocarbylene group R^{s2} is preferably saturated while it may be straight, branched or cyclic. Examples thereof include methylene, ethylene, propylene, butylene, and pentylene.

The hydrocarbyl group R^{s3} or R^{s6} may be saturated or unsaturated and straight, branched or cyclic. Examples thereof include saturated hydrocarbyl groups and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as n-undecyl, n-dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group represented by R^{sa} is preferably saturated while it may be straight, branched or cyclic. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (4A) to (4E) are shown below, but not limited thereto. Herein R^{B} is as defined above.

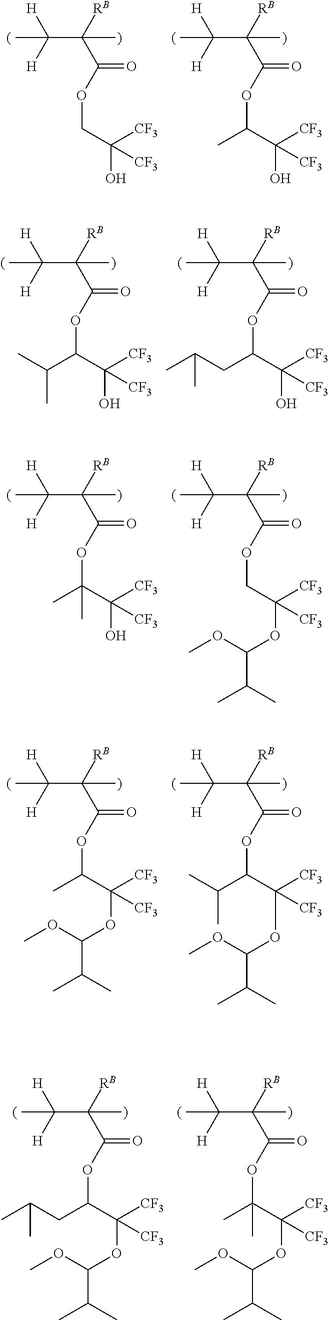

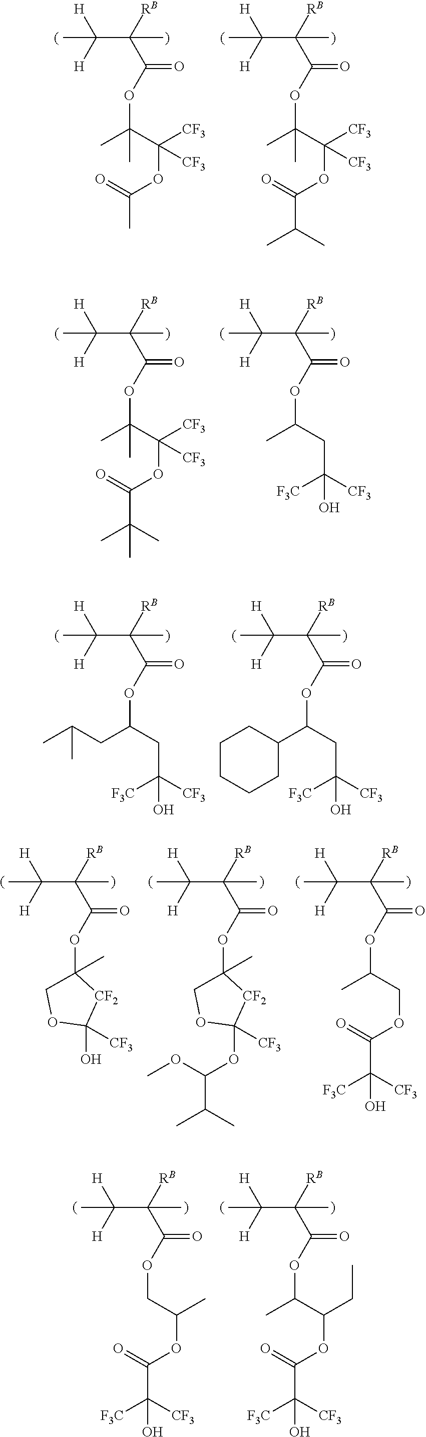

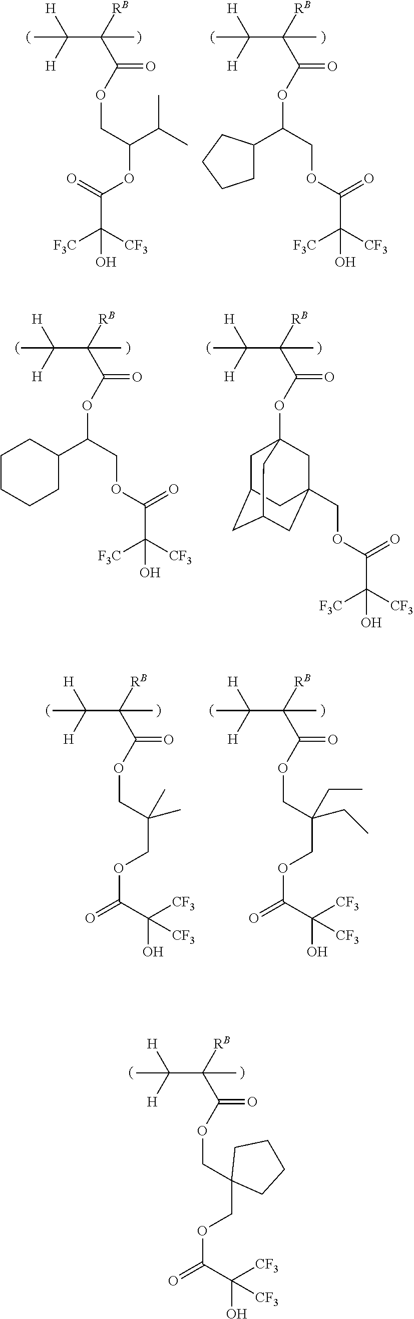

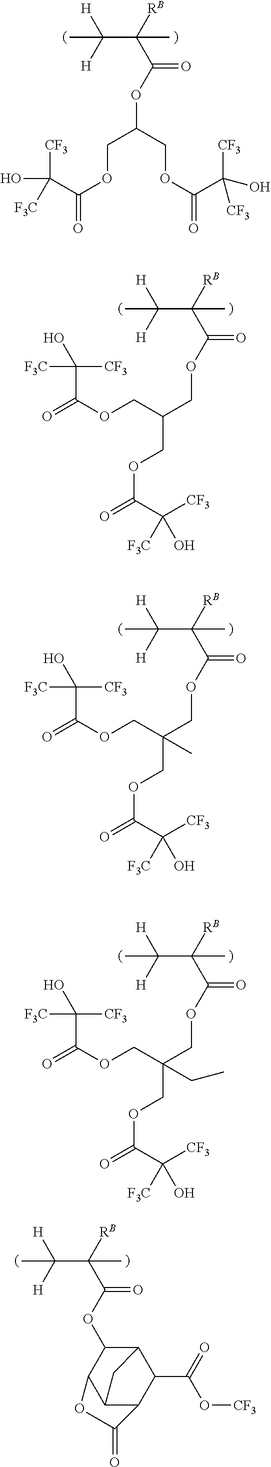

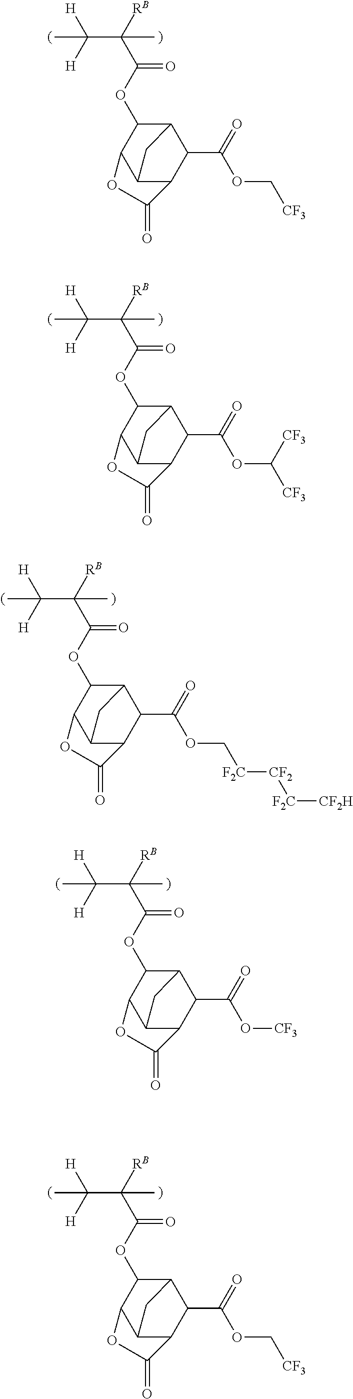

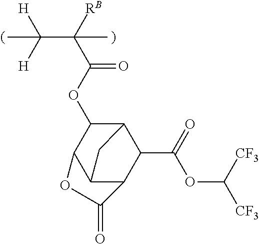

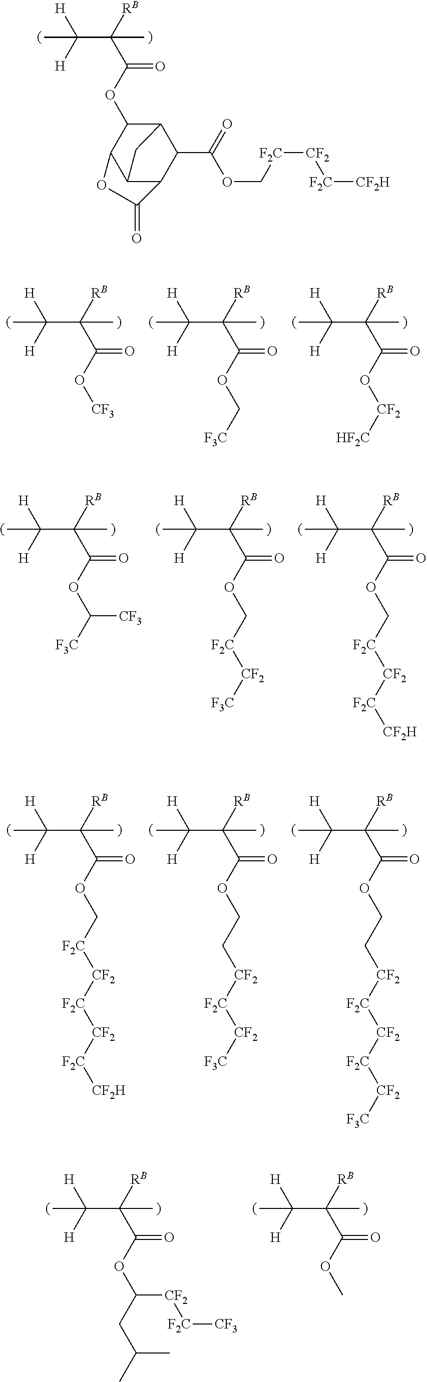

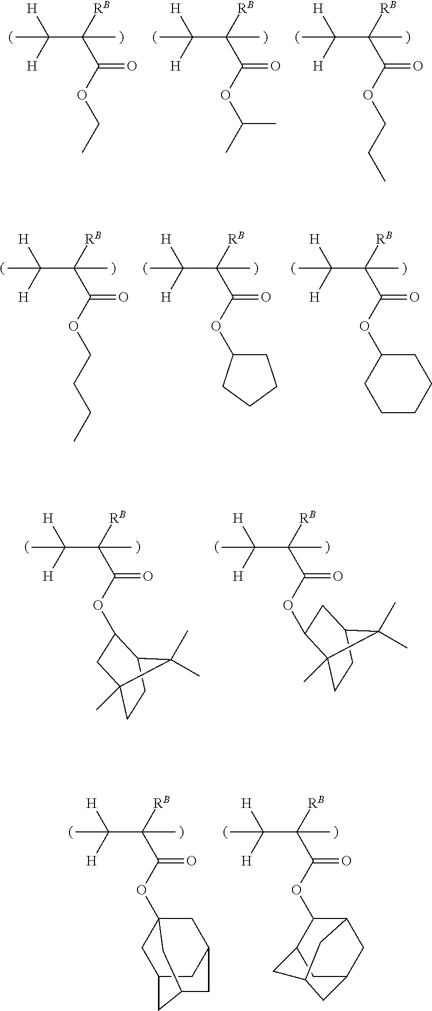

The polymeric surfactant may further contain repeat units other than the repeat units having formulae (4A) to (4E). Typical other repeat units are those derived from methacrylic acid and α-trifluoromethylacrylic acid derivatives. In the polymeric surfactant, the content of the repeat units having formulae (4A) to (4E) is preferably at least 20 mol %, more preferably at least 60 mol %, most preferably 100 mol % of the overall repeat units.

Preferably the polymeric surfactant has a Mw of 1,000 to 500,000, more preferably 3,000 to 100,000 and a Mw/Mn of 1.0 to 2.0, more preferably 1.0 to 1.6.

The polymeric surfactant may be synthesized, for example, by dissolving an unsaturated bond-containing monomer or monomers, from which repeat units having formulae (4A) to (4E) and optional other repeat units are derived, in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of the polymeric surfactant, any of well-known chain transfer agents such as dodecylmercaptan and 2-mercaptoethanol may be used for the purpose of adjusting molecular weight. An appropriate amount of the chain transfer agent is 0.01 to 10 mol % based on the total moles of monomers to be polymerized.

When the resist composition contains the surfactant (F), it is preferably used in an amount of 0.1 to 50 parts by weight, more preferably 0.5 to 10 parts by weight per 80 parts by weight of the base polymer (C). As long as the amount of the surfactant is at least 0.1 part by weight, the receding contact angle of resist film surface with water is fully improved. As long as the amount of the surfactant is up to 50 parts by weight, the dissolution rate of resist film surface in developer is so low that the resulting small-size pattern may maintain a sufficient height. The surfactant (F) may be used alone or in admixture.

### (G) Other Components

The resist composition may further comprise other components, for example, a compound which is decomposed with an acid to generate another acid (i.e., acid amplifier compound), organic acid derivative, fluorinated alcohol, and a compound with Mw≤3,000 adapted to change its solubility in developer under the action of acid (i.e., dissolution inhibitor). Each of the other components may be used alone or in admixture.

The acid amplifier compound is described in JP-A 2009-269953 and JP-A 2010-215608. The acid amplifier compound is preferably used in an amount of 0 to 5 parts, more preferably 0 to 3 parts by weight per 80 parts by weight of the base polymer. An extra amount of the acid amplifier compound can make the acid diffusion control difficult and cause degradations to resolution and pattern profile. With respect to the organic acid derivative, fluorinated alcohol and dissolution inhibitor, reference should be made to JP-A 2009-269953 and JP-A 2010-215608.

### Process

A further embodiment of the invention is a pattern forming process comprising the steps of applying the resist composition defined above onto a substrate to form a resist film thereon, exposing the resist film to high-energy radiation, and developing the exposed resist film in a developer to form a resist pattern.

The substrate used herein may be selected from, for example, substrates for IC fabrication, e.g., Si, SiO₂, SiN, SiON, TiN, WSi, BPSG, SOG, and organic antireflective coating, and substrates for mask circuit fabrication, e.g., Cr, CrO, CrON, MoSi2, and SiO₂.

The resist composition is first applied onto a substrate by a suitable coating technique such as spin coating. The coating is prebaked on a hotplate preferably at a temperature of 60 to 150° C. for 1 to 10 minutes, more preferably at 80 to 140° C. for 1 to 5 minutes to form a resist film of 0.05 to 2 μm thick.

Then the resist film is exposed to high-energy radiation, for example, i-line, KrF excimer laser, ArF excimer laser, EB or EUV. On use of KrF or ArF excimer laser or EUV, the resist film is exposed through a mask having the desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0, typically water between the resist film and the projection lens may be employed if desired. In the case of immersion lithography, a protective film which is insoluble in water may be formed on the resist film.

While the water-insoluble protective film serves to prevent any components from being leached out of the resist film and to improve water slippage at the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, and more preferably at 80 to 140° C. for 1 to 3 minutes.

Finally, development is carried out using as the developer an aqueous alkaline solution, such as a 0.1 to 5 wt %, preferably 2 to 3 wt %, aqueous solution of tetramethylammonium hydroxide (TMAH), this being done by a conventional method such as dip, puddle, or spray development for a period of 0.1 to 3 minutes, and preferably 0.5 to 2 minutes. In this way the exposed region of resist film is dissolved away, forming the desired pattern on the substrate.

After formation of the resist film, deionized water rinsing may be carried out for extracting the acid generator and the like from the film surface or washing away particles, or after exposure, rinsing may be carried out for removing water droplets left on the resist film.

A pattern may also be formed by a double patterning process. The double patterning process includes a trench process of processing an underlay to a 1:3 trench pattern by a first step of exposure and etching, shifting the position, and forming a 1:3 trench pattern by a second step of exposure for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, an alkaline aqueous solution is often used as the developer. Instead, the negative tone development technique wherein the unexposed region of resist film is dissolved in an organic solvent developer is also applicable. In the organic solvent development, the organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture of two or more.

### EXAMPLES

Examples of the invention are given below by way of illustration and not by way of limitation. The abbreviation “pbw” is parts by weight. Analysis is made by time-of-flight mass spectrometry (TOF-MS) using the instrument: MALDI TOF-MS S3000 by JEOL Ltd.

### Synthesis of Onium Salts

### Example 1-1 Synthesis of Onium Salt NSQ-1

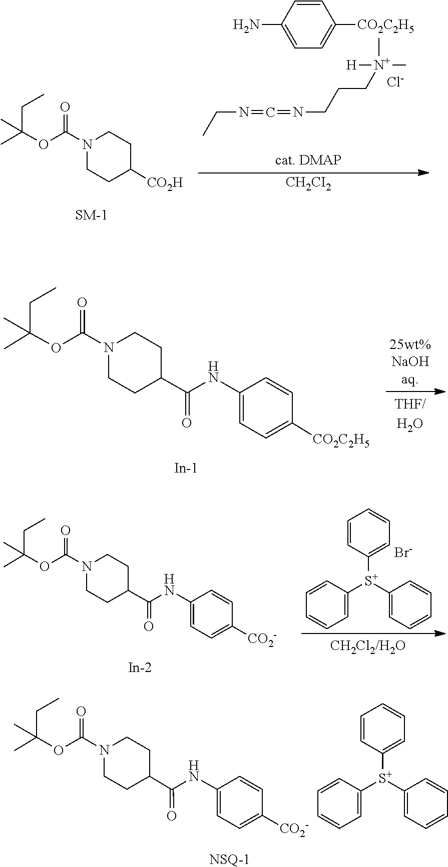

### (1) Synthesis of Intermediate In-1

In nitrogen atmosphere, a reactor was charged with 24.3 g of Compound SM-1, 17.3 g of ethyl 4-aminobenzoate, 1.2 g of 4-dimethylaminopyridine (DMAP), and 100 g of methylene chloride and cooled in an ice bath. While the internal temperature was maintained below 20° C., 23.0 g of 1-ethyl-3-(3-dimethylaminopropyl)carbodiimide hydrochloride was added in powder form. At the end of addition, the reaction mixture was warmed up to room temperature and aged for 12 hours. At the end of aging, water was added to quench the reaction. This was followed by ordinary aqueous work-up, solvent distillation, and recrystallization from hexane. Intermediate In-1 was obtained as white crystals (amount 36.7 g, yield 94%).

### (2) Synthesis of Intermediate In-2

In nitrogen atmosphere, 36.7 g of Intermediate In-1 was dissolved in 120 g of THF, to which 15.8 g of 25 wt % sodium hydroxide aqueous solution was added dropwise. At the end of addition, the reaction mixture was heated at 40° C. and aged for 4 hours. The reaction system was cooled to room temperature. After the solvent was distilled off, diisopropyl ether was added for recrystallization. Intermediate In-2 was obtained as white crystals (amount 31.1 g, yield 86%).

### (3) Synthesis of Onium Salt NSQ-1

In nitrogen atmosphere, 19.2 g of Intermediate In-2 and 20.6 g of triphenylsulfonium bromide were mixed with 100 g of methylene chloride and 80 g of water. The mixture was stirred at room temperature for 2 hours. This was followed by ordinary aqueous work-up and solvent distillation. Onium Salt NSQ-1 was obtained as oily matter (amount 28.4 g, yield 91%).

The results of TOF-MS of Onium Salt NSQ-1 are shown below.

MALDI TOF-MS:
positive M⁺263 (corresponding to C₁₈H₁₅S⁺)
negative M⁻ 361 (corresponding to C₁₉H₂₅N₂O₅⁻)

### Examples 1-2 to 1-7 Synthesis of Onium Salts NSQ-2 to NSQ-7

Onium salts NSQ-2 to NSQ-7, shown below, were synthesized using the corresponding reactants and well-known organic chemistry reaction.

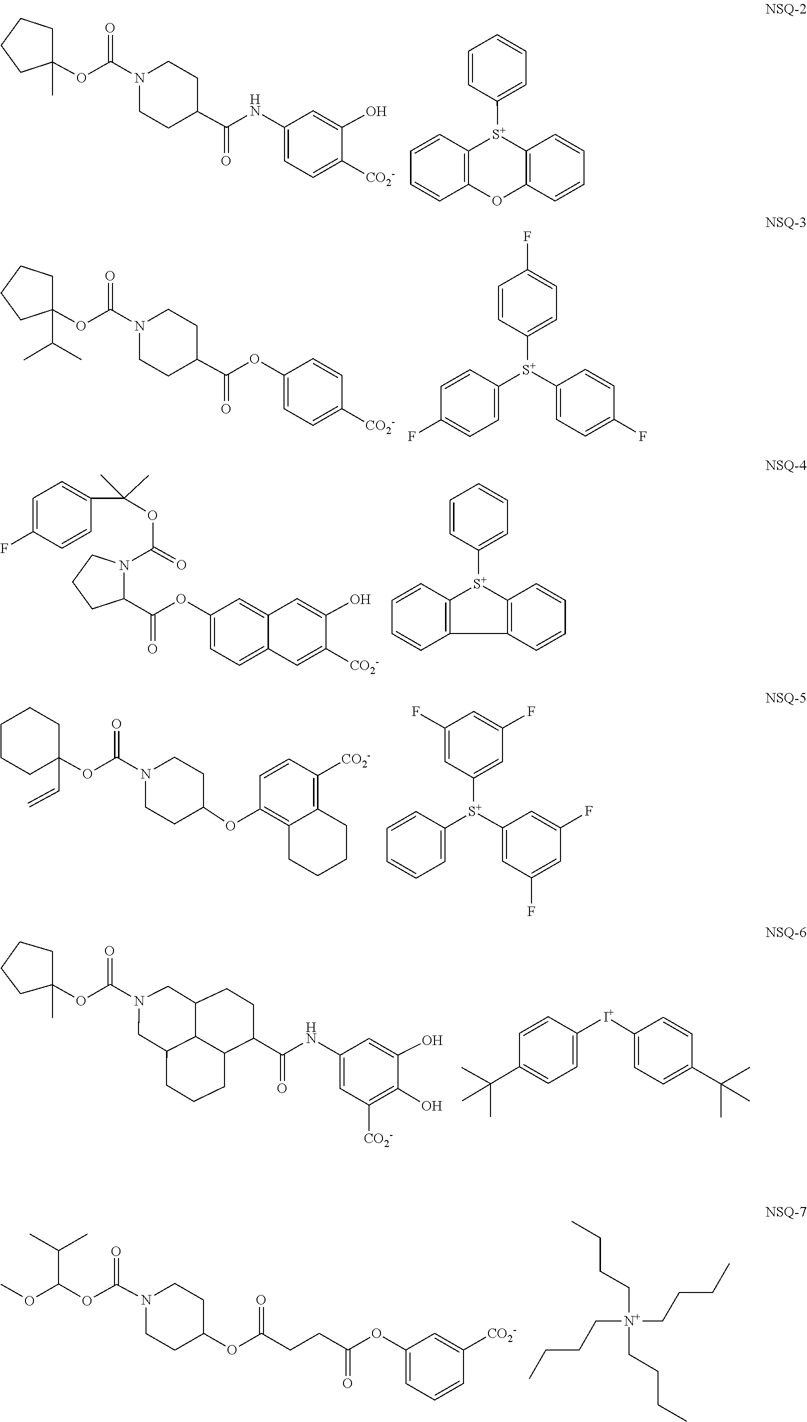

### [2] Synthesis of Base Polymer

Base polymers P-1 to P-5 were synthesized by combining monomers, performing copolymerization reaction in MEK solvent, pouring the reaction solution to hexane for precipitation, washing the solid precipitate with hexane, isolation and drying. The polymer was analyzed for composition by ¹H-NMR spectroscopy and for Mw and Mw/Mn by GPC versus polystyrene standards using DMF solvent.

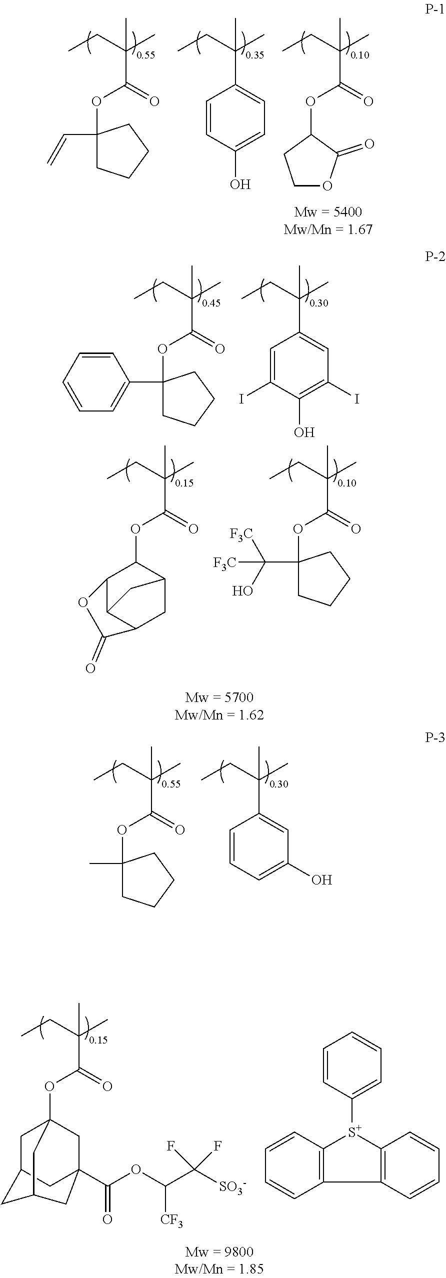

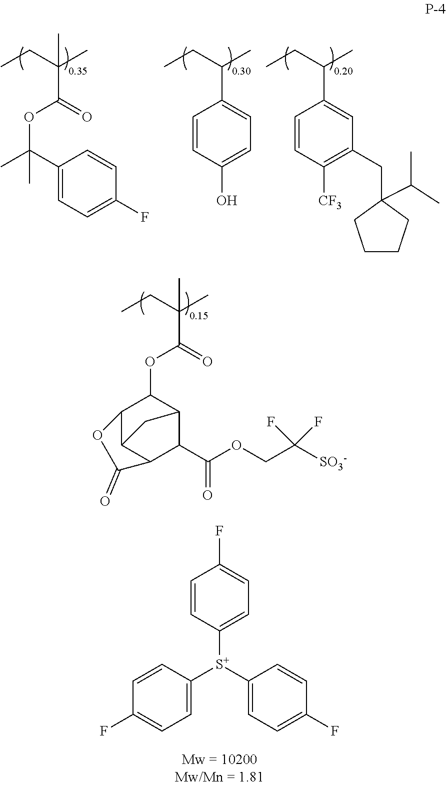

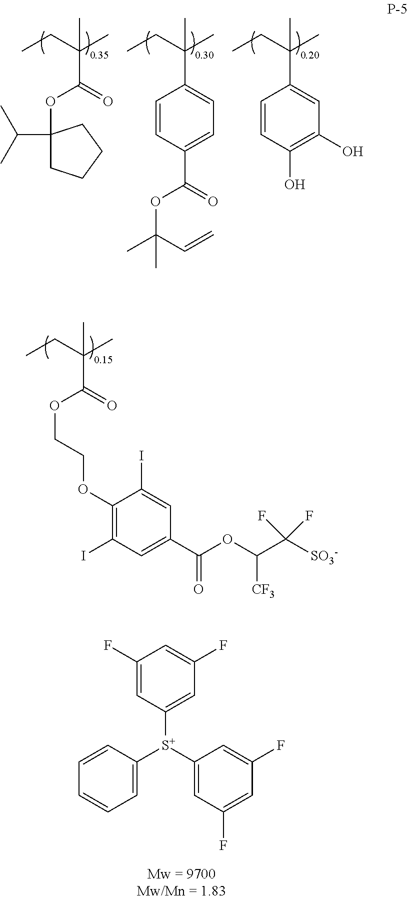

### [3] Preparation of Resist Composition

### Examples 2-1 to 2-20 and Comparative Examples 1-1 to 1-12

A resist composition was prepared by dissolving an inventive onium salt (NSQ-1 to NSQ-6) or comparative quencher (SQ-A to SQ-D, AQ-A), base polymer (P-1 to P-5), and photoacid generator (PAG-1, PAG-2) in an organic solvent containing 100 ppm of surfactant FC-4430 (3M) in accordance with the formulation shown in Tables 1 and 2, and filtering the solution through a Teflon® filter with a pore size of 0.2 μm.

The components in Tables 1 and 2 are identified below.

### Organic Solvent

PGMEA: propylene glycol monomethyl ether acetate
DAA: diacetone alcohol
   Photoacid generators: PAG-1 and PAG-2

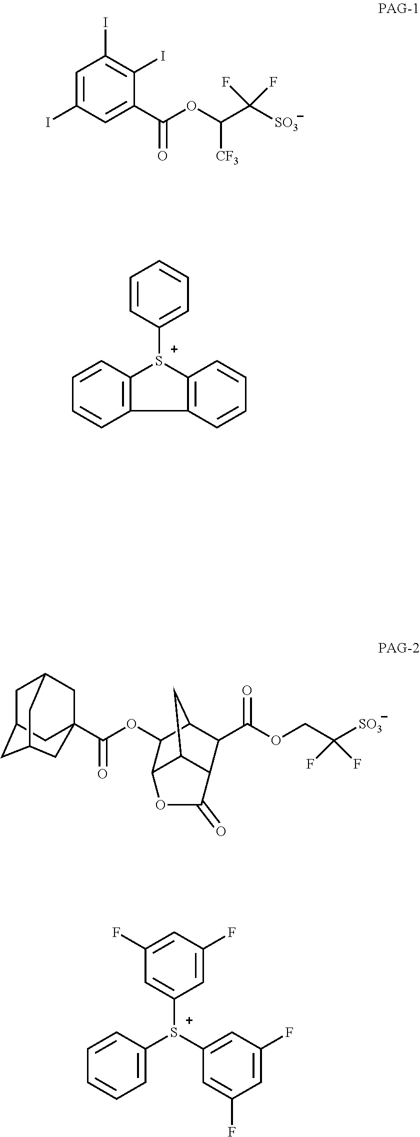

### Comparative Quenchers: SQ-A to SQ-D and AQ-A

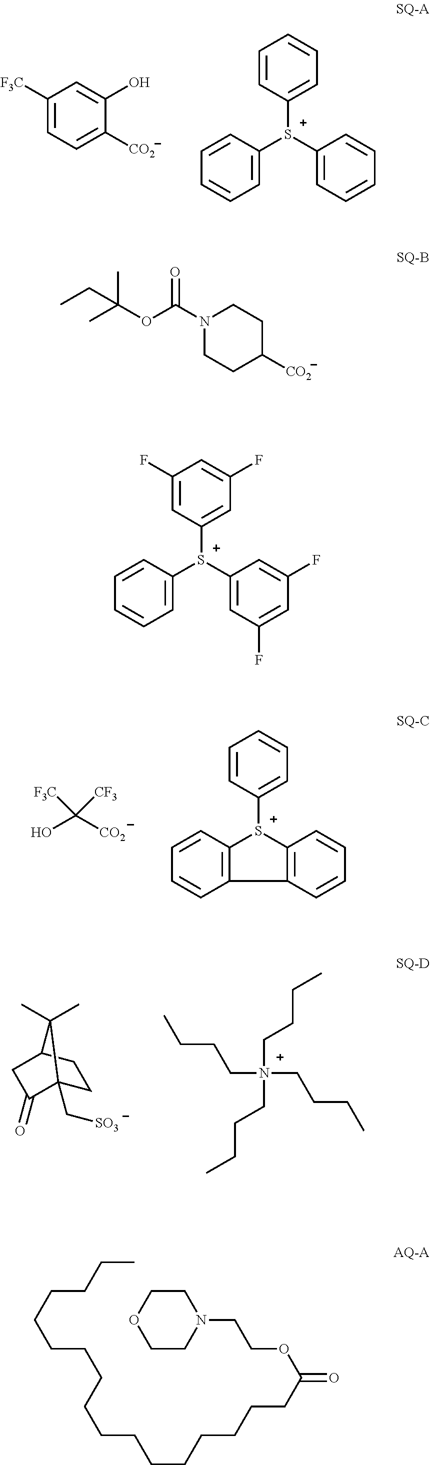

TABLE 1

| | | | Base | Photoacid | | | |
|---|---|---|---|---|---|---|---|
| | | Resist | polymer | generator | Quencher | Solvent 1 | Solvent 2 |
| | | composition | (pbw) | (pbw) | (pbw) | (pbw) | (pbw) |
| Example | 2-1 | R-1 | P-1 (80) | PAG-1 (30.4) | NSQ-1 (4.5) | PGMEA (3000) | DAA (900) |
| | 2-2 | R-2 | P-1 (80) | PAG-2 (25.8) | NSQ-2 (4.3) | PGMEA (3000) | DAA (900) |
| | 2-3 | R-3 | P-1 (80) | PAG-1 (28.4) | NSQ-3 (4.5) | PGMEA (3000) | DAA (900) |
| | 2-4 | R-4 | P-1 (80) | PAG-2 (24.8) | NSQ-4 (4.5) | PGMEA (3000) | DAA (900) |
| | 2-5 | R-5 | P-2 (80) | PAG-1 (30.4) | NSQ-1 (4.5) | PGMEA (3000) | DAA (900) |
| | 2-6 | R-6 | P-2 (80) | PAG-1 (29.4) | NSQ-2 (4.3) | PGMEA (3000) | DAA (900) |
| | 2-7 | R-7 | P-2 (80) | PAG-2 (25.8) | NSQ-5 (4.2) | PGMEA (3000) | DAA (900) |
| | 2-8 | R-8 | P-2 (80) | PAG-2 (24.8) | NSQ-6 (4.1) | PGMEA (3000) | DAA (900) |
| | 2-9 | R-9 | P-3 (80) | — | NSQ-1 (4.5) | PGMEA (3000) | DAA (900) |
| | 2-10 | R-10 | P-3 (80) | — | NSQ-4 (4.2) | PGMEA (3000) | DAA (900) |
| | 2-11 | R-11 | P-3 (80) | — | NSQ-5 (4.2) | PGMEA (3000) | DAA (900) |
| | 2-12 | R-12 | P-3 (80) | PAG-1 (10.4) | NSQ-1 (4.4) | PGMEA (3000) | DAA (900) |
| | 2-13 | R-13 | P-4 (80) | — | NSQ-1 (4.5) | PGMEA (3000) | DAA (900) |
| | 2-14 | R-14 | P-4 (80) | — | NSQ-2 (4.7) | PGMEA (3000) | DAA (900) |
| | 2-15 | R-15 | P-4 (80) | — | NSQ-7 (4.1) | PGMEA (3000) | DAA (900) |
| | 2-16 | R-16 | P-4 (80) | PAG-2 (8.4) | NSQ-1 (4.3) | PGMEA (3000) | DAA (900) |
| | 2-17 | R-17 | P-5 (80) | — | NSQ-1 (4.5) | PGMEA (3000) | DAA (900) |
| | 2-18 | R-18 | P-5 (80) | — | NSQ-2 (4.3) | PGMEA (3000) | DAA (900) |
| | 2-19 | R-19 | P-5 (80) | — | NSQ-5 (4.7) | PGMEA (3000) | DAA (900) |
| | 2-20 | R-20 | P-5 (80) | PAG-1 (6.4) | NSQ-1 (4.6) | PGMEA (3000) | DAA (900) |

TABLE 2

| | | | Base | Photoacid | | | |
|---|---|---|---|---|---|---|---|
| | | Resist | polymer | generator | Quencher | Solvent 1 | Solvent 2 |
| | | composition | (pbw) | (pbw) | (pbw) | (pbw) | (pbw) |
| Comparative | 1-1 | CR-1 | P-1 (80) | PAG-1 (30.4) | SQ-A (4.4) | PGMEA (3000) | DAA (900) |
| Example | 1-2 | CR-2 | P-1 (80) | PAG-2 (25.8) | SQ-B (4.3) | PGMEA (3000) | DAA (900) |
| | 1-3 | CR-3 | P-1 (80) | PAG-1 (28.4) | SQ-C (4.6) | PGMEA (3000) | DAA (900) |
| | 1-4 | CR-4 | P-1 (80) | PAG-2 (24.8) | AQ-A (4.8) | PGMEA (3000) | DAA (900) |
| | 1-5 | CR-5 | P-2 (80) | PAG-1 (29.4) | SQ-B (4.3) | PGMEA (3000) | DAA (900) |
| | 1-6 | CR-6 | P-2 (80) | PAG-2 (25.8) | SQ-D (3.3) | PGMEA (3000) | DAA (900) |
| | 1-7 | CR-7 | P-3 (80) | — | SQ-B (4.3) | PGMEA (3000) | DAA (900) |
| | 1-8 | CR-8 | P-3 (80) | PAG-1 (10.4) | SQ-A (4.6) | PGMEA (3000) | DAA (900) |
| | 1-9 | CR-9 | P-4 (80) | — | SQ-A (4.8) | PGMEA (3000) | DAA (900) |
| | 1-10 | CR-10 | P-4 (80) | PAG-2 (8.4) | AQ-A (4.5) | PGMEA (3000) | DAA (900) |
| | 1-11 | CR-11 | P-5 (80) | — | SQ-A (4.8) | PGMEA (3000) | DAA (900) |
| | 1-12 | CR-12 | P-5 (80) | PAG-1 (6.4) | SQ-B (4.3) | PGMEA (3000) | DAA (900) |

### [4] EUV Lithography Test 1

### Examples 3-1 to 3-20 and Comparative Examples 2-1 to 2-12

Each of the chemically amplified resist compositions (R-1 to R-20, CR-1 to CR-12) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 (ASML, NA 0.33, σ 0.9/0.6, dipole illumination), the resist film was exposed to EUV through a mask bearing a line-and-space (LS) pattern having a width of 18 nm and a pitch of 36 nm (on-wafer size) while changing the dose at a pitch of 1 mJ/cm² and the focus at a pitch of 0.020 rim. The resist film was baked (PEB) at the temperature shown in Tables 3 and 4 for 60 seconds. This was followed by puddle development in a 2.38 wt % TMAH aqueous solution for 30 seconds, rinsing with a surfactant-containing rinse fluid, and spin drying. A positive LS pattern was obtained.

The LS pattern was observed under CD-SEM (CG6300, Hitachi High-Technologies Corp.) and evaluated for sensitivity, exposure latitude (EL), LWR, depth of focus (DOF), and collapse limit by the following methods. The results are shown in Tables 5 and 6.

### Evaluation of Sensitivity

The optimum dose Eop (mJ/cm²) which provided an LS pattern with a line width of 18 nm and a pitch of 36 nm was determined and reported as sensitivity.

### Evaluation of EL

The exposure dose which provided a LS pattern with a space width of 18 nm±10% (i.e., 16.2 to 19.8 nm) was determined. EL (%) is calculated from the exposure doses according to the following equation:

$$EL ( \% ) = ( \vert E 1 - E 2 \vert / Eop ) \times 100$$

wherein E1 is an optimum exposure dose which provides a LS pattern with a line width of 16.2 nm and a pitch of 36 nm, E2 is an optimum exposure dose which provides a LS pattern with a line width of 19.8 nm and a pitch of 36 nm, and Eop is an optimum exposure dose which provides a LS pattern with a line width of 18 nm and a pitch of 36 nm. A larger value indicates better performance.

### Evaluation of LWR

For the LS pattern formed by exposure at the optimum dose Eop, the line width was measured at 10 longitudinally spaced apart points, from which a 3-fold value (36) of the standard deviation (6) was determined and reported as LWR. A smaller value of 36 indicates a pattern having small roughness and uniform line width.

### Evaluation of DOF

As an index of DOF, a range of focus which provided a LS pattern with a size of 18 nm±10% (i.e., 16.2 to 19.8 nm) was determined. A greater value indicates a wider DOF.

### Evaluation of Collapse Limit of Line Pattern

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 3

| | | Resist | PEB temp. | Sensitivity | EL | LWR | DOF | Collapse limit |
|---|---|---|---|---|---|---|---|---|
| | | composition | (° C.) | (mJ/cm²) | (%) | (nm) | (nm | (nm) |
| Example | 3-1 | R-1 | 100 | 33 | 17 | 2.9 | 120 | 10.2 |
| | 3-2 | R-2 | 100 | 32 | 18 | 2.8 | 110 | 10.7 |
| | 3-3 | R-3 | 105 | 31 | 18 | 2.9 | 100 | 10.9 |
| | 3-4 | R-4 | 105 | 32 | 17 | 2.8 | 120 | 10.7 |
| | 3-5 | R-5 | 95 | 33 | 19 | 3 | 110 | 10.7 |
| | 3-6 | R-6 | 105 | 32 | 18 | 3.1 | 120 | 10.9 |
| | 3-7 | R-7 | 100 | 30 | 17 | 2.8 | 110 | 11.2 |
| | 3-8 | R-8 | 100 | 31 | 18 | 2.9 | 120 | 11.3 |
| | 3-9 | R-9 | 100 | 27 | 18 | 2.9 | 110 | 10.9 |
| | 3-10 | R-10 | 105 | 28 | 17 | 2.8 | 120 | 11.4 |
| | 3-11 | R-11 | 100 | 28 | 19 | 2.7 | 120 | 10.9 |
| | 3-12 | R-12 | 95 | 29 | 18 | 2.9 | 120 | 11.6 |
| | 3-13 | R-13 | 100 | 27 | 17 | 3 | 110 | 11.2 |
| | 3-14 | R-14 | 100 | 28 | 18 | 2.9 | 110 | 10.8 |
| | 3-15 | R-15 | 105 | 27 | 18 | 2.8 | 120 | 11.3 |
| | 3-16 | R-16 | 100 | 28 | 17 | 3 | 100 | 10.9 |
| | 3-17 | R-17 | 105 | 29 | 18 | 2.8 | 120 | 11.4 |
| | 3-18 | R-18 | 105 | 28 | 18 | 2.9 | 110 | 10.8 |
| | 3-19 | R-19 | 105 | 28 | 18 | 2.8 | 120 | 11.8 |
| | 3-20 | R-20 | 100 | 27 | 17 | 2.7 | 110 | 11.1 |

TABLE 4

| | | Resist | PEB temp. | Sensitivity | EL | LWR | DOF | Collapse limit |
|---|---|---|---|---|---|---|---|---|
| | | composition | (° C.) | (mJ/cm²) | (%) | (nm) | (nm) | (nm) |
| Comparative | 2-1 | CR-1 | 100 | 35 | 14 | 3.5 | 80 | 13.6 |
| Example | 2-2 | CR-2 | 100 | 36 | 15 | 4 | 70 | 14.7 |
| | 2-3 | CR-3 | 105 | 38 | 14 | 3.9 | 90 | 14.6 |
| | 2-4 | CR-4 | 105 | 36 | 13 | 3.8 | 80 | 14.3 |
| | 2-5 | CR-5 | 100 | 37 | 15 | 4.1 | 90 | 14.2 |
| | 2-6 | CR-6 | 95 | 35 | 14 | 3.9 | 90 | 14.3 |
| | 2-7 | CR-7 | 105 | 27 | 14 | 3.7 | 80 | 14.2 |
| | 2-8 | CR-8 | 100 | 26 | 15 | 4.2 | 80 | 13.8 |
| | 2-9 | CR-9 | 105 | 27 | 14 | 3.9 | 90 | 13.6 |
| | 2-10 | CR-10 | 100 | 28 | 15 | 4 | 80 | 13.5 |
| | 2-11 | CR-11 | 105 | 27 | 16 | 3.7 | 90 | 13.2 |
| | 2-12 | CR-12 | 110 | 26 | 15 | 3.8 | 90 | 13.7 |

It is demonstrated in Tables 3 and 4 that resist compositions within the scope of the invention exhibit a high sensitivity, improved lithography properties, and resistance to pattern collapse.

### [5] EUV Lithography Test 2

### Examples 4-1 to 4-20 and Comparative Examples 3-1 to 3-12

Each of the chemically amplified resist compositions (R-1 to R-20, CR-1 to CR-12) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 60 nm thick. Using an EUV scanner NXE3300 (ASML, NA 0.33, σ 0.9/0.6, dipole illumination), the resist film was exposed to EUV through a mask bearing a hole pattern having a pitch of 44 nm+20% bias (on-wafer size). The resist film was baked (PEB) on a hotplate at the temperature shown in Tables 5 and 6 for 60 seconds. This was followed by development in a 2.38 wt % TMAH aqueous solution for 30 seconds. Hole patterns with a size of 22 nm were obtained in Examples 4-1 to 4-18 and Comparative Examples 3-1 to 3-10 whereas dot patterns with a size of 22 nm were obtained in Examples 4-19 and 4-20 and Comparative Examples 3-11 and 3-12.

The hole or dot pattern was observed under CD-SEM (CG6300, Hitachi High-Technologies Corp.). The exposure dose that provides a hole or dot pattern having a size of 22 nm was determined and reported as sensitivity. The size of 50 holes or dots at that dose was measured, from which a 3-fold value (36σ) of the standard deviation (σ) was computed and reported as CDU. The results are also shown in Tables 5 and 6.

TABLE 5

| | | Resist | PEB temp. | Sensitivity | CDU |
|---|---|---|---|---|---|
| | | composition | (° C.) | (mJ/cm²) | (nm) |
| Example | 4-1 | R-1 | 90 | 31 | 3.1 |
| | 4-2 | R-2 | 85 | 32 | 3.3 |
| | 4-3 | R-3 | 90 | 30 | 3.2 |
| | 4-4 | R-4 | 90 | 30 | 3 |
| | 4-5 | R-5 | 85 | 29 | 2.9 |
| | 4-6 | R-6 | 90 | 31 | 2.8 |
| | 4-7 | R-7 | 95 | 31 | 2.9 |
| | 4-8 | R-8 | 90 | 30 | 3 |
| | 4-9 | R-9 | 85 | 28 | 3.1 |
| | 4-10 | R-10 | 90 | 27 | 3.1 |
| | 4-11 | R-11 | 90 | 28 | 3 |
| | 4-12 | R-12 | 85 | 27 | 2.9 |
| | 4-13 | R-13 | 90 | 28 | 3 |
| | 4-14 | R-14 | 90 | 27 | 2.9 |
| | 4-15 | R-15 | 85 | 27 | 2.9 |
| | 4-16 | R-16 | 90 | 26 | 3.1 |
| | 4-17 | R-17 | 90 | 28 | 2.9 |
| | 4-18 | R-18 | 95 | 27 | 2.8 |
| | 4-19 | R-19 | 90 | 27 | 2.9 |
| | 4-20 | R-20 | 90 | 28 | 2.9 |

TABLE 6

| | | Resist | PEB temp. | Sensitivity | CDU |
|---|---|---|---|---|---|
| | | composition | (° C.) | (mJ/cm²) | (nm) |
| Comparative | 3-1 | CR-1 | 85 | 33 | 3.5 |
| Example | 3-2 | CR-2 | 90 | 32 | 4.1 |
| | 3-3 | CR-3 | 90 | 34 | 3.9 |
| | 3-4 | CR-4 | 85 | 33 | 3.8 |
| | 3-5 | CR-5 | 90 | 34 | 4 |
| | 3-6 | CR-6 | 85 | 32 | 3.8 |
| | 3-7 | CR-7 | 90 | 30 | 3.9 |
| | 3-8 | CR-8 | 90 | 29 | 4.1 |
| | 3-9 | CR-9 | 85 | 28 | 4 |
| | 3-10 | CR-10 | 85 | 29 | 3.9 |
| | 3-11 | CR-11 | 90 | 29 | 3.8 |
| | 3-12 | CR-12 | 85 | 30 | 3.7 |

It is demonstrated in Tables 5 and 6 that chemically amplified resist compositions comprising quenchers within the scope of the invention offer a high sensitivity and improved CDU either when the compositions are of positive or negative tone.

Japanese Patent Application No. 2023-024819 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An onium salt having the formula (1): 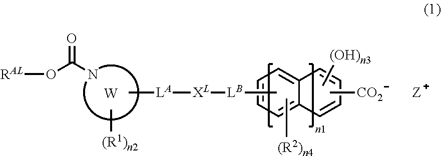
wherein n1 is 0 or 1, n2 is an integer of 0 to 6, n3 is an integer of 0 to 3, n4 is an integer of 0 to 4,
W is a C₂-C₂₀ nitrogen-containing aliphatic heterocycle which may contain a heteroatom,
L^{A} and L^{B} are each independently a single bond, ether bond, ester bond, amide bond, sulfonic ester bond, carbonate bond or carbamate bond,
X^{L} is a single bond or a C₁-C₄₀ hydrocarbylene group which may contain a heteroatom,
R¹ is a C₁-C₂₀ hydrocarbyl group which may contain a heteroatom, and when n2 is 2 or more, a plurality of R¹ may bond together to form a ring with the carbon atoms on W to which they are attached,
R² is halogen or a C₁-C₂₀ hydrocarbyl group which may contain a heteroatom, and when n4 is 2 or more, a plurality of R² may bond together to form a ring with the carbon atoms on the aromatic ring to which they are attached,
R^{AL} forms an acid labile group with the adjacent —O—, and
Z⁺ is an onium cation.

2. The onium salt of claim 1 wherein R^{AL} is a group having the formula (AL-1) or (AL-2): 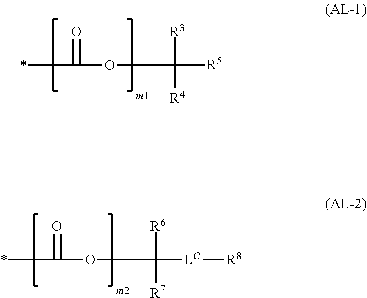
wherein L^{C} is —O— or —S—,
R³, R⁴ and R⁵ are each independently a C₁-C₁₀ hydrocarbyl group, any two of R³, R⁴ and R⁵ may bond together to form a ring,
R⁶ and R⁷ are each independently hydrogen or a C₁-C₁₀ hydrocarbyl group, R⁸ is a C₁-C₂₀ hydrocarbyl group in which —CH₂— may be replaced by —O— or —S—, R⁷ and R⁸ may bond together to form a C₃-C₂₀ heterocyclic group with the carbon atom and L^{C} to which they are attached, —CH₂— in the heterocyclic group may be replaced by —O— or —S—,
m1 and m2 are each independently 0 or 1, and
* designates a point of attachment to the adjacent —O—.

3. The onium salt of claim 1 wherein Z⁺ is an onium cation having any one of the formulae (cation-1) to (cation-3): 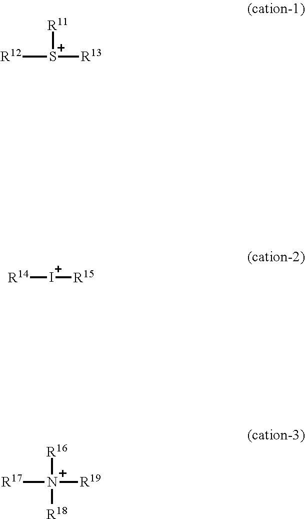
wherein R¹¹ to R¹⁹ are each independently a C₁-C₃₀ hydrocarbyl group which may contain a heteroatom, R¹¹ and R¹² may bond together to form a ring with the sulfur atom to which they are attached.

4. The onium salt of claim 1, having the formula (1A): 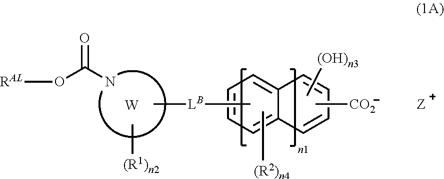
wherein n1 to n4, W, L^{B}, R¹, R², R^{AL} and Z⁺ are as defined above.

5. The onium salt of claim 4, having the formula (1B): 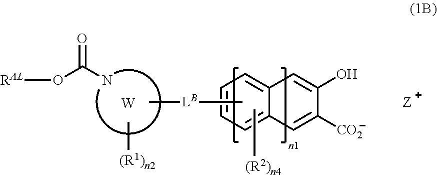
wherein n1, n2, n4, W, L^{B}, R¹, R², R^{AL} and Z⁺ are as defined above.

6. A quencher comprising the onium salt of claim 1.

7. A resist composition comprising the quencher of claim 6.

8. The resist composition of claim 7, further comprising an organic solvent.

9. The resist composition of claim 7, further comprising a base polymer comprising repeat units having the formula (a1): 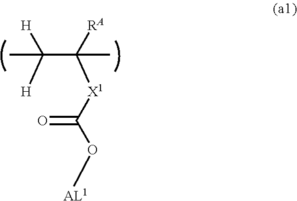
wherein R^{A} is hydrogen, fluorine, methyl or trifluoromethyl,
X¹ is a single bond, phenylene group, naphthylene group or *—C(═O)—O—X¹¹—, the phenylene group and naphthylene group may be substituted with an optionally fluorinated C₁-C₁₀ alkoxy moiety or halogen, X¹¹ is a C₁-C₁₀ saturated hydrocarbylene group which may contain a hydroxy moiety, ether bond, ester bond or lactone ring, a phenylene group or naphthylene group, * designates a point of attachment to the carbon atom in the backbone, and
AL¹ is an acid labile group.

10. The resist composition of claim 9 wherein the base polymer further comprises repeat units having the formula (a2): 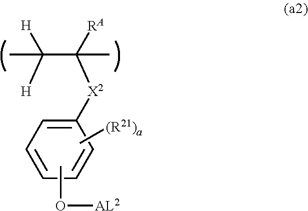
wherein R^{A} is hydrogen, fluorine, methyl or trifluoromethyl,
X² is a single bond or *—C(═O)—O—, wherein * designates a point of attachment to the carbon atom in the backbone,
R²¹ is halogen, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
AL² is an acid labile group, and
a is an integer of 0 to 4.

11. The resist composition of claim 9 wherein the base polymer further comprises repeat units having the formula (b1) or (b2): 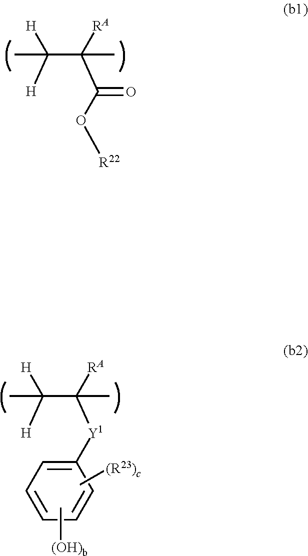
wherein R^{A} is each independently hydrogen, fluorine, methyl or trifluoromethyl,
Y¹ is a single bond or *—C(═O)—O— wherein * designates a point of attachment to the carbon atom in the backbone,
R²² is hydrogen, or a C₁-C₂₀ group containing at least one moiety selected from hydroxy moiety other than phenolic hydroxy, cyano moiety, carbonyl moiety, carboxy moiety, ether bond, ester bond, sulfonic ester bond, carbonate bond, lactone ring, sultone ring, and carboxylic anhydride (—C(═O)—O—C(═O)—),
R²³ is halogen, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
b is an integer of 1 to 4, c is an integer of 0 to 4, and b+c is from 1 to 5.

12. The resist composition of claim 9 wherein the base polymer further comprises repeat units of at least one type selected from repeat units having the formulae (c1) to (c4): 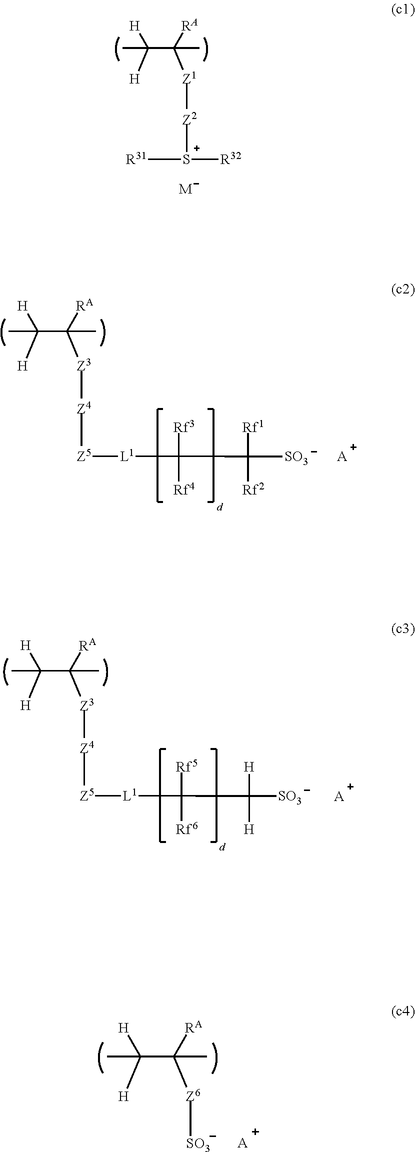
wherein R^{A} is each independently hydrogen, fluorine, methyl or trifluoromethyl,
Z¹ is a single bond or phenylene group,
Z² is *—C(═O)—O—Z²¹—, *—C(═O)—NH—Z²¹—, or *—O—Z²¹—, wherein Z²¹ is a C₁-C₆ aliphatic hydrocarbylene group, phenylene, or divalent group obtained by combining the foregoing, which may contain a carbonyl moiety, ester bond, ether bond or hydroxy moiety,
Z³ is each independently a single bond, phenylene group, naphthylene group or *—C(═O)—O—Z³¹—, wherein Z³¹ is a C₁-C₁₀ aliphatic hydrocarbylene group which may contain a hydroxy moiety, ether bond, ester bond or lactone ring, or a phenylene group or naphthylene group,
Z⁴ is each independently a single bond, *—Z⁴¹—C(═O)—O—, *—C(═O)—NH—Z⁴¹— or *—O—Z⁴¹—, wherein Z⁴¹ is a C₁-C₂₀ hydrocarbylene group which may contain a heteroatom, Z⁵ is each independently a single bond, *—Z⁵¹—C(═O)—O—, *—C(═O)—NH—Z⁵¹— or *—O—Z⁵¹—, wherein Z⁵¹ is a C₁-C₂₀ hydrocarbylene group which may contain a heteroatom,
Z⁶ is a single bond, methylene, ethylene, phenylene, fluorinated phenylene, trifluoromethyl-substituted phenylene, *—C(═O)—O—Z⁶¹—, *—C(═O)—N(H)—Z⁶¹— or *—O—Z⁶¹—, wherein Z⁶¹ is a C₁-C₆ aliphatic hydrocarbylene group, phenylene, fluorinated phenylene or trifluoromethyl-substituted phenylene group, which may contain a carbonyl moiety, ester bond, ether bond or hydroxy moiety,
* designates a point of attachment to the carbon atom in the backbone,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, ether bond, ester bond, carbonyl group, sulfonic ester bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine or a C₁-C₆ fluorinated saturated hydrocarbyl group,
Rf⁵ and Rf⁶ are each independently hydrogen, fluorine or a C₁-C₆ fluorinated saturated hydrocarbyl group, excluding that all Rf⁵ and Rf⁶ are hydrogen at the same time,
M⁻ is a non-nucleophilic counter ion,
A⁺ is an onium cation, and
d is an integer of 0 to 3.

13. The resist composition of claim 7, further comprising a photoacid generator.

14. The resist composition of claim 7, further comprising a quencher other than the quencher.

15. The resist composition of claim 7, further comprising a surfactant.

16. A process for forming a pattern comprising the steps of applying the resist composition of claim 7 to a substrate to form a resist film thereon, exposing the resist film to high-energy radiation, and developing the exposed resist film in a developer.

17. The process of claim 16 wherein the high-energy radiation is KrF excimer laser, ArF excimer laser, EB, or EUV of wavelength 3 to 15 nm.
